(19) United States
(12) Patent Application Publication
(10) Pub. No.: US 2023/0303572 A1
(43) Pub. Date: Sep. 28, 2023

(54) 18-MC SALT FORMS

(71) Applicant: Mind Medicine, Inc., New York, NY (US)

(72) Inventors: Jeanne BONELLE, Castro Valley, CA (US); Daniel Emil LEVY, San Mateo, CA (US); Ann NEWMAN, Kure Beach, NC (US); Stephen E. SCHNEIDER, Raleigh, NC (US)

(73) Assignee: Mind Medicine, Inc., New York, NY (US)

(21) Appl. No.: 18/177,073

(22) Filed: Mar. 1, 2023

Related U.S. Application Data
(60) Provisional application No. 63/317,632, filed on Mar. 8, 2022.

(51) Int. Cl. C07D 471/22

(52) U.S. Cl. CPC C07D 471/22; C07B 2200/13

(57) ABSTRACT

A composition including a salt of 18-MC, wherein the salt is chosen from gentisate, hydrobromide, besylate, napadisylate, hydrochloride, sulfate, oxalate, maleate, mesylate, and tosylate. A composition including a polymorph of 18-MC.

### BACKGROUND OF THE INVENTION

### 1. Technical Field

The present invention relates to compositions of salts and polymorphs of 18-methoxycoronaridine.

### 2. Background Art

18-methoxycoronaridine (18-MC) is a derivative of ibogaine with the chemical formula of C₂₂H₂₈N₂O₃. The freebase 18-MC is a synthetic coronaridine congener and a specific negative allosteric modulator (antagonist) of α3β4 nicotinic cholinergic receptors; it indirectly modulates the dopaminergic mesolimbic pathway via blockade of α3β4 nicotinic receptors in the habenulo-interpeduncular pathway and the basolateral amygdala (Glick et al., 2008). Animal studies have demonstrated that 18-MC significantly reduces drug self-administration in a number of substance use models (nicotine, alcohol, morphine, cocaine and methamphetamine) at dosages as low as 10 mg/kg i.p. (Glick et al., 1994; Rezvani et al., 1995; Glick et al., 1996; Glick et al., 1998; Glick et al., 2000a). More recently, 18-MC has been shown in an animal model to attenuate effects of the environmental cues responsible for stimulating cocaine-seeking or “craving” behaviors (Polston et al., 2012, and U.S. Pat. Application No. 14/387,339 to Glick, et al.). This property of 18-MC could potentially help address the craving component of human addictive behaviors.

U.S. Pat. Application No. 14/387,339 to Glick, et al. discloses methods of preventing drug relapse, especially during cue inducement, by administering an effective amount of an α3β4 nicotinic antagonist (18- Methoxycoronaridine) to a mammal, after an initial period of drug use, and preventing a relapse of drug use. It was shown that rats conditioned with a musical cue show increased drug-seeking behaviors with cocaine when compared to control groups. Pharmaceutically acceptable HCl salts of 18-MC are mentioned, however, polymorphism is not disclosed. Preparation of the 18-MC HCl salt has been briefly described in the literature (Acta Crystallographica Section E, Structure Reports, ISSN 1600-5368, Acta Cryst. (2012). E68, o1041). However, few details are provided and conflicting information is provided regarding the solvent system. Understanding and control of polymorphism is required in order to develop a robust and scalable API manufacturing process resulting in a stable material that is suitable for drug product manufacturing.

Therefore, there remains a need for salts and polymorphs of 18-MC.

### SUMMARY OF THE INVENTION

The present invention provides for a composition including a salt of 18-MC, wherein the salt is chosen from gentisate, hydrobromide, besylate, napadisylate, hydrochloride, sulfate, oxalate, maleate, mesylate, and tosylate.

The present invention provides for a composition including a polymorph of 18-MC, wherein the polymorph is chosen from HCl salt Type A, HCl salt Type B, HCl salt Type C, HCl salt Type D, HCl salt Type E, HCl salt Type F, HCl salt Type G, HCl salt Type H, HCl salt Type I, HCl salt Type J, HCl salt Type K, HCl salt Type L, HCl salt Type M, HCl salt Type N, HCl salt Type O, HCl salt Type P, HCl salt Type Q, HCl salt Type R, HCl salt Type S, HCl salt Type T, HCl salt Type U, HCl salt Type V, sulfate salt Type A, sulfate salt Type B, sulfate salt Type C, sulfate salt Type D, sulfate salt Type E, sulfate salt Type F, oxalate salt Type A, oxalate salt Type B, maleate salt Type A, mesylate salt Type A, mesylate salt Type B, mesylate salt Type C, HBr salt Type A, HBr salt Type B, HBr salt Type C, HBr salt Type D, tosylate salt Type A, tosylate salt Type B, tosylate salt Type C, tosylate salt Type D, tosylate salt Type E, tosylate salt Type F, tosylate salt Type G, tosylate salt Type H, tosylate salt Type I, besylate salt Type A, besylate salt Type B, besylate salt Type C, napadisylate salt Type A, napadisylate salt Type B, napadisylate salt Type C, napadisylate salt Type D, and gentisate salt Type A.

### DESCRIPTION OF THE DRAWINGS

Other advantages of the present invention are readily appreciated as the same becomes better understood by reference to the following detailed description when considered in connection with the accompanying drawings wherein:

FIG. 1 is an XRPD overlay of new salt hits from screening;

FIG. 2A is an XRPD overlay of starting material (824509-01-A) and reference lot 819246-01-A, FIG. 2B shows TGA/DSC curves of starting material (824509-01-A), FIG. 2C is an overlay of HPLC chromatograms of starting material (824509-01-A) and a blank, FIG. 2D is an XRPD pattern of starting material (824509-20-A), FIG. 2E is a TGA curve of starting material (824509-20-A), FIG. 2F is a HPLC chromatogram of starting material (824509-20-A, ), FIG. 2G is an XRPD overlay of starting material (824509-20-B) and reference, FIG. 2H shows TGA/DSC curves of starting material (824509-20-B), and FIG. 21 is a HPLC chromatogram of starting material (824509-20-B), FIG. 2J is an XRPD overlay of freebase samples relative to a reference diffractogram FIG. 2K shows TGA/DSC curves of freebase Type A (824509-03-A), FIG. 2L is a HPLC chromatogram of freebase Type A (824509-03-A), FIG. 2M shows TGA/DSC curves of freebase Type A (824509-21-A), FIG. 2N is a HPLC chromatogram of freebase Type A (824509-21-A), FIGS. 20 shows TGA/DSC curves of freebase Type A (824509-24-A), and FIG. 2P is a HPLC chromatogram of freebase Type A (824509-24-A),

FIG. 3A is an XRPD pattern of gentisate Type A (824511-01-C9), FIG. 3B shows TGA/DSC curves of gentisate Type A (824511-01-C9), FIG. 3C is a ¹H NMR spectrum of gentisate Type A (824511-01-C9), FIG. 3D is a HPLC chromatogram of gentisate Type A (824511-01-C9), FIG. 3E is an XRPD pattern of HBr salt Type A (824511-01-E10), FIG. 3F shows TGA/DSC curves of HBr salt Type A (824511-01-E10), FIG. 3G is ¹H NMR spectrum of HBr salt Type A (824511-01-E10), FIG. 3H is a HPLC chromatogram of HBr salt Type A (824511-01-E10), FIG. 31 is an XRPD overlay of HBr salt Type B and re-prepared batch, FIG. 3J shows TGA/DSC curves of HBr salt Type B (824511-10-A1), FIG. 3K is a ¹H NMR spectrum of HBr salt Type B (824511-10-A1), FIG. 3L is a HPLC chromatogram of HBr salt Type B (824511-10-A1), FIG. 3M is an XRPD pattern of napadisylate Type A (824511-30-B7), FIG. 3N shows TGA/DSC curves of napadisylate Type A (824511-30-B7), FIGURE is a ¹H NMR spectrum of napadisylate Type A (824511-30-B7), FIG. 3P is a HPLC chromatogram of napadisylate Type A (824511-30-B7), FIG. 3Q is an XRPD pattern of napadisylate Type B (824511-36-A7), FIG. 3R shows TGA/DSC curves of napadisylate Type B (824511-36-A7), FIG. 3S is a ¹H NMR spectrum of napadisylate Type B (824511-36-A7), FIG. 3T is a HPLC chromatogram of napadisylate Type B (824511-36-A7), FIG. 3U is an XRPD overlay of napadisylate Type C batches, FIG. 3V shows TGA/DSC curves of napadisylate Type C (824511-44-B2), FIG. 3W is a ¹H NMR spectrum of napadisylate Type C (824511-44-B2), FIG. 3X is a HPLC chromatogram of napadisylate Type C (824511-44-B2), FIG. 3Y is an XRPD pattern of napadisylate Type D (824511-44-B1), FIG. 3Z shows TGA/DSC curves of napadisylate Type D (824511-44-B1), FIG. 3AA is a ¹H NMR spectrum of napadisylate Type D (824511-44-B1), FIG. 3AB is a HPLC chromatogram of napadisylate Type D (824511-44-B1), FIG. 3AC is an XRPD overlay of besylate Type A batches, FIG. 3AD shows TGA/DSC curves of besylate Type A (824511-35-A1), FIG. 3AE is a ¹H NMR spectrum of besylate Type A (824511-35-A1), FIG. 3AF is a HPLC chromatogram of besylate Type A (824511-35-A1), FIG. 3AG is an XRPD pattern of besylate Type B (824511-36-A10), FIG. 3AH shows TGA/DSC curves of besylate Type B (824511-36-A10), FIG. 3AI is a ¹H NMR spectrum of besylate Type B (824511-36-A10), and FIG. 3AJ is a HPLC chromatogram of besylate Type B (824511-36-A10);

FIG. 4 is a diagram showing inter-conversion relationship of HCl salt forms of 18-MC;

FIG. 5A is an XRPD pattern of HCl salt Type M (824509-05-A4), FIG. 5B shows TGA/DSC curves of HCl salt Type M (824509-05-A4), FIG. 5C is a ¹H NMR spectrum of HCl salt Type M (824509-05-A4), FIG. 5D is a HPLC chromatogram of HCl salt Type M (824509-05-A4), FIG. 5E is a XRPD overlay of HCl salt Type M (824509-05-A4) before and after storage, and FIG. 5F is a VT-XRPD overlay of HCl salt Type M (824509-05-A4);

FIG. 6A is an XRPD pattern of HCl salt Type P (824509-10-A3), FIG. 6B shows TGA/DSC curves of HCl salt Type P (824509-10-A3), FIG. 6C is a ¹H NMR spectrum of HCl salt Type P (824509-10-A3), FIG. 6D is a HPLC chromatogram of HCl salt Type P (824509-10-A3), FIG. 6E is a VT-XRPD overlay of HCl salt Type P (824509-10-A3), FIG. 6F is an XRPD overlay of HCl salt Type P (824509-10-A3) after heating, and FIG. 6G is a ¹H NMR spectrum of HCl salt Type P after 80° C. heating;

FIG. 7A is an XRPD pattern of HCl salt Type Q (824509-16-A4), FIG. 7B is an XRPD pattern of HCl salt Type R (824509-16-A3), FIG. 7C is an XRPD overlay of HCl salt Type Q (824509-16-A4) and R (824509-16-A3), FIG. 7D shows TGA/DSC curves of HCl salt Type Q (824509-16-A4), FIG. 7E is a ¹H NMR spectrum of HCl salt Type Q (824509-16-A4), FIG. 7F is a HPLC chromatogram of HCl salt Type Q (824509-16-A4), FIG. 7G is a VT-XRPD overlay of HCl salt Type Q (824509-16-A4), FIG. 7H is an XRPD overlay of HCl salt Type Q after cooling back in VT-XRPD test, FIG. 7I is a ¹H NMR spectrum of HCl salt Type Q after VT-XRPD test, FIG. 7J shows TGA/DSC curves of HCl salt Type R (824509-16-A3), FIG. 7K is a ¹H NMR spectrum of HCl salt Type R (824509-16-A3), FIG. 7L is a HPLC chromatogram of HCl salt Type R (824509-16-A3), FIG. 7M is an XRPD overlay of HCl salt Type R (824509-16-A3) before and after heating, and FIG. 7N is a ¹H NMR spectrum of HCl salt Type R after 170° C. heating;

FIG. 8A is an XRPD pattern of HCl salt Type S (824509-25-A2), FIG. 8B shows TGA/DSC curves of HCl salt Type S (824509-29-A3), FIG. 8C is a ¹H NMR spectrum of HCl salt Type S (824509-29-A3), FIG. 8D is a HPLC chromatogram of HCl salt Type S (824509-29-A3), FIG. 8E is an XRPD overlay of HCl salt Type S (824509-39-A2) before and after heating, and FIG. 8F is a ¹H NMR spectrum of HCl salt Type S after 150° C. heating;

FIG. 9A is an XRPD pattern of HCl salt Type T (824509-25-A3), FIG. 9B is an XRPD overlay of HCl salt Type T (824509-39-A1) and reference, FIG. 9C shows TGA/DSC curves of HCl salt Type T (824509-39-A1), FIG. 9D is a ¹H NMR spectrum of HCl salt Type T (824509-39-A1), FIG. 9E is a HPLC chromatogram of HCl salt Type T (824509-39-A1), FIG. 9F is an XRPD overlay of HCl salt Type T (824509-39-A1) before and after heating, and FIG. 9G is a ¹H NMR spectrum of HCl salt Type T after 150° C. heating;

FIG. 10A is an XRPD pattern of HCl salt Type U (824509-29-B4), FIG. 10B shows TGA/DSC curves of HCl salt Type U (824509-29-B4), FIG. 10C is ¹H NMR spectrum of HCl salt Type U (824509-29-B4), FIG. 10D is HPLC chromatogram of HCl salt Type U (824509-29-B4), FIG. 10E is an XRPD overlay of HCl salt Type U (824509-29-B4) before after heating, and FIG. 10F is a ¹H NMR spectrum of HCl salt Type U after 150° C. heating;

FIG. 11A is an XRPD pattern of HCl salt Type V (824509-25-A4), and FIG. 11B is an XRPD overlay of HCl salt Type V (824509-25-A4) after air drying;

FIG. 12A is an XRPD pattern of HCl salt Type A+L (824509-05-A1), FIG. 12B is an XRPD overlay of HCl salt Type A+L (824509-05-A1) before and after RT storage, FIG. 12C is an XRPD pattern of HCl salt Type A+N (824509-10-A1), FIG. 12D is an XRPD overlay of HCl salt Type A+N (824509-10-A1) before and after storage at RT, FIG. 12E is an XRPD pattern of HCl salt Type A+O (824509-10-A2), and FIG. 12F is an XRPD overlay of HCl salt Type A+O (824509-10-A2) before and after storage at RT;

FIG. 13 is an XRPD overlay of sulfate forms;

FIG. 14A is an XRPD overlay of sulfate Type A (824511-04-A) and reference, FIG. 14B shows TGA/DSC curves of sulfate Type A (824511-04-A), FIG. 14C is a ¹H NMR spectrum of sulfate Type A (824511-04-A), FIG. 14D is a HPLC chromatogram of sulfate Type A (824511-04-A), and FIG. 14E is a VT-XRPD overlay of sulfate Type A (824511-04-A);

FIG. 15A is an XRPD pattern of sulfate Type D (824511-12-A17), FIG. 15B shows TGA/DSC curves of sulfate Type D (824511-12-A17), FIG. 15C is a ¹H NMR spectrum of sulfate Type D (824511-12-A17), FIG. 15D is a HPLC chromatogram of sulfate Type D (824511-12-A17), FIG. 15E is a VT-XRPD overlay of sulfate Type D (824511-12-A17), FIG. 15F is an XRPD pattern of sulfate Type F (824511-12-A17_N₂ Back_30.0° C.), FIG. 15G is an XRPD pattern of sulfate Type E (824511-11-A3-0315), FIG. 15H shows TGA/DSC curves of sulfate Type E (824511-11-A3-0315), FIG. 15I is an XRPD overlay of sulfate Type E (824511-11-A3-100C) before and after heating, FIG. 15J is a ¹H NMR spectrum of sulfate Type E (824511-11-A3-100C) after heating, FIG. 15K is an XRPD pattern of sulfate Type B (824511-11-A3), FIG. 15L shows TGA/DSC curves of sulfate Type B (824511-11-A3), FIG. 15M is a ¹H NMR spectrum of sulfate Type B (824511-11-A3), FIG. 15N is a HPLC chromatogram of sulfate Type B (824511-11-A3), FIG. 15O is an XRPD overlay of HCl salt Type B (824511-11-A3) before and after heating, FIG. 15P shows TGA curves overlay of sulfate Type B (824511-11-A3) before and after storage, FIG. 15Q is an XRPD pattern of sulfate Type C (824511-11-A4), and FIG. 15R is an XRPD overlay of sulfate Type C (824511-11-A4) before and after air drying;

FIG. 16A is an XRPD overlay of oxalate Type A (819246-23-A20) and Type B (824511-04-C), FIG. 16B shows TGA/DSC curves of oxalate Type B (824511-04-C), FIG. 16C is a ¹H NMR spectrum of oxalate Type B (824511-04-C), and FIG. 16D is a HPLC chromatogram of oxalate Type B (824511-04-C);

FIG. 17 is an XRPD overlay of obtained mesylate Forms A, B, and C;

FIG. 18A is an XRPD pattern of mesylate Type A (824511-23-B), FIG. 18B is an XRPD overlay of re-prepared mesylate Type A (824511-23-B) and reference, FIG. 18C shows TGA/DSC curves of mesylate Type A (824511-23-B), FIG. 18D is a ¹H NMR spectrum of re-prepared mesylate Type A (824511-23-B), FIG. 18E is a HPLC chromatogram of mesylate Type A (824511-23-B), and FIG. 18F is a VT-XRPD overlay of mesylate Type A (824511-23-B);

FIG. 19 is an XRPD overlay of HBr salt forms;

FIG. 20A is an XRPD pattern of HBr salt Type A (824511-29-B), FIG. 20B is an XRPD overlay of HBr salt Type A (824511-29-B) and reference, FIG. 20C shows TGA/DSC curves of HBr salt Type A (824511-29-B), FIG. 20D is a HPLC chromatogram of HBr salt Type A (824511-29-B), FIG. 20E is a VT-XRPD overlay of HBr salt Type A (824511-29-B), FIG. 20F is an XRPD pattern of HBr salt Type B (824511-10-A1), FIG. 20G is an XRPD overlay of HBr salt Type B batches, FIG. 20H shows TGA/DSC curves of HBr salt Type B (824511-10-A1), FIG. 20I is a ¹H NMR spectrum of HBr salt Type B (824511-10-A1), FIG. 20J is a HPLC chromatogram of HBr salt Type B (824511-10-A1), FIG. 20K is an XRPD overlay of HBr salt Type B before and after storage, FIG. 20L is an XRPD pattern of HBr salt Type C (824511-39-A3), FIG. 20M shows TGA/DSC curves of HBr salt Type C (824511-39-A3), FIG. 20N is ¹H NMR spectrum of HBr salt Type C (824511-39-A3), FIGURE is HPLC chromatogram of HBr salt Type C, FIG. 20P is an XRPD overlay of heating experiments for HBr salt Type C, FIG. 20Q is a ¹H NMR spectrum of HBr salt Type C after heating (824511-39-A3-H150), FIG. 20R is an XRPD pattern of HBr salt Type D (824511-39-A12), FIG. 20S shows TGA/DSC curves of HBr salt Type D, FIG. 20T is a ¹H NMR spectrum of HBr salt Type D, FIG. 20U is HPLC chromatogram of HBr salt Type D, FIG. 20V is an XRPD overlay of HBr salt Type D in heating experiments, and FIG. 20W is a ¹H NMR spectrum of HBr salt Type D after heating;

FIG. 21 is an XRPD overlay of obtained forms of tosylate;

FIG. 22A is an XRPD overlay of tosylate Type B and C, FIG. 22B is a HPLC chromatogram of tosylate Type B, FIG. 22C is an XRPD pattern of tosylate Type A, FIG. 22D shows TGA/DSC curves of tosylate Type A, FIG. 22E is a¹H NMR spectrum of tosylate Type A (824528-06-A3), FIG. 22F is a HPLC chromatogram of tosylate Type A (824528-06-A3), FIG. 22G is a VT-XRPD overlay of tosylate Type A (824528-09-A2), FIG. 22H is an XRPD overlay of tosylate Type A (824528-06-A3) before after storage, FIGURE is an XRPD pattern of tosylate Type I (824528-09-A2_N2_Back to_30° C.), FIG. 22J is an XRPD pattern of tosylate Type C (824511-23-A), FIG. 22K shows TGA/DSC curves of tosylate Type C (824511-23-A), FIG. 22L is a ¹H NMR spectrum of tosylate Type C (824511-23-A), FIG. 22M is a HPLC chromatogram of tosylate Type C (824511-23-A), FIG. 22N is a VT-XRPD overlay of tosylate Type C (824511-23-A), FIGURE is an XRPD overlay of tosylate Type D exposure to air for 30 min, and FIG. 22P is an XRPD pattern of tosylate Type D (824511-23-A-RE_N2_60min_30.0° C.);

FIG. 23A is an XRPD pattern of tosylate Type E (824528-05-A9), FIG. 23B shows TGA/DSC curves of tosylate Type E (824528-05-A9), FIG. 23C is a ¹H NMR spectrum of tosylate Type E (824528-05-A9), FIG. 23D is a HPLC chromatogram of tosylate Type E (824528-05-A9), FIG. 23E is a XRPD overlay of tosylate Type E (824528-05-A9) before and after heating, FIG. 23F is a ¹H NMR spectrum of sample from 101° C. heating experiments of tosylate Type E, FIG. 23G is an XRPD pattern of tosylate Type F (824528-06-B1), FIG. 23H is an XRPD overlay of tosylate Type F (824528-06-B1) and reference, FIGURE shows TGA/DSC curves of tosylate Type F (824528-06-B1), FIG. 23J is a ¹H NMR spectrum of tosylate Type F (824528-06-B1), FIG. 23K is a HPLC chromatogram of tosylate Type F (824528-06-B1), FIG. 23L is an XRPD overlay of tosylate Type F (824528-06-B1) before and after heating, FIG. 23M is a ¹H NMR spectrum of sample from 90° C. heating experiments of tosylate Type F, FIG. 23N is an XRPD pattern of tosylate Type G (824528-06-A1), FIGURE is an XRPD overlay of tosylate Type G (824528-06-A1) and reference, FIG. 23P shows TGA/DSC curves of tosylate Type G (824528-06-A1), FIG. 23Q is a ¹H NMR spectrum of tosylate Type G (824528-06-A1), FIG. 23R is a HPLC chromatogram of tosylate Type G (824528-06-A1), FIG. 23S is an XRPD overlay of tosylate Type G (824528-06-A1) after heating, and FIG. 23T is a ¹H NMR spectrum of sample from 100° C. heating experiments of tosylate Type G;

FIG. 24A is an XRPD pattern of tosylate Type H (824528-05-A12), and FIG. 24B is an XRPD overlay of tosylate Type H (824528-06-A3) after air drying;

FIG. 25 is an XRPD overlay of obtained forms of besylate;

FIG. 26A is an XRPD pattern of besylate Type A (824511-35-A1), FIG. 26B is an XRPD overlay of besylate Type A (824511-35-A1) and reference, FIG. 26C shows TGA/DSC curves of besylate Type A (824511-35-A1), FIG. 26D is a ¹H NMR spectrum of besylate Type A (824511-35-A1), FIG. 26E is a HPLC chromatogram of besylate Type A (824511-35-A1), FIG. 26F is a VT-XRPD overlay of besylate Type A (824529-04-A5), FIG. 26G is an XRPD pattern of besylate Type C (824529-04-A5_N2 Back_30.0° C.), FIG. 26H is an XRPD pattern of besylate Type B (824511-44-C2), FIGURE is an XRPD overlay of besylate Type B (824511-44-C2) and reference, FIG. 26J shows TGA/DSC curves of besylate Type B (824511-44-C2), FIG. 26K is a ¹H NMR spectrum of besylate Type B (824511-44-C2), FIG. 26L is a HPLC chromatogram of besylate Type B (824511-44-C2), and FIG. 26M is a VT-XRPD overlay of besylate Type B (824511-44-C2); and

FIG. 27 shows XPRD data for maleate Type A.

### DETAILED DESCRIPTION OF THE INVENTION

The present invention provides for salts and polymorphs of 18-MC. The polymorphs can be crystalline or amorphous.

The salts can include gentisate, hydrobromide, besylate, napadisylate, hydrochloride, sulfate, oxalate, maleate, mesylate, and tosylate.

The polymorphs can include HCl salt Type A, HCl salt Type B, HCl salt Type C, HCl salt Type D, HCl salt Type E, HCl salt Type F, HCl salt Type G, HCl salt Type H, HCl salt Type I, HCl salt Type J, HCl salt Type K, HCl salt Type L, HCl salt Type M, HCl salt Type N, HCl salt Type O, HCl salt Type P, HCl salt Type Q, HCl salt Type R, HCl salt Type S, HCl salt Type T, HCl salt Type U, HCl salt Type V, sulfate salt Type A, sulfate salt Type B, sulfate salt Type C, sulfate salt Type D, sulfate salt Type E, sulfate salt Type F, oxalate salt Type A, oxalate salt Type B, maleate salt Type A, mesylate salt Type A, mesylate salt Type B, mesylate salt Type C, HBr salt Type A, HBr salt Type B, HBr salt Type C, HBr salt Type D, tosylate salt Type A, tosylate salt Type B, tosylate salt Type C, tosylate salt Type D, tosylate salt Type E, tosylate salt Type F, tosylate salt Type G, tosylate salt Type H, tosylate salt Type I, besylate salt Type A, besylate salt Type B, besylate salt Type C, napadisylate salt Type A, napadisylate salt Type B, napadisylate salt Type C, napadisylate salt Type D, and gentisate salt Type A.

Crystalline gentisate salt Type A can be characterized by an x-ray powder diffraction pattern having peaks expressed as 20 at about 10.3, about 11.1, about 16.3, about 20.6, about 21.0, and about 27.8. Crystalline HBr salt Type A can be characterized by an x-ray powder diffraction pattern having peaks expressed as 20 at about 8.6, about 13.1, about 19.1, about 19.9, about 26.1, and about 26.3. Crystalline HBr salt Type B can be characterized by an x-ray powder diffraction pattern having peaks expressed as 20 at about 7.5, about 15.0, about 21.2, about 21.9, about 24.1, and about 30.3. Crystalline besylate salt Type A can be characterized by an x-ray powder diffraction pattern having peaks expressed as 20 at about 7.4, about 8.1, about 14.3, about 14.7, about 19.7, and about 22.7. Crystalline besylate salt Type B can be characterized by an x-ray powder diffraction pattern having peaks expressed as 20 at about 8.3, about 9.8, about 16.6, about 17.7, about 18.4, and about 18.7. Crystalline napadisylate salt Type A can be characterized by an x-ray powder diffraction pattern having peaks expressed as 20 at about 7.6, about 8.1, about 12.2, about 12.7, about 14.6, and about 17.5.

Crystalline napadisylate salt Type B can be characterized by an x-ray powder diffraction pattern having peaks expressed as 20 at about 8.2, about 10.6, about 17.8, about 19.3, about 20.0, and about 21.3. Crystalline napadisylate salt Type C can be characterized by an x-ray powder diffraction pattern having peaks expressed as 20 at about 7.3, about 9.6, about 15.2, about 18.4, about 19.1, and about 24.5. Crystalline napadisylate salt Type D can be characterized by an x-ray powder diffraction pattern having peaks expressed as 20 at about 7.3, about 7.5, about 15.0, about 17.8, about 18.0, and about 22.5. Crystalline HCl salt Type A and is characterized by an x-ray powder diffraction pattern having peaks expressed as 20 at about 8.8, about 11.0, about 13.4, about 16.2, about 16.5, and about 16.8. Crystalline sulfate salt Type A can be characterized by an x-ray powder diffraction pattern having peaks expressed as 20 at about 5.2, about 10.5, about 13.8, about 15.7, about 18.3, and about 20.4.

Crystalline maleate salt Type A can be characterized by an x-ray powder diffraction pattern having peaks expressed as 20 at about 7.9, about 14.3, about 14.7, about 15.9, about 18.3, and about 19.1. Crystalline tosylate salt Type A can be characterized by an x-ray powder diffraction pattern having peaks expressed as 20 at about 8.6, about 10.4, about 11.8, about 17.9, about 18.2, and about 21.0. Crystalline tosylate salt Type B can be characterized by an x-ray powder diffraction pattern having peaks expressed as 20 at about 7.4, about 7.6, about 9.6, about 11.6, about 14.9, and about 15.3. Crystalline mesylate salt Type A can be characterized by an x-ray powder diffraction pattern having peaks expressed as 20 at about 8.1, about 9.2, about 13.0, about 16.9, about 18.2, and about 21.1. Crystalline oxalate salt Type A can be characterized by an x-ray powder diffraction pattern having peaks expressed as 20 at about 6.0, about 9.1, about 13.6, about 15.8, about 18.2, and about 21.8. Crystalline oxalate salt Type B can be characterized by an x-ray powder diffraction pattern having peaks expressed as 20 at about 7.7, about 11.8, about 13.7, about 16.7, about 17.7, and about 18.9. The freebase can be characterized by an x-ray powder diffraction pattern having peaks expressed as 20 at about 11.0, about 11.7, about 14.0, about 15.5, about 18.3, and about 21.3.

A formal, broad salt and polymorph screen was conducted resulting in at least 10 pharmaceutically relevant salts. Most of these exhibit polymorphism that was further characterized as described in the EXAMPLES below. The hydrochloride salt exhibits at least 22 different forms. Form A is the most thermodynamically stable form. Unexpectedly, however, Form J was isolated on up to ~100 g scale when isolated from HCl/EtOAc. Thus, appropriate control of the manufacturing process is critical to obtain the desired salt and polymorph.

The current standard process for making 18-MC (isolation from dioxane/HCI) ensures the controlled isolation of Form A.

The invention is further described in detail by reference to the following experimental examples. These examples are provided for the purpose of illustration only, and are not intended to be limiting unless otherwise specified. Thus, the invention should in no way be construed as being limited to the following examples, but rather, should be construed to encompass any and all variations which become evident as a result of the teaching provided herein.

### Example 1 - Salt Screening of 18-MC

Applicant performed an extended salt screening and collected data of alternative salts.

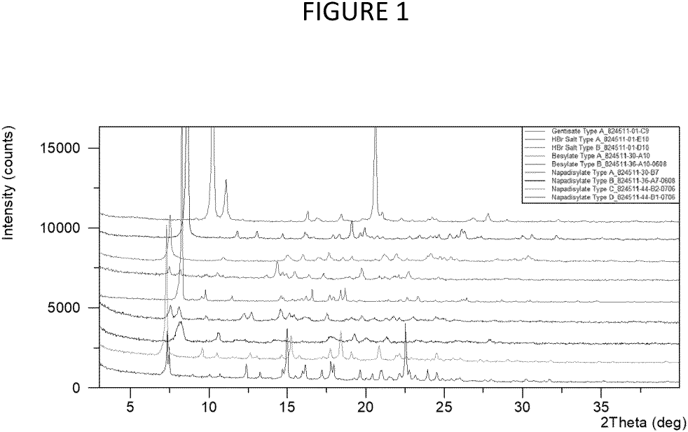

18-MC Freebase was first isolated from 18-MC HCl salt and then used for salt screening. In the screening, a total of 100 experiments were performed using 20 acids and 5 solvents. Resulting solids were characterized by X-ray powder diffraction (XRPD). Based on XRPD comparison results, new forms were then characterized by thermogravimetric analysis (TGA), differential scanning calorimetry (DSC), proton nuclear magnetic resonance (¹H NMR) high-performance liquid chromatography (HPLC) and/or ion chromatography (IC). As the results showed, a total of 16 salt forms were discovered from screening and form re-preparation, including gentisate Type A, HBr salt Types A and B, besylate Types A and B and napadisylate Types A, B, C′ and D. XRPD patterns are shown in FIG. 1 and characterization results are summarized in TABLE 1A. Additional Salt screening results are summarized in TABLE 1B. A summary of all salts and salt forms produced during the study are in TABLE 5B.

To summarize, a total of 16 new salt forms were found and fully characterized in the extended salt screening.

TABLE 1A

Characterization summary of salt hits

| Solid form (ID) | TGA loss (%) | Endothermic signal (°C., peak) | Solvent residual (wt%) | Molar ratio (acid/freebase) | HPLC purity (area %) |
|---|---|---|---|---|---|
| Gentisate Type A (824511-01-C9) | 2.7 (up to 150° C.) | 181.9 | 1.1 (CHCl₃) | 1.0 | 99.79 |
| HBr salt Type A (824511-01-E10) | 1.5 (up to 150° C.) | 208.5 | 0.6 (IPA) | 1.0 | 99.72 |
| HBr salt Type B* (824511-10-A1) | 14.2 (up to 150° C.) | 104.3, 140.3, 177.4^{#} | 10.8 (1,4-dioxane) | 0.9 | 99.27 |
| Besylate Type A (824511-35-A1) | 1.1% (up to 90° C.) | 117.4, 131.8 | Not detected | 1.0 | 99.71 |
| | 3.3% (90° C. to 130° C.) | | | | |
| Besylate Type B (824511-36-A10) | 0.4% (up to 80° C.) | 177.5, 179.3 | Not detected | 1.0 | 99.73 |
| Napadisylate Type A (824511-30-B7) | 3.5% (up to 70° C.) 3.6% | 96.6, 163.0, 198.5 | 0.26 (Acetone) | 0.6 | 99.21 |
| | (from 70° C. to 120° C.) | | | | |
| Napadisylate Type B (824511-36-A7) | 6.0% (up to 80° C.) | 81.7, 206.0 | 8.17 (IPA) | 0.6 | 99.66 |
| Napadisylate Type C (824511-44-B2) | 6.7% (up to 100° C.) | 71.2, 117.0, 191.0 | 8.91 (1,4-Dioxane) | 0.7 | 98.42 |
| Napadisylate Type D (824511-44-B1) | 6.9% (up to 100° C.) | 53.9, 89.0, 178.3 | Not detected | 0.7 | 99.56 |

*: Partially converted to HBr salt Type A after storage at RT for ~20 days.
#: Exothermic signal.

TABLE 1B

Characterization summary of salt hits

| Solid form (ID:819246-) | TGA loss (%) | Endothermic signal (°C, peak) | Solvent residual (wt%) | Molar ratio (acid/freebase) | HPLC purity (area%) |
|---|---|---|---|---|---|
| HCl salt Type A (819246-01-A) | 1.4 (150° C.) | 211.6 | NA | 1.0 | 99.39 |
| Sulfate Type A (819246-23-A2) | 5.8 (150° C.) | 150.4, 185.5 | NA | 1.1 | 99.51 |
| Maleate Type A (819246-23-A4) | 0.8 (150° C.) | 180.4 | NA* | 0.9 | 99.84 |
| Tosylate Type A (819246-23-A18) | 4.2 (120° C.) | 72.9, 114.1, 145.3 | 2.7 (EtOAc) | 0.9 | 99.56 |
| Tosylate Type B (819246-23-D18) | 5.8 (120° C.) | 93.9, 119.0, 183.5 | NA^{#} | 1.0 | 99.80 |
| Mesylate Type A (819246-23-A19) | 3.1 (120° C.) | 76.0, 161.1 | Not detected | 1.1 | 99.75 |
| Oxalate Type A (819246-23-A20) | 1.5 (120° C.) | 170.6 | NA | 0.9 | 99.66 |

NA: No data available.
*: Signal of solvent in ¹H NMR results was interfered by baseline fluctuation.
#: Signal of solvent in ¹H NMR results was overlapped with API.

TABLE 1C

XRPD peak list of 18-MC Hydrochloride Type A (824509-01-A)

| Pos. [°2θ] | Height [cts] | FWHM Left [°2θ] | d-spacing [Å] | Rel. Int. [%] |
|---|---|---|---|---|
| 8.7799 | 7072.73 | 0.1535 | 10.07 | 100.00 |
| 11.0366 | 1923.53 | 0.1279 | 8.02 | 27.20 |
| 11.9442 | 207.99 | 0.1023 | 7.41 | 2.94 |
| 13.3791 | 1816.53 | 0.1279 | 6.62 | 25.68 |
| 15.0012 | 786.19 | 0.1535 | 5.91 | 11.12 |
| 16.2228 | 1495.30 | 0.1023 | 5.46 | 21.14 |
| 16.4500 | 1367.71 | 0.1023 | 5.39 | 19.34 |
| 16.8106 | 1653.21 | 0.1535 | 5.27 | 23.37 |
| 17.3798 | 124.85 | 0.1023 | 5.10 | 1.77 |
| 18.1119 | 574.54 | 0.1279 | 4.90 | 8.12 |
| 18.4266 | 1012.59 | 0.1535 | 4.82 | 14.32 |
| 19.2817 | 535.80 | 0.1535 | 4.60 | 7.58 |
| 19.9424 | 621.63 | 0.1279 | 4.45 | 8.79 |
| 20.2152 | 1154.12 | 0.1279 | 4.39 | 16.32 |
| 20.6874 | 898.12 | 0.1791 | 4.29 | 12.70 |
| 21.5520 | 902.41 | 0.1535 | 4.12 | 12.76 |
| 22.3521 | 425.08 | 0.1535 | 3.98 | 6.01 |
| 23.0721 | 394.57 | 0.1535 | 3.85 | 5.58 |
| 23.3391 | 231.10 | 0.1023 | 3.81 | 3.27 |
| 23.6343 | 355.16 | 0.1279 | 3.76 | 5.02 |
| 24.7539 | 1220.89 | 0.1535 | 3.60 | 17.26 |
| 25.5809 | 540.83 | 0.1279 | 3.48 | 7.65 |
| 25.9108 | 1213.25 | 0.1535 | 3.44 | 17.15 |
| 26.3606 | 425.59 | 0.1535 | 3.38 | 6.02 |
| 26.5846 | 550.08 | 0.1023 | 3.35 | 7.78 |
| 27.0788 | 169.54 | 0.1279 | 3.29 | 2.40 |
| 28.3565 | 59.96 | 0.2558 | 3.15 | 0.85 |
| 29.5132 | 150.79 | 0.1535 | 3.03 | 2.13 |
| 29.8709 | 297.61 | 0.0768 | 2.99 | 4.21 |
| 30.0708 | 279.84 | 0.1279 | 2.97 | 3.96 |
| 30.5068 | 369.71 | 0.1023 | 2.93 | 5.23 |
| 30.8279 | 786.13 | 0.1535 | 2.90 | 11.11 |
| 31.8040 | 136.15 | 0.1023 | 2.81 | 1.92 |
| 32.5997 | 231.88 | 0.1535 | 2.75 | 3.28 |
| 32.9807 | 122.78 | 0.1535 | 2.72 | 1.74 |
| 33.2925 | 101.04 | 0.1791 | 2.69 | 1.43 |
| 34.5690 | 144.48 | 0.1535 | 2.59 | 2.04 |
| 35.4156 | 107.53 | 0.2047 | 2.53 | 1.52 |
| 36.3033 | 172.98 | 0.1535 | 2.47 | 2.45 |
| 37.2414 | 70.70 | 0.1535 | 2.41 | 1.00 |
| 37.8530 | 167.15 | 0.1023 | 2.38 | 2.36 |
| 38.8227 | 124.20 | 0.2047 | 2.32 | 1.76 |

### Freebase Isolation

### Characterization of Starting Material

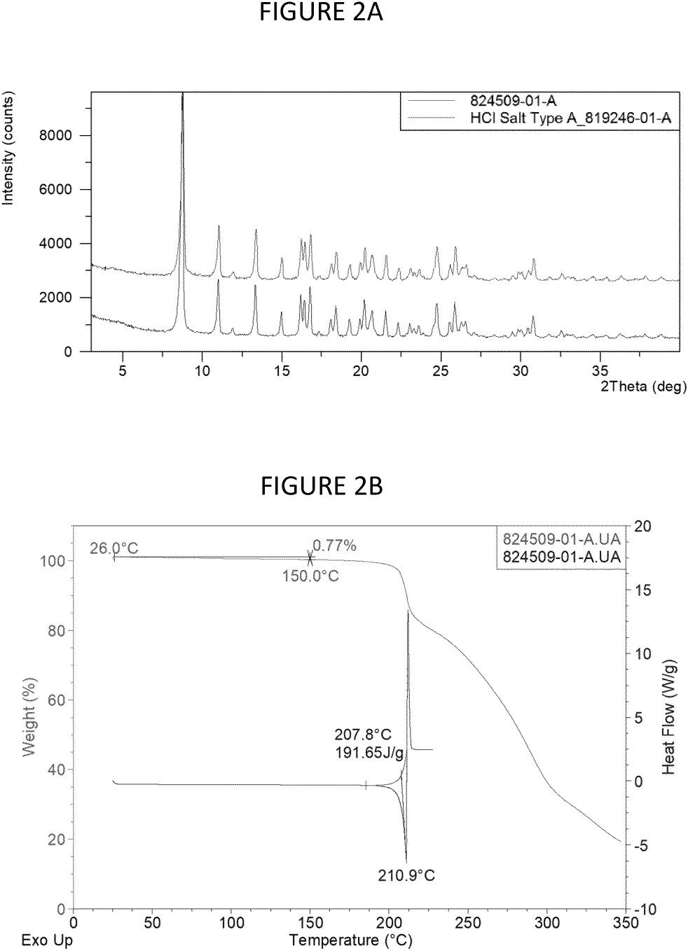

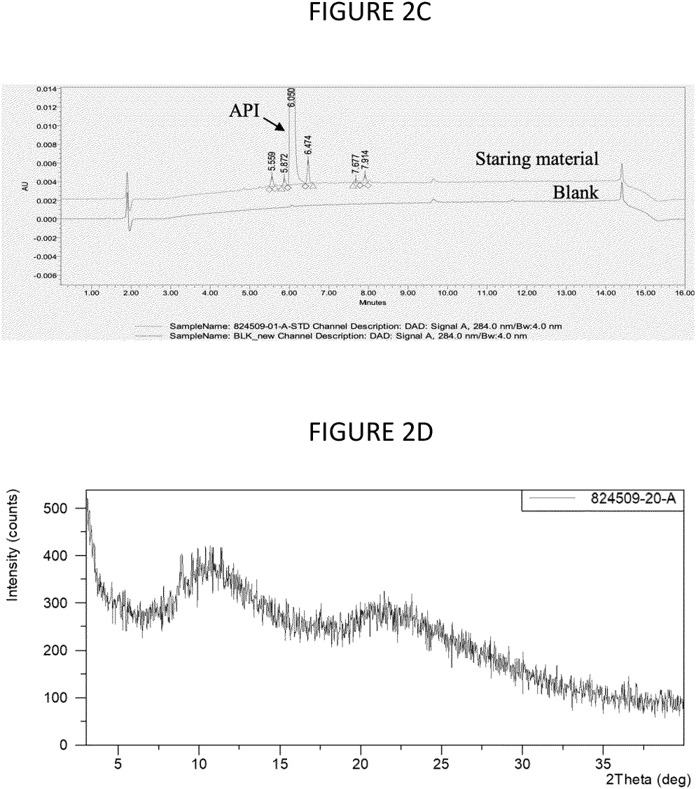

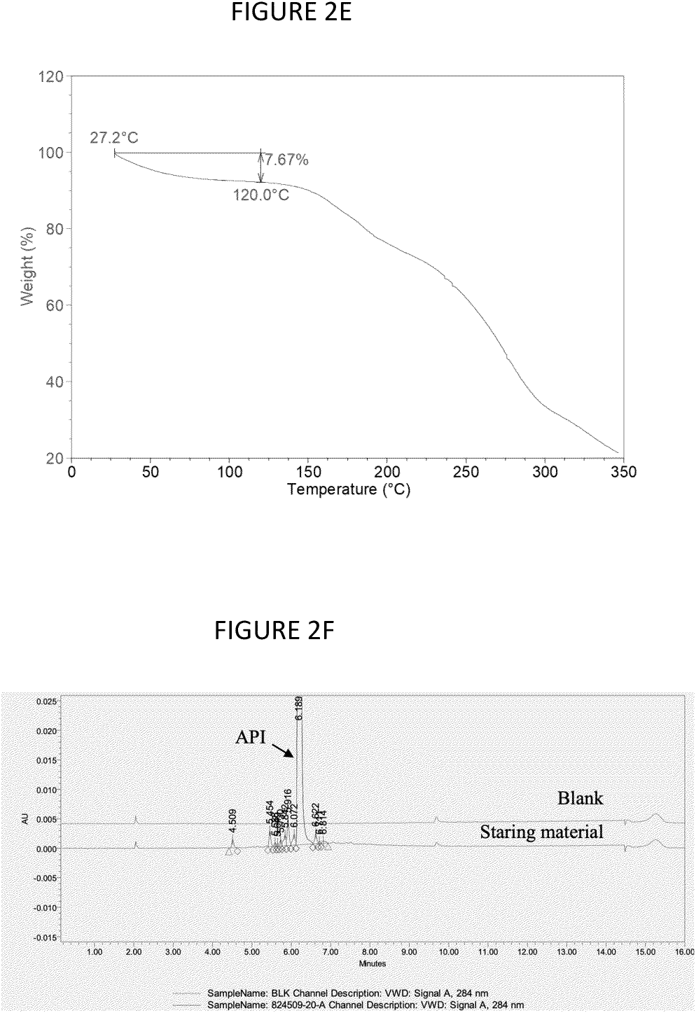

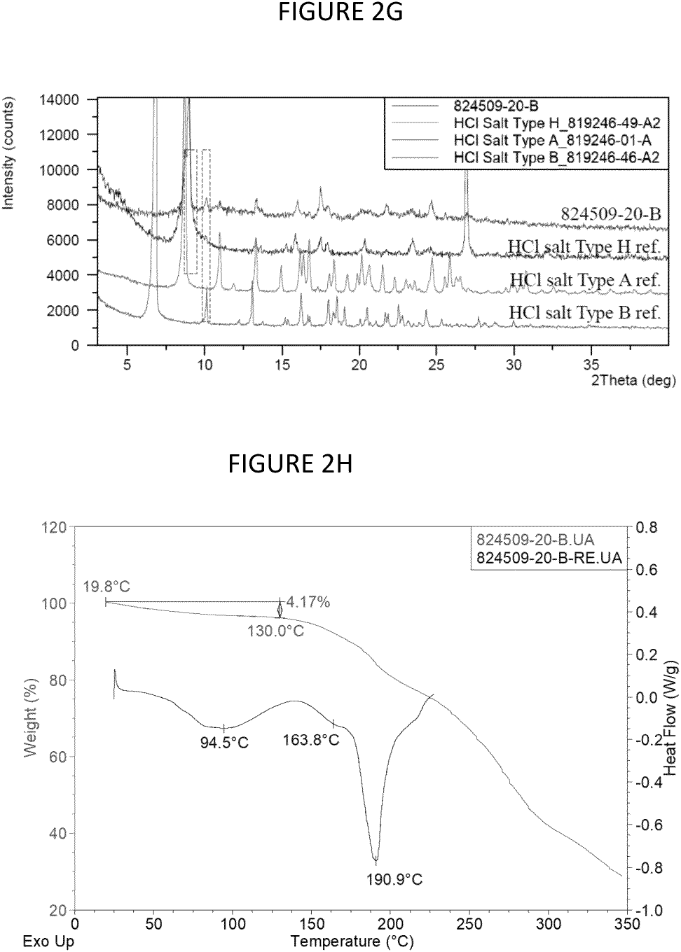

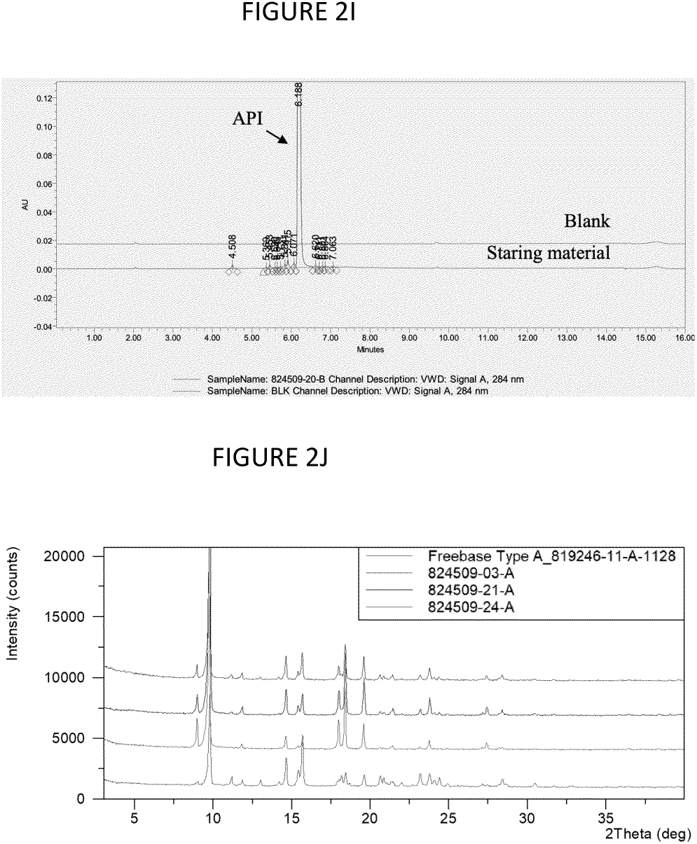

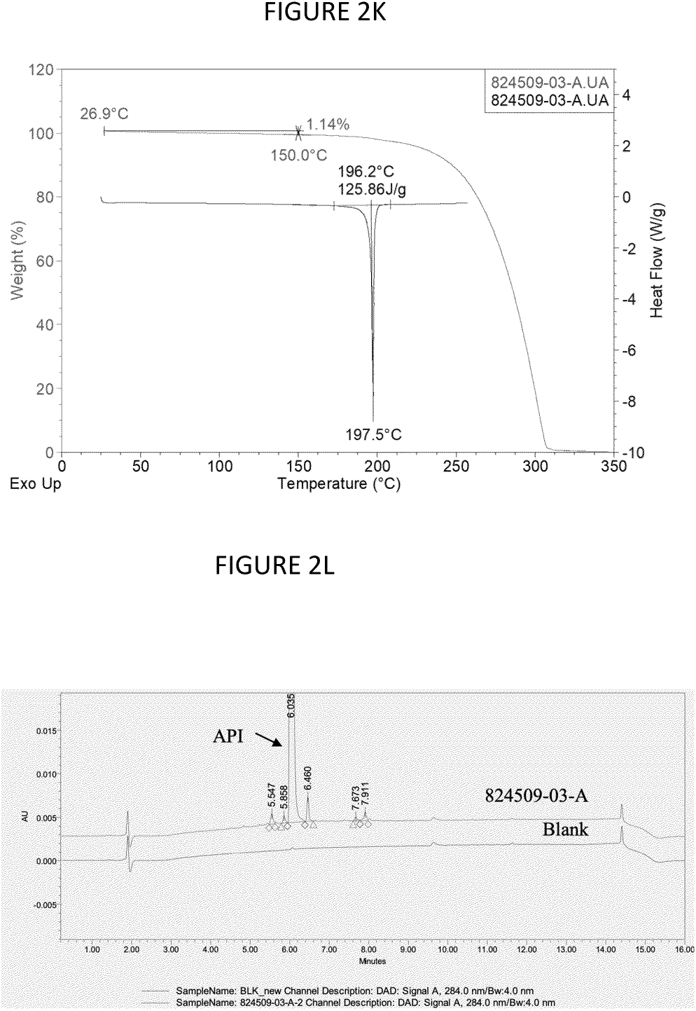

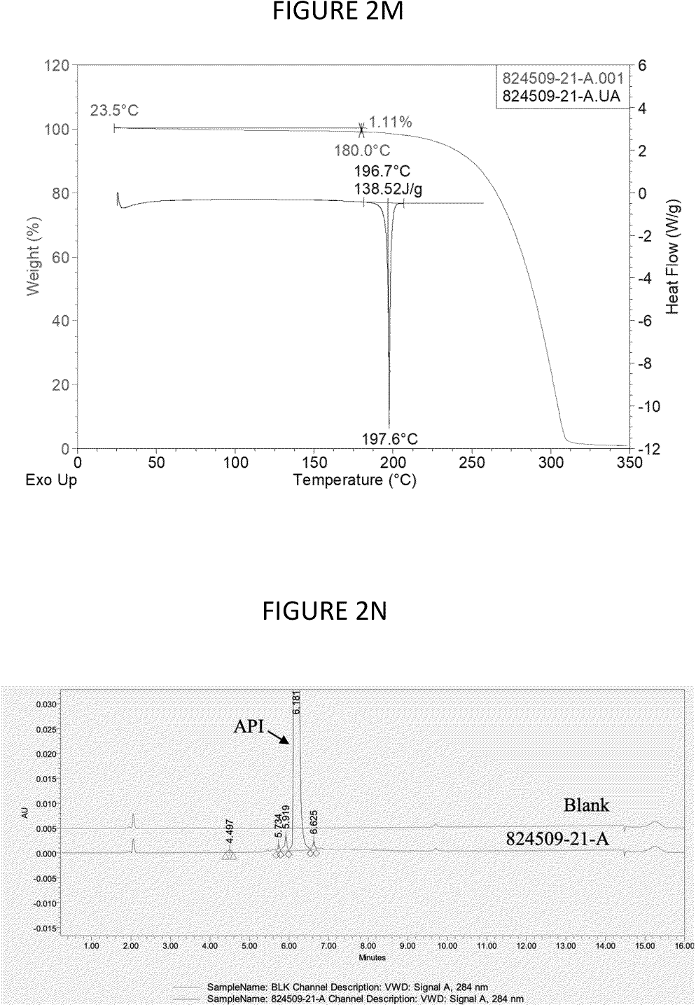

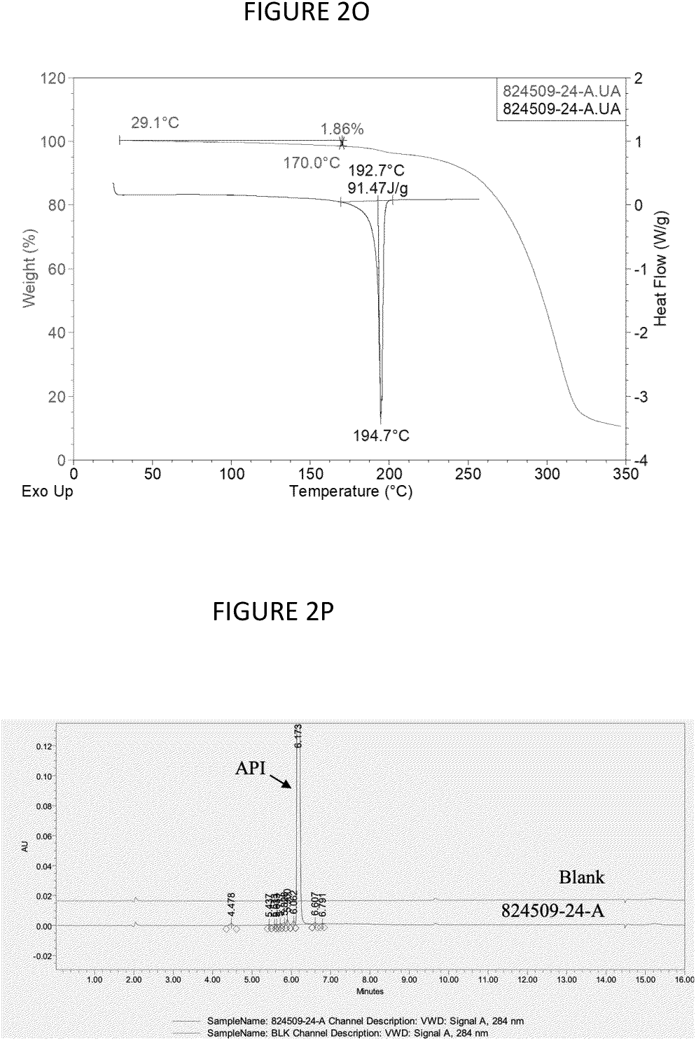

Three batches of starting material of 18-MC HCl salt were characterized by XRPD, TGA, DSC and HPLC/IC. Characterization results are summarized in TABLE 2A and displayed from FIGS. 2A to 21. As the characterization results showed, the three batches represent different HCl salt polymorphs.

TABLE 2A

Summary of 18-MC HCl salt starting material

| Batch Number | XRPD | TGA loss (%) | Endothermic signal (°C, peak) | HPLC purity (area%) |
|---|---|---|---|---|
| 824509-01-A | HCl salt Type A | 0.8% up to 150° C. | 210.9 | 99.33 |
| 824509-20-A | Amorphous | 7.7% up to 120° C. | -- | 97.85 |
| 824509-20-B | HCl salt Type H+A+B | 4.2% up to 130.0° C. | 94.5, 168.3, 190.9 | 97.02 |

--: no data available.

As shown in FIG. 2A and TABLE 2B, starting material (824509-01-A) was crystalline and conformed to HCl salt Type A. TGA/DSC results in FIG. 2B showed that a weight loss of 0.8% up to 150° C. and one endothermic signal at 207.8° C. (onset) was observed before decomposition. The Cl- content of the material was determined as 8.73% (theoretical Cl- content for mono HCl salt is 8.77%), and HPLC purity was 99.33 area% (FIG. 2C and TABLE 2C).

TABLE 2B

XRPD peak list of starting material (824509-01-A)

| Pos. [°2theta] | Height [cts] | FWHM Left [°2theta] | d-spacing [Å] | Rel. Int. [%] |
|---|---|---|---|---|
| 8.7799 | 7072.73 | 0.1535 | 10.07 | 100.00 |
| 11.0366 | 1923.53 | 0.1279 | 8.02 | 27.20 |
| 11.9442 | 207.99 | 0.1023 | 7.41 | 2.94 |
| 13.3791 | 1816.53 | 0.1279 | 6.62 | 25.68 |
| 15.0012 | 786.19 | 0.1535 | 5.91 | 11.12 |
| 16.2228 | 1495.30 | 0.1023 | 5.46 | 21.14 |
| 16.4500 | 1367.71 | 0.1023 | 5.39 | 19.34 |
| 16.8106 | 1653.21 | 0.1535 | 5.27 | 23.37 |
| 17.3798 | 124.85 | 0.1023 | 5.10 | 1.77 |
| 18.1119 | 574.54 | 0.1279 | 4.90 | 8.12 |
| 18.4266 | 1012.59 | 0.1535 | 4.82 | 14.32 |
| 19.2817 | 535.80 | 0.1535 | 4.60 | 7.58 |
| 19.9424 | 621.63 | 0.1279 | 4.45 | 8.79 |
| 20.2152 | 1154.12 | 0.1279 | 4.39 | 16.32 |
| 20.6874 | 898.12 | 0.1791 | 4.29 | 12.70 |
| 21.5520 | 902.41 | 0.1535 | 4.12 | 12.76 |
| 22.3521 | 425.08 | 0.1535 | 3.98 | 6.01 |
| 23.0721 | 394.57 | 0.1535 | 3.85 | 5.58 |
| 23.3391 | 231.10 | 0.1023 | 3.81 | 3.27 |
| 23.6343 | 355.16 | 0.1279 | 3.76 | 5.02 |
| 24.7539 | 1220.89 | 0.1535 | 3.60 | 17.26 |
| 25.5809 | 540.83 | 0.1279 | 3.48 | 7.65 |
| 25.9108 | 1213.25 | 0.1535 | 3.44 | 17.15 |
| 26.3606 | 425.59 | 0.1535 | 3.38 | 6.02 |
| 26.5846 | 550.08 | 0.1023 | 3.35 | 7.78 |
| 27.0788 | 169.54 | 0.1279 | 3.29 | 2.40 |
| 28.3565 | 59.96 | 0.2558 | 3.15 | 0.85 |
| 29.5132 | 150.79 | 0.1535 | 3.03 | 2.13 |
| 29.8709 | 297.61 | 0.0768 | 2.99 | 4.21 |
| 30.0708 | 279.84 | 0.1279 | 2.97 | 3.96 |
| 30.5068 | 369.71 | 0.1023 | 2.93 | 5.23 |
| 30.8279 | 786.13 | 0.1535 | 2.90 | 11.11 |
| 31.8040 | 136.15 | 0.1023 | 2.81 | 1.92 |
| 32.5997 | 231.88 | 0.1535 | 2.75 | 3.28 |
| 32.9807 | 122.78 | 0.1535 | 2.72 | 1.74 |
| 33.2925 | 101.04 | 0.1791 | 2.69 | 1.43 |
| 34.5690 | 144.48 | 0.1535 | 2.59 | 2.04 |
| 35.4156 | 107.53 | 0.2047 | 2.53 | 1.52 |
| 36.3033 | 172.98 | 0.1535 | 2.47 | 2.45 |
| 37.2414 | 70.70 | 0.1535 | 2.41 | 1.00 |
| 37.8530 | 167.15 | 0.1023 | 2.38 | 2.36 |
| 38.8227 | 124.20 | 0.2047 | 2.32 | 1.76 |

TABLE 2C

| # | RRT | Area (%) | # | RRT | Area (%) |
|---|---|---|---|---|---|
| 1 | 0.92 | 0.14 | 4 | 1.07 | 0.26 |
| 2 | 0.97 | 0.09 | 5 | 1.27 | 0.05 |
| 3 | 1.00 | 99.33 | 6 | 1.31 | 0.13 |

As shown in FIG. 2D, the sample (824509-20-A,) was amorphous. TGA result in FIG. 2E showed a weight loss of 7.7% up to 120° C. HPLC/IC results showed that the molar ratio of hydrochloric acid to 18-MC freebase was determined as 1.1:1 and HPLC purity was 97.85 area% (FIG. 2F and TABLE 2D).

TABLE 2D

HPLC results of starting material (824509-20-A)

| # | RRT | Area(%) | # | RRT | Area(%) |
|---|---|---|---|---|---|
| 1 | 0.73 | 0.18 | 7 | 0.96 | 0.51 |
| 2 | 0.88 | 0.29 | 8 | 0.98 | 0.29 |
| 3 | 0.90 | 0.07 | 9 | 1.00 | 97.85 |
| 4 | 0.91 | 0.05 | 10 | 1.07 | 0.23 |
| 5 | 0.93 | 0.12 | 11 | 1.08 | 0.05 |
| 6 | 0.94 | 0.28 | 12 | 1.10 | 0.09 |

As shown in FIG. 2G, the sample (824509-20-B) was similar to HCl salt Type H, with extra peaks similar to HCl salt Type A and B (marked in red frame). TGA/DSC curves in FIG. 2H showed a weight loss of 4.2% up to 130.0° C. and three endothermic signals at 94.5° C., 163.8° C. and 190.9° C. (peak temperature). HPLC/IC results showed that the molar ratio of acid/freebase was determined as 1.1:1 and HPLC purity was 97.02 area% (FIG. 21 and TABLE 2E).

TABLE 2E

HPLC results of starting material (824509-20-B)

| # | RRT | Area (%) | # | RRT | Area (%) |
|---|---|---|---|---|---|
| 1 | 0.73 | 0.29 | 9 | 0.98 | 0.48 |
| 2 | 0.87 | 0.05 | 10 | 1.00 | 97.02 |
| 3 | 0.88 | 0.24 | 11 | 1.07 | 0.26 |
| 4 | 0.90 | 0.08 | 12 | 1.08 | 0.06 |
| 5 | 0.91 | 0.08 | 13 | 1.10 | 0.13 |
| 6 | 0.93 | 0.12 | 14 | 1.11 | 0.12 |
| 7 | 0.94 | 0.37 | 15 | 1.14 | 0.06 |
| 8 | 0.96 | 0.63 | -- | -- | -- |

### Freebase Isolation

A detailed procedure for the preparation of 18-MC Freebase from 18-MC HCl Salt can be found below, and characterization results of prepared freebase batches are summarized in TABLE 2F. Additional data are shown in FIGS. 2J to 2P and TABLE 2F to 21.

TABLE 2F

Summary of prepared freebase samples

| Batch. ID (82450 9-) | Starting material Batch ID | Scale | Crystal form | TGA loss (%, up to) | Endother mic signal (°C., onset) | HPLC purity (area%) | Ion conte nt (Cl⁻, %) |
|---|---|---|---|---|---|---|---|
| | 824509 - | | | | | | |
| 03-A | 01-A | 9-g | Freeba se Type A | 1.1 (150° C.) | 196.2 | 99.31 | <0.24 |
| 21-A | 20-A | 3-g | | 1.1 (180° C.) | 196.7 | 99.47 | <0.15 |
| 24-A | 20-B | 6.5-g | | 1.9 (170° C.) | 192.7 | 98.23 | Not detect ed |

For freebase Type A (824509-03-A), the Cl- contents was determined to be less than 0.24%. TGA/DSC curves from FIG. 2K showed that up to 150° C., a TGA weight loss of 1.1 % was observed and one endothermic signal around 196.2° C. (onset) was detected. HPLC chromatograms from FIG. 2L displayed the HPLC purity was around 99.31 area% (TABLE 2G).

TABLE 2G

HPLC results of freebase Type A (824509-03-A)

| # | RRT | Area(%) | # | RRT | Area(%) |
|---|---|---|---|---|---|
| 1 | 0.92 | 0.15 | 4 | 1.07 | 0.27 |
| 2 | 0.97 | 0.09 | 5 | 1.27 | 0.05 |
| 3 | 1.00 | 99.31 | 6 | 1.31 | 0.13 |

For freebase Type A (824509-21-A), the Cl- contents was determined to be less than 0.15%. TGA/DSC curves from FIG. 2M showed that up to 180° C., a TGA weight loss of 1.1 % was observed and one endothermic signal around 196.7° C. (onset) were detected. HPLC chromatograms from FIG. 2N displayed that the HPLC purity was determined to be 99.45 area% (TABLE 2H).

TABLE 2H

HPLC results of freebase Type A (824509-21-A)

| # | RRT | Area (%) | # | RRT | Area (%) |
|---|---|---|---|---|---|
| 1 | 0.73 | 0.05 | 4 | 1.00 | 99.47 |
| 2 | 0.93 | 0.09 | 5 | 1.07 | 0.14 |
| 3 | 0.96 | 0.25 | -- | -- | -- |

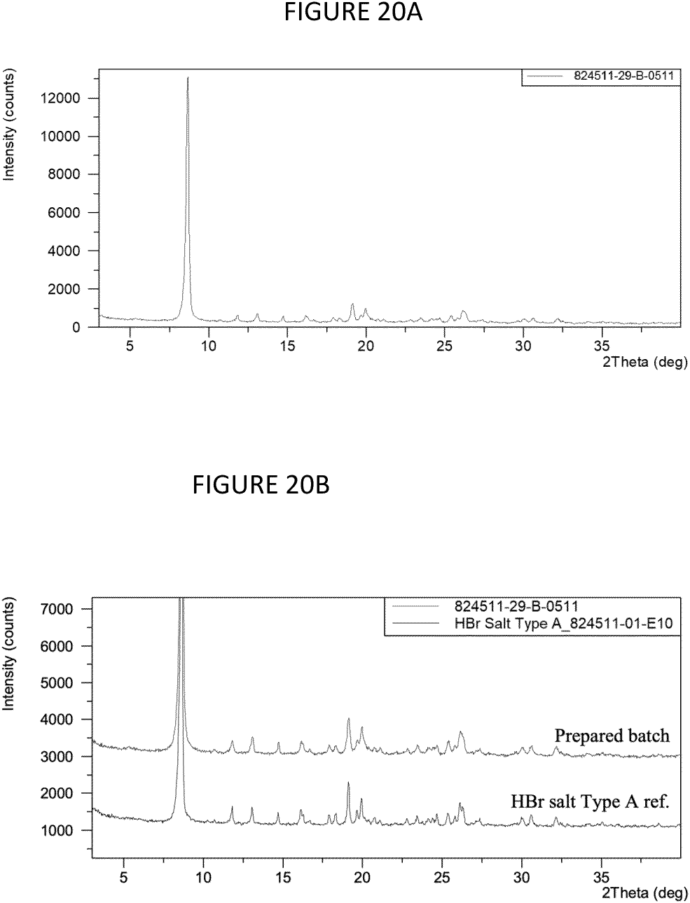

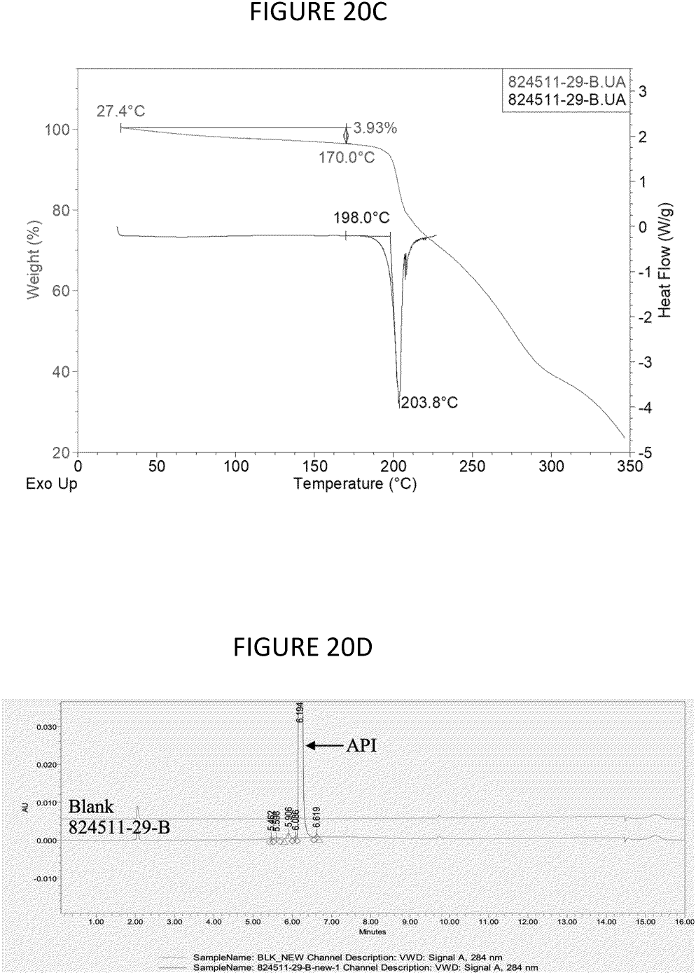

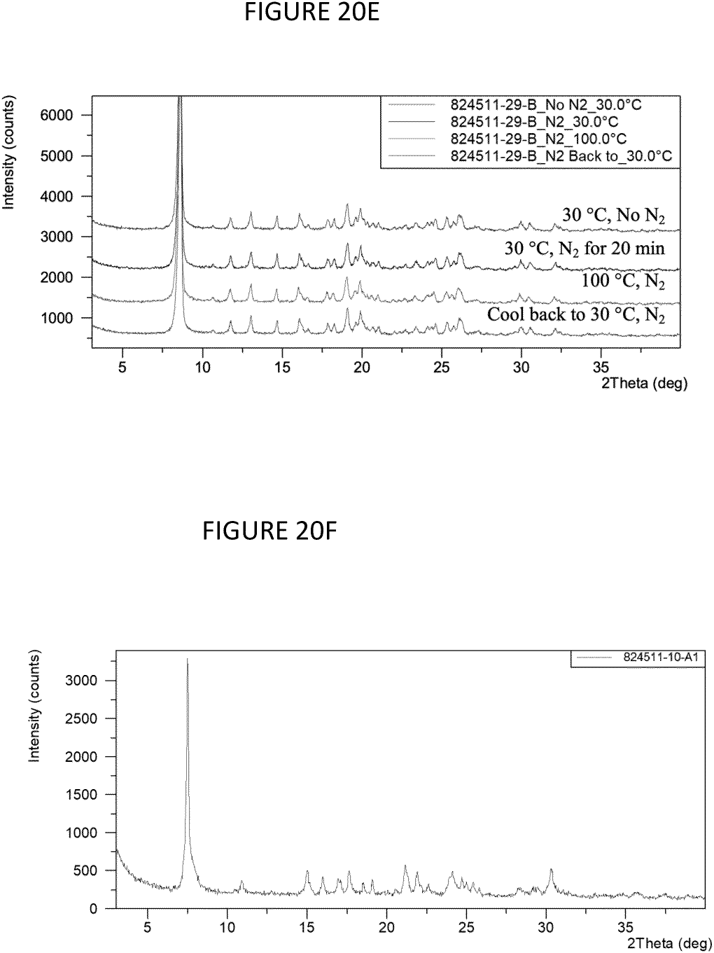

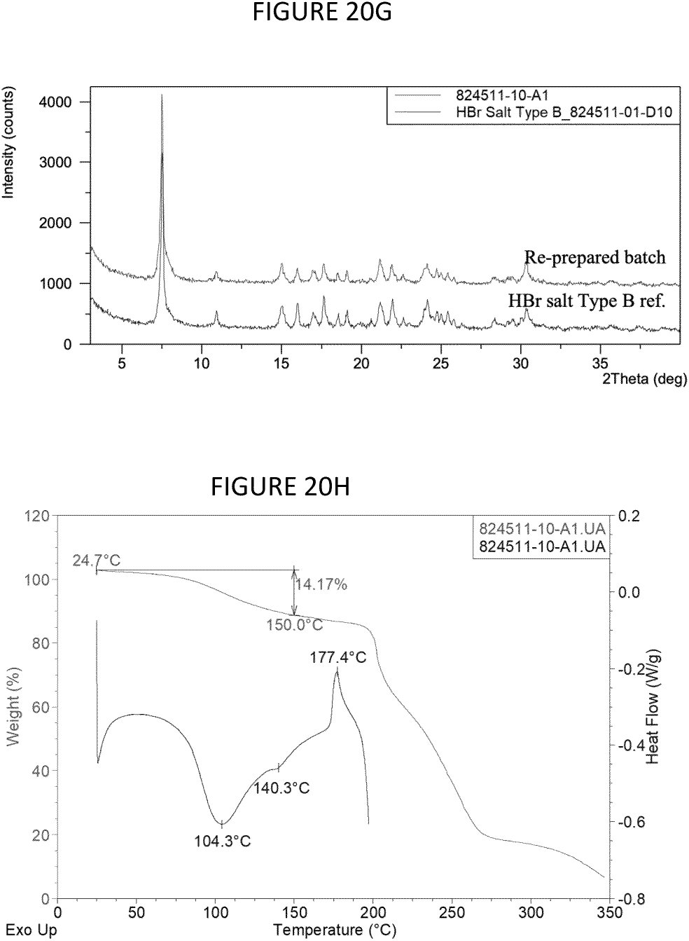

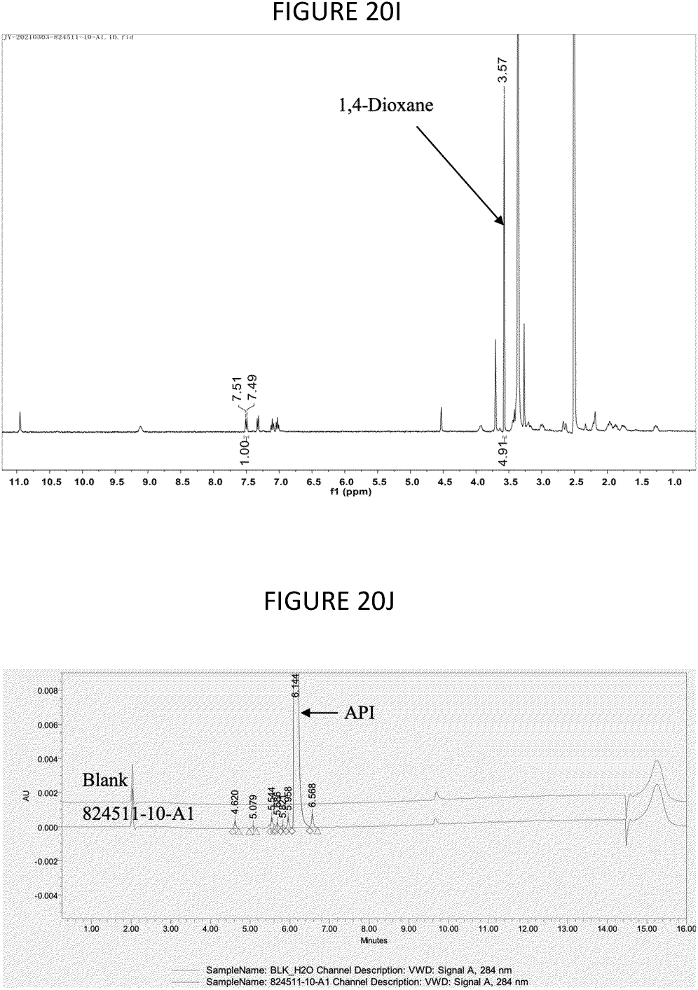

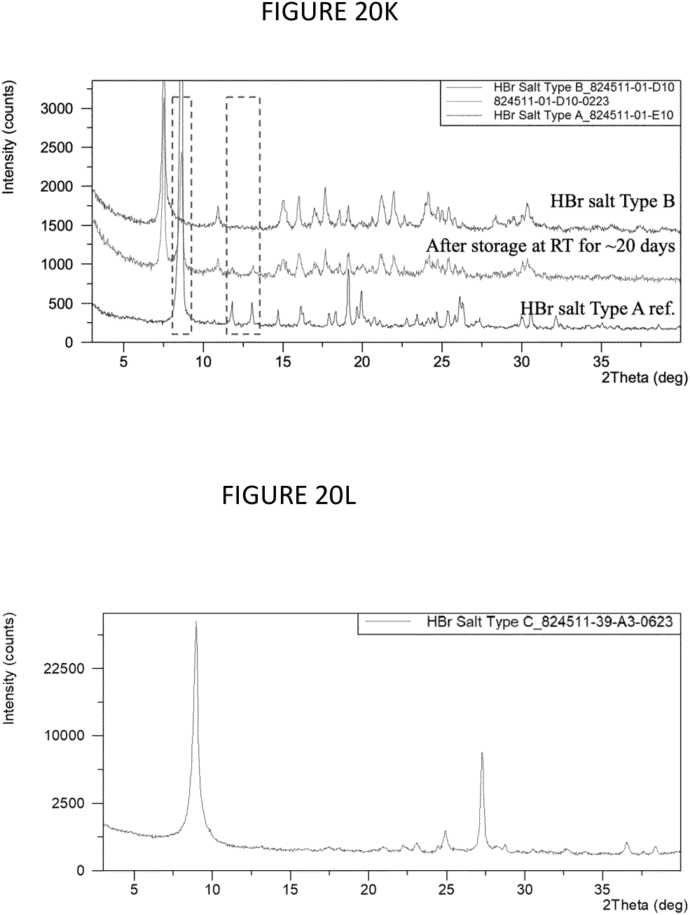

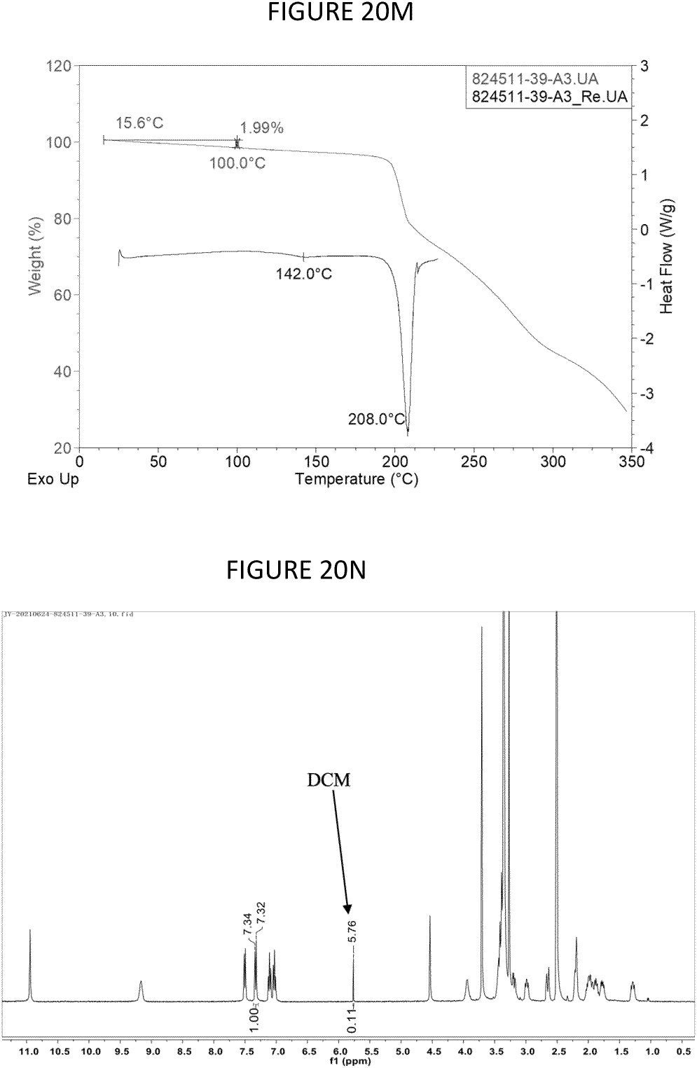

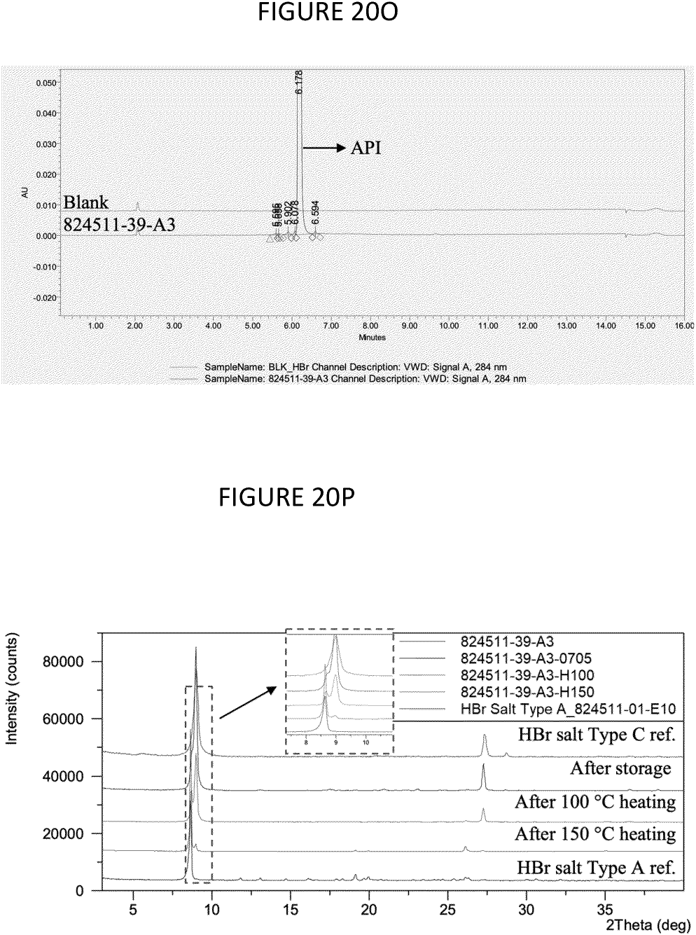

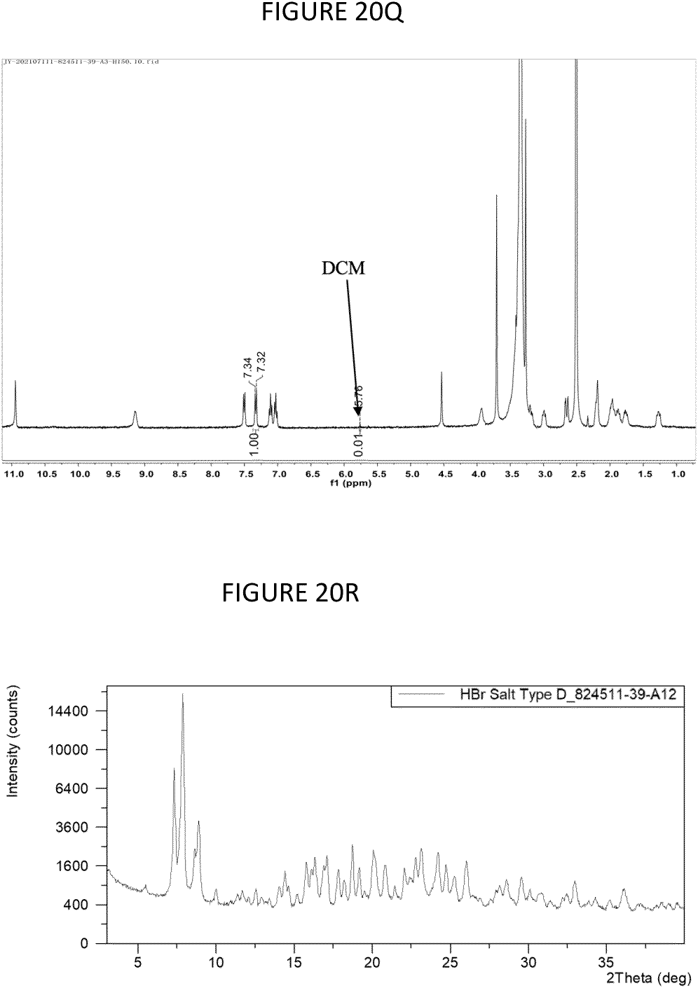

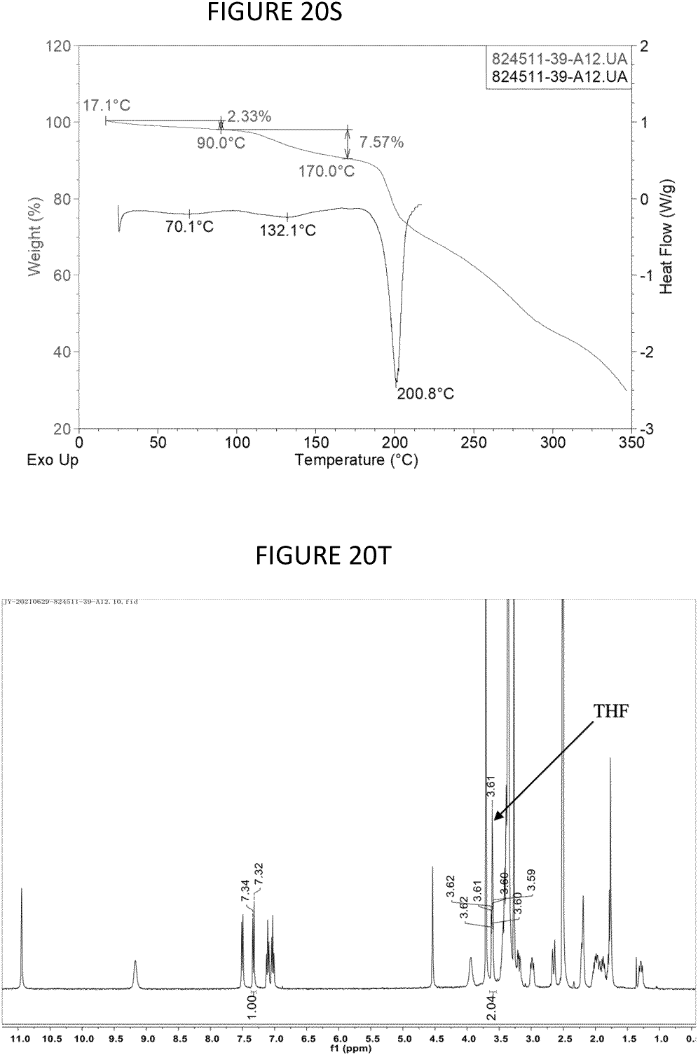

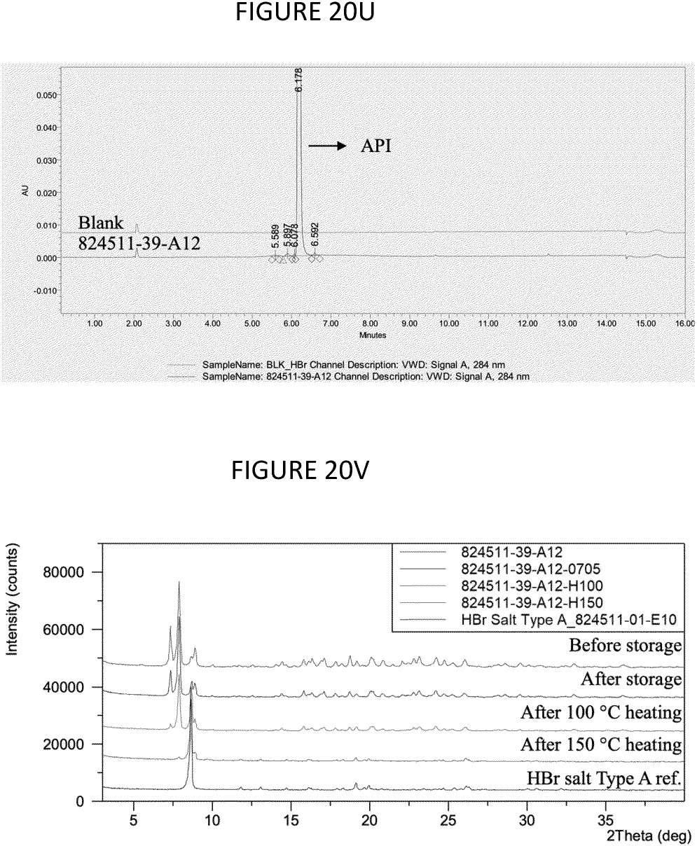

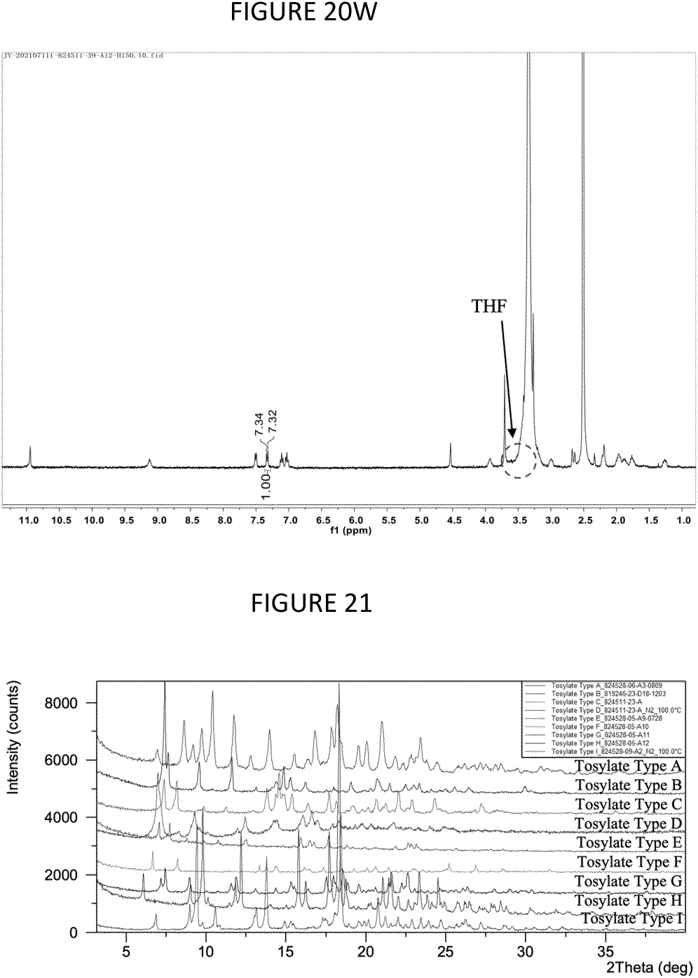

For freebase Type A (824509-24-A), no Cl- residual in the sample was detected. TGA/DSC curves from FIGS. 20 showed that up to 170° C., a TGA weight loss of 1.9% was observed and one endothermic signal around 192.7° C. (onset) was detected. HPLC chromatograms from FIG. 2P displayed that the HPLC purity was determined to be 98.23 area% (TABLE 21).

TABLE 2I

HPLC results of freebase Type A (824509-24-A)

| # | RRT | Area (%) | # | RRT | Area (%) |
|---|---|---|---|---|---|
| 1 | 0.73 | 0.26 | 7 | 0.96 | 0.41 |
| 2 | 0.88 | 0.07 | 8 | 0.98 | 0.37 |
| 3 | 0.90 | 0.07 | 9 | 1.00 | 98.23 |
| 4 | 0.91 | 0.06 | 10 | 1.07 | 0.15 |
| 5 | 0.93 | 0.11 | 11 | 1.10 | 0.05 |
| 6 | 0.94 | 0.23 | -- | -- | -- |

### Salt Screening

Using prepared freebase Type A (batch 824509-03-A and 824509-24-A) as starting material, a total of 100 experiments of salt screening were conducted with 20 acids and different solvent systems. For the CHCl₃ system, a stock solution of freebase was first prepared by dissolving ~150 mg of freebase Type A sample in 3.75 mL of CHCl₃. Then the corresponding acids (charge molar ratio of acid/freebase=1:1) were added in 0.5 mL of stock solutions and slurried at RT. For the remaining solvent systems (IPAc, MIBK, 1,4-dioxane, IPA, DCM/EtOAc (1:1, v/v), acetone/H20 (9:1, v/v) and ACN/THF (3:1, v/v)), about 20 mg of freebase Type A sample was weighed into each HPLC vial and mixed with corresponding solid acids (charge molar ratio of acid/freebase=1:1). About 0.5 mL of solvents were added into the vial and the mixture was transferred to slurry at RT. Liquid acids were first diluted with 0.25 mL solvent and then added into freebase solution (freebase in 0.25 mL solvent). After slurry for about 5 days, precipitates were separated by centrifugation and the resulting solids were vacuum-dried at RT overnight (4 to 15 hours). If oil or gel-like samples were obtained after slurry, the samples were transferred to temperature cycling (one cycle: ramp to 50° C. at a rate of 4.5° C./min, keep the temperature at 50° C. for 30 min; cool down to 5° C. at a rate of 0.1° C./min and keep the temperature at 5° C. for 30 min. 4 cycles were conducted). If clear solution was obtained, it was transferred to slurry at 5° C. If it was still clear, the solution was transferred to slurry at -20° C. If there was still no precipitate, the clear solution was transferred to evaporation at RT or anti-solvent addition to induce precipitation. After vacuum drying at RT, all the resulting dry solids were tested by XRPD.

As the XRPD comparison results in TABLE 3A and TABLE 3B shows, a total of 9 salt forms were discovered, including gentisate Type A, HBr salt Types A and B, besylate Types A and B and napadisylate Types A, B, C, and D. XRPD overlay was displayed in FIG. 1. The characterization data of these salt hits was summarized in TABLE 1A.

TABLE 3A

Summary of salt screening results

| # | Solvent Co-forme r | IPAc | MIBK | CHCl₃ | 1,4-Dioxane | IPA |
|---|---|---|---|---|---|---|
| 0 | Blank | FBA | FBA ¹ | FBA³ | FBA⁴ | FBA |
| 1 | H₃PO₄ | Amorphous | Amorphous¹ | Gel⁵ | Gel⁵ | FBA ¹ |
| 2 | L-Tartaric acid | FBA+acid | Amorphous¹ | Acid¹ | Gel⁷ | FBA |
| 3 | Fumaric acid | FBA+acid+ extra peak | FBA+acid+ extra peak | Acid¹ | Acid+extra peak¹ | FBA+acid+ extra peak |
| 4 | Citric acid | FBA+acid+ extra peak | Acid¹ | Gel² | Gel⁷ | FBA+extra peak |
| 5 | L-Malic acid | FBA ¹ | FBA+extra peak¹ | Gel² | Gel⁷ | FBA+extra peak |
| 6 | Hippuric acid | FBA+extra peak | FBA+ acid | Acid¹ | Acid¹ | FBA+extra peak |
| 7 | Acetic acid | FBA+extra peak¹ | FBA+extra peak¹ | FBA³ | FBA⁴ | FBA+extra peak |
| 8 | Malonic acid | FBA¹ | Gel⁶ | Gel³ | Gel¹ | FBA+extra peak |
| 9 | Gentisic acid | Gentisate Type A | Gentisate Type A¹ | Gentisate Type A | Gel⁷ | FBA |
| 1 0 | HBr | HBr salt Type A | HBr salt Type A | Low crystallinit y¹ | HBr salt Type B | HBr salt Type A |

FBA: freebase Type A. 1: transfer clear solution to slurry at 5° C. 2: transfer clear solution to slurry at 5° C. → slurry at -20° C. → temperature cycling (5~50° C.). 3: transfer clear solution to slurry at 5° C. → slurry at -20° C. → anti-solvent addition. 4: transfer clear solution to slurry at 5° C. → anti-solvent addition. 5: transfer oil/gel to temperature cycling (5~50° C.). 6: transfer clear solution to slurry at 5° C. → slurry at -20° C. → evaporation at RT. 7: transfer clear solution to slurry at 5° C. → anti-solvent addition → temperature cycling (5~50° C.).

TABLE 3B

Summary of additional salt screening results

| # | Solvent Co-former | DCM/EtOAc (1:1, v/v) (A) | Acetone/H₂O (9:1, v/v) (B) | ACN/THF (3:1, v/v) (C) | IPA (D) | 1,4-Dioxane (E) |
|---|---|---|---|---|---|---|
| 0 | Blank | FBA | FBA | FBA | FBA³ | FBA⁵ |
| 1 | D-Gluconic acid | FBA | FBA | FBA | FBA³ | Oil⁷ |
| 2 | Lactic acid | FBA² | FBA | FBA | FBA³ | FBA⁷ |
| 3 | Succinic acid | FBA | FBA | FBA | FBA³ | FBA+acid⁷ |
| 4 | Adipic acid | FBA | FBA | FBA | FBA³ | Amorphous⁵ |
| 5 | Benzoic acid | FBA | FBA | FBA | FBA+acid³ | FBA⁷ |
| 6 | Lauric acid | FBA² | FBA | FBA | FBA+acid³ | FBA+acid⁶ |
| 7 | Naphthalene-1,5-disulfonic acid | Napadisylate Type A | Napadisylate Type A | Napadisylate Type A | Napadisylate Type B | Napadisylate Type C^{4, 8} |
| 8 | 2-Hydroxyethanesulfonic acid | FBA | Gel¹ | FBA² | FBA³ | Oil⁷ |
| 9 | Tartaric acid | FBA | FBA | FBA² | FBA³ | Oil⁷ |
| 10 | Benzenesulfonic acid | Besylate Type A² | Gel¹ | Amorphous² | Besylate Type B | Besylate Type A |

FBA: freebase Type A.1: transfer clear solution to 5° C. slurry → slurry at -20° C. → anti-solvent (EtOAc) addition → temperature cycling (5~50° C.).2: transfer clear solution to 5° C. → slurry at -20° C. → temperature cycling (5~50° C.) → slurry at -20° C.3: transfer to slurry at 50° C. → add 25 µL H₂O →slurry at RT.4: transfer to slurry at 50° C.5: transfer clear solution to anti-solvent addition (EtOAc) → evaporation at RT.6: transfer clear solution to anti-solvent (n-heptane) addition.7: transfer clear solution to anti-solvent (n-heptane) addition → temperature cycling (5~50° C.). 8: Type D observed upon repreparation of Type C

### Characterization of Salt Hits

### Gentisate

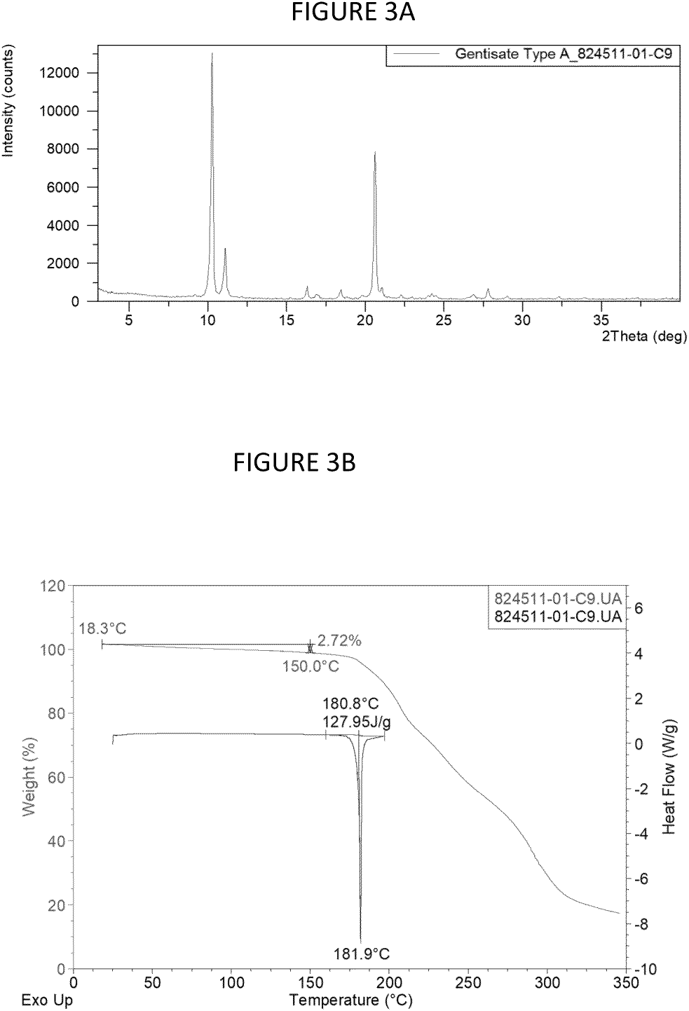

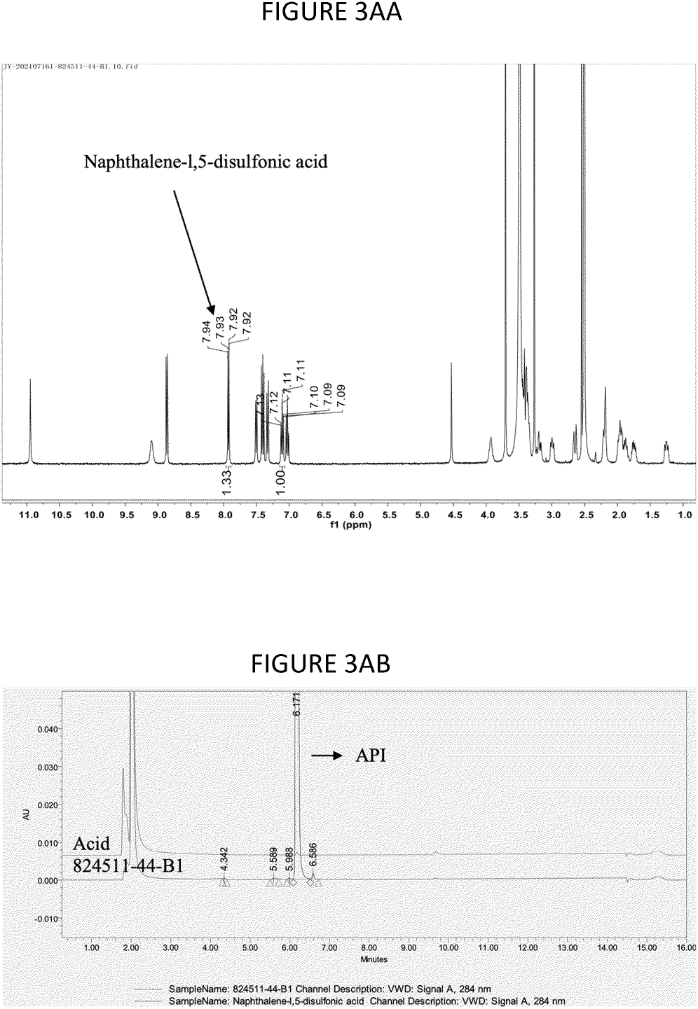

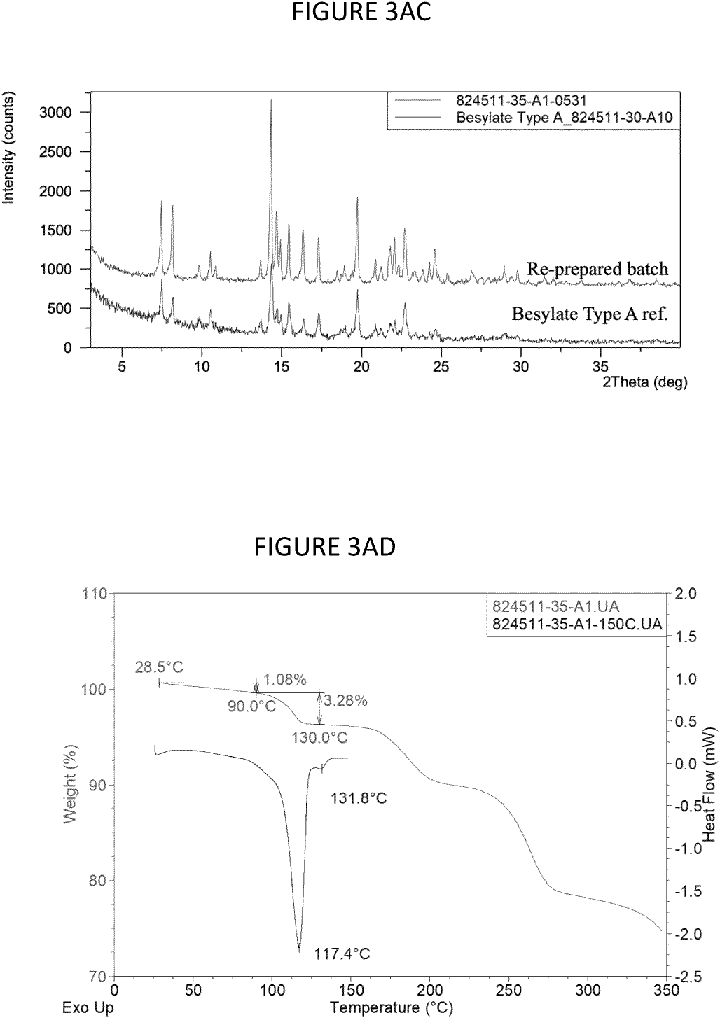

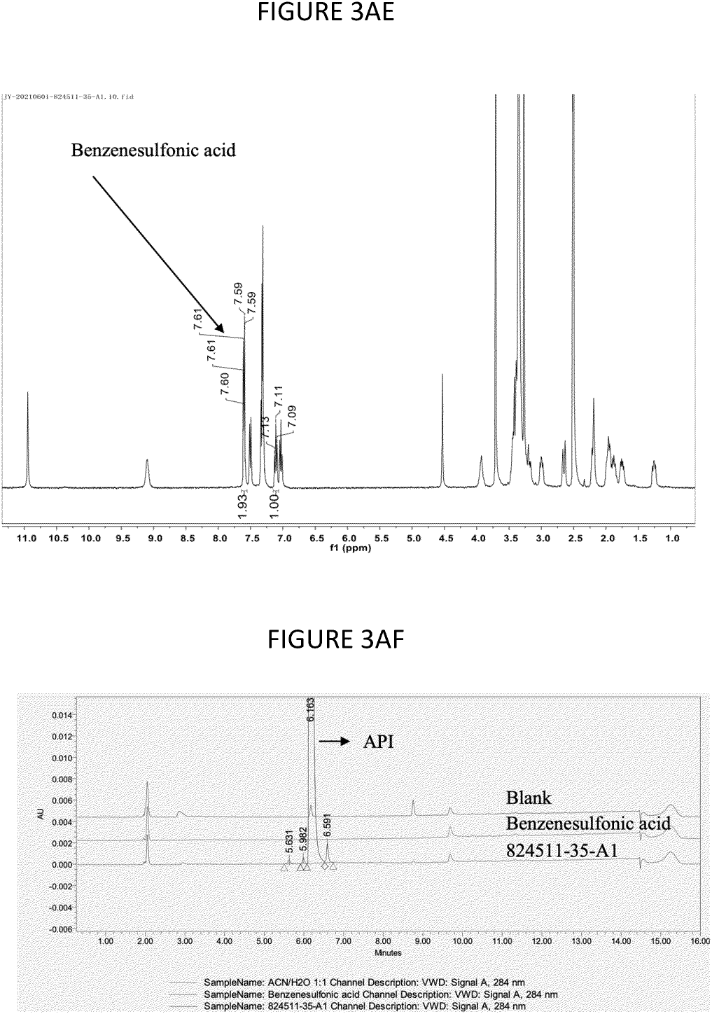

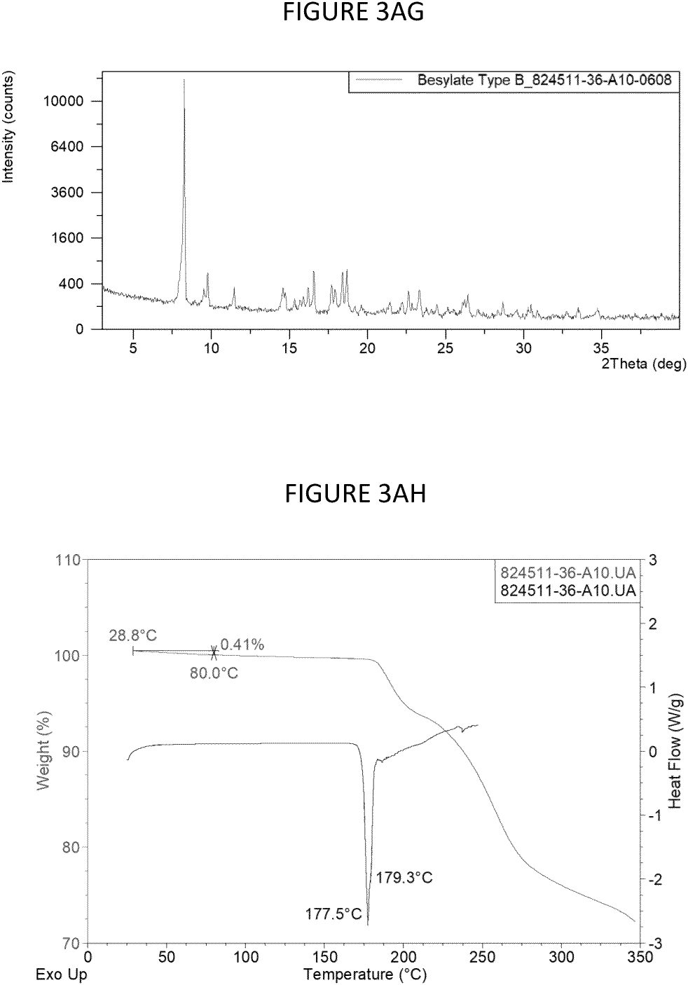

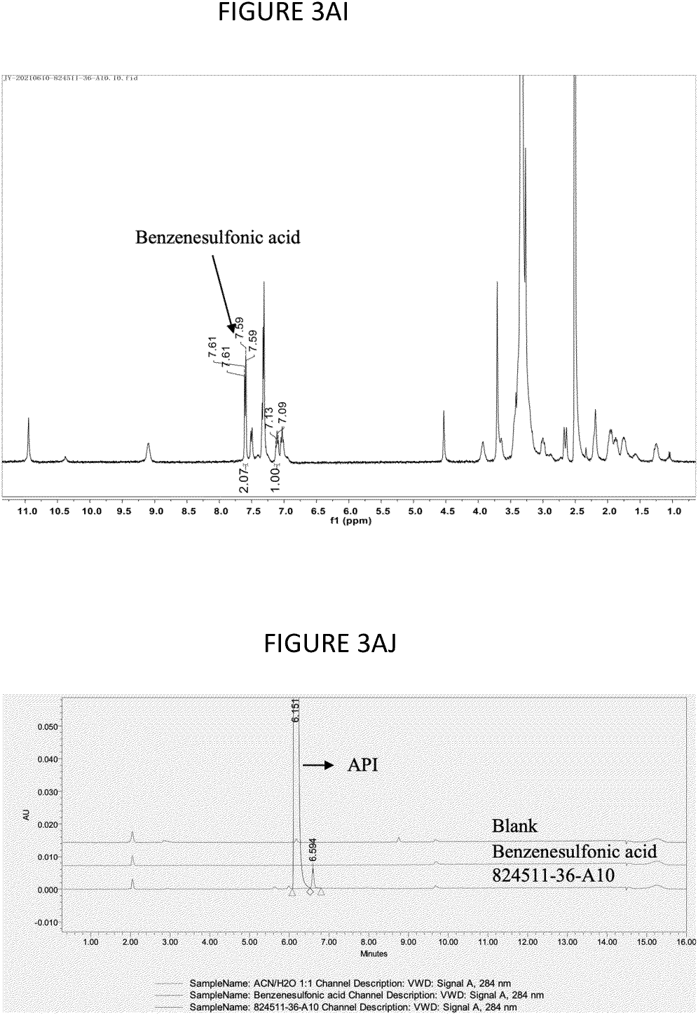

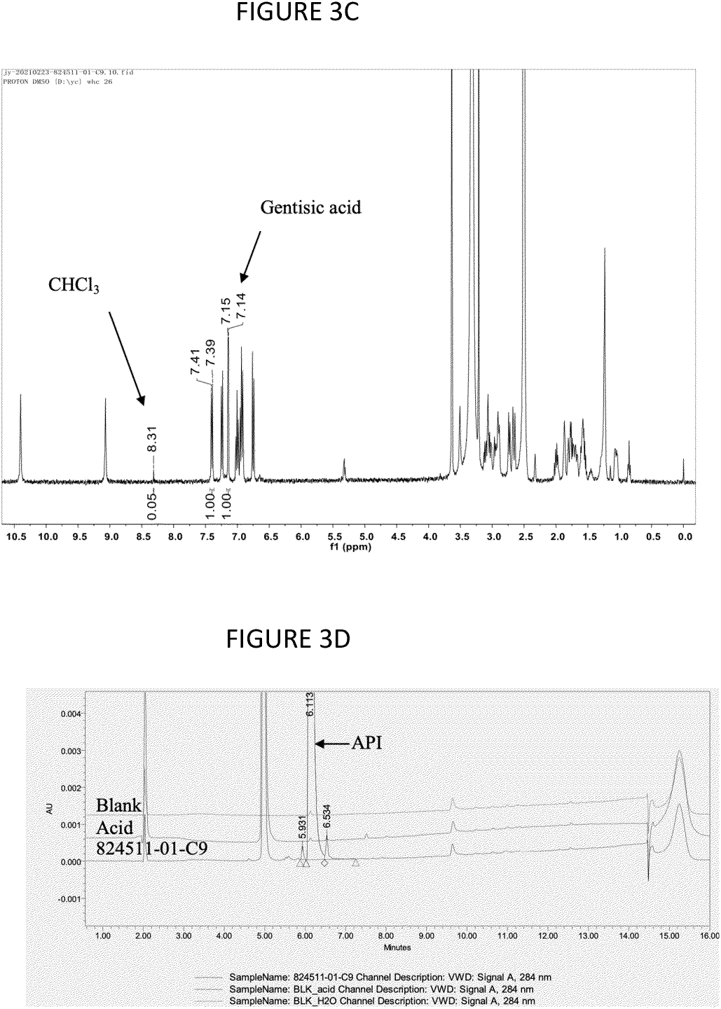

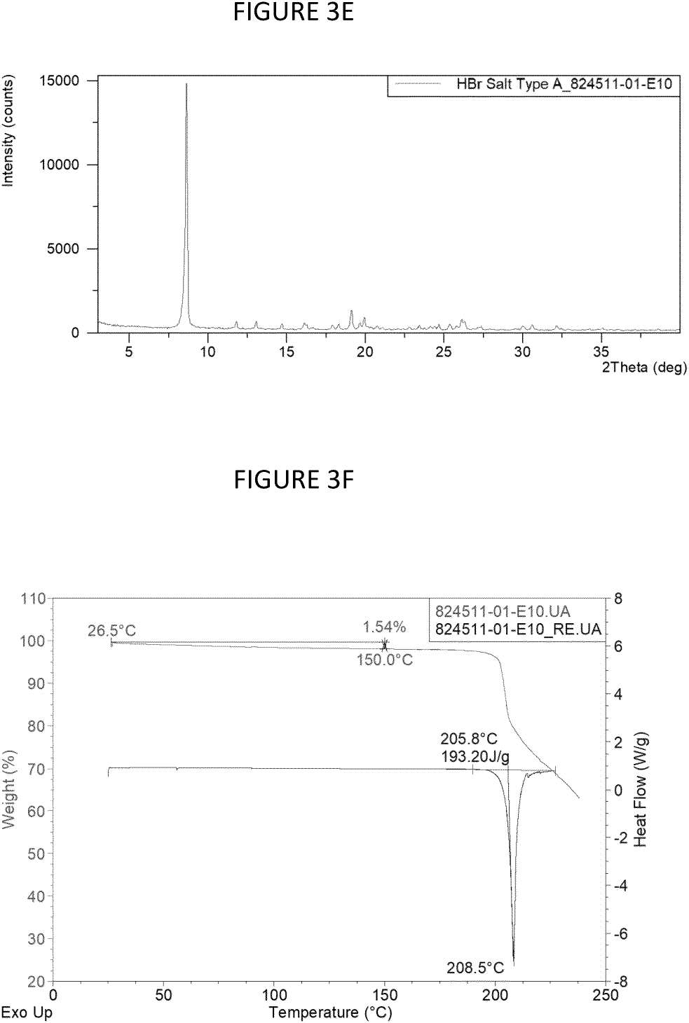

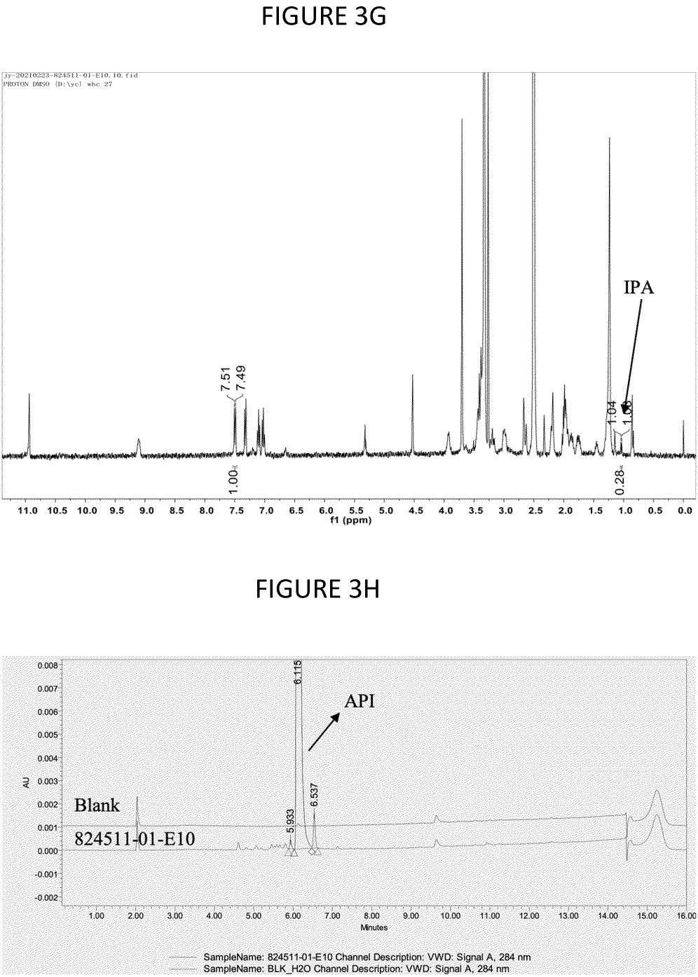

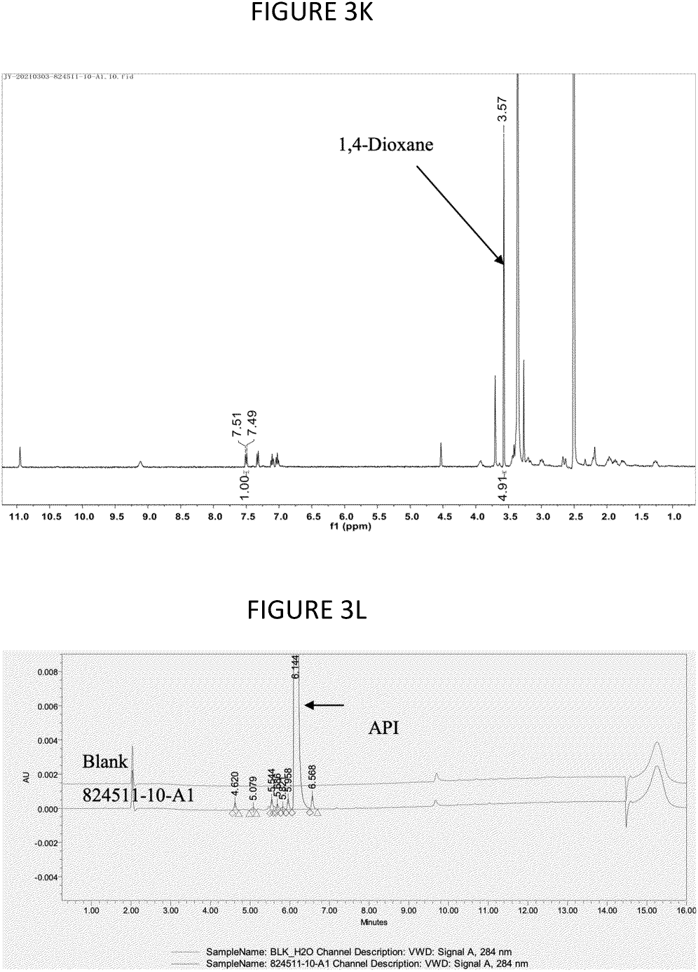

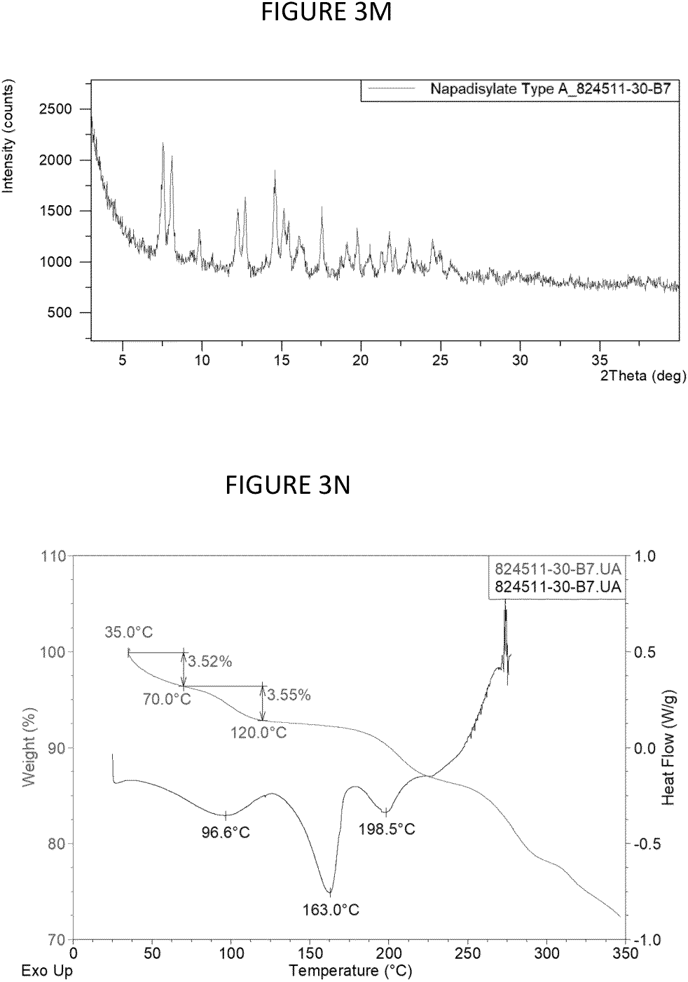

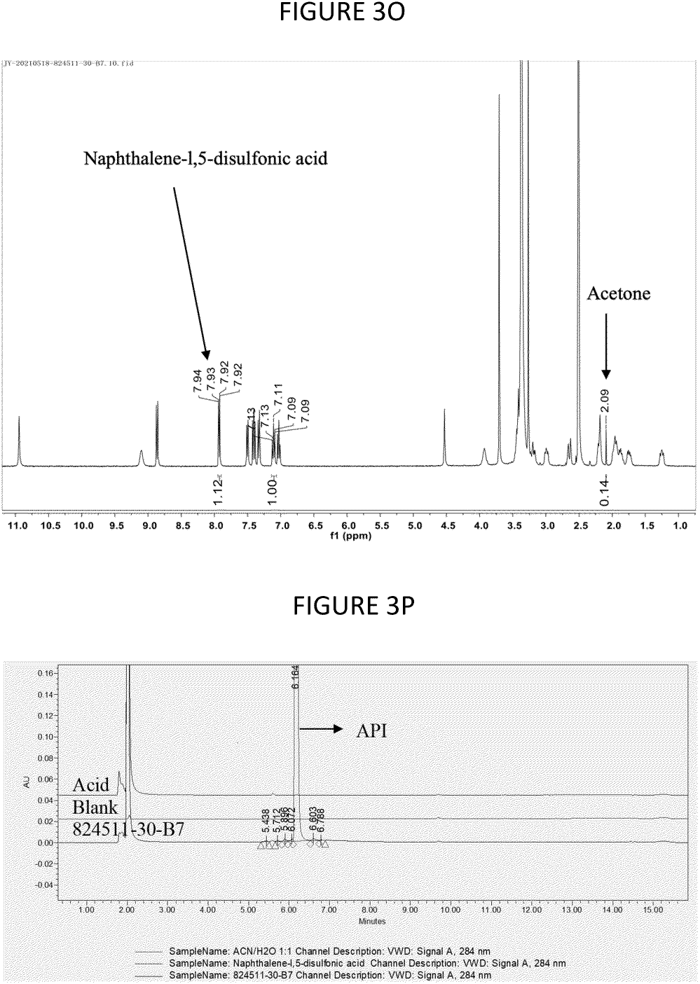

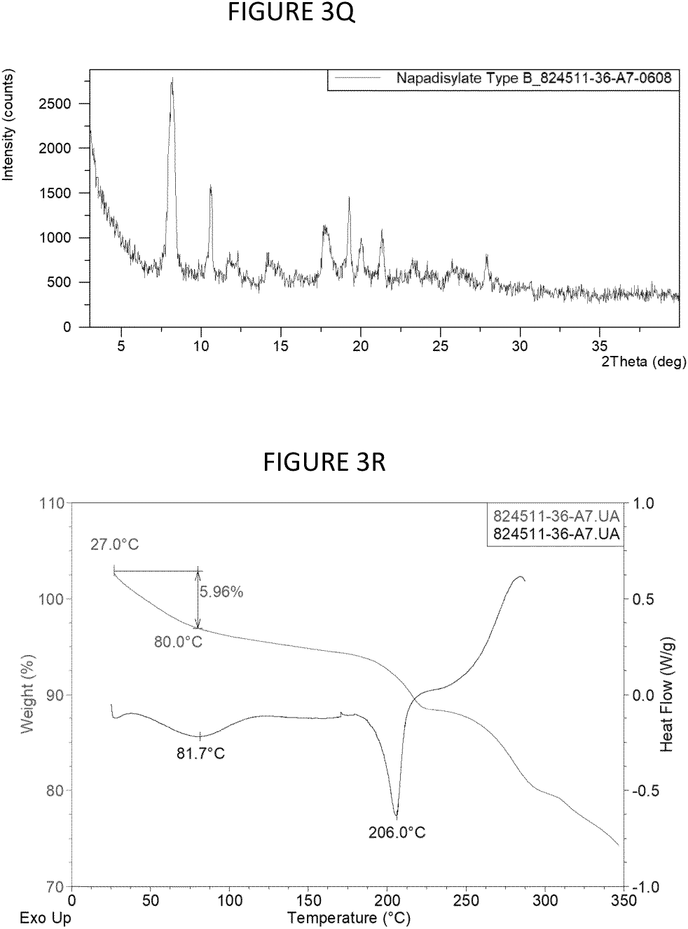

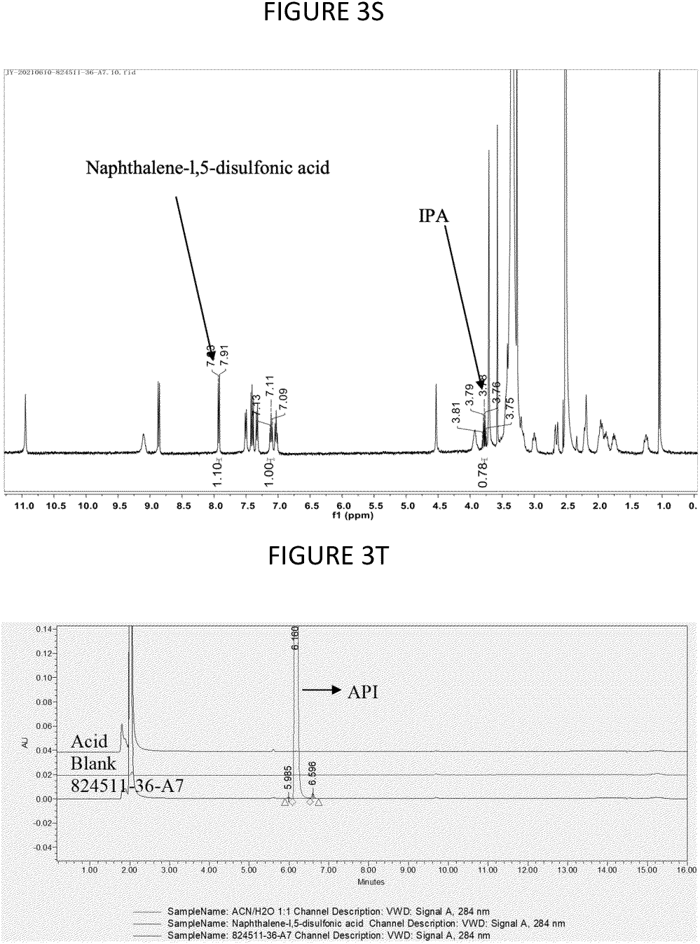

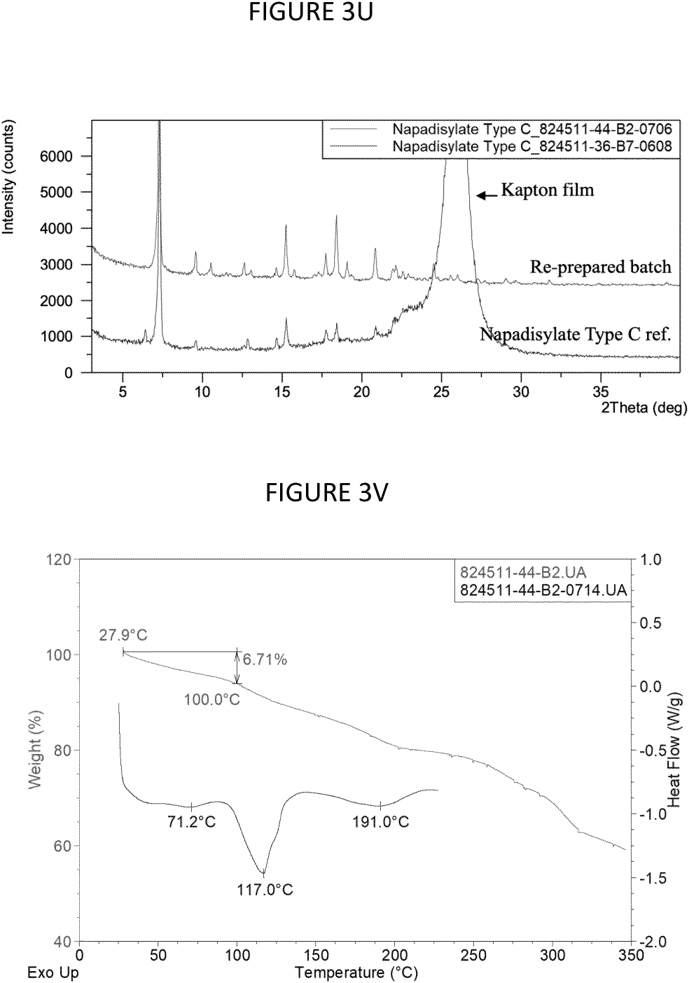

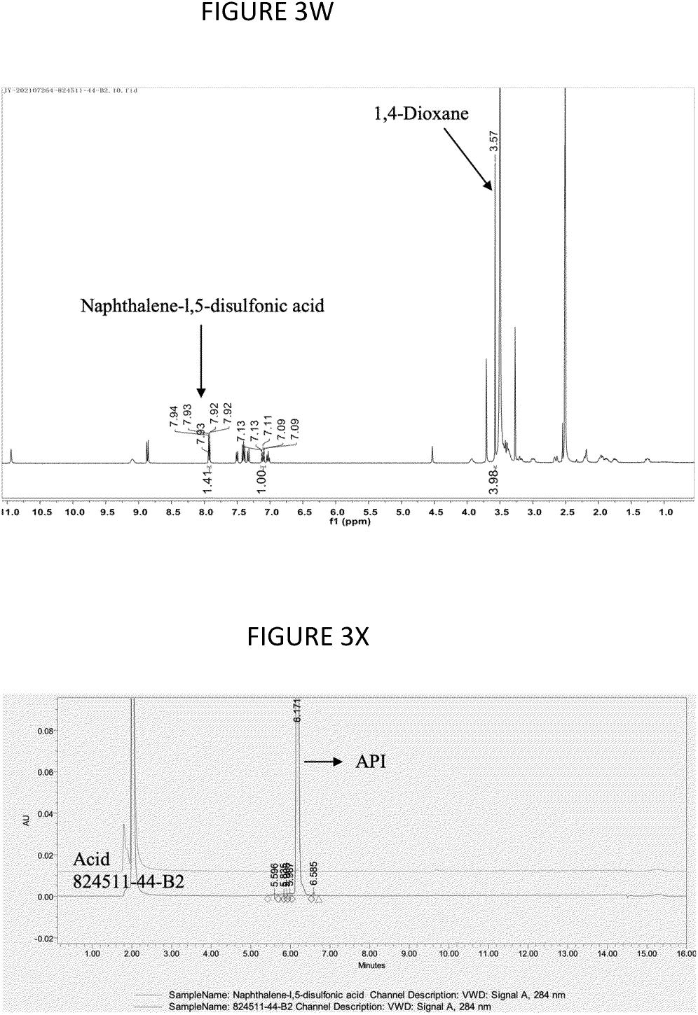

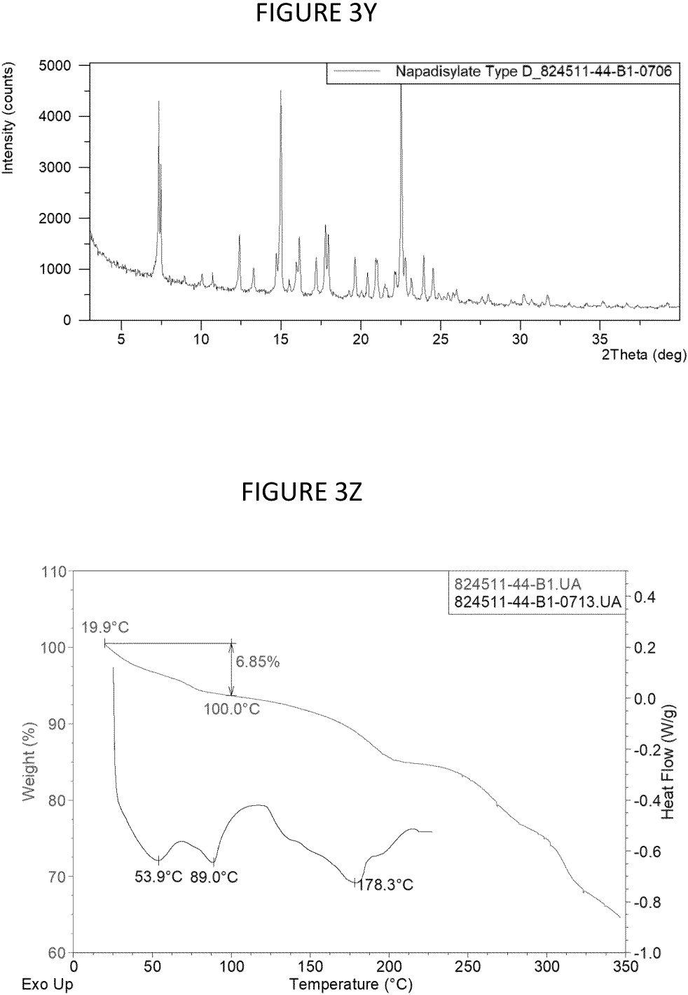

Gentisate Type A (824511-01-C9) was obtained by stirring 8.3 mg gentisic acid in 0.5 mL stock solution of freebase (40 mg/mL, charge molar ratio of acid to freebase was 1:1) in CHCl₃ at RT for about 5 days. Resulting solids were isolated by centrifugation and vacuum dried at RT overnight. The XRPD pattern was displayed in FIG. 3A and TABLE 4A. TGA/DSC curves in FIG. 3B showed that a weight loss of 2.7% up to 150° C. and one endothermic signal at 181.9° C. (peak) were detected. ¹H NMR spectrum in FIG. 3C showed that peaks of gentisic acid and CHCl₃ were observed. The molar ratio of acid/freebase was 1.0:1. The molar ratio of CHCl₃/API was 0.05:1 (theoretical weight=1.1 wt%). HPLC purity of the sample was determined as 99.79 area% (FIG. 3D and TABLE 4B).

TABLE 4A

XRPD peak list of gentisate Type A (824511-01-C9)

| Pos. [°2θ] | Height [cts] | FWHM Left [°2θ] | d-spacing [Å] | Rel. Int. [%] |
|---|---|---|---|---|
| 9.1817 | 127.34 | 0.1279 | 9.63 | 1.01 |
| 10.2605 | 12597.83 | 0.1279 | 8.62 | 100.00 |
| 11.0881 | 2626.30 | 0.1279 | 7.98 | 20.85 |
| 12.1337 | 77.17 | 0.1535 | 7.29 | 0.61 |
| 15.2280 | 62.83 | 0.1023 | 5.82 | 0.50 |
| 16.2933 | 657.24 | 0.1023 | 5.44 | 5.22 |
| 16.8740 | 226.16 | 0.0768 | 5.25 | 1.80 |
| 17.0382 | 170.44 | 0.0768 | 5.20 | 1.35 |
| 18.4405 | 467.94 | 0.1279 | 4.81 | 3.71 |
| 19.7916 | 176.25 | 0.1279 | 4.49 | 1.40 |
| 20.6077 | 7559.33 | 0.1279 | 4.31 | 60.01 |
| 21.0428 | 580.06 | 0.1023 | 4.22 | 4.60 |
| 22.2794 | 203.58 | 0.1279 | 3.99 | 1.62 |
| 22.9700 | 104.87 | 0.1023 | 3.87 | 0.83 |
| 23.9986 | 181.27 | 0.0768 | 3.71 | 1.44 |
| 24.2274 | 285.43 | 0.1023 | 3.67 | 2.27 |
| 24.4887 | 162.37 | 0.1279 | 3.64 | 1.29 |
| 26.8573 | 217.89 | 0.1023 | 3.32 | 1.73 |
| 27.7942 | 522.40 | 0.1535 | 3.21 | 4.15 |
| 29.0094 | 146.08 | 0.1023 | 3.08 | 1.16 |
| 32.2908 | 102.77 | 0.2047 | 2.77 | 0.82 |
| 33.9085 | 52.38 | 0.3070 | 2.64 | 0.42 |
| 37.2758 | 65.75 | 0.1535 | 2.41 | 0.52 |
| 39.1351 | 50.32 | 0.1535 | 2.30 | 0.40 |

TABLE 4B

HPLC results of gentisate Type A (824511-01-C9)

| # | RRT | Area (%) |
|---|---|---|
| 1 | 0.97 | 0.06 |
| 2 | 1.00 | 99.79 |
| 3 | 1.07 | 0.15 |

### HBr Salt

### HBr Salt Type A

HBr salt Type A (824511-01-E10) was obtained by diluting 11.0 µL HBr (~40% aqueous solution) in 0.25 mL IPA and suspending 19.8 mg freebase in 0.25 mL IPA at RT, then adding acid solution to freebase suspension (charge molar ratio of acid to freebase was 1:1) and slurry at RT for about 5 days. Resulting solids were isolated by centrifugation and vacuum dried at RT overnight. The XRPD pattern was displayed in FIG. 3E and TABLE 4C. As TGA/DSC curves in FIG. 3F shown, a weight loss of 1.5% up to 150° C. and one endothermic signal at 208.5° C. (peak) were detected. ¹H NMR spectrum in FIG. 3G showed that peak of IPA was observed. The molar ratio of IPA/API was 0.05:1 (theoretical 0.6 wt%). HPLC/IC results showed that the molar ratio of acid/freebase was 1.0:1 and HPLC purity was 99.72 area% (FIG. 3H and TABLE 4D).

TABLE 4C

XRPD peak list of HBr salt Type A (824511-01-E10)

| Pos. [°2θ] | Height [cts] | FWHM Left [°2θ] | d-spacing [Å] | Rel. Int. [%] |
|---|---|---|---|---|
| 8.6294 | 14255.69 | 0.1023 | 10.25 | 100.00 |
| 11.8002 | 433.74 | 0.1023 | 7.50 | 3.04 |
| 12.4169 | 6.18 | 0.8187 | 7.13 | 0.04 |
| 13.0640 | 441.13 | 0.1023 | 6.78 | 3.09 |
| 14.6945 | 296.47 | 0.1023 | 6.03 | 2.08 |
| 16.1237 | 401.23 | 0.1023 | 5.50 | 2.81 |
| 16.6371 | 83.21 | 0.1535 | 5.33 | 0.58 |
| 17.9081 | 250.55 | 0.1023 | 4.95 | 1.76 |
| 18.3027 | 275.61 | 0.1279 | 4.85 | 1.93 |
| 19.1154 | 1151.39 | 0.1279 | 4.64 | 8.08 |
| 19.6463 | 395.32 | 0.0768 | 4.52 | 2.77 |
| 19.9306 | 715.76 | 0.1279 | 4.45 | 5.02 |
| 20.3683 | 121.41 | 0.1279 | 4.36 | 0.85 |
| 20.7449 | 195.03 | 0.1023 | 4.28 | 1.37 |
| 21.0862 | 134.23 | 0.1023 | 4.21 | 0.94 |
| 22.0711 | 53.01 | 0.1535 | 4.03 | 0.37 |
| 22.7830 | 166.90 | 0.1279 | 3.90 | 1.17 |
| 23.4362 | 257.95 | 0.1023 | 3.80 | 1.81 |
| 24.1245 | 188.84 | 0.0768 | 3.69 | 1.32 |
| 24.4047 | 196.43 | 0.0768 | 3.65 | 1.38 |
| 24.6693 | 310.73 | 0.0768 | 3.61 | 2.18 |
| 25.3634 | 321.94 | 0.1535 | 3.51 | 2.26 |
| 25.7979 | 208.71 | 0.1023 | 3.45 | 1.46 |
| 26.1123 | 612.21 | 0.1023 | 3.41 | 4.29 |
| 26.3017 | 480.81 | 0.1023 | 3.39 | 3.37 |
| 27.3438 | 183.88 | 0.1023 | 3.26 | 1.29 |
| 29.9968 | 199.53 | 0.1279 | 2.98 | 1.40 |
| 30.5894 | 295.20 | 0.1535 | 2.92 | 2.07 |
| 32.1529 | 254.02 | 0.1279 | 2.78 | 1.78 |
| 32.9009 | 36.26 | 0.1535 | 2.72 | 0.25 |
| 34.1878 | 54.31 | 0.2558 | 2.62 | 0.38 |
| 35.0546 | 114.55 | 0.1023 | 2.56 | 0.80 |
| 35.5848 | 45.29 | 0.1791 | 2.52 | 0.32 |
| 36.0200 | 55.49 | 0.2047 | 2.49 | 0.39 |
| 38.6095 | 82.03 | 0.1279 | 2.33 | 0.58 |

TABLE 4D

HPLC results of HBr salt Type A (824511-01-E10)

| # | RRT | Area (%) |
|---|---|---|
| 1 | 0.97 | 0.06 |
| 2 | 1.00 | 99.72 |
| 3 | 1.07 | 0.23 |

### HBr Salt Type B

HBr salt Type B (824511-01-D10) was obtained by diluting 11.0 µL HBr (~40% aqueous solution) in 0.25 mL 1,4-dioxane and suspending 20.1 mg freebase in 0.25 mL 1,4-dioxane at RT, then adding acid solution to freebase suspension (charge molar ratio of acid to freebase was 1:1) and slurry at RT for about one week. Resulting solids were isolated by centrifugation and vacuum dried at RT overnight. Another batch of HBr salt Type B (824511-10-A1) was prepared using the same method, and the XRPD overlay was displayed in FIG. 3I with XRPD peak list shown in TABLE 4E. As TGA/DSC curves in FIG. 3J showed, a weight loss of 14.2% up to 150° C., two endothermic signals at 104.3° C. (peak), 140.3° C. (peak) and one exothermic signal at 177.4° C. (peak) were detected. ¹H NMR spectrum in FIG. 3K showed that a peak of 1,4-dioxane was observed. The molar ratio of 1,4-dioxane/API was 0.6:1 (theoretical 10.8 wt%). HPLC/IC results showed that the molar ratio of acid/freebase was determined as 0.9:1 and HPLC purity was 99.27 area% (FIG. 3L and TABLE 4F).

TABLE 4E

XRPD peak list of HBr salt Type B (824511-10-A1)

| Pos. [°2θ] | Height [cts] | FWHM Left [°2θ] | d-spacing [Å] | Rel. Int. [%] |
|---|---|---|---|---|
| 7.5029 | 2970.00 | 0.1023 | 11.78 | 100.00 |
| 10.9066 | 161.08 | 0.1279 | 8.11 | 5.42 |
| 15.0145 | 311.24 | 0.1279 | 5.90 | 10.48 |
| 15.9789 | 222.39 | 0.1023 | 5.55 | 7.49 |
| 16.9544 | 204.96 | 0.0768 | 5.23 | 6.90 |
| 17.6381 | 304.55 . | 0.1279 | 5.03 | 10.25 |
| 18.5133 | 132.76 | 0.1279 | 4.79 | 4.47 |
| 19.1127 | 186.48 | 0.1279 | 4.64 | 6.28 |
| 20.5876 | 62.04 | 0.1535 | 4.31 | 2.09 |
| 21.1695 | 393.29 | 0.1023 | 4.20 | 13.24 |
| 21.9105 | 306.00 | 0.1279 | 4.06 | 10.30 |
| 22.6171 | 147.40 | 0.1023 | 3.93 | 4.96 |
| 24.1238 | 321.90 | 0.0768 | 3.69 | 10.84 |
| 24.7214 | 243.90 | 0.1023 | 3.60 | 8.21 |
| 25.0050 | 170.78 | 0.1023 | 3.56 | 5.75 |
| 25.4176 | 176.61 | 0.1023 | 3.50 | 5.95 |
| 25.7943 | 87.46 | 0.1535 | 3.45 | 2.94 |
| 28.3150 | 85.45 | 0.2558 | 3.15 | 2.88 |
| 29.4915 | 103.39 | 0.1535 | 3.03 | 3.48 |
| 30.3344 | 318.47 | 0.1791 | 2.95 | 10.72 |
| 35.6563 | 55.44 | 0.3070 | 2.52 | 1.87 |
| 37.4665 | 43.13 | 0.3070 | 2.40 | 1.45 |

TABLE 4F

HPLC results of HBr salt Type B (824511-10-A1)

| # | RRT | Area(%) | # | RRT | Area(%) |
|---|---|---|---|---|---|
| 1 | 0.75 | 0.09 | 5 | 0.95 | 0.06 |
| 2 | 0.83 | 0.05 | 6 | 0.97 | 0.13 |
| 3 | 0.90 | 0.13 | 7 | 1.00 | 99.27 |
| 4 | 0.93 | 0.09 | 8 | 1.07 | 0.17 |

### Napadisylate

### Napadisylate Type A

Napadisylate Type A (824511-30-B7) was obtained by slurry ~20.0 mg freebase and 16.2 mg naphthalene-1,5-disulfonic acid (charge molar ratio of acid to freebase was 1:1) in 0.5 mL acetone/H₂O (9:1, v/v) at RT for about one week. Resulting solids were isolated by centrifugation and vacuum drying at RT overnight. The XRPD pattern was displayed in FIG. 3M and TABLE 4G. As TGA/DSC curves in FIG. 3N showed, a weight loss of 3.5% up to 70° C., 3.6% from 70° C. up to 120° C. and three endothermic signals at 96.6° C., 163.0° C. and 198.5° C. (peak) were observed. ¹H NMR spectrum in FIGURE showed that the peak of naphthalene-I,5-disulfonic acid and acetone were observed. The molar ratio of acid/API was 0.6:1, the molar ratio of acetone/API was 0.02:1 (theoretical 0.26 wt%). HPLC purity was 99.21 area% (FIG. 3P and TABLE 4H).

TABLE 4G

XRPD peak list of napadisylate Type A (824511-30-B7)

| Pos. [°2θ] | Height [cts] | FWHM Left [°2θ] | d-spacing [Å] | Rel. Int. [%] |
|---|---|---|---|---|
| 7.5590 | 369.15 | 0.1535 | 11.70 | 100.00 |
| 8.0918 | 331.47 | 0.1279 | 10.93 | 89.79 |
| 9.8237 | 116.89 | 0.1535 | 9.00 | 31.66 |
| 12.2391 | 199.64 | 0.1535 | 7.23 | 54.08 |
| 12.7037 | 244.74 | 0.1279 | 6.97 | 66.30 |
| 14.5713 | 329.16 | 0.1791 | 6.08 | 89.17 |
| 15.1337 | 209.69 | 0.0768 | 5.85 | 56.80 |
| 15.4305 | 168.88 | 0.1023 | 5.74 | 45.75 |
| 16.1110 | 121.79 | 0.1535 | 5.50 | 32.99 |
| 17.5413 | 206.43 | 0.1279 | 5.06 | 55.92 |
| 19.0937 | 100.64 | 0.1535 | 4.65 | 27.26 |
| 19.7584 | 152.65 | 0.1023 | 4.49 | 41.35 |
| 20.5482 | 77.15 | 0.1535 | 4.32 | 20.90 |
| 21.2856 | 82.37 | 0.1535 | 4.17 | 22.31 |
| 21.7766 | 151.54 | 0.1535 | 4.08 | 41.05 |
| 22.1355 | 84.14 | 0.1279 | 4.02 | 22.79 |
| 23.0237 | 123.09 | 0.2047 | 3.86 | 33.34 |
| 24.4909 | 115.98 | 0.2047 | 3.63 | 31.42 |
| 24.9477 | 79.18 | 0.2047 | 3.57 | 21.45 |
| 25.6560 | 48.52 | 0.2558 | 3.47 | 13.14 |

TABLE 4H

HPLC results of napadisylate Type A (824511-30-B7)

| # | RRT | Area (%) | # | RRT | Area (%) |
|---|---|---|---|---|---|
| 1 | 0.88 | 0.09 | 5 | 1.00 | 99.21 |
| 2 | 0.93 | 0.10 | 6 | 1.07 | 0.22 |
| 3 | 0.96 | 0.19 | 7 | 1.10 | 0.05 |
| 4 | 0.99 | 0.15 | -- | -- | -- |

### Napadisylate Type B

Napadisylate Type B (824511-36-A7) was obtained by slurry ~20.0 mg freebase and 16.1 mg naphthalene-1,5-disulfonic acid (charge molar ratio 1:1) in 0.5 mL IPA at RT for about 3 days and then transfer to slurry at 50° C. for about 4 days. Resulting solids were isolated by centrifugation and vacuum drying at RT overnight. The XRPD pattern was displayed in FIG. 3Q and TABLE 41. As TGA/DSC curves in FIG. 3R showed, a weight loss of 6.0% up to 80° C. and two endothermic signals at 81.7° C. and 206.0° C. (peak) were observed. ¹H NMR spectrum in FIG. 3S showed that the peaks of naphthalene-I,5-disulfonic acid and IPA were observed. The molar ratio of acid/API was 0.6:1, the molar ratio of IPA/API was 0.78:1 (theoretical 8.17 wt%). HPLC purity was 99.66 area% (FIG. 3T and TABLE 4J).

TABLE 4I

XRPD peak list of napadisylate Type B (824511-36-A7)

| Pos. [°2θ] | Height [cts] | FWHM Left [°2θ] | d-spacing [Å] | Rel. Int. [%] |
|---|---|---|---|---|
| 8.1854 | 566.07 | 0.4605 | 10.80 | 100.00 |
| 10.6098 | 249.36 | 0.1535 | 8.34 | 44.05 |
| 11.9690 | 50.61 | 0.6140 | 7.39 | 8.94 |
| 14.2853 | 58.16 | 0.4093 | 6.20 | 10.27 |
| 17.7501 | 154.55 | 0.2558 | 5.00 | 27.30 |
| 19.2908 | 241.70 | 0.1535 | 4.60 | 42.70 |
| 20.0441 | 126.65 | 0.2558 | 4.43 | 22.37 |
| 21.3366 | 138.12 | 0.2558 | 4.16 | 24.40 |
| 23.3454 | 63.10 | 0.3070 | 3.81 | 11.15 |
| 25.9193 | 59.57 | 0.8187 | 3.44 | 10.52 |
| 27.9045 | 84.51 | 0.1791 | 3.20 | 14.93 |

TABLE 4J

HPLC results of napadisylate Type B (824511-36-A7)

| # | RRT | Area (%) |
|---|---|---|
| 1 | 0.97 | 0.07 |
| 2 | 1.00 | 99.66 |
| 3 | 1.07 | 0.27 |

### Napadisylate Type C

Napadisylate Type C (824511-36-B7) was obtained by slurry ~20.0 mg freebase and 16.1 mg naphthalene-1,5-disulfonic acid (charge molar ratio of acid to freebase was 1:1) in 0.5 mL 1,4-dioxane for about 3 days and then transfer to slurry at 50° C. for about 4 days. Resulting solids were isolated by centrifugation and vacuum drying at RT overnight. The XRPD overlay was displayed in FIG. 3U, with XRPD peak list of (824511-44-B2) shown in TABLE 4K. As TGA/DSC curves in FIG. 3V showed, a weight loss of 6.7% up to 100° C. and three endothermic signals at 71.2° C., 117.0° C. and 191.0° C. (peak) were observed. ¹H NMR spectrum in FIG. 3W showed that the peak of naphthalene-I,5-disulfonic acid and 1,4-dioxane were observed. The molar ratio of acid/API was 0.7:1, the molar ratio of 1,4-dioxane/API was 0.50:1 (theoretical 8.91 wt%). HPLC purity was 98.42 area% (FIG. 3X and TABLE 4L).

TABLE 4 K

XRPD peak list of napadisylate Type C (824511-44-B2)

| Pos. [°2θ] | Height [cts] | FWHM Left [°2θ] | d-spacing [Å] | Rel. Int. [%] |
|---|---|---|---|---|
| 7.2923 | 7789.88 | 0.1023 | 12.12 | 100.00 |
| 9.5730 | 588.37 | 0.0768 | 9.24 | 7.55 |
| 10.5235 | 346.18 | 0.1023 | 8.41 | 4.44 |
| 11.5995 | 38.97 | 0.4093 | 7.63 | 0.50 |
| 12.6140 | 369.12 | 0.1023 | 7.02 | 4.74 |
| 13.0370 | 179.69 | 0.0768 | 6.79 | 2.31 |
| 14.6313 | 272.05 | 0.1023 | 6.05 | 3.49 |
| 15.2391 | 1451.54 | 0.1023 | 5.81 | 18.63 |
| 15.7681 | 210.88 | 0.0768 | 5.62 | 2.71 |
| 17.2893 | 197.05 | 0.0768 | 5.13 | 2.53 |
| 17.7433 | 693.00 | 0.1279 | 5.00 | 8.90 |
| 18.4071 | 1792.78 | 0.1279 | 4.82 | 23.01 |
| 19.0781 | 504.02 | 0.1023 | 4.65 | 6.47 |
| 19.3685 | 120.24 | 0.1023 | 4.58 | 1.54 |
| 20.8494 | 888.33 | 0.1535 | 4.26 | 11.40 |
| 21.9545 | 328.65 | 0.1279 | 4.05 | 4.22 |
| 22.1514 | 437.61 | 0.0768 | 4.01 | 5.62 |
| 22.5845 | 252.68 | 0.1023 | 3.94 | 3.24 |
| 22.9194 | 204.74 | 0.0768 | 3.88 | 2.63 |
| 23.9406 | 92.65 | 0.1535 | 3.72 | 1.19 |
| 24.5175 | 507.11 | 0.1279 | 3.63 | 6.51 |
| 25.5819 | 182.67 | 0.1023 | 3.48 | 2.35 |
| 26.0179 | 209.95 | 0.1023 | 3.42 | 2.70 |
| 27.2933 | 108.14 | 0.1791 | 3.27 | 1.39 |
| 27.7339 | 71.55 | 0.1023 | 3.22 | 0.92 |
| 29.0583 | 116.66 | 0.1279 | 3.07 | 1.50 |
| 29.6691 | 82.88 | 0.1535 | 3.01 | 1.06 |
| 31.7774 | 116.40 | 0.1279 | 2.82 | 1.49 |
| 33.2665 | 16.70 | 0.8187 | 2.69 | 0.21 |
| 34.8785 | 29.34 | 0.2047 | 2.57 | 0.38 |
| 39.1347 | 68.99 | 0.1535 | 2.30 | 0.89 |

TABLE 4L

HPLC results of napadisylate Type C (824511-44-B2)

| # | RRT | Area (%) | # | RRT | Area (%) |
|---|---|---|---|---|---|
| 1 | 0.91 | 0.42 | 4 | 0.97 | 0.34 |
| 2 | 0.95 | 0.31 | 5 | 1.00 | 98.42 |
| 3 | 0.96 | 0.24 | 6 | 1.07 | 0.28 |

### Napadisylate Type D

Napadisylate Type D (824511-44-B1) was discovered in the re-preparation trial of napadisylate Type C by slurry 20.2 mg freebase and 16.0 mg naphthalene-1,5-disulfonic acid (charge molar ratio of acid to freebase was 1:1) in 0.5 mL 1,4-dioxane at RT for 8 days. Resulting solids were isolated by centrifugation and vacuum drying at RT overnight. The XRPD overlay was displayed in FIG. 3Y with peak list of (824511-44-B1) shown in TABLE 4 M. As TGA/DSC curves in FIG. 3Z showed, a weight loss of 6.9% up to 100° C. and three endothermic signals at 53.9° C., 89.0° C. and 178.3° C. (peak) were observed. ¹H NMR spectrum in FIG. 3AA showed that the peak of naphthalene-I,5-disulfonic acid was observed. The molar ratio of acid/API was 0.7:1. No obvious solvent residual was detected. HPLC purity was 99.56 area% (FIG. 3AB and TABLE 4N).

TABLE 4 M

XRPD peak list of napadisylate Type D (824511-44-B1)

| Pos. [°2θ] | Height [cts] | FWHM Left [°2θ] | d-spacing [Å] | Rel. Int. [%] |
|---|---|---|---|---|
| 7.3319 | 2506.87 | 0.0512 | 12.06 | 76.59 |
| 7.4840 | 1494.15 | 0.0512 | 11.81 | 45.65 |
| 8.9448 | 119.46 | 0.0768 | 9.89 | 3.65 |
| 10.0466 | 187.59 | 0.0768 | 8.80 | 5.73 |
| 10.7074 | 233.86 | 0.0512 | 8.26 | 7.14 |
| 12.3882 | 799.70 | 0.0768 | 7.15 | 24.43 |
| 13.2679 | 377.40 | 0.0768 | 6.67 | 11.53 |
| 14.7102 | 568.09 | 0.0768 | 6.02 | 17.36 |
| 14.9843 | 2932.69 | 0.0768 | 5.91 | 89.59 |
| 15.5238 | 198.90 | 0.1023 | 5.71 | 6.08 |
| 15.9717 | 499.36 | 0.0768 | 5.55 | 15.26 |
| 16.1445 | 891.35 | 0.0768 | 5.49 | 27.23 |
| 17.1984 | 544.27 | 0.0768 | 5.16 | 16.63 |
| 17.7834 | 1100.74 | 0.1023 | 4.99 | 33.63 |
| 17.9621 | 905.73 | 0.0768 | 4.94 | 27.67 |
| 19.2654 | 106.28 | 0.1023 | 4.61 | 3.25 |
| 19.6425 | 591.45 | 0.0768 | 4.52 | 18.07 |
| 20.0385 | 117.70 | 0.1023 | 4.43 | 3.60 |
| 20.4185 | 389.09 | 0.0768 | 4.35 | 11.89 |
| 20.9791 | 545.30 | 0.1791 | 4.23 | 16.66 |
| 21.4872 | 219.31 | 0.1023 | 4.14 | 6.70 |
| 22.1496 | 410.55 | 0.1279 | 4.01 | 12.54 |
| 22.5396 | 3273.30 | 0.1023 | 3.94 | 100.00 |
| 22.7764 | 655.92 | 0.0768 | 3.90 | 20.04 |
| 23.1601 | 338.30 | 0.1023 | 3.84 | 10.34 |
| 23.9454 | 709.78 | 0.1023 | 3.72 | 21.68 |
| 24.2248 | 31.42 | 0.4093 | 3.67 | 0.96 |
| 24.5318 | 488.72 | 0.1023 | 3.63 | 14.93 |
| 24.8942 | 137.22 | 0.1023 | 3.58 | 4.19 |
| 25.4625 | 149.49 | 0.1023 | 3.50 | 4.57 |
| 25.7943 | 149.62 | 0.1023 | 3.45 | 4.57 |
| 25.9994 | 195.59 | 0.1023 | 3.43 | 5.98 |
| 26.7835 | 44.54 | 0.2558 | 3.33 | 1.36 |
| 27.5913 | 103.70 | 0.1279 | 3.23 | 3.17 |
| 27.9790 | 146.44 | 0.1023 | 3.19 | 4.47 |
| 29.5306 | 45.23 | 0.3070 | 3.02 | 1.38 |
| 30.2156 | 170.98 | 0.1279 | 2.96 | 5.22 |
| 30.7076 | 99.44 | 0.1023 | 2.91 | 3.04 |
| 31.3724 | 66.97 | 0.0768 | 2.85 | 2.05 |
| 31.6955 | 177.89 | 0.1279 | 2.82 | 5.43 |
| 33.0614 | 46.17 | 0.1535 | 2.71 | 1.41 |
| 34.1417 | 53.25 | 0.1535 | 2.63 | 1.63 |
| 35.1767 | 95.08 | 0.1279 | 2.55 | 2.90 |
| 36.6647 | 62.87 | 0.1535 | 2.45 | 1.92 |
| 37.3309 | 34.34 | 0.2047 | 2.41 | 1.05 |
| 39.2135 | 68.69 | 0.2047 | 2.30 | 2.10 |

TABLE 4N

HPLC results of napadisylate Type D (824511-44-B1)

| # | RRT | Area (%) | # | RRT | Area (%) |
|---|---|---|---|---|---|
| 1 | 0.70 | 0.05 | 4 | 1.00 | 99.56 |
| 2 | 0.91 | 0.08 | 5 | 1.07 | 0.26 |
| 3 | 0.97 | 0.06 | -- | -- | -- |

### Besylate

### Besylate Type A

Besylate Type A (824511-30-A10) was obtained by slurry ~20.0 mg freebase and 8.9 mg benzenesulfonic acid (charge molar ratio of acid to freebase was 1:1) in 0.5 mL DCM/EtOAc (1:1, v/v) at RT for about 4 days and transferred to stir at 50° C. for about 1 day, then transferred to stir at -20° C. for about 3 days. Resulting solids were isolated by centrifugation and vacuum drying at RT overnight. Another batch of besylate Type A (824511-35-A1) was prepared using the same method and characterized. The XRPD overlay was displayed in FIG. 3AC with peak list of (824511-35-A1) shown in TABLE 40. TGA/DSC curves in FIG. 3AD showed a weight loss of 1.1% up to 90.0° C. and a weight loss of 3.3% from 90° C. to 130° C., two endothermic signals were observed with a major peak at 117.4° C. and a minor peak131.8° C. (peak). ¹H NMR result in FIG. 3AE showed that the peak of benzenesulfonic acid was observed. The molar ratio of acid/API was 1.0:1. No obvious solvent residual was detected. HPLC purity was 99.71 area% (FIG. 3AF and TABLE 4P).

TABLE 40

XRPD peak list of besylate Type A (824511-35-A1)

| Pos. [°2θ] | Height [cts] | FWHM Left [°2θ] | d-spacing [Å] | Rel. Int. [%] |
|---|---|---|---|---|
| 7.4431 | 891.88 | 0.0768 | 11.88 | 40.14 |
| 8.1434 | 909.53 | 0.0768 | 10.86 | 40.94 |
| 9.8300 | 176.86 | 0.1023 | 9.00 | 7.96 |
| 10.5242 | 358.07 | 0.0768 | 8.41 | 16.12 |
| 10.8498 | 171.83 | 0.0768 | 8.15 | 7.73 |
| 13.6585 | 218.31 | 0.0768 | 6.48 | 9.83 |
| 14.3217 | 2221.79 | 0.1023 | 6.18 | 100.00 |
| 14.6730 | 867.51 | 0.1023 | 6.04 | 39.05 |
| 14.9148 | 518.65 | 0.1023 | 5.94 | 23.34 |
| 15.4400 | 726.17 | 0.1023 | 5.74 | 32.68 |
| 16.3331 | 663.21 | 0.1023 | 5.43 | 29.85 |
| 17.3066 | 558.84 | 0.1023 | 5.12 | 25.15 |
| 18.4645 | 132.51 | 0.0768 | 4.81 | 5.96 |
| 18.9234 | 207.91 | 0.1023 | 4.69 | 9.36 |
| 19.3998 | 141.96 | 0.0768 | 4.58 | 6.39 |
| 19.7308 | 1068.59 | 0.1023 | 4.50 | 48.10 |
| 20.8616 | 284.45 | 0.0768 | 4.26 | 12.80 |
| 21.2321 | 200.17 | 0.1023 | 4.18 | 9.01 |
| 21.7877 | 422.82 | 0.1791 | 4.08 | 19.03 |
| 22.0626 | 553.36 | 0.1023 | 4.03 | 24.91 |
| 22.3157 | 216.79 | 0.0768 | 3.98 | 9.76 |
| 22.7191 | 683.70 | 0.1535 | 3.91 | 30.77 |
| 23.3333 | 153.95 | 0.1023 | 3.81 | 6.93 |
| 23.8426 | 176.58 | 0.1023 | 3.73 | 7.95 |
| 24.2543 | 264.11 | 0.1023 | 3.67 | 11.89 |
| 24.5928 | 437.92 | 0.1023 | 3.62 | 19.71 |
| 25.3924 | 104.08 | 0.0768 | 3.51 | 4.68 |
| 26.9154 | 144.24 | 0.0768 | 3.31 | 6.49 |
| 27.4939 | 66.16 | 0.1535 | 3.24 | 2.98 |
| 27.9458 | 62.74 | 0.1535 | 3.19 | 2.82 |
| 28.9432 | 224.35 | 0.1023 | 3.08 | 10.10 |
| 29.3926 | 86.26 | 0.1535 | 3.04 | 3.88 |
| 29.7806 | 167.15 | 0.1023 | 3.00 | 7.52 |
| 30.4462 | 32.52 | 0.3070 | 2.94 | 1.46 |
| 31.4551 | 109.57 | 0.1279 | 2.84 | 4.93 |
| 32.0164 | 62.17 | 0.1535 | 2.80 | 2.80 |
| 33.7600 | 37.59 | 0.1535 | 2.66 | 1.69 |
| 36.1350 | 41.70 | 0.1535 | 2.49 | 1.88 |
| 36.8211 | 63.61 | 0.2558 | 2.44 | 2.86 |
| 38.4625 | 81.52 | 0.0768 | 2.34 | 3.67 |

TABLE 4P

HPLC results of besylate Type A (I824511-35-A1)

| # | RRT | Area (%) | # | RRT | Area (%) |
|---|---|---|---|---|---|
| 1 | 0.91 | 0.05 | 3 | 1.00 | 99.71 |
| 2 | 0.97 | 0.05 | 4 | 1.07 | 0.18 |

### Besylate Type B

Besylate Type B (824511-36-A10) was obtained by slurry ~20.0 mg freebase and 8.7 mg benzenesulfonic acid (charge molar ratio of acid to freebase was 1:1) in 0.5 mL IPA at RT overnight and then transferred to slurry at 50° C. for about 4 days. Resulting solids were isolated by centrifugation and vacuum drying at RT overnight. The XRPD result was displayed in FIG. 3AG and TABLE 4Q. TGA/DSC curves in FIG. 3AH showed a weight loss of 0.4% up to 80.0° C. and two endothermic signals at 177.5° C. and 179.3° C. (peak). ¹H NMR result in FIG. 3AI showed that the peak of benzenesulfonic acid was observed. The molar ratio of acid/API was 1.0:1. No obvious solvent residual was detected. HPLC purity was 99.73 area% (FIG. 3AJ and TABLE 4R).

TABLE 4Q

XRPD peak list of besylate Type B (824511-36-A10)

| Pos. [°2θ] | Height [cts] | FWHM Left [°2θ] | d-spacing [Å] | Rel. Int. [%] |
|---|---|---|---|---|
| 8.2720 | 11140.66 | 0.0768 | 10.69 | 100.00 |
| 9.5242 | 193.65 | 0.0768 | 9.29 | 1.74 |
| 9.7729 | 493.56 | 0.0768 | 9.05 | 4.43 |
| 11.4652 | 248.20 | 0.1023 | 7.72 | 2.23 |
| 14.6001 | 254.22 | 0.0768 | 6.07 | 2.28 |
| 14.7345 | 189.71 | 0.0768 | 6.01 | 1.70 |
| 15.3275 | 105.16 | 0.1023 | 5.78 | 0.94 |
| 15.6908 | 93.73 | 0.1023 | 5.65 | 0.84 |
| 15.8950 | 143.97 | 0.0768 | 5.58 | 1.29 |
| 16.1968 | 273.61 | 0.1023 | 5.47 | 2.46 |
| 16.5774 | 583.79 | 0.0768 | 5.35 | 5.24 |
| 17.7101 | 312.52 | 0.1023 | 5.01 | 2.81 |
| 17.9500 | 222.06 | 0.0768 | 4.94 | 1.99 |
| 18.3988 | 559.01 | 0.0768 | 4.82 | 5.02 |
| 18.6806 | 641.70 | 0.0768 | 4.75 | 5.76 |
| 19.1893 | 46.86 | 0.0768 | 4.63 | 0.42 |
| 19.6376 | 44.03 | 0.1535 | 4.52 | 0.40 |
| 21.4408 | 96.77 | 0.1023 | 4.14 | 0.87 |
| 22.2068 | 83.28 | 0.1535 | 4.00 | 0.75 |
| 22.6423 | 211.21 | 0.1023 | 3.93 | 1.90 |
| 22.8628 | 86.66 | 0.0768 | 3.89 | 0.78 |
| 23.3376 | 246.52 | 0.1023 | 3.81 | 2.21 |
| 24.4476 | 73.49 | 0.1023 | 3.64 | 0.66 |
| 25.1382 | 39.90 | 0.1535 | 3.54 | 0.36 |
| 25.4857 | 27.88 | 0.1535 | 3.50 | 0.25 |
| 26.1108 | 105.39 | 0.0768 | 3.41 | 0.95 |
| 26.4320 | 202.37 | 0.1023 | 3.37 | 1.82 |
| 27.0663 | 44.57 | 0.1023 | 3.29 | 0.40 |
| 28.3572 | 37.85 | 0.0768 | 3.15 | 0.34 |
| 28.6795 | 103.18 | 0.1023 | 3.11 | 0.93 |
| 29.5350 | 34.17 | 0.2047 | 3.02 | 0.31 |
| 30.2940 | 55.66 | 0.0768 | 2.95 | 0.50 |
| 30.4840 | 75.00 | 0.0768 | 2.93 | 0.67 |
| 30.8844 | 37.71 | 0.0768 | 2.90 | 0.34 |
| 32.7701 | 26.36 | 0.1535 | 2.73 | 0.24 |
| 33.4882 | 64.02 | 0.1023 | 2.68 | 0.57 |
| 34.7265 | 48.23 | 0.2047 | 2.58 | 0.43 |
| 36.1472 | 10.36 | 0.3582 | 2.48 | 0.09 |
| 38.8126 | 13.42 | 0.3070 | 2.32 | 0.12 |

TABLE 4R

HPLC results of besylate Type B (824511-36-A10)

| # | RRT | Area (%) |
|---|---|---|
| 1 | 1.00 | 99.73 |
| 2 | 1.07 | 0.27 |

### Procedure of Freebase Isolation

Detailed procedure of freebase isolation is summarized in TABLE 5A.

TABLE 5A

Detailed procedure of freebase isolation

| | |
|---|---|
| Scale | Procedure |
| 9-g (824509-03-A) | 1. Dissolve 9.0 g of HCl salt Type A (824509-01-A) in 120 mL of water, and add 120 mL of DCM. |
| | 2. Add 13.5 mL of aqueous ammonium hydroxide solution (25%~28%). |
| | 3. Slurry at RT for ~17 hrs to obtain a pH of 10.35, then separate layers. |
| | 4. Add additional 150 mL of water to wash the organic layer. |
| | 5. Add additional 150 mL of DCM to wash the aqueous layer. |
| | 6. Collect the organic layers and dry organic layer over anhydrous sodium sulfate. |
| | 7. Filter and concentrate organic layer to dryness. |
| | 8. Vacuum dry the solids at RT for ~1 hr. |
| | 9. Collect the 18-MC freebase (7.98 g, yield*=97.5%). |

### Summary of Salts and Salt Forms

TABLE 5B summarizes the salts and salt forms produced and the corresponding reports with additional details.

TABLE 5B

Summary of 18-MC salts and salt forms

| | | | | |
|---|---|---|---|---|
| Salt | Form (ID) | Designation | Molar ratio (acid:FB) | TGA wt loss % |
| Freebase | Type A (819246-09-A) | Anhydrate | NA | 1.1 (150° C.) |
| Besylate | Type A (824511-35-A1) | Hydrate | 1:1 | 4.4 (130° C.) |
| Besylate | Type B (824511-36-A10) | Anhydrate | 1:1 | 0.4 (80° C.) |
| Besylate | Type C (824529-04-A5_N2 Back 30° C.) | Anhydrate | NA | NA |
| Gentisate | Type A (824511-01-C9) | Hydrate/ anhydrate | 1:1 | 2.7 (150° C.) |
| HBr salt | Type A (824511-01-E10) | Anhydrate | 1:1 | 1.5 (150° C.) |
| HBr salt | Type B (824511-10-A1) | Solvate/hydrate | 0.9:1 | 14.2 (150° C.) |
| HBr salt | Type C (824511-39-A3) | Hydrate/ anhydrate | 1:1 | 2.0 (100° C.) |
| HBr salt | Type D (824511-39-A12) | THF solvate | 1.1:1 | 2.3 (90° C.) |
| HCl salt | Type A (819246-01-A) | Anhydrate | 1:1 | 1.4 (150° C.) |
| HCl salt | Type B (819246-46-A2) | 1-BuOH solvate 0.9 mol | NA | 15.9 (150° C.) |
| HCl salt | Type C (819246-47-A7) | Chloroform solvate 0.8 mol | NA | 17.8 (150° C.) |
| HCl salt | Type D (819246-46-A13) | Metastable | NA | NA |
| HCl salt | Type E (819246-43-A5) | Metastable | NA | NA |
| HCl salt | Type F (819246-43-A8) | Hydrate | 1:1 | 5.2 (100° C.) |
| HCl salt | Type G (819246-48-A2) | Metastable hydrate/solvate | NA | 5.2 (120° C.) |
| HCl salt | Type H (819246-49-A2) | Anhydrate | 1:1 | 2.7 (120° C.) |
| HCl salt | Type I (819246-49-A5) | Chloroform solvate 0.4 mol | NA | 11.6 (170° C.) |
| HCl salt | Type J (819246-49-A6) | Metastable | NA | 7.6 (140° C.) |
| HCl salt | Type K (819246-43-A8_N2_165.0° C.) | Anhydrate | NA | NA |
| HCl salt | Type L (824509-05-A1) | Metastable, mixture with HCl Type A | NA | NA |
| HCl salt | Type M (824509-05-A4) | 1,4-Dioxane solvate 1 mol | 1:1 | 14.6 (180° C.) |
| HCl salt | Type N (824509-10-A1) | Metastable, mixture with HCl Type A | NA | NA |
| HCl salt | Type O (824509-10-A2) | Metastable, mixture with HCl Type A | NA | NA |
| HCl salt | Type P (824509-10-A3) | TFE solvate 0.9 mol | 0.9:1 | 23.7 (150° C.) |
| HCl salt | Type Q (824509-16-A4) | Isopentanol solvate 0.7 mol | 1:1 | 15.3 (150° C.) |
| HCl salt | Type R (824509-16-A3) | 2-BuOH solvate 0.8 mol | 1:1 | 15.0 (150° C.) |
| HCl salt | Type S (824509-29-A3) | Cyclohexanone solvate 0.15 mol | 1:1 | 2.8 (150° C.) |
| HCl salt | Type T (824509-39-A1) | Propionic acid solvante 0.6 mol | 1:1 | 11.1 (150° C.) |
| HCl salt | Type U (824509-29-B4) | Benzyl alcohol solvate 1 mol | 0.9:1 | 18.9 (120° C.) |
| HCl salt | Type V (824509-25-A4) | Metastable | NA | NA |
| Maleate | Type A (819246-23-A4) | Anhydrate | 0.9:1 | 0.8 (150° C.) |
| Mesylate | Type A (824511-23-B) | Anhydrate | 1:1 | 1.7 (80° C.) |
| Mesylate | Type B (824511-32-A1) | Metastable | NA | NA |
| Mesylate | Type C (824511-32-A2) | Metastable | NA | NA |
| Napadisyl ate | Type A (824511-30-B7) | Hydrate/ anhydrate | 0.6:1 | 7.1 (120° C.) |
| Napadisyl ate | Type B (824511-36-A7) | Unidentified | 0.6:1 | 6.0 (80° C.) |
| Napadisyl ate | Type C (824511-44-B2) | Unidentified | 0.7:1 | 6.7 (100° C.) |
| Napadisyl ate | Type D (824511-44-B1) | Hydrate/ anhydrate | 0.7:1 | 6.9 (100° C.) |
| Oxalate | Type A (819246-23-A20) | Possible anhydrate | 0.9:1 | 1.5 (120° C.) |
| Oxalate | Type B (824511-04-C) | Anhydrate | 1:1 | 1.1 (150° C.) |
| Sulfate | Type A (824511-04-A) | Anhydrate | 1:1 | 1.6 (120° C.) |
| Sulfate | Type B (824511-11-A3) | ACN solvate 0.6 mol | 0.9:1 | 6.6 (100° C.) |
| Sulfate | Type C (824511-11-A4) | Metastable | NA | NA |
| Sulfate | Type D (824511-12-A17) | Hydrate | 1:1 | 2.3 (100° C.) |
| Sulfate | Type E (824511-11-A3-0315) | Hydrate/ anhydrate | NA | 3 (120° C.) |
| Sulfate | Type F (824511-12-A17_N2 Back_30.0° C.) | Anhydrate | NA | NA |
| Tosylate | Type A (819246-23-A18) | Hydrate | 0.9:1 | 4.2 (120° C.) |
| Tosylate | Type B (819246-23-D18) | Hydrate | 1:1 | 5.8 (120° C.) |
| Tosylate | Type C (824511-23-A) | Hydrate | 1:1 | 4.6 (110° C.) |
| Tosylate | Type D (824511-23-ARE_N2_60min_30.0° C.) | Anhydrate | NA | NA |
| Tosylate | Type E (824528-05-A9) | 1,4-Dioxane solvate 0.8 mol | 0.9:1 | 14.5 (130° C.) |
| Tosylate | Type F (824528-06-B1) | CHCl₃ solvate 0.4 mol | 1:1 | 17.4 (100° C.) |
| Tosylate | Type G (824528-06-A1) | Anisole solvate 0.5 mol | 0.9:1 | 8.2 (120° C.) |
| Tosylate | Type H (824528-05-A12) | Metastable | NA | NA |

### Instruments and Methods

### XPRD

For XRPD analysis, a PANalytical Empyrean and X′ Pert3 X-ray powder diffract meter was used. The XRPD parameters used are listed in TABLE 5C.

TABLE 5C

Parameters for XPRD test

| | | |
|---|---|---|
| Parameters | XRPD | |
| Model | Empyrean | X′ Pert³ |
| Test mode | Reflection | Reflection |
| Sample holder | Zero background | Zero background |
| X-Ray wavelength | Cu, kα, Kα1 (Å): 1.540598, Kα2 (Å): 1.544426 Kα2/Kα1 intensity ratio: 0.50 | Cu, kα, Kα1 (Å): 1.540598, Kα2 (Å): 1.544426 Kα2/Kα1 intensity ratio: 0.50 |
| X-Ray tube setting | 45 kV, 40 mA | 45 kV, 40 mA |
| Divergence slit | Automatic or ⅛° | ⅛° |
| Scan mode | Continuous | Continuous |
| Scan range (°2TH) | 3-40 | 3-40 |
| Scan step time (s) | 17.8 | 46.7 |
| Step size (°2TH) | 0.0167 | 0.0263 |
| Test Time (s) | 5 min 30 s | 5 min 04 s |

### TGA and DSC

TGA data was collected using a TA Q5000/Discovery TGA 5500 from TA Instruments. DSC was performed using a Discovery DSC 2500 from TA Instruments. Detailed parameters used are listed in TABLE 5D.

TABLE 5D

Parameters for TGA and DSC test

| | | |
|---|---|---|
| Parameters | TGA | DSC |
| Method | Ramp | Ramp |
| Sample pan | Aluminum, open | Aluminum, crimped (no pinhole) |
| Temperature | RT - desired temperature | 25° C. - desired temperature |
| Heating rate | 10° C./min | 10° C./min |
| Purge gas | N₂ | N₂ |

### HPLC

Agilent 1260 with DAD detector was utilized and detailed chromatographic condition is listed in TABLE 5E.

TABLE 5E

Chromatographic conditions and parameters for purity and solubility test

| | | |
|---|---|---|
| Parameter(s) | Value | |
| Instrument | Agilent 1260 with DAD detector | |
| Column | Phenomenex Gemini 3 µm NX-C18, 150×4.6 mm, 3 µm | |
| Mobile phase | A: 0.05% TFA in H₂O | |
| | B: 0.05% TFA in ACN | |
| Gradient table | Time (min) | %B |
| | 0.0 | 10 |
| | 11.0 | 95 |
| | 12.0 | 95 |
| | 12.1 | 10 |
| | 16.0 | 10 |
| Flow rate | 1.0 mL/min | |
| Injection volume | 10 µL | |
| Detector wavelength | UV at 284 nm | |
| Column temperature | 25° C. | |

### IC

Thermo Scientific™ Dionex™ Aquion™ Ion Chromatography (IC) System 1100 with conductivity detector was utilized and detailed chromatographic condition is listed in TABLE 5F.

TABLE 5F

Chromatographic conditions and parameters for ion content test

| | |
|---|---|
| Parameter(s) | Value |
| Instrument | ThermoFisher ICS-1100 |
| Column | lonPac AS18 Analytical Column (4 × 250 mm) |
| Mobile phase | 25 mM NaOH |
| Injection volume | 25 µL |
| Flow rate | 1.0 mL/min |
| Cell Temp. | 35° C. |
| Column Temp. | 35° C. |
| Current | 80 mA |
| Run Time | 7 mins (Cl⁻), 15 mins (Br) |

### Solution ¹H NMR

Solution ¹H NMR was collected on a Bruker 400 MHz NMR Spectrometer using DMSO-d₆ as the solvent.

### Conclusion

An extended salt screening was performed for compound 18-MC using prepared freebase material. As the results showed, a total of 9 salt forms were found and characterized, including gentisate Type A, HBr salt Types A and B, besylate Types A and B and napadisylate Types A, B, C, and D.

### Example 2 - Polymorph Screening of HCl Salt

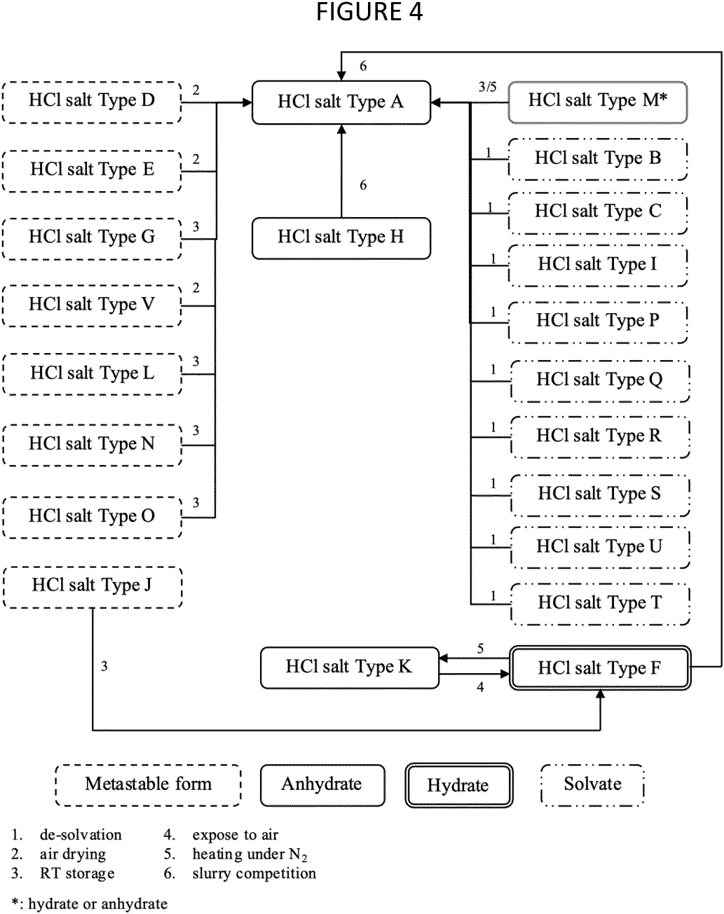

Starting from HCl salt materials with different forms, a total of 100 polymorph screening experiments was performed using different crystallization methods including salt formation by liquid vapor diffusion/slurry at elevated temperature, evaporation at RT/high temperatures, slurry and reverse anti-solvent addition at different temperatures, slow/crash cooling, polymer induced crystallization and grinding. All the resulting solids were isolated for XRPD test, and new forms were further characterized by TGA, DSC, ¹H NMR and HPLC/ IC. As the results showed, 11 new forms (HCl salt Types L to V) were discovered. Further identification results showed that HCl salt Types M, P, Q, R, S, T, and U were solvates (which converted to HCl salt Type A after desolvation), HCl salt Types L, N, O,, and V were metastable forms (which converted to HCl salt Type A after air drying or room temperature (RT) storage). Characterization results of different HCl salt forms were summarized in TABLE 6A to 6C. Inter-conversion relationship among different forms were displayed in FIG. 4.

To summarize, an additional polymorph screening was performed for 18-MC HCl salt. As the results showed, 11 new forms were obtained. Among the forms obtained, HCl salt Type A was thermodynamically more stable than other anhydrates at RT.

TABLE 6A

Characterization summary of HCl salt forms- anhydrates and hydrate

| Solid form (ID) | Crystallinity | TGA loss, % | Endothermic peak, °C. | Molar ratio^{&} | Form change after treatment | Identified form |
|---|---|---|---|---|---|---|
| HCl salt Type A (819246-01-A) | High | 1.4 (up to 150° C.) | 211.6 | 1:1 | -- | Anhydrate |
| HCl salt Type H (819246-49-A2) | High | 2.7 (up to 120° C.) | 69.9, 214.3 | 1:1 | Freebase Type A+extra peak* | Anhydrate |
| HCl salt Type K (819246-43-A8_N2_165.0° C.) | High | -- | -- | -- | HCl salt Type F^{#} | Anhydrate |
| HCl salt Type F (819246-43-A8) | High | 5.2 (up to 100° C.) | 98.4, 211.4 | 1:1 | HCl salt Type K* | Hydrate |

--: No data collected.
^{&}: Acid to freebase.
^{#}: Exposure to ambient conditions (~50%RH).
*: Heat to 165~170° C. under N₂.

TABLE 6B

Characterization summary of HCl salt forms- solvates

| Solid form (ID) | Crystallinity | TGA loss, % | Endothermic peak, °C. | Molar ratio^{&} (solvent wt%) | Form after desolvation | Identified result |
|---|---|---|---|---|---|---|
| HCl salt Type B (819246-46-A2-AIRDRY) | High | 15.9 (up to 150° C.) | 129.8, 217.2 | -- (13.6) | HCl salt Type A | 1-BuOH solvate |
| HCl salt Type C (819246-47-A7) | Low | 17.8 (up to 150° C.) | 114.6, 211.9 | -- (18.7) | HCl salt Type A | Chloroform solvate |
| HCl salt Type M (824509-05-A4) | High | 14.6 (up to 180° C.) | 170.3 | 1.0 (8.92) | HCl salt Type A** | 1,4-Dioxane solvate |
| HCl salt Type I (819246-49-A5) | High | 11.6 (up to 170° C.) | 151.8, 215.7 | (9.8) | HCl salt Type A | Chloroform solvate |
| HCl salt Type P (824509-10-A3) | High | 23.7 (up to 150° C.) | 122.7*, 202.3 | 0.9 (21.1) | HCl salt Type A | 2.2,2-trifluoroethanol solvate |
| HCl salt Type Q (824509-16-A4) | High | 15.3 (up to 130° C.) | 82.9, 141.9 | 0.7 (11.6) | HCl salt Type A+freebase Type A | Isopentanol solvate |
| HCl salt Type R (824509-16-A3) | High | 15.0 (up to 150° C.) | 141.8 | 0.8 (13.4) | HCl salt Type A | 2-BuOH solvate |
| HCl salt Type S (824509-29-A3) | High | 2.8% (up to 150° C.) | 98.0, 208.0 | 0.2 (3.5) | HCl salt Type A | Cyclohexanone solvate |
| HCl salt Type U (824509-29-B4) | High | 18.9 % (up to 120° C.) | 132.9 | 1.0 (21.1) | HCl salt Type A | Benzyl alcohol solvate |
| HCl salt Type T (824509-39-A1) | High | 11.1% (up to 150° C.) | 91.6, 112.9, 135.5, 190.8, 207.4 | 0.63 (10.3) | HCl salt Type A | Propionic acid solvate |

--: No data collected.
^{&}: Acid to freebase.
**: RT storage or heat to 100° C. under N₂.

TABLE 6C

Characterization summary of HCl salt forms- metastable forms

| | | | | | |
|---|---|---|---|---|---|
| Solid form (ID) | Crystallinity | TGA loss, % | Endothermic peak, °C. | Form after treatment | Identified result |
| HCl salt Type D (819246-46-A13) | Low | -- | -- | HCl salt Type A** | Metastable |
| HCl salt Type E (819246-43-A5) | High | -- | -- | HCl salt Type A** | Metastable |
| HCl salt Type G (819246-48-A2) | High | 5.2 (up to 120° C.) | 108.7, 215.9 | HCl salt Type A^{#} | Metastable |
| HCl salt Type J (819246-49-A6) | High | 7.6 (up to 140° C.) | 87.6, 195.6 | HCl salt Type F^{#} | Metastable |
| HCl salt Type V (824509-25-A4) | High | -- | -- | HCl salt Type A** | Metastable |
| HCl salt Type L* (824509-05-A1) | High | -- | -- | HCl salt Type A^{#} | Metastable |
| HCl salt Type N* (824509-10-A1) | High | -- | -- | HCl salt Type A^{#} | Metastable |
| HCl salt Type O* (824509-10-A2) | High | -- | -- | HCl salt Type A^{#} | Metastable |

--: No data collected due to form change after storage/drying.
*: Only the new form mixed with HCl salt Type A was obtained in the screening and re-preparation.
**: Air drying.
^{#}: RT storage.

### Polymorph Screening

Using freebase Type A (824509-03-A), HCl salt Type A (824509-01-A and 824509-11-B), the low crystallinity HCl salt (824509-12-E) and amorphous (824509-20-A) as the starting material, a total of 100 polymorph screening experiments were conducted via various crystallization methods. Results of polymorph screening is summarized in TABLE 7A, TABLE 7B and TABLE 7C. XRPD results showed that a total of 11 forms (HCl salt Types L to V) were obtained in polymorph screening and characterization. The screening details are further detailed below.

TABLE 7A

Summary of polymorph screening experiments of HCl salt (1^{st})

| Method | No. of Experiment | Results |
|---|---|---|
| Salt formation (liquid vapor diffusion at RT) | 4 | HCl salt Type M, HCl salt Type A+L, oil |
| Salt formation (slurry at 60° C.) | 6 | HCl salt Type A, gel |
| Evaporation at 60° C. | 10 | HCl salt Type A, HCl salt Type A+extra peak, HCl salt Type J+freebase Type A, HCl salt Type G+freebase Type A+extra peak, HCl salt Type A+freebase Type A, freebase Type A, amorphous |
| Evaporation at 80° C. | 5 | HCl salt Type E+freebase Type A, amorphous, clear solution |
| Slurry at 60° C. | 15 | HCl salt Type A/C/Q/R, HCl salt Type A+extra peak, clear solution |
| Reverse anti-solvent addition | 10 | HCl salt Type A/A+N/A+O, HCl salt Type A+extra peak, HCl salt Type C+extra peak, oil |
| Salt formation (liquid vapor diffusion at RT) | 4 | HCl salt Type M, HCl salt Type A+L, oil |
| Total | 50 | HCl salt Type A/C/M/P/Q/R/A+L/A+N/A+O, HCl salt Type A+extra peak, HCl salt Type J+freebase Type A, HCl salt Type G+freebase Type A+extra peak, HCl salt Type A+freebase Type A, freebase Type A, amorphous, oil, clear solution. |

TABLE 7B

Summary of polymorph screening experiments of HCl salt (2^{nd})

| Method | No. of Experiment | Results |
|---|---|---|
| Slurry at -20° C. | 5 | HCl salt Type A/R/S/T/U/V/A+F |
| Slurry at 100° C. | 5 | HCl salt Type A |
| Cooling from 50° C. to 5° C. | 5 | HCl salt Type F+A+extra peak/HCl salt Type A/clear solution/gel |
| Evaporation at RT | 5 | HCl salt Type A/clear solution |
| Revers anti-solvent addition at 5° C. | 5 | HCl salt Type A/A+extra peak/oil |
| Total | 25 | HCl salt Type A/R/S/T/U/V/A+F/A+extra peak/gel/oil/clear solution |

TABLE 7C

Summary of polymorph screening experiments of HCl salt (3^{rd)}

| Method | No. of Experiment | Results |
|---|---|---|
| Polymer induced slurry at 5° C./50° C. | 2 | HCl salt Type F+extra peak, HCl Salt Type A |
| Crash cooling | 10 | HCl salt Type A/T/U/F, clear solution |
| Reverse anti-solvent addition at -20° C. | 10 | HCl salt Type T/U+extra peak, HCl salt Type F+T, clear solution, clear solution |
| Liquid assisted grinding | 3 | HCl salt Type A, gel |
| Total | 25 | HCl Salt Type A/T/U/F+extra peak, gel, clear solution |

### Solvates

### HCl Salt Type M

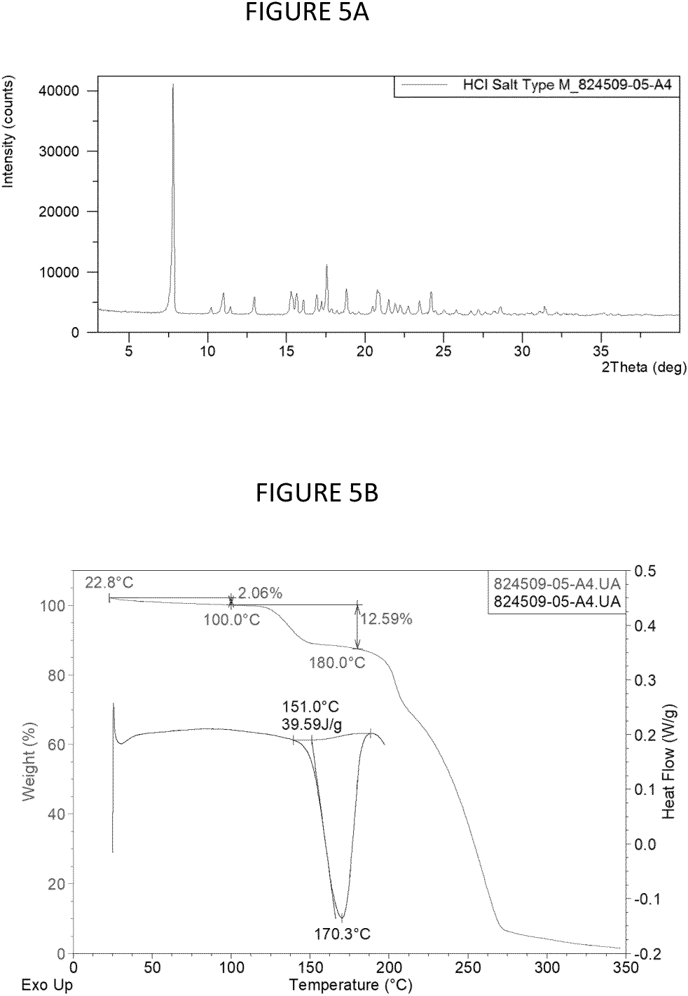

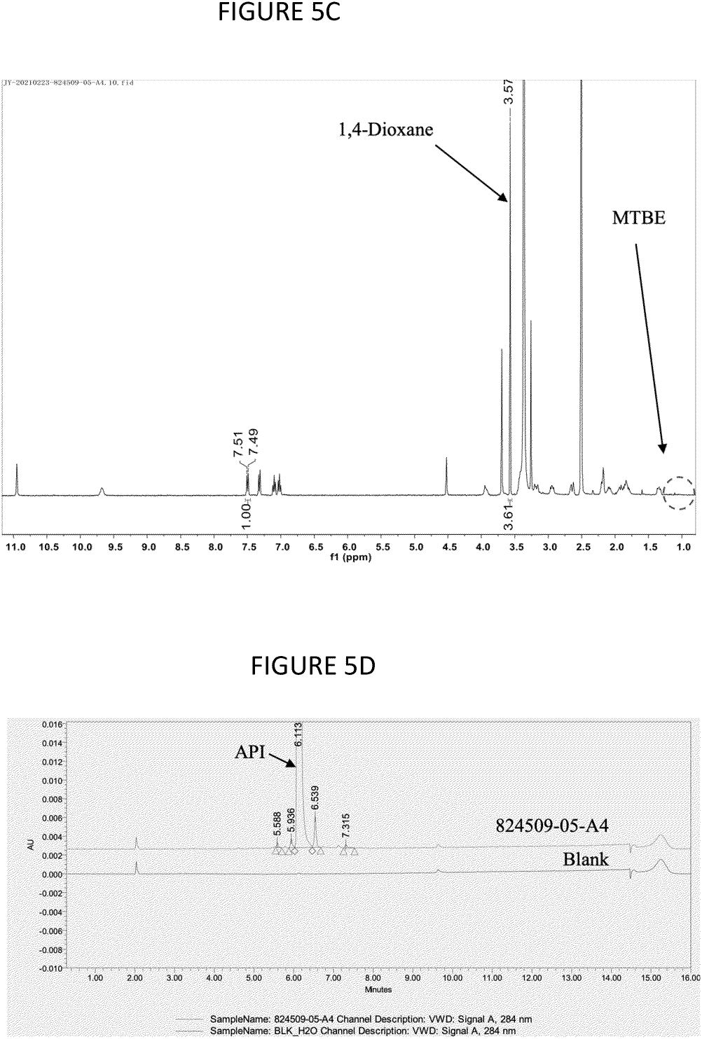

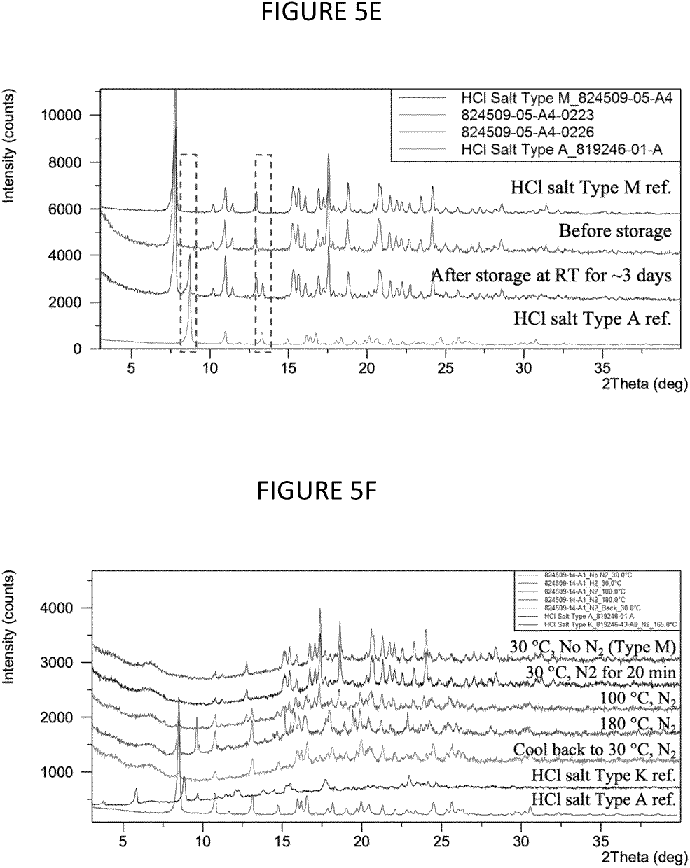

HCl salt Type M (824509-05-A4) was obtained by salt formation through liquid vapor diffusion in 1,4-dioxane/MTBE. The detailed procedure was as follows: dissolve 20.0 mg freebase in 1.0 mL 1,4-dioxane at RT in a 4-mL vial. Dilute 1 mL HCŀEtOAc solution (conc. of HCl was 2 mol/L) by 3 mL MTBE in a 20-mL vial. Put the uncapped 4-mL vial into the 20-mL vial and keep the capped 20-mL vial at RT for about 4 days. Solids were isolated by air dried for characterization. The XRPD result is displayed in FIG. 5A and TABLE 7D. TGA/DSC results in FIG. 5B showed a weight loss of 2.1% up to 100° C. and a weight loss of 12.6% from 100° C. to 180° C., DSC result showed one endothermic signal at 170.3° C. (peak). ¹H NMR result in FIG. 5C showed that the peak of 1,4-dioxane was observed. The molar ratio of 1,4-dioxane/API was 0.5:1 (theoretical weight=8.92 wt%). HPLC/IC results showed that the molar ratio of acid/freebase was determined as 1.0:1 and HPLC purity was 99.45 area% (FIG. 5D and TABLE 7E).

In FIG. 5E, XRPD overlay showed that after storing HCl salt Type M at RT for ~3 days, the extra peaks of HCl salt Type A (marked in red frame) were observed. VT-XRPD results in FIG. 5F showed that after N₂-drying for 20 min at 30° C., no form change was observed for HCl salt Type M. After heating sample to 100° C. under N₂ protection, peaks of HCl salt Type A were observed. After heating to 180° C. under N₂ protection, most diffraction peaks were consistent with HCl salt Type A with extra peak similar to HCl salt Type K. After cooling back to 30° C. under N₂ protection, most diffraction peaks were consistent with HCl salt Type A. Considering the obvious solvent amount in the sample, HCl salt Type M was speculated as a 1,4-dioxane solvate that converted to Type A upon desolvation.

TABLE 7D

XRPD peak list of HCl salt Type M (824509-05-A4)

| Pos. [°2θ] | Height [cts] | FWHM Left [°2θ] | d-spacing [Å] | Rel. Int. [%] |
|---|---|---|---|---|
| 7.7879 | 11326.18 | 0.1023 | 11.35 | 100.00 |
| 10.2034 | 338.97 | 0.1023 | 8.67 | 2.99 |
| 10.9794 | 1064.88 | 0.1023 | 8.06 | 9.40 |
| 11.4319 | 403.84 | 0.1023 | 7.74 | 3.57 |
| 12.9458 | 893.47 | 0.1023 | 6.84 | 7.89 |
| 15.2929 | 1127.64 | 0.1535 | 5.79 | 9.96 |
| 15.6403 | 1054.00 | 0.1279 | 5.67 | 9.31 |
| 16.0670 | 741.21 | 0.1023 | 5.52 | 6.54 |
| 16.9073 | 995.43 | 0.1279 | 5.24 | 8.79 |
| 17.2181 | 627.89 | 0.0768 | 5.15 | 5.54 |
| 17.5521 | 2449.59 | 0.1279 | 5.05 | 21.63 |
| 17.8639 | 310.93 | 0.0768 | 4.97 | 2.75 |
| 18.1888 | 208.73 | 0.1023 | 4.88 | 1.84 |
| 18.8049 | 1316.00 | 0.1279 | 4.72 | 11.62 |
| 19.1967 | 106.64 | 0.1023 | 4.62 | 0.94 |
| 19.5929 | 155.74 | 0.1279 | 4.53 | 1.38 |
| 20.4628 | 410.58 | 0.1023 | 4.34 | 3.63 |
| 20.7772 | 1225.84 | 0.1023 | 4.28 | 10.82 |
| 20.9025 | 1075.56 | 0.1023 | 4.25 | 9.50 |
| 21.4825 | 785.14 | 0.1279 | 4.14 | 6.93 |
| 21.9031 | 567.78 | 0.1279 | 4.06 | 5.01 |
| 22.2232 | 480.80 | 0.1535 | 4.00 | 4.25 |
| 22.7305 | 441.00 | 0.1279 | 3.91 | 3.89 |
| 23.4474 | 716.24 | 0.1023 | 3.79 | 6.32 |
| 24.1867 | 1175.60 | 0.1279 | 3.68 | 10.38 |
| 24.4411 | 199.64 | 0.1023 | 3.64 | 1.76 |
| 25.0065 | 244.26 | 0.1535 | 3.56 | 2.16 |
| 25.7795 | 258.27 | 0.1023 | 3.46 | 2.28 |
| 26.7067 | 203.37 | 0.1279 | 3.34 | 1.80 |
| 27.1999 | 271.24 | 0.1279 | 3.28 | 2.39 |
| 27.6270 | 140.66 | 0.0768 | 3.23 | 1.24 |
| 28.1617 | 201.35 | 0.1279 | 3.17 | 1.78 |
| 28.5906 | 408.23 | 0.1535 | 3.12 | 3.60 |
| 29.5030 | 85.03 | 0.1535 | 3.03 | 0.75 |
| 29.9198 | 49.26 | 0.1535 | 2.99 | 0.43 |
| 30.3134 | 86.32 | 0.1791 | 2.95 | 0.76 |
| 30.5670 | 132.42 | 0.1023 | 2.92 | 1.17 |
| 31.0784 | 174.18 | 0.1535 | 2.88 | 1.54 |
| 31.4248 | 382.53 | 0.1279 | 2.85 | 3.38 |
| 32.1696 | 149.08 | 0.1279 | 2.78 | 1.32 |
| 32.6176 | 93.49 | 0.1023 | 2.75 | 0.83 |
| 33.4389 | 62.27 | 0.1279 | 2.68 | 0.55 |
| 35.1532 | 105.53 | 0.1791 | 2.55 | 0.93 |
| 35.6045 | 74.06 | 0.1279 | 2.52 | 0.65 |
| 36.0944 | 53.96 | 0.1535 | 2.49 | 0.48 |
| 37.2081 | 54.16 | 0.1535 | 2.42 | 0.48 |
| 37.8211 | 65.91 | 0.1535 | 2.38 | 0.58 |

TABLE 7E

HPLC results of HCl salt Type M (824509-05-A4)

| # | RRT | Area (%) | # | RRT | Area (%) |
|---|---|---|---|---|---|
| 1 | 0.91 | 0.05 | 4 | 1.07 | 0.35 |
| 2 | 0.97 | 0.10 | 5 | 1.20 | 0.05 |
| 3 | 1.00 | 99.45 | -- | -- | -- |

### HCl Salt Type P

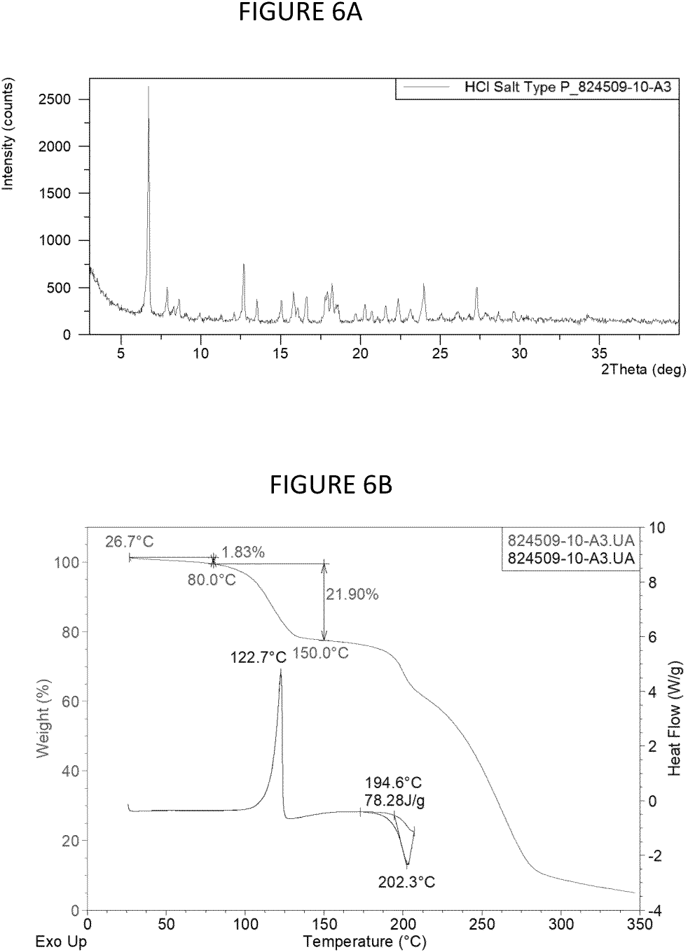

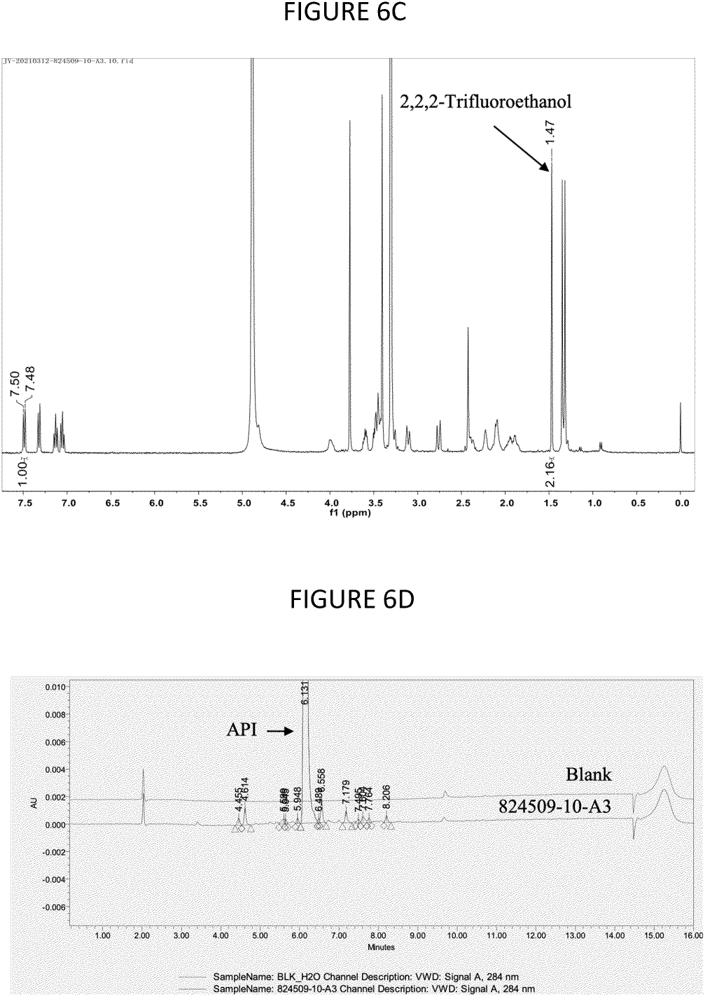

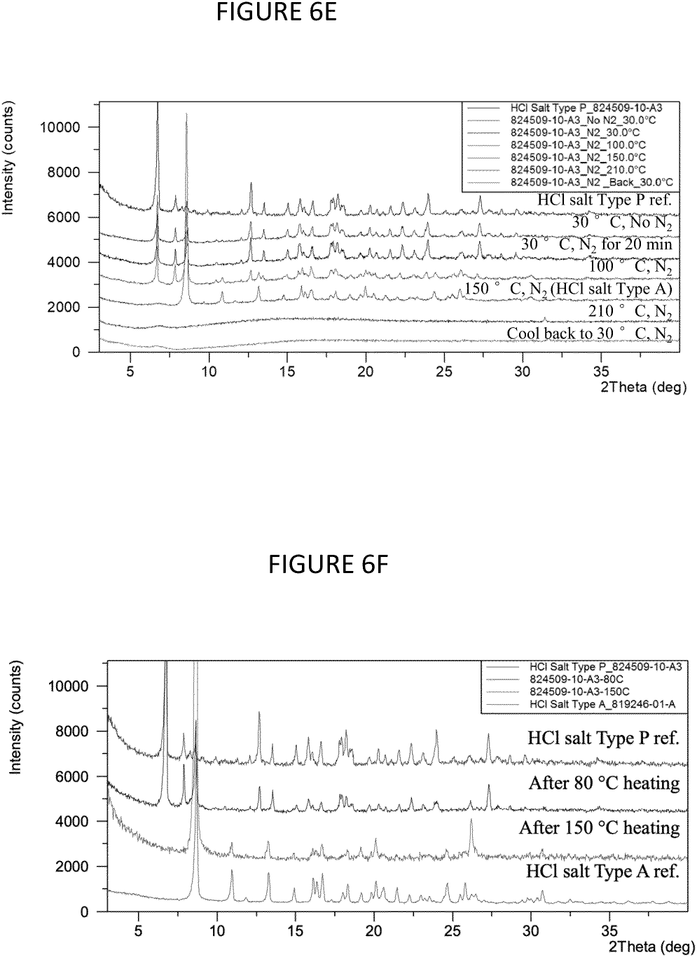

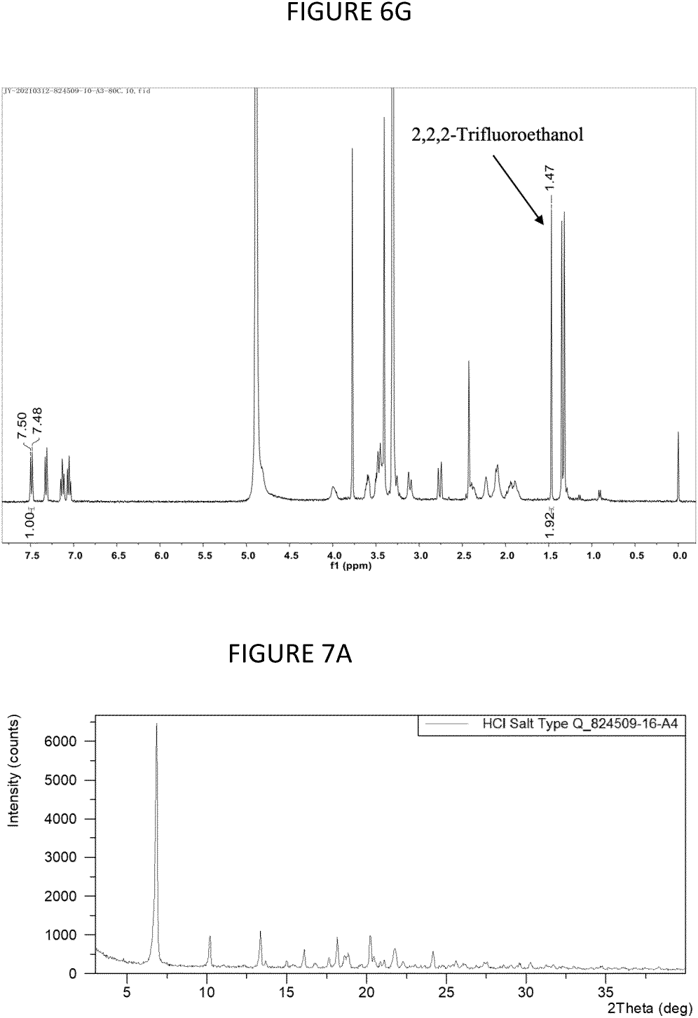

HCl salt Type P (824509-10-A3) was obtained by adding 1.0 mL 2,2,2-trifluoroethanol solution (conc. of HCl salt was ~20 mg/mL) in 9.0 mL MIBK directly at RT and stir at RT for 4 days. Since no solids precipitated after RT stirring, the clear solution was transferred to stir at 5° C. overnight, -20° C. overnight and evaporation at RT for about 3 weeks. The resulting solids were centrifuged and air dried for characterization. The XRPD result is displayed in FIG. 6A and TABLE 8A. TGA/DSC results in FIG. 6B showed a weight loss of 1.8% up to 80° C. and a weight loss of 21.9% from 80° C. to 150° C., DSC results showed one exothermic signal at 122.7° C. (peak) and one endothermic signal at 202.3° C. (peak). ¹H NMR spectrum in FIG. 6C showed that the peak of 2,2,2-trifluoroethanol was observed. The molar ratio of 2,2,2-trifluoroethanol/API was 1.1:1 (theoretical weight=21.1 wt%). HPLC/IC results showed that the molar ratio of acid/freebase was determined as 0.9:1 and HPLC purity was 98.71 area% (FIG. 6D and TABLE 8B).

The VT-XRPD result in FIG. 6E showed that after N₂-drying for 20 min at 30° C., no form change was observed for HCl salt Type P. After heating to 100° C. and 150° C. under N₂ protection, form change to HCl salt Type A was observed. After heating to 210° C. under N₂ protection, amorphous sample was observed. XRPD overlay in FIG. 6F showed that after heating HCl salt Type P to 80° C. and cooling back to RT, no obvious form change was observed. After 150° C. heating, HCl salt Type P converted to HCl salt Type A. ¹H NMR result in FIG. 6G showed that the peak of 2,2,2-trifluoroethanol was still observed after 80° C. heating, and molar ratio of 2,2,2-trifluoroethanol/API was 1.0:1 (theoretical weight=19.2 wt%). Combined with the results of heating experiments and VT-XRPD, HCl salt Type P was speculated to be a 2,2,2-trifluoroethanol solvate that converted to Type A upon desolvation.

TABLE 8A

XRPD peak list of HCl salt Type P (824509-10-A3)

| Pos. [°2θ] | Height [cts] | FWHM Left [°2θ] | d-spacing [Å] | Rel. Int. [%] |
|---|---|---|---|---|
| 6.7200 | 2280.94 | 0.1023 | 13.15 | 100.00 |
| 7.8697 | 302.99 | 0.1023 | 11.23 | 13.28 |
| 8.2743 | 102.70 | 0.1023 | 10.69 | 4.50 |
| 8.6046 | 193.81 | 0.0768 | 10.28 | 8.50 |
| 9.0490 | 46.23 | 0.1535 | 9.77 | 2.03 |
| 9.9192 | 65.51 | 0.1791 | 8.92 | 2.87 |
| 11.2604 | 33.26 | 0.1535 | 7.86 | 1.46 |
| 12.0835 | 74.73 | 0.1023 | 7.32 | 3.28 |
| 12.6827 | 598.41 | 0.1023 | 6.98 | 26.24 |
| 13.5171 | 236.02 | 0.0768 | 6.55 | 10.35 |
| 15.0403 | 229.78 | 0.1023 | 5.89 | 10.07 |
| 15.8125 | 317.49 | 0.1279 | 5.60 | 13.92 |
| 16.0562 | 148.67 | 0.1023 | 5.52 | 6.52 |
| 16.6110 | 267.19 | 0.1023 | 5.34 | 11.71 |
| 17.9305 | 315.26 | 0.0768 | 4.95 | 13.82 |
| 18.2191 | 395.59 | 0.1023 | 4.87 | 17.34 |
| 18.6252 | 144.48 | 0.1023 | 4.76 | 6.33 |
| 19.6873 | 75.24 | 0.1279 | 4.51 | 3.30 |
| 20.2711 | 176.63 | 0.1023 | 4.38 | 7.74 |
| 20.7131 | 109.07 | 0.1023 | 4.29 | 4.78 |
| 21.0853 | 54.67 | 0.1279 | 4.21 | 2.40 |
| 21.5805 | 162.90 | 0.1279 | 4.12 | 7.14 |
| 22.3644 | 235.16 | 0.1279 | 3.98 | 10.31 |
| 23.1245 | 127.21 | 0.1279 | 3.85 | 5.58 |
| 23.9864 | 385.29 | 0.1023 | 3.71 | 16.89 |
| 25.0607 | 64.16 | 0.1279 | 3.55 | 2.81 |
| 26.0737 | 84.59 | 0.2047 | 3.42 | 3.71 |
| 26.8085 | 47.90 | 0.1535 | 3.33 | 2.10 |
| 27.2882 | 350.58 | 0.1279 | 3.27 | 15.37 |
| 27.8555 | 73.69 | 0.2558 | 3.20 | 3.23 |
| 28.6578 | 93.82 | 0.1023 | 3.12 | 4.11 |
| 29.6142 | 80.12 | 0.0768 | 3.02 | 3.51 |
| 30.0982 | 40.38 | 0.1535 | 2.97 | 1.77 |
| 34.3179 | 50.92 | 0.3070 | 2.61 | 2.23 |

TABLE 8B

HPLC results of HCl salt Type P (824509-10-A3)

| # | RRT | Area (%) | # | RRT | Area (%) |
|---|---|---|---|---|---|
| 1 | 0.73 | 0.10 | 8 | 1.07 | 0.26 |
| 2 | 0.75 | 0.20 | 9 | 1.17 | 0.15 |
| 3 | 0.91 | 0.10 | 10 | 1.22 | 0.06 |
| 4 | 0.92 | 0.05 | 11 | 1.24 | 0.12 |
| 5 | 0.97 | 0.06 | 12 | 1.27 | 0.07 |
| 6 | 1.00 | 98.71 | 13 | 1.34 | 0.09 |
| 7 | 1.06 | 0.05 | -- | -- | -- |

### HCl Salt Type Q and R

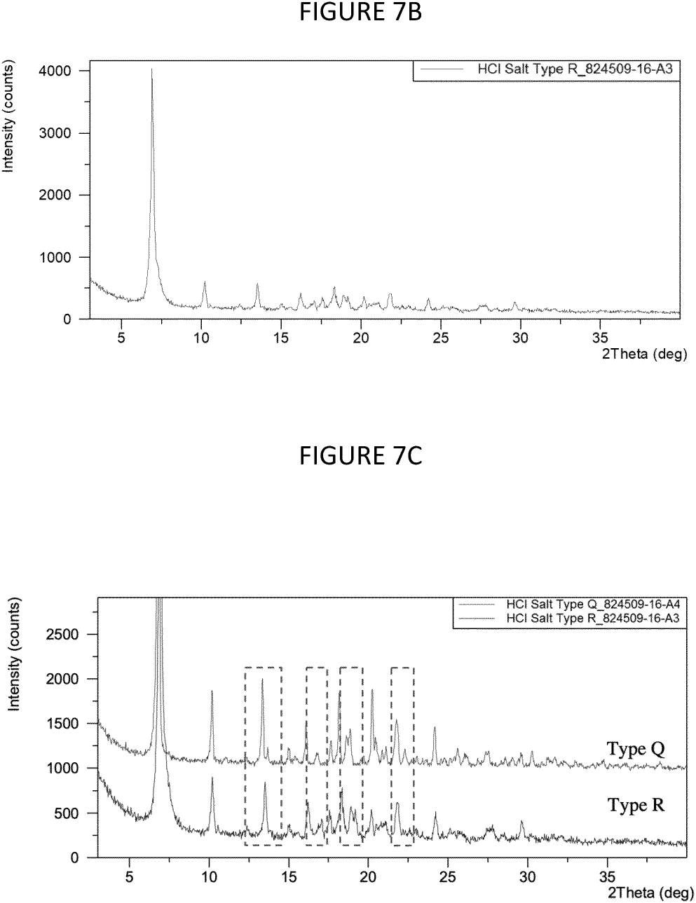

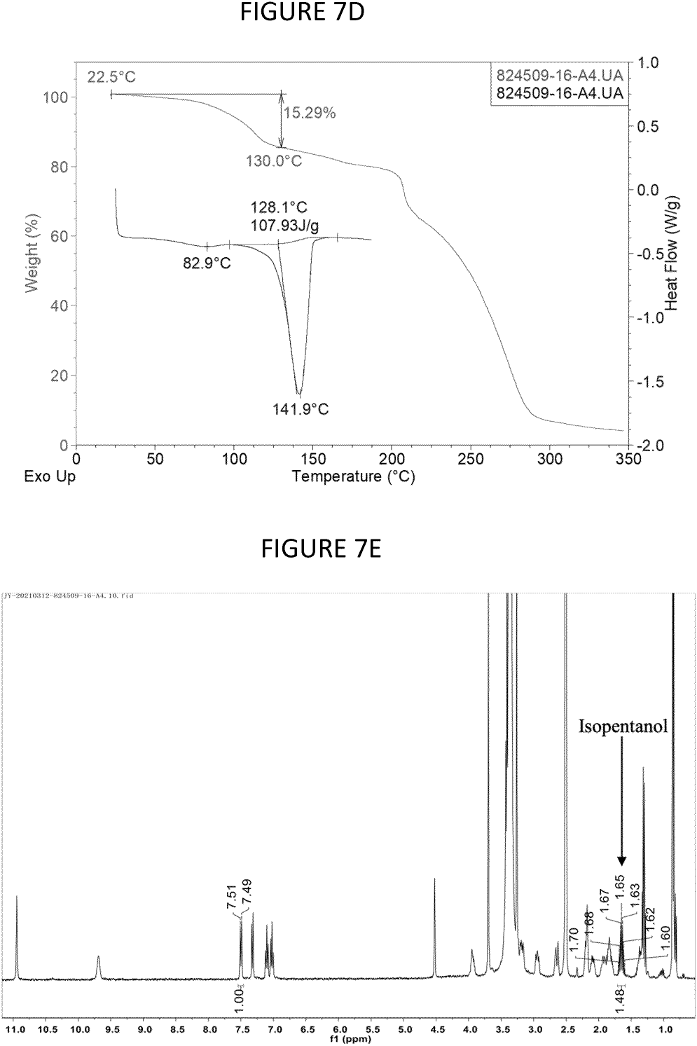

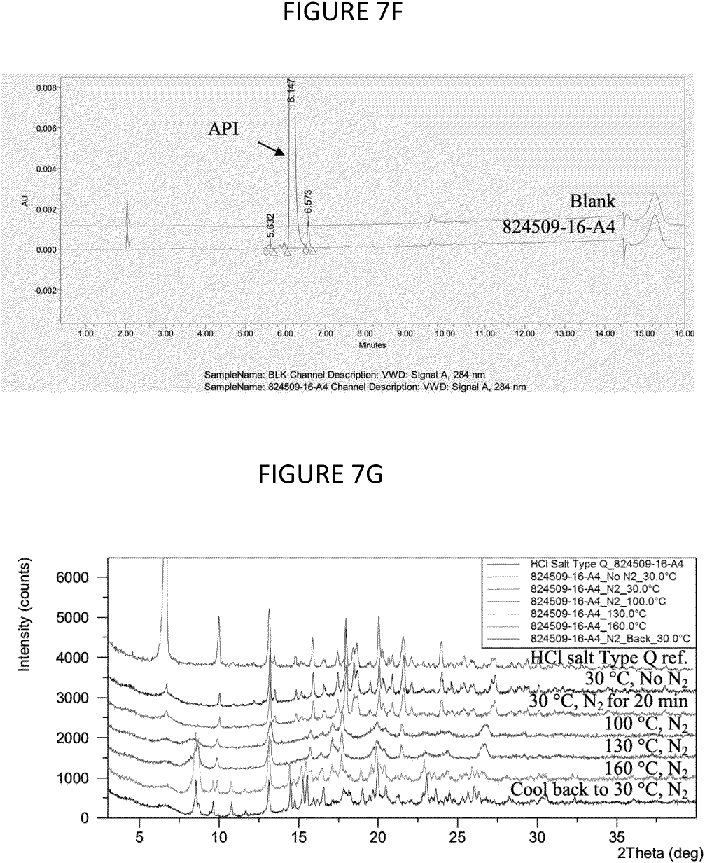

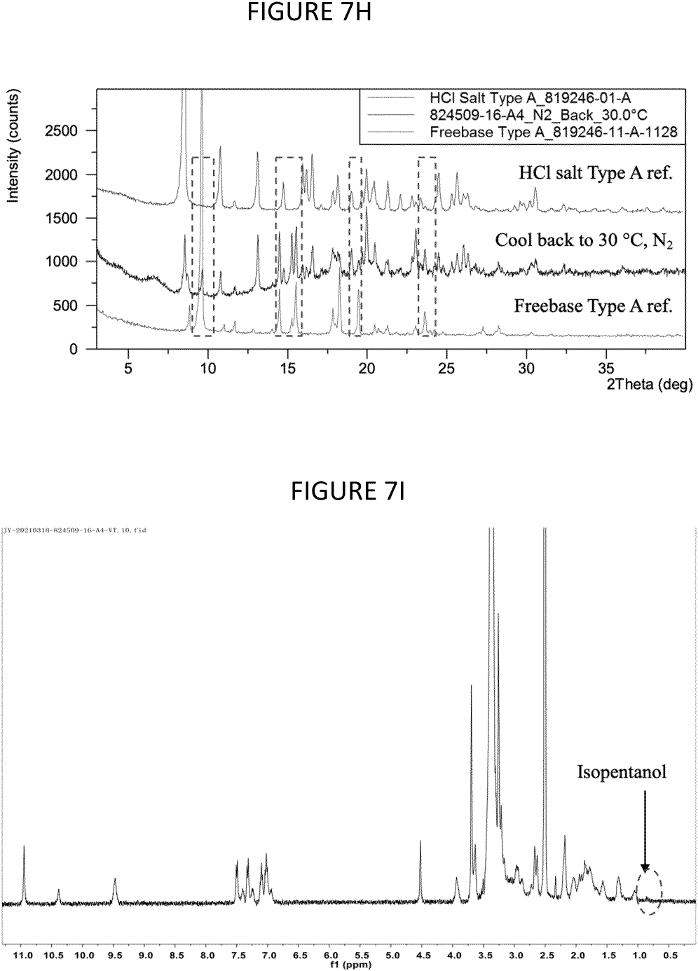

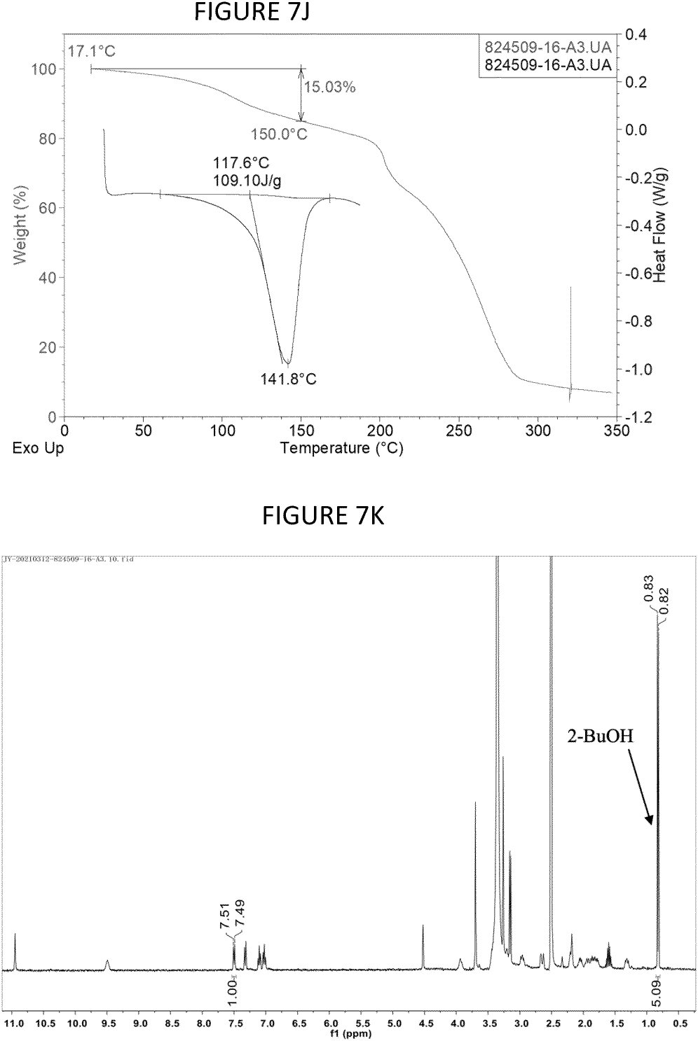

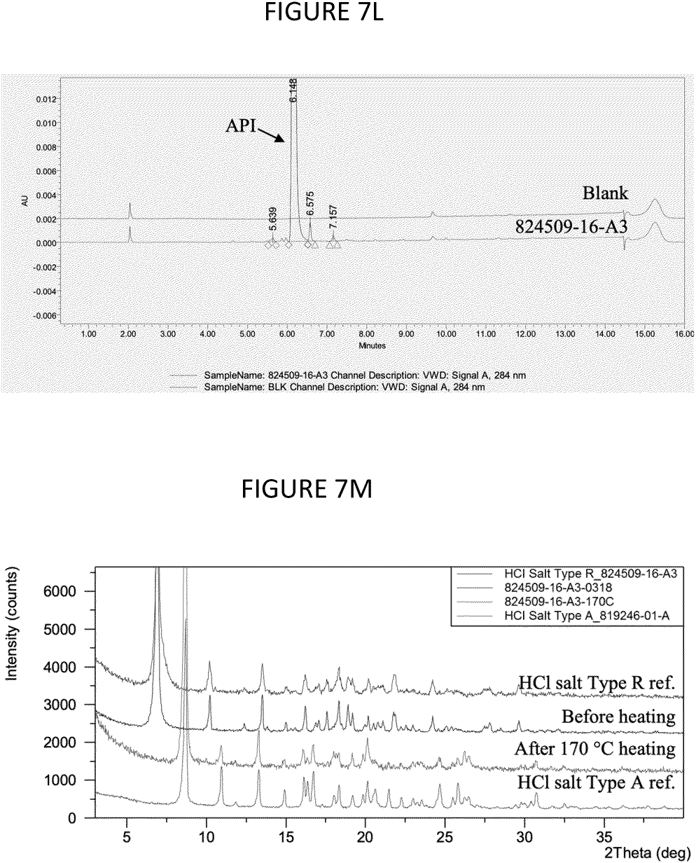

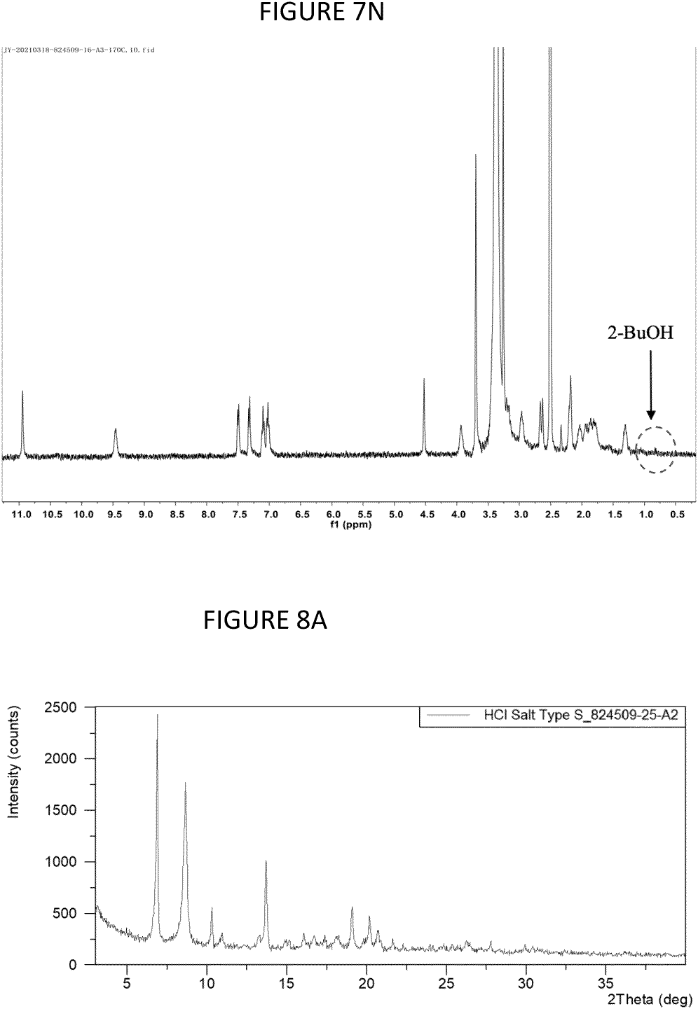

HCl salt Type Q (824509-16-A4) was obtained by slurrying 20.3 mg HCl salt Type A (824509-12-E) in 0.5 mL isopentanol at 60° C. for about 4 days. Resulting solids were isolated by centrifugation and air drying. HCl salt Type R (824509-16-A3) was obtained from 2-BuOH via the same method. XRPD results were shown in FIGS. 7A to 7C, TABLE 9A and TABLE 9B. The two forms showed similar XRPD patterns with differences marked in FIG. 7C and peak list of (824509-16-A4) was shown in TABLE 9A.

For HCl salt Type Q (824509-16-A4), TGA/DSC results in FIG. 7D showed a weight loss of 15.3% up to 130° C. and two endothermic signals at 82.9° C. and 141.9° C. (peak). ¹H NMR result in FIG. 7E showed that the peak of isopentanol was observed. The molar ratio of isopentanol/API was 0.7:1 (theoretical weight=11.6 wt%). HPLC/IC results showed that the molar ratio of acid/freebase was determined as 1.0:1 and HPLC purity was 99.74 area% (FIG. 7F and TABLE 9C). VT-XRPD results in FIG. 7G showed that after N₂-drying for 20 min at 30° C., no form change was observed for HCl salt Type Q. After heating HCl salt Type Q to 100° C., 130° C., 160° C. and cooling back to 30° C. under N₂ protection, form change to a mixture of HCl salt Type A and freebase Type A (FIG. 7H, peak of freebase Type A was marked in red frame) was observed. In FIG. 7I, ¹H NMR result showed that after VT-XRPD test, no signal of isopentanol was observed. Combined with the VT-XRPD results, HCl salt Type Q was speculated as an isopentanol solvate.

For HCl salt Type R (824509-16-A3), TGA/DSC results in FIG. 7J showed a weight loss of 15.0% up to 150° C. and one endothermic signal at 141.8° C. (peak). ¹H NMR result in FIG. 7K showed that the peak of 2-BuOH was observed, the molar ratio of 2-BuOH/API was 0.8:1 (theoretical weight=13.4 wt%). HPLC/IC results showed that the molar ratio of acid/freebase was determined as 1.0:1 and HPLC purity was 99.64 area% (FIG. 7L and TABLE 9D). XRPD results in FIG. 7M showed that after heating to 170° C. and cooling back to ambient condition, HCl salt Type R converted to HCl salt Type A. In FIG. 7N, ¹H NMR result showed that after 170° C. heating, no peak of 2-BuOH was observed. Combined with the results of heating experiment, HCl salt Type R was speculated as a 2-BuOH solvate that converted to Type A upon desolvation.

TABLE 9A

XRPD peak list of HCl salt Type Q (824509-16-A4)

| Pos. [°2θ] | Height [cts] | FWHM Left [°2θ] | d-spacing [Å] | Rel. Int. [%] |
|---|---|---|---|---|
| 6.8367 | 6087.57 | 0.1023 | 12.93 | 100.00 |
| 10.1865 | 793.00 | 0.1023 | 8.68 | 13.03 |
| 11.0375 | 41.39 | 0.1535 | 8.02 | 0.68 |
| 12.3258 | 34.88 | 0.1535 | 7.18 | 0.57 |
| 13.3516 | 945.98 | 0.1023 | 6.63 | 15.54 |
| 13.6793 | 165.23 | 0.0768 | 6.47 | 2.71 |
| 15.0080 | 157.18 | 0.1023 | 5.90 | 2.58 |
| 15.4155 | 63.48 | 0.1535 | 5.75 | 1.04 |
| 16.0891 | 468.31 | 0.1023 | 5.51 | 7.69 |
| 16.7806 | 99.85 | 0.2303 | 5.28 | 1.64 |
| 17.6519 | 251.02 | 0.1023 | 5.02 | 4.12 |
| 18.1664 | 776.25 | 0.1023 | 4.88 | 12.75 |
| 18.6301 | 303.78 | 0.1023 | 4.76 | 4.99 |
| 18.8633 | 366.55 | 0.1279 | 4.70 | 6.02 |
| 19.6942 | 93.74 | 0.0768 | 4.51 | 1.54 |
| 20.2423 | 839.30 | 0.1279 | 4.39 | 13.79 |
| 20.4550 | 281.74 | 0.0768 | 4.34 | 4.63 |
| 20.8601 | 153.01 | 0.0768 | 4.26 | 2.51 |
| 21.1052 | 202.59 | 0.1023 | 4.21 | 3.33 |
| 21.7668 | 502.65 | 0.1791 | 4.08 | 8.26 |
| 22.2880 | 168.04 | 0.1279 | 3.99 | 2.76 |
| 23.0403 | 83.87 | 0.0768 | 3.86 | 1.38 |
| 24.1656 | 437.19 | 0.1279 | 3.68 | 7.18 |
| 24.7948 | 75.31 | 0.1023 | 3.59 | 1.24 |
| 25.1430 | 75.68 | 0.1023 | 3.54 | 1.24 |
| 25.6020 | 194.87 | 0.1279 | 3.48 | 3.20 |
| 26.1026 | 111.33 | 0.3070 | 3.41 | 1.83 |
| 27.3740 | 160.75 | 0.0768 | 3.26 | 2.64 |
| 27.5340 | 156.40 | 0.1023 | 3.24 | 2.57 |
| 28.5977 | 101.09 | 0.1023 | 3.12 | 1.66 |
| 29.0527 | 89.92 | 0.1535 | 3.07 | 1.48 |
| 29.5873 | 153.29 | 0.1023 | 3.02 | 2.52 |
| 30.2655 | 170.71 | 0.1023 | 2.95 | 2.80 |
| 31.2581 | 77.12 | 0.1023 | 2.86 | 1.27 |
| 31.6937 | 110.99 | 0.1279 | 2.82 | 1.82 |
| 32.3320 | 58.50 | 0.2558 | 2.77 | 0.96 |
| 33.0029 | 58.42 | 0.1535 | 2.71 | 0.96 |
| 34.7312 | 69.73 | 0.2047 | 2.58 | 1.15 |
| 35.2024 | 29.10 | 0.1535 | 2.55 | 0.48 |
| 36.1220 | 40.92 | 0.3070 | 2.49 | 0.67 |
| 38.2988 | 59.07 | 0.2047 | 2.35 | 0.97 |

TABLE 9B

XRPD peak list of HCl salt Type R (824509-16-A3)

| Pos. [°2θ] | Height [cts] | FWHM Left [°2θ] | d-spacing [Å] | Rel. Int. [%] |
|---|---|---|---|---|
| 6.9149 | 3640.27 | 0.1535 | 12.78 | 100.00 |
| 10.1976 | 387.30 | 0.1535 | 8.67 | 10.64 |
| 12.4141 | 49.90 | 0.1535 | 7.13 | 1.37 |
| 13.5143 | 411.69 | 0.1279 | 6.55 | 11.31 |
| 15.0076 | 72.56 | 0.1535 | 5.90 | 1.99 |
| 16.2228 | 261.60 | 0.2558 | 5.46 | 7.19 |
| 17.0711 | 132.10 | 0.1023 | 5.19 | 3.63 |
| 17.5707 | 195.60 | 0.1023 | 5.05 | 5.37 |
| 18.3226 | 345.73 | 0.1023 | 4.84 | 9.50 |
| 18.9057 | 229.42 | 0.1279 | 4.69 | 6.30 |
| 19.1782 | 219.39 | 0.1023 | 4.63 | 6.03 |
| 20.1889 | 202.72 | 0.1535 | 4.40 | 5.57 |
| 21.0391 | 111.89 | 0.1535 | 4.22 | 3.07 |
| 21.8279 | 266.75 | 0.2303 | 4.07 | 7.33 |
| 22.9810 | 66.20 | 0.2047 | 3.87 | 1.82 |
| 24.2142 | 170.26 | 0.0768 | 3.68 | 4.68 |
| 25.0611 | 34.09 | 0.8187 | 3.55 | 0.94 |
| 25.7841 | 47.56 | 0.3070 | 3.46 | 1.31 |
| 27.7604 | 90.17 | 0.1535 | 3.21 | 2.48 |
| 28.4655 | 29.63 | 0.3070 | 3.14 | 0.81 |
| 29.6346 | 135.29 | 0.1279 | 3.01 | 3.72 |

TABLE 9C

HPLC results of HCl salt Type Q (824509-16-A4)

| # | RRT | Area (%) |
|---|---|---|
| 1 | 0.92 | 0.07 |
| 2 | 1.00 | 99.74 |
| 3 | 1.07 | 0.20 |

TABLE 9D

HPLC results of HCl salt Type R (824509-16-A3)

| # | RRT | Area (%) |
|---|---|---|
| 1 | 0.92 | 0.08 |
| 2 | 1.00 | 99.64 |
| 3 | 1.07 | 0.21 |
| 4 | 1.16 | 0.07 |

### HCl Salt Type S

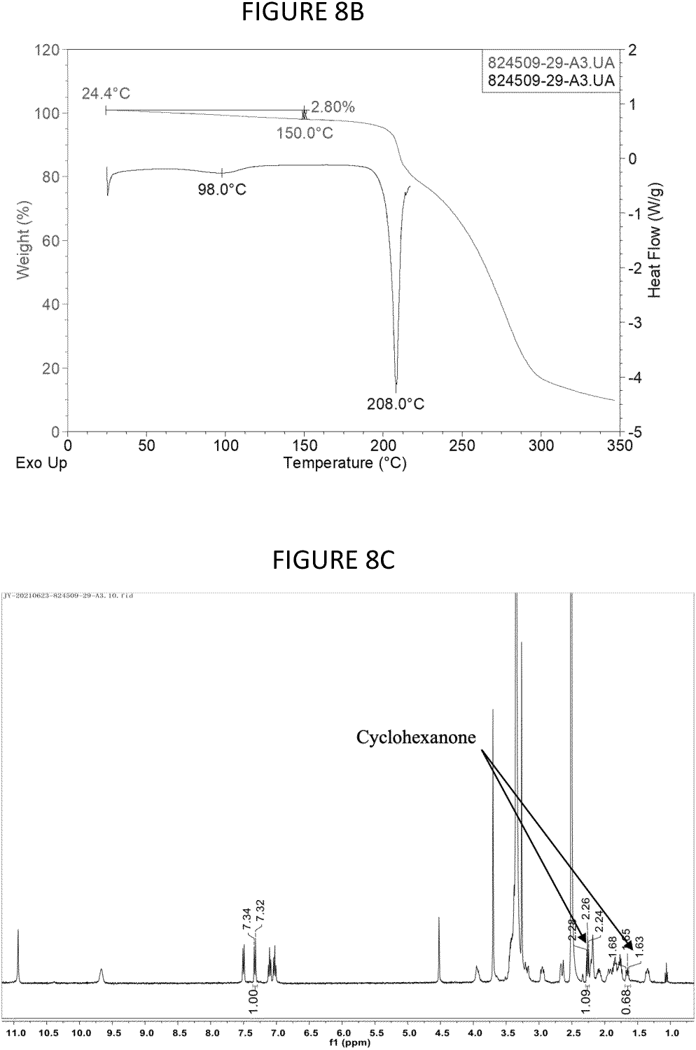

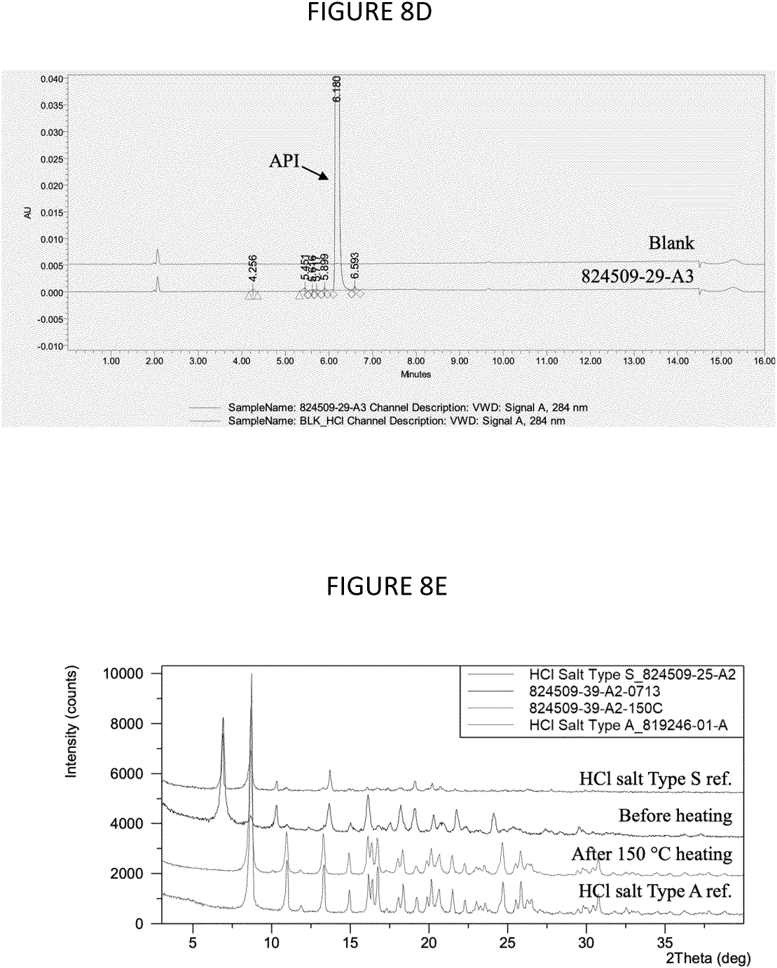

HCl salt Type S (824509-25-A2) was obtained by slurrying 19.7 mg amorphous HCl salt in 0.5 mL cyclohexanone at -20° C. for about 4 days. The resulting solids were isolated by centrifugation and the wet cake was tested by XRPD. The Type S sample turned to be gel like after air drying at RT It was re-prepared (824509-29-A3) by slurrying amorphous HCl salt in cyclohexanone at -20° C. for 7 days, and vacuum drying at RT for ~4 hours. The XRPD results are displayed in FIG. 8A and TABLE 10A. TGA/DSC results in FIG. 8B showed a weight loss of 2.8% up to 150° C., DSC result showed two endothermic signals at 98.0° C. and 208.0° C. (peak). ¹H NMR results in FIG. 8C showed that the peak of cyclohexanone was observed. The molar ratio of cyclohexanone/API was 0.15:1 (theoretical weight=3.51 wt%). HPLC/IC results showed that the molar ratio of acid/freebase was determined as 1.0:1 and HPLC purity was 99.20 area% (FIG. 8D and TABLE 10B).

XRPD results in FIG. 8E showed that after heating Type S sample (824509-39-A2) to 150° C., most diffraction peaks were consistent with HCl salt Type A. Using DMSO-d₆ as solvent, ¹H NMR result in FIG. 8F showed that after 150° C. heating, no peak of cyclohexanone was observed. Combined with the results of heating experiment and no obvious form transition signal in DSC, it was only obtained in a cyclohexanone system, HCl salt Type S was possibly a cyclohexanone solvate. Since the molar ratio of solvent to API was a bit low, it could be a channel solvate which may have a nonstoichiometric amount of solvent.

TABLE 10A

XRPD peak list of HCl salt Type S (824509-25-A2)

| Pos. [°2θ] | Height [cts] | FWHM Left [°2θ] | d-spacing [Å] | Rel. Int. [%] |
|---|---|---|---|---|
| 6.8818 | 2141.72 | 0.1023 | 12.84 | 100.00 |
| 8.6571 | 1545.41 | 0.1279 | 10.21 | 72.16 |
| 10.3087 | 350.67 | 0.0768 | 8.58 | 16.37 |
| 10.9371 | 125.09 | 0.1023 | 8.09 | 5.84 |
| 13.2665 | 113.73 | 0.1535 | 6.67 | 5.31 |
| 13.6955 | 832.25 | 0.1023 | 6.47 | 38.86 |
| 14.9129 | 64.46 | 0.1535 | 5.94 | 3.01 |
| 15.1596 | 76.27 | 0.1023 | 5.84 | 3.56 |
| 16.0782 | 128.70 | 0.1023 | 5.51 | 6.01 |
| 16.7165 | 122.36 | 0.2047 | 5.30 | 5.71 |
| 17.4065 | 112.06 | 0.1023 | 5.09 | 5.23 |
| 18.1695 | 108.72 | 0.3070 | 4.88 | 5.08 |
| 19.0986 | 402.54 | 0.1279 | 4.65 | 18.79 |
| 20.1946 | 311.82 | 0.1023 | 4.40 | 14.56 |
| 20.7143 | 178.10 | 0.0768 | 4.29 | 8.32 |
| 21.6597 | 108.00 | 0.1023 | 4.10 | 5.04 |
| 23.9704 | 69.65 | 0.0768 | 3.71 | 3.25 |
| 25.3494 | 56.22 | 0.1535 | 3.51 | 2.62 |
| 26.2623 | 93.36 | 0.1535 | 3.39 | 4.36 |
| 27.7703 | 97.96 | 0.0768 | 3.21 | 4.57 |
| 29.9192 | 66.51 | 0.1535 | 2.99 | 3.11 |
| 30.4027 | 50.78 | 0.1535 | 2.94 | 2.37 |

TABLE 10B

HPLC results of HCl salt Type S (824509-29-A3)

| # | RRT | Area (%) | # | RRT | Area (%) |
|---|---|---|---|---|---|
| 1 | 0.69 | 0.11 | 5 | 0.95 | 0.13 |
| 2 | 0.88 | 0.20 | 6 | 1.00 | 99.20 |
| 3 | 0.91 | 0.09 | 7 | 1.07 | 0.18 |
| 4 | 0.93 | 0.09 | -- | -- | -- |

### HCl Salt Type T

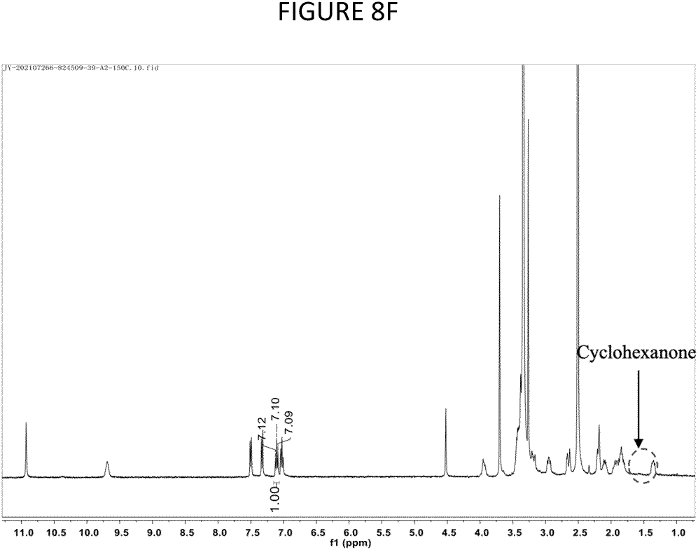

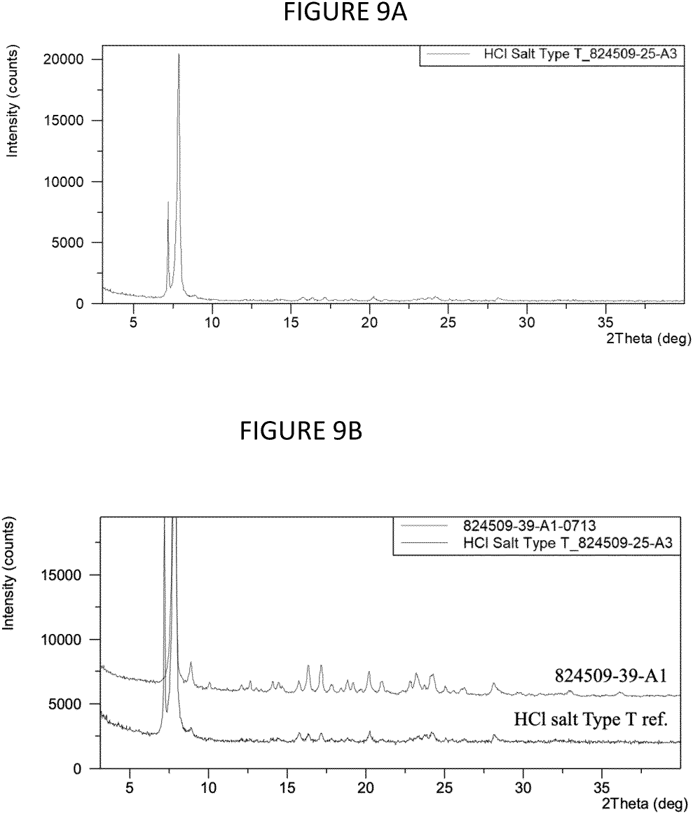

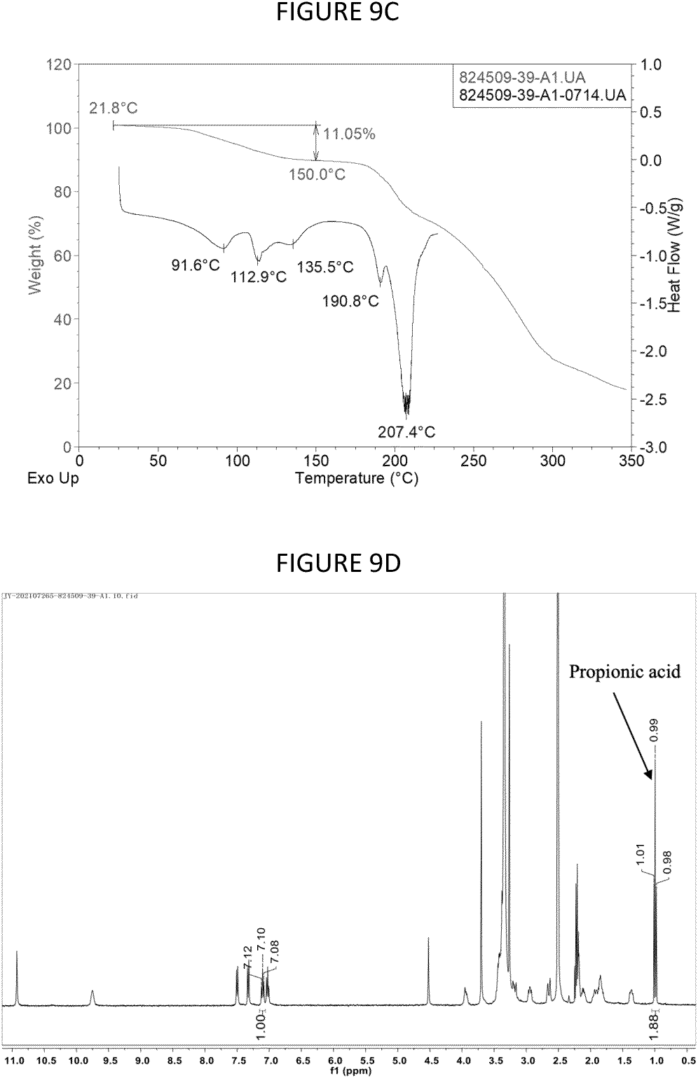

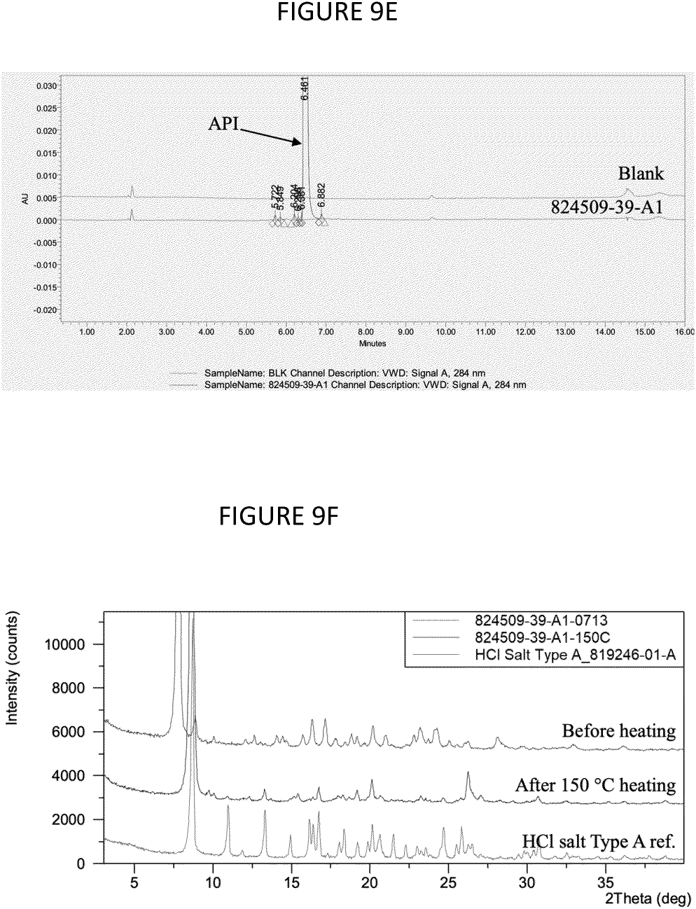

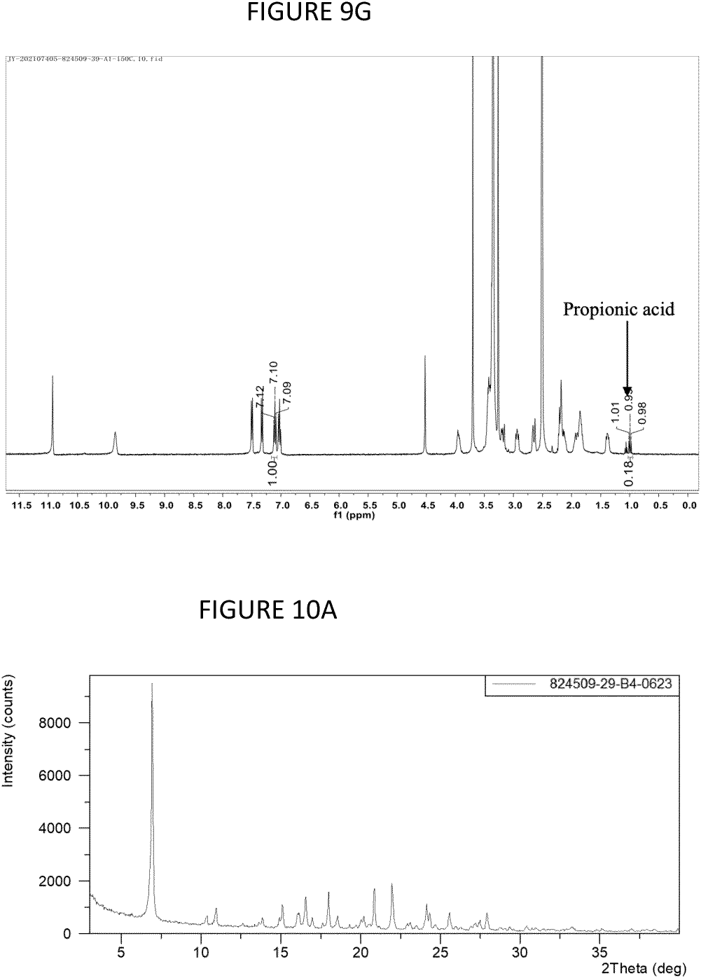

HCl salt Type T (824509-39-A1) was obtained by slurrying 40 mg HCl salt amorphous in 0.5 mL propionic acid at -20° C. for 4 days and drying at RT with silica gel. The XRPD results are displayed in FIG. 9A, FIG. 9B and TABLE 11A. TGA/DSC results in FIG. 9C showed a weight loss of 11.1% up to 150° C., DSC results showed five exothermic signals at 91.6° C., 112.9° C., 135.5° C., 190.8° C. and 207.4° C. (peak). ¹H NMR spectrum in FIG. 9D showed that the peak of propionic acid was observed. The molar ratio of propionic acid/API was 0.63:1 (theoretical weight=10.29 wt%). HPLC/IC results showed that the molar ratio of acid/freebase was determined as 1.0:1 and HPLC purity was 99.38 area% (FIG. 9E and TABLE 11B).

XRPD results in FIG. 9F showed that after heating HCl salt Type T sample to 150° C. and cooling back to RT, most diffraction peaks were consistent with HCl salt Type A. ¹H NMR result in FIG. 9G showed that amount of propionic acid decreased significantly (molar ratio of propionic acid/API was 0.06:1, theoretical weight=1.09 wt%). Combined with the results of heating experiment, HCl salt Type T was speculated as a propionic acid solvate that desolvated to Type A.

TABLE 11A

XRPD peak list of HCl salt Type T (824509-25-A3)

| Pos. [°2θ] | Height [cts] | FWHM Left [°2θ] | d-spacing [Å] | Rel. Int. [%] |
|---|---|---|---|---|
| 7.1830 | 3066.20 | 0.0768 | 12.31 | 35.83 |
| 7.8513 | 8556.73 | 0.1535 | 11.26 | 100.00 |
| 8.8658 | 142.87 | 0.2047 | 9.97 | 1.67 |
| 14.0275 | 31.81 | 0.1535 | 6.31 | 0.37 |
| 15.7626 | 135.92 | 0.1535 | 5.62 | 1.59 |
| 16.3362 | 111.98 | 0.2047 | 5.43 | 1.31 |
| 17.1460 | 124.06 | 0.1791 | 5.17 | 1.45 |
| 17.8005 | 44.21 | 0.1535 | 4.98 | 0.52 |
| 18.8161 | 57.85 | 0.1535 | 4.72 | 0.68 |
| 20.2187 | 148.29 | 0.1023 | 4.39 | 1.73 |
| 20.9912 | 30.04 | 0.3070 | 4.23 | 0.35 |
| 23.2949 | 75.33 | 0.2558 | 3.82 | 0.88 |
| 23.7709 | 99.64 | 0.2047 | 3.74 | 1.16 |
| 24.1995 | 141.46 | 0.2558 | 3.68 | 1.65 |
| 26.2438 | 40.70 | 0.2047 | 3.40 | 0.48 |
| 28.1464 | 100.40 | 0.1279 | 3.17 | 1.17 |

TABLE 11B

HPLC results of HCl salt Type T (824509-39-A1)

| # | RRT | Area (%) | # | RRT | Area (%) |
|---|---|---|---|---|---|
| 1 | 0.89 | 0.14 | 5 | 0.99 | 0.06 |
| 2 | 0.91 | 0.09 | 6 | 1.00 | 99.38 |
| 3 | 0.96 | 0.15 | 7 | 1.07 | 0.14 |
| 4 | 0.97 | 0.05 | -- | -- | -- |

### HCl Salt Type U

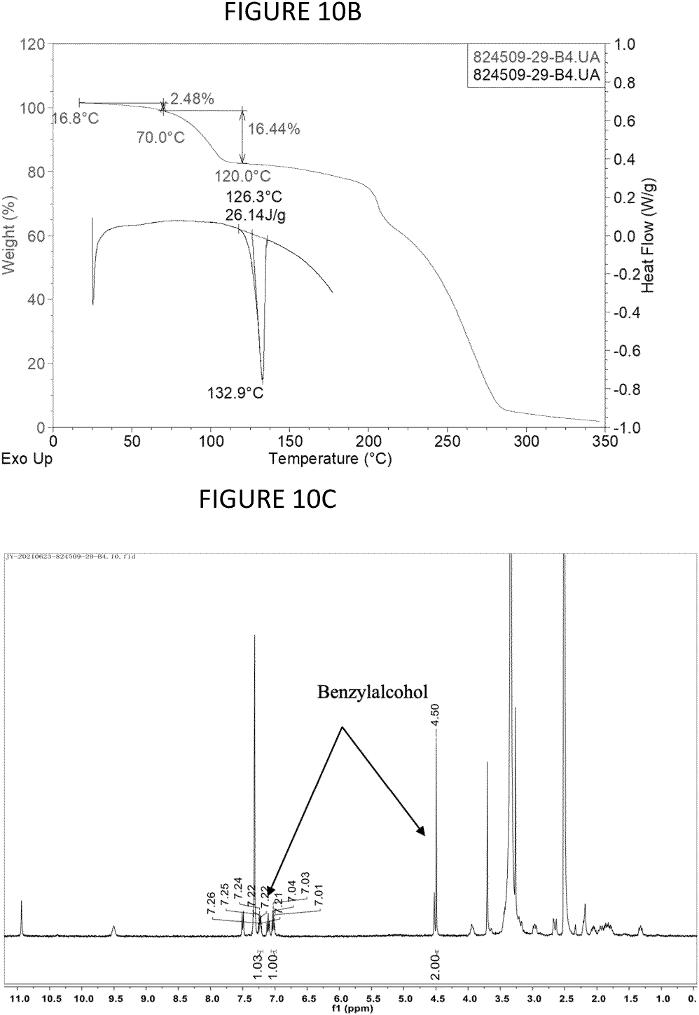

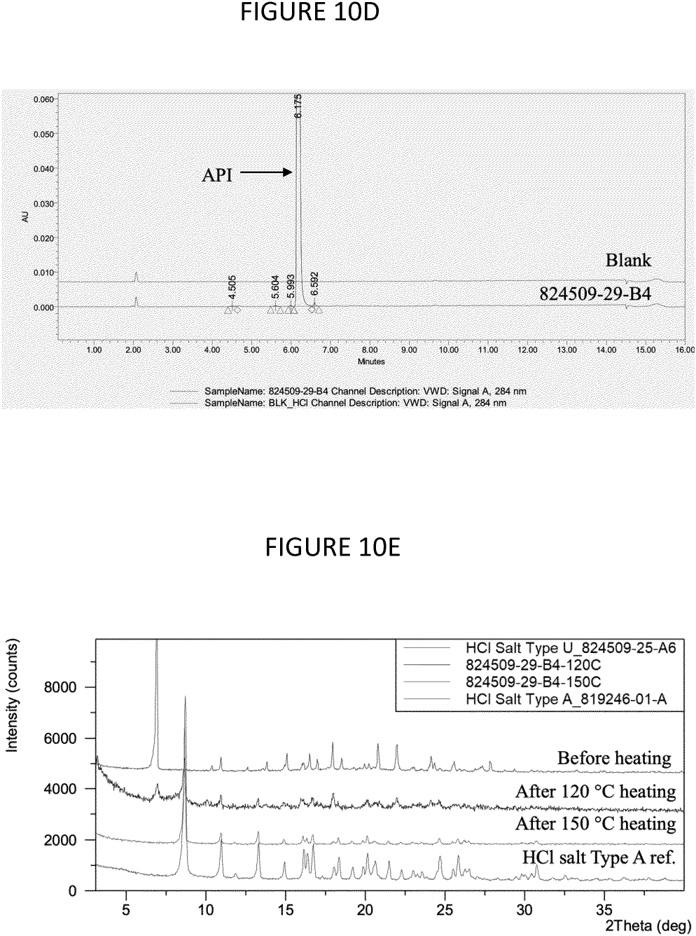

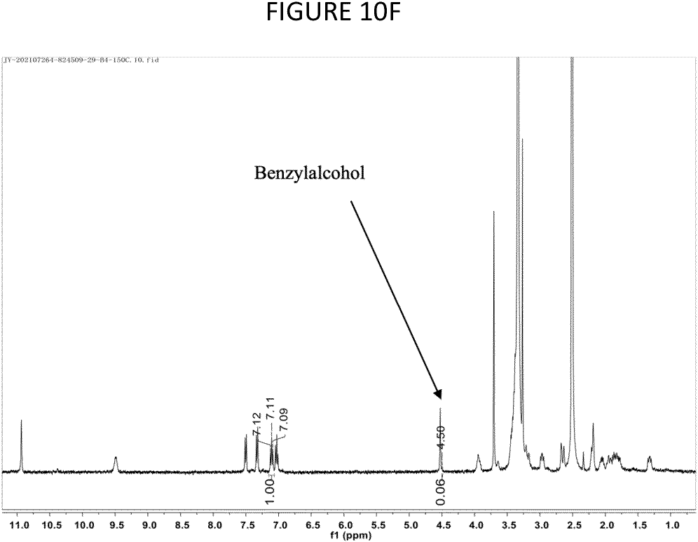

HCl salt Type U (824509-29-B4) was obtained by slurry 40 mg HCl salt amorphous in 0.5 mL benzylalcohol/methyl acetate (1:1, v/v) at -20° C. for 3 days, and isolated by centrifugation and vacuum dried at 50° C. ~3 hrs, and this sample was used for characterization. The XRPD results were displayed in FIG. 10A, and TABLE 12A. TGA/DSC results in FIG. 10B showed a weight loss of 2.5% up to 70° C. and a weight loss of 16.4% from 70° C. up to 120° C., DSC result showed one endothermic signal at 132.9° C. (peak). ¹H NMR spectrum in FIG. 10C showed that the peak of benzyl alcohol was observed. The molar ratio of benzyl alcohol/API was 1.0:1 (theoretical weight=21.08 wt%). HPLC/IC results showed that the molar ratio of acid/freebase was determined as 0.9:1 and HPLC purity was 99.67 area% (FIG. 10D and TABLE 12B).

XRPD results in FIG. 10E showed that after heating HCl salt Type U to 120° C., an extra peak representative of HCl salt Type A was observed, and after heating to 150° C., most diffraction peaks were consistent with HCl salt Type A. ¹H NMR result in FIG. 10F showed that after heating to 150° C., the peak of benzyl alcohol in the sample was decreased significantly (molar ratio of benzyl alcohol/API was 0.03:1, theoretical weight=0.79 wt%). Combined with the results of heating experiment, HCl salt Type U was speculated to be a benzyl alcohol solvate that converted to Type A upon desolvation.

TABLE 12A

XRPD peak list of HCl salt Type U (824509-29-B4)

| Pos. [°2θ] | Height [cts] | FWHM Left [°2θ] | d-spacing [Å] | Rel. Int. [%] |
|---|---|---|---|---|
| 6.9258 | 8407.24 | 0.1023 | 12.76 | 100.00 |
| 10.3439 | 319.38 | 0.1023 | 8.55 | 3.80 |
| 10.9234 | 654.34 | 0.0768 | 8.10 | 7.78 |
| 12.6085 | 132.01 | 0.1023 | 7.02 | 1.57 |
| 13.6269 | 151.06 | 0.0768 | 6.50 | 1.80 |
| 13.8378 | 338.35 | 0.1023 | 6.40 | 4.02 |
| 14.9045 | 358.59 | 0.0512 | 5.94 | 4.27 |
| 15.1010 | 835.45 | 0.1023 | 5.87 | 9.94 |
| 16.0463 | 535.52 | 0.0768 | 5.52 | 6.37 |
| 16.1420 | 570.66 | 0.0512 | 5.49 | 6.79 |
| 16.5518 | 1172.82 | 0.1279 | 5.36 | 13.95 |
| 16.9699 | 394.40 | 0.1023 | 5.22 | 4.69 |
| 17.6223 | 186.34 | 0.0768 | 5.03 | 2.22 |
| 17.9822 | 1319.12 | 0.1023 | 4.93 | 15.69 |
| 18.5515 | 463.05 | 0.1023 | 4.78 | 5.51 |
| 19.3093 | 154.21 | 0.0768 | 4.60 | 1.83 |
| 19.7151 | 123.56 | 0.0768 | 4.50 | 1.47 |
| 20.0277 | 319.61 | 0.0768 | 4.43 | 3.80 |
| 20.2052 | 427.36 | 0.0768 | 4.40 | 5.08 |
| 20.8588 | 1537.45 | 0.1279 | 4.26 | 18.29 |
| 21.9709 | 1691.57 | 0.1023 | 4.05 | 20.12 |
| 22.9237 | 197.23 | 0.0768 | 3.88 | 2.35 |
| 23.0990 | 241.42 | 0.1023 | 3.85 | 2.87 |
| 23.5033 | 114.37 | 0.1279 | 3.79 | 1.36 |
| 24.1348 | 944.21 | 0.1023 | 3.69 | 11.23 |
| 24.3242 | 606.32 | 0.0768 | 3.66 | 7.21 |
| 24.6759 | 176.76 | 0.1279 | 3.61 | 2.10 |
| 25.5610 | 604.10 | 0.1279 | 3.48 | 7.19 |
| 25.9623 | 114.83 | 0.1279 | 3.43 | 1.37 |
| 26.3213 | 70.50 | 0.1535 | 3.39 | 0.84 |
| 26.9095 | 133.90 | 0.1279 | 3.31 | 1.59 |
| 27.1807 | 254.45 | 0.1535 | 3.28 | 3.03 |
| 27.4601 | 335.66 | 0.1023 | 3.25 | 3.99 |
| 27.9229 | 631.73 | 0.1535 | 3.20 | 7.51 |
| 28.7283 | 106.48 | 0.1279 | 3.11 | 1.27 |
| 29.0600 | 56.36 | 0.1279 | 3.07 | 0.67 |
| 29.3460 | 135.18 | 0.1023 | 3.04 | 1.61 |
| 30.4158 | 164.84 | 0.1535 | 2.94 | 1.96 |
| 30.7500 | 89.28 | 0.0768 | 2.91 | 1.06 |
| 30.9821 | 89.88 | 0.1535 | 2.89 | 1.07 |
| 31.4468 | 52.08 | 0.1535 | 2.84 | 0.62 |
| 33.2667 | 145.34 | 0.1791 | 2.69 | 1.73 |
| 34.8239 | 50.93 | 0.1535 | 2.58 | 0.61 |
| 35.1246 | 85.48 | 0.1791 | 2.55 | 1.02 |
| 37.0058 | 90.00 | 0.1535 | 2.43 | 1.07 |
| 37.8543 | 43.55 | 0.2047 | 2.38 | 0.52 |
| 38.4008 | 50.94 | 0.2047 | 2.34 | 0.61 |

TABLE 12B

HPLC results of HCl salt Type U (824509-29-B4)

| # | RRT | Area (%) |
|---|---|---|
| 1 | 0.73 | 0.06 |
| 2 | 0.91 | 0.07 |
| 3 | 0.97 | 0.06 |
| 4 | 1.00 | 99.67 |
| 5 | 1.07 | 0.15 |

### Metastable Forms

### HCl Salt Type V

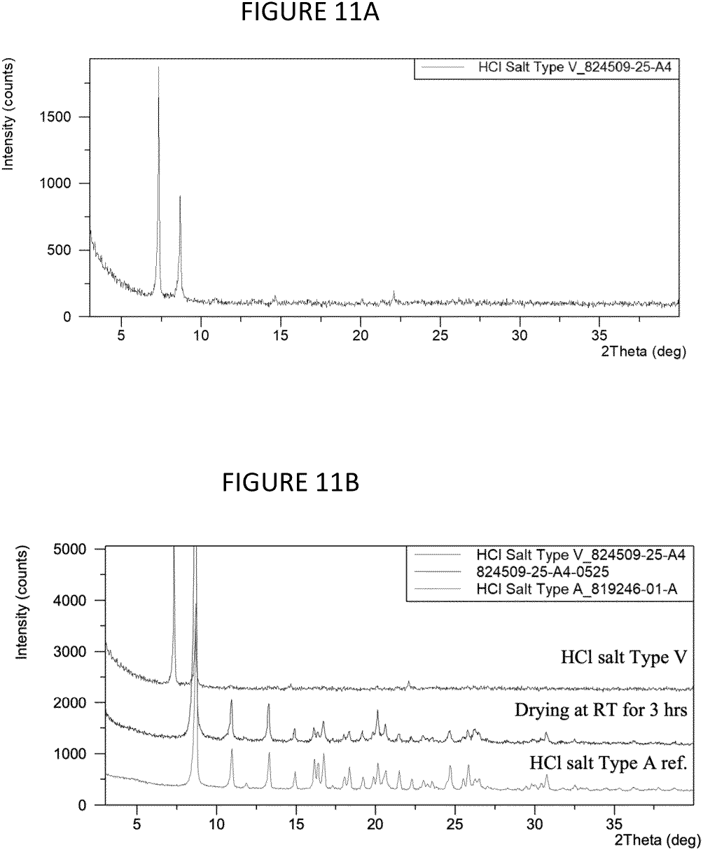

HCl salt Type V (824509-25-A4) was obtained by slurry 20.0 mg HCl salt amorphous in 0.5 mL trifluoroethanol/m-xylene (1:1, v/v) at -20° C. overnight, then the clear solution was transferred to anti-solvent addition (EtOAc, 4.5 mL) at RT and stir at 5 and -20° C. to produce more solids for 5 days. Resulting solids were isolated by centrifugation and air dried in desiccator at RT with silica gel for ~3 hrs. The XRPD overlay of the wet sample was displayed in FIG. 11A and peak list of (824509-25-A4) was shown in TABLE 13. XRPD result in FIG. 11B showed that after air drying the wet cake for 3 hrs, the sample converted to HCl salt Type A completely. Since HCl salt Type V was not obtained in the re-preparation trials (details refer to Section 4.6), no more characterization data was collected.

TABLE 13

XRPD peak list of HCl salt Type V (824509-25-A4)

| Pos. [°2θ] | Height [cts] | FWHM Left [°2θ] | d-spacing [Å] | Rel. Int. [%] |
|---|---|---|---|---|
| 7.3300 | 1675.34 | 0.1023 | 12.06 | 100.00 |
| 8.6726 | 772.55 | 0.1023 | 10.20 | 46.11 |
| 10.9060 | 28.44 | 0.3070 | 8.11 | 1.70 |
| 14.6667 | 52.37 | 0.0768 | 6.04 | 3.13 |
| 21.3841 | 22.11 | 0.2047 | 4.16 | 1.32 |
| 22.0888 | 84.38 | 0.0768 | 4.02 | 5.04 |

### HCl Salt Type L/N/O Mixtures

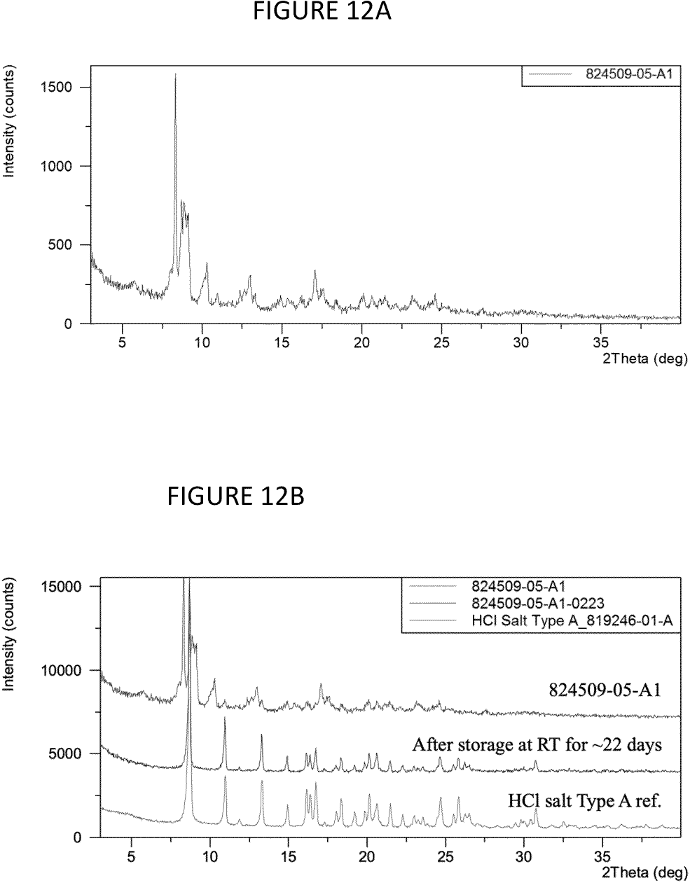

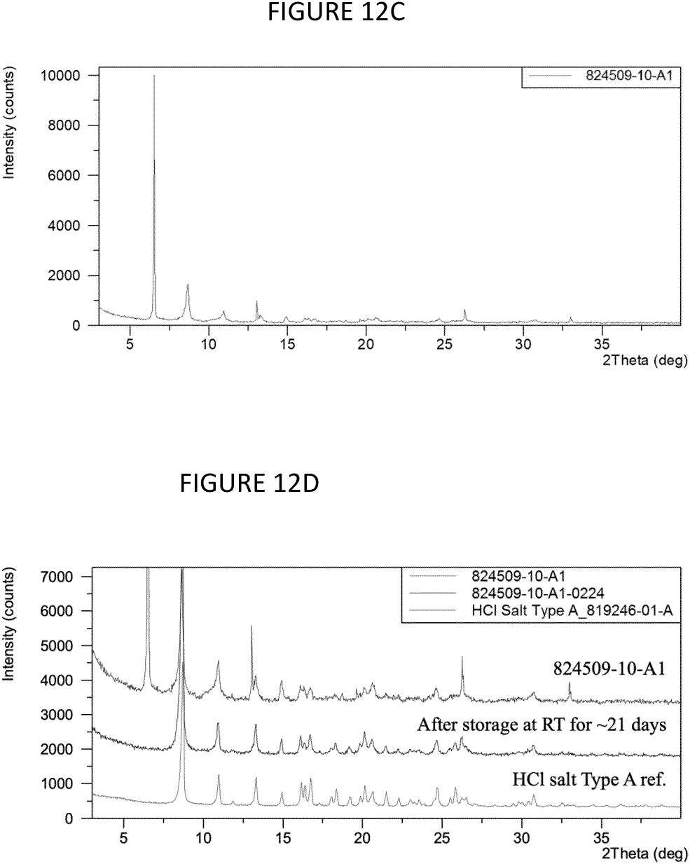

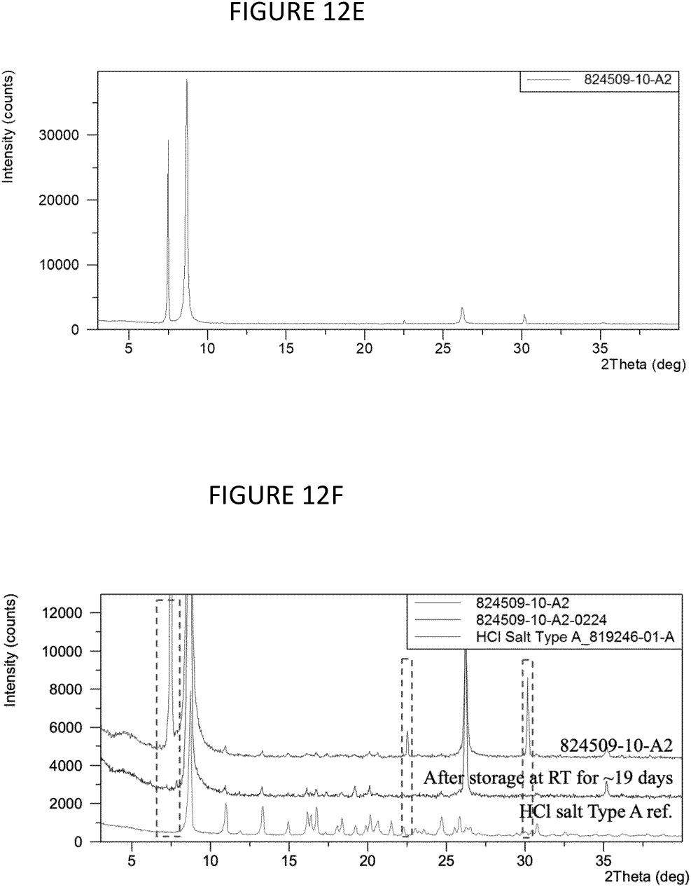

Sample (824509-05-A1, FIG. 12A and TABLE 14A) was obtained by salt formation through liquid vapor diffusion in MIBK/n-pentane system. The detailed procedure was as follows: dissolve 19.9 mg freebase in 1.0 mL MIBK at RT in a 4-mL vial. Dilute 1 mL HCŀEtOAc solution (conc. of HCl was 2 mol/L) by 3 mL n-pentane in a 20-mL vial. Put the uncapped 4-mL vial into the 20-mL vial and keep the capped 20-mL vial at RT for about 4 days. Solids were isolated by centrifugation and air dried for characterization.

XRPD pattern in FIG. 12B showed that the sample (824509-05-A1) had strong extra peaks compared with HCl salt Type A, which was assigned as HCl salt Type A+L. After storing the sample (824509-05-A1) at RT for ~22 days, it converted to HCl salt Type A completely. Since no pure Type L was obtained in the screening, no more characterization was performed.

TABLE 14A

XRPD peak list of sample HCl salt Type A+L (824509-05-A1)

| Pos. [°2θ] | Height [cts] | FWHM Left [°2θ] | d-spacing [Å] | Rel. Int. [%] |
|---|---|---|---|---|
| 8.3084 | 1460.11 | 0.0768 | 10.64 | 100.00 |
| 8.6902 | 615.03 | 0.1023 | 10.18 | 42.12 |
| 8.8606 | 605.73 | 0.1023 | 9.98 | 41.49 |
| 9.0908 | 510.56 | 0.1023 | 9.73 | 34.97 |
| 10.2770 | 226.81 | 0.1023 | 8.61 | 15.53 |
| 10.9446 | 63.28 | 0.1023 | 8.08 | 4.33 |
| 12.3765 | 91.21 | 0.1023 | 7.15 | 6.25 |
| 12.9727 | 198.54 | 0.1791 | 6.82 | 13.60 |
| 14.9087 | 73.22 | 0.1535 | 5.94 | 5.01 |
| 15.3694 | 66.72 | 0.1535 | 5.77 | 4.57 |
| 16.1839 | 59.69 | 0.2047 | 5.48 | 4.09 |
| 17.0667 | 244.07 | 0.1279 | 5.20 | 16.72 |
| 17.5711 | 133.50 | 0.1023 | 5.05 | 9.14 |
| 18.3944 | 53.55 | 0.1535 | 4.82 | 3.67 |
| 20.0839 | 85.11 | 0.3070 | 4.42 | 5.83 |
| 20.6384 | 90.47 | 0.1279 | 4.30 | 6.20 |
| 21.4503 | 82.45 | 0.2558 | 4.14 | 5.65 |
| 22.1298 | 47.46 | 0.3070 | 4.02 | 3.25 |
| 23.1698 | 85.69 | 0.2047 | 3.84 | 5.87 |
| 24.5975 | 110.49 | 0.1023 | 3.62 | 7.57 |
| 27.5450 | 24.92 | 0.3070 | 3.24 | 1.71 |

HCl salt Type A+N (824509-10-A1, FIG. 12C and TABLE 14B) was obtained by adding 1.0 mL 2,2,2-trifluoroethanol solution (conc. of HCl salt was ~20 mg/mL) in 9.0 mL toluene directly at RT and stir at RT for 1 days. Since no solids precipitated after RT stirring, the clear solution was transferred to stir at 5° C. overnight, -20° C. overnight and evaporation at RT for about 5 days to dryness. XRPD pattern in FIG. 12D showed that the sample (824509-10-A1) had some strong extra peaks compared with HCl salt Type A, which was assigned as HCl salt Type A+N. After storage the sample (824509-10-A1) at RT for ~21 days, it converted to HCl salt Type A completely. Since pure Type N was not obtained, additional characterization was not performed.

TABLE 14B

XRPD peak list of HCl salt Type A+N (824509-10-A1)

| Pos. [°2θ] | Height [cts] | FWHM Left [°2θ] | d-spacing [Å] | Rel. Int. [%] |
|---|---|---|---|---|
| 6.5178 | 9308.03 | 0.0768 | 13.56 | 100.00 |
| 8.6470 | 1443.74 | 0.1279 | 10.23 | 15.51 |
| 10.9326 | 424.22 | 0.1279 | 8.09 | 4.56 |
| 13.0505 | 815.04 | 0.0512 | 6.78 | 8.76 |
| 13.2894 | 268.12 | 0.1279 | 6.66 | 2.88 |
| 14.9033 | 222.17 | 0.1535 | 5.94 | 2.39 |
| 16.1002 | 177.75 | 0.1023 | 5.51 | 1.91 |
| 16.7126 | 142.17 | 0.1535 | 5.30 | 1.53 |
| 18.2852 | 60.10 | 0.1535 | 4.85 | 0.65 |
| 18.7161 | 90.80 | 0.1023 | 4.74 | 0.98 |
| 19.6712 | 91.02 | 0.1535 | 4.51 | 0.98 |
| 20.1310 | 139.72 | 0.1535 | 4.41 | 1.50 |
| 20.6381 | 192.37 | 0.2047 | 4.30 | 2.07 |
| 24.6266 | 146.91 | 0.1535 | 3.62 | 1.58 |
| 26.2764 | 394.91 | 0.0768 | 3.39 | 4.24 |
| 30.7334 | 87.69 | 0.1535 | 2.91 | 0.94 |
| 33.0067 | 169.09 | 0.0768 | 2.71 | 1.82 |

HCl salt Type A+O (824509-10-A2, FIG. 12E and TABLE 14C) was obtained by reverse anti-solvent addition in 2,2,2-trifluoroethanol/THF systems. XRPD result showed that the sample (824509-10-A2) had some strong extra peaks compared with HCl salt Type A, assigned as HCl salt Type A+O. After storing the sample (824509-10-A2) at RT for ~19 days, it converted to HCl salt Type A completely (FIG. 12F). Since pure Type O was not obtained, additional characterization was not performed.

TABLE 14C

XRPD peak list of sample HCl salt Type A+O (824509-10-A2)

| Pos. [°2θ] | Height [cts] | FWHM Left [°2θ] | d-spacing [Å] | Rel. Int. [%] |
|---|---|---|---|---|
| 7.4751 | 25692.02 | 0.0768 | 11.83 | 68.04 |
| 8.6672 | 37762.42 | 0.1279 | 10.20 | 100.00 |
| 10.9322 | 165.97 | 0.0768 | 8.09 | 0.44 |
| 13.2752 | 98.69 | 0.1023 | 6.67 | 0.26 |
| 14.9439 | 53.12 | 0.1791 | 5.93 | 0.14 |
| 16.3660 | 30.10 | 0.6140 | 5.42 | 0.08 |
| 16.7016 | 66.13 | 0.1023 | 5.31 | 0.18 |
| 17.4006 | 43.82 | 0.1535 | 5.10 | 0.12 |
| 19.1397 | 33.42 | 0.3070 | 4.64 | 0.09 |
| 20.0950 | 83.92 | 0.1535 | 4.42 | 0.22 |
| 20.5882 | 56.14 | 0.1535 | 4.31 | 0.15 |
| 22.5148 | 488.23 | 0.1023 | 3.95 | 1.29 |
| 26.2088 | 2500.46 | 0.1791 | 3.40 | 6.62 |
| 30.1642 | 1415.50 | 0.1279 | 2.96 | 3.75 |
| 35.1644 | 172.71 | 0.1535 | 2.55 | 0.46 |

### Characterization Information of HCl Salt Type A to K

A summary of information about HCl salt Type A to K is shown in TABLE 15.

TABLE 15

- Characterization summary of HCl salt Type A to K

| Solid form (ID: 819246-) | Crystallinity | TGA loss, % | Endotherm ic peak, °C. | Form after heating | Speculat ed form | Molar ratio^{&} |
|---|---|---|---|---|---|---|
| HCl salt Type A (01-A) | High | 1.4 (up to 150° C.) | 211.6 | -- | Anhydrate | 1:1 |
| HCl salt Type H (49-A2) | High | 2.7 (up to 120° C.) | 69.9, 214.3 | Freebase Type A+extra peak | Anhydrate | 1:1 |
| HCl salt Type K (43-A8_N2_165.0° C.) | High | -- | -- | -- | Anhydrate | -- |
| HCl salt Type F (43-A8) | High | 5.2 (up to 100° C.) | 98.4, 211.4 | HCl salt Type K^{#} | Hydrate | 1:1 |
| HCl salt Type B (46-A2-AlRDRY) | High | 15.9 (up to 150° C.) | 129.8, 217.2 | HCl salt Type A | 1-BuOH solvate | -- |
| HCl salt Type C (47-A7) | Low | 17.8 (up to 150° C.) | 114.6, 211.9 | HCl salt Type A | Chlorofor m solvate | -- |
| HCl salt Type l (49-A5) | High | 11.6 (up to 170° C.) | 151.8, 215.7 | HCl salt Type A | Chlorofor m solvate | -- |
| HCl salt Type D (46-A13) | Low | -- | -- | HCl salt Type A* | Metastabl e | -- |
| HCl salt Type E (43-A5) | High | -- | -- | HCl salt Type A** | Metastabl e | -- |
| HCl salt Type G (48-A2) | High | 5.2 (up to 120° C.) | 108.7, 215.9 | HCl salt Type A** | Metastabl e | -- |
| HCl salt Type J (49-A6) | High | 7.6 (up to 140° C.) | 87.6, 195.6 | HCl salt Type F* | Metastabl e | -- |

### Conclusion

An additional polymorph screening was performed for 18-MC HCl salt. As the characterization results showed, 11 new forms were obtained. Among the forms obtained, HCl salt Type A was still the most thermodynamically stable form at RT based on desolvation and physical form conversion data.

### Example 3 - Polymorph Screening of 18-MC Sulfate

18-MC sulfate Type A was first obtained as described in Example 1. Polymorph screening of 18-MC sulfate was performed to better understand polymorphism of the salt.

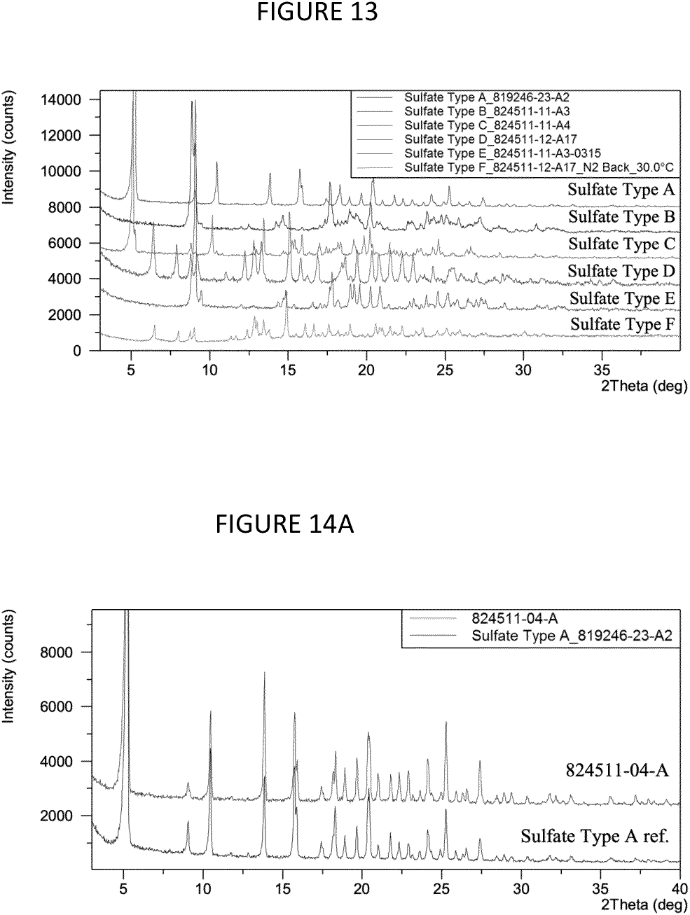

Sulfate material was first prepared using 18-MC freebase and used as starting material for polymorph screening. In the screening, different crystallization methods including temperature cycling and slurry conversion at different temperatures were used, and a total of 30 experiments were conducted. Solids from screening were isolated for XRPD. New forms were further characterized by TGA, DSC and HPLC/IC. As the characterization and identification results showed, five new forms (sulfate Type B-F) were discovered. Identification results indicated that sulfate Type A and Type F were anhydrates, Type D was a hydrate, Type E was a hydrate or anhydrate, Type B was an ACN solvate (converted to sulfate Type E after storage) and Type C was a metastable form (converted to sulfate Type A after air drying). Characterization results are summarized in TABLE 15 and XRPD patterns of different forms are displayed in FIG. 13.

To summarize, a brief polymorph screen was performed and a total of six forms of the sulfate were discovered.

TABLE 15

Characterization summary of sulfate salt forms

| | | | | | | |
|---|---|---|---|---|---|---|
| Solid form (ID: 824511-) | TGA loss (%) | Endotherm (°C., peak) | Solvent residual (wt%) | Molar ratio^{#} (acid/FB) | HPLC purity (area%) | Speculated form |
| Sulfate Type A (04-A) | 1.6 (up to 120° C.) | Overlapped signals around 176° C. | Not detected | 1.0 | 99.77 | Anhydrate^{&} |
| Sulfate Type B (11-A3) | 6.6 (up to 100° C.) | 65.4, 115.9, 146.8, 188.4 | 5.4 (ACN) | 0.9 | 99.64 | ACN solvate |
| Sulfate Type C* (11-A4) | -- | -- | -- | -- | -- | Metastable |
| Sulfate Type D (12-A17) | 2.3 (up to 100° C.) | 76.1, 194.9 | Not detected | 1.0 | 99.79 | Hydrate |
| Sulfate Type E (11-A3-0315) | 3.0 (up to 120° C.) | 74.1, 116.8, 147.0 | 0.3 (ACN) | -- | -- | Hydrate/ Anhydrate^{&&} |
| Sulfate Type F (12-A17_N2 Back_30.0° C.) | -- | -- | -- | -- | -- | Anhydrate |

FB: Freebase.
--: No data collected.
*: Converted to sulfate Type A after air drying at RT.
^{#}: Determined by HPLC/IC.
^{&}: Identified in Example 1.
^{&&}: No further identification was performed due to the failure of re-preparation.

### Preparation of Sulfate Starting Material

The preparation procedure of sulfate Type A (824511-04-A) was as follows: 2 g of freebase Type A (824509-01-A was weighed, 20 mL of EtOAc was added to prepare a suspension. 1.36 mL of 4 M H₂SO₄ was diluted by 20 mL of EtOAc. The diluted H₂SO₄ solution was added into the suspension under magnetic stirring dropwise. The sample was transferred to temperature cycling (50° C.~5° C., 3 cycles, one cycle: heat to 50° C. at 4.5° C./min, isothermal at 50° C. for 30 min; cool to 5° C. at 0.1° C./min, isothermal at 5° C. for 30 min; keep slurry at 5° C. at last). After XRPD confirmation, the solids were isolated by vacuum filtration and vacuum dried at RT for one day. As a result, about 1.77 g of sulfate Type A (824511-04-A) was obtained.

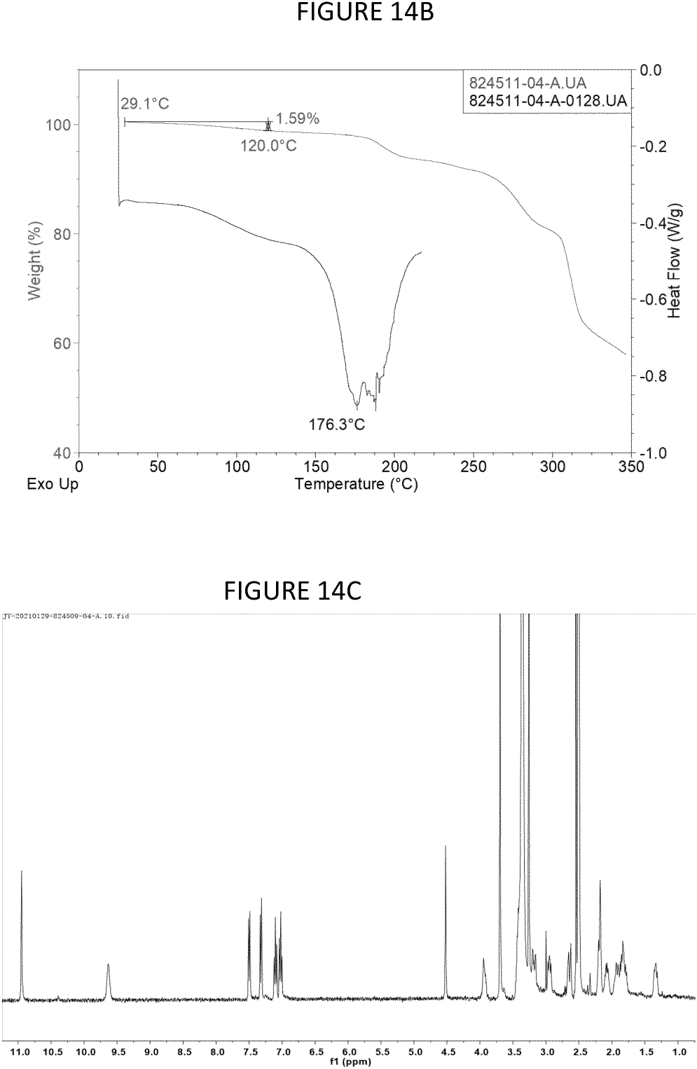

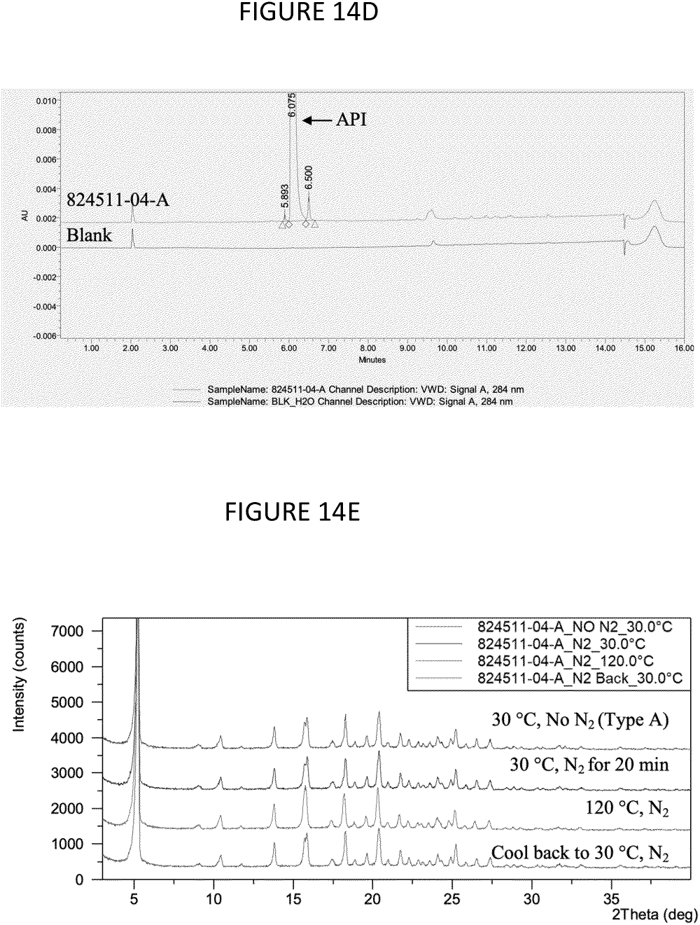

The XRPD pattern of prepared sulfate Type A in FIG. 14A was consistent with sulfate Type A reference (819246-23-A2). TGA/DSC curves in FIG. 14B showed a weight loss of 1.6% up to 120.0° C. and overlapped endothermic peaks around 176.3° C. (peak). ¹H NMR (DMSO-d₆ as solvent) result in FIG. 14C showed that EtOAc was not observed. The molar ratio of acid/freebase was determined as 0.97:1 by HPLC/IC and HPLC purity was tested to be 99.77 area% (FIG. 14D and TABLE 16A).

VT-XRPD results in FIG. 14E showed that after N₂-drying sulfate Type A (824511-04-A) for 20 min at 30° C. or heating sample to 120° C. and cooling back to 30° C. under N₂ protection, no obvious form change was observed, indicating sulfate Type A was an anhydrate.

Approximate solubility values of sulfate Type A (824511-04-A) were estimated in 10 solvents to guide the solvent selection in the polymorph screening, with results summarized in TABLE 16B.

TABLE 16A

HPLC results of sulfate Type A (824511-04-A)

| # | RRT | Area (%) |
|---|---|---|
| 1 | 0.97 | 0.05 |
| 2 | 1.00 | 99.77 |
| 3 | 1.07 | 0.18 |

TABLE 16B

Approximate solubility of sulfate Type A (824511-04-A) at RT

| | | | |
|---|---|---|---|
| Solvent | Solubility (mg/mL) | Solvent | Solubility (mg/mL) |
| MeOH | S>44.0 | DCM | S<2.1 |
| EtOH | 20.0<S<40.0 | EtOAc | S<2.0 |
| ACN | 20.0<S<40.0 | THF | S<1.9 |
| Acetone | 1.8<S<6.0 | CHCl₃ | S<1.9 |
| Toluene | S<2.2 | n-Heptane | S<1.8 |

Procedure: weigh ~2 mg solids into each 3-mL glass vial, add in corresponding solvent stepwise and sonicate or oscillate to see if solids dissolved completely. Stop adding solvent till the solids dissolves or total volume reaches 1.0 mL. Calculate the approximate solubility based on solvent volume.

XPRD data for sulfate Type A is in TABLE 16C.

TABLE 16C

| Pos. [°2Th.] | Height [cts] | FWHM Left [°2Th.] | d-spacing [Å] | Rel. Int. [%] |
|---|---|---|---|---|
| 5.224348 | 10929.060000 | 0.102336 | 16.91570 | 100.00 |
| 9.055523 | 459.518100 | 0.102336 | 9.76585 | 4.20 |
| 10.455820 | 1585.901000 | 0.102336 | 8.46089 | 14.51 |
| 11.731510 | 27.578460 | 0.307008 | 7.54357 | 0.25 |
| 12.835660 | 54.438060 | 0.102336 | 6.89703 | 0.50 |
| 13.847610 | 1224.717000 | 0.076752 | 6.39520 | 11.21 |
| 15.727110 | 1339.621000 | 0.102336 | 5.63491 | 12.26 |
| 15.889380 | 700.755900 | 0.051168 | 5.57772 | 6.41 |
| 17.413050 | 237.139000 | 0.102336 | 5.09295 | 2.17 |
| 18.306910 | 745.692800 | 0.102336 | 4.84625 | 6.82 |
| 18.910470 | 321.548500 | 0.102336 | 4.69291 | 2.94 |
| 19.663210 | 443.482300 | 0.102336 | 4.51492 | 4.06 |
| 20.449690 | 941.759200 | 0.179088 | 4.34303 | 8.62 |
| 21.006930 | 206.101700 | 0.102336 | 4.22906 | 1.89 |
| 21.778680 | 394.644900 | 0.102336 | 4.08092 | 3.61 |
| 22.319300 | 257.197300 | 0.102336 | 3.98328 | 2.35 |
| 22.914440 | 228.688700 | 0.127920 | 3.88115 | 2.09 |
| 23.157600 | 64.132550 | 0.076752 | 3.84095 | 0.59 |
| 23.630900 | 126.427000 | 0.102336 | 3.76508 | 1.16 |
| 24.131820 | 440.452900 | 0.127920 | 3.68805 | 4.03 |
| 24.918580 | 141.397100 | 0.102336 | 3.57336 | 1.29 |
| 25.270260 | 738.168800 | 0.127920 | 3.52442 | 6.75 |
| 25.878620 | 120.266600 | 0.102336 | 3.44293 | 1.10 |
| 26.328880 | 86.977580 | 0.076752 | 3.38507 | 0.80 |
| 26.553350 | 173.170700 | 0.127920 | 3.35696 | 1.58 |
| 27.410920 | 321.158500 | 0.153504 | 3.25385 | 2.94 |
| 28.436440 | 56.381220 | 0.127920 | 3.13879 | 0.52 |
| 28.928690 | 90.999190 | 0.102336 | 3.08650 | 0.83 |
| 29.378920 | 65.776550 | 0.153504 | 3.04021 | 0.60 |
| 30.391790 | 56.503970 | 0.153504 | 2.94116 | 0.52 |
| 31.773970 | 81.261820 | 0.153504 | 2.81630 | 0.74 |
| 33.196520 | 79.884060 | 0.179088 | 2.69880 | 0.73 |
| 35.726540 | 53.472470 | 0.179088 | 2.51327 | 0.49 |
| 37.200240 | 50.777340 | 0.204672 | 2.41702 | 0.46 |
| 37.974580 | 34.716400 | 0.127920 | 2.36950 | 0.32 |
| 39.246350 | 19.367600 | 0.255840 | 2.29560 | 0.18 |

### Polymorph Screening

Using prepared sulfate Type A (824511-04-A, anhydrate) as the starting material, a total of 30 polymorph screening experiments were conducted via various crystallization methods. Results of polymorph screening is summarized in TABLE 17A. Results showed that a total of 6 forms (sulfate Type A~F) were obtained in the polymorph screening, including 2 anhydrates (sulfate Type A/F), 1 hydrate (sulfate Type D), 1 solvate (sulfate Type B), and 1 metastable form (sulfate Type C). Sulfate Type E was a hydrate or anhydrate, while no further identification was conducted due to the failure of re-preparation trials. Characterization data of obtained forms was summarized in TABLE 15 and the XRPD overlays of these forms were displayed in FIG. 13.

TABLE 17A

Summary of polymorph screening experiments of sulfate

| Method | No. of Experiment | Results |
|---|---|---|
| Temperature Cycling | 5 | Sulfate Type A/B/C |
| Slurry at RT | 20 | Sulfate Type A/B/D, amorphous, gel |
| Slurry at 50° C. | 5 | Sulfate Type A/B |
| Total | 30 | Sulfate Type A/B/C/D, amorphous, gel |

Note: Sulfate Type E and F were found during form identification.

### Anhydrate and Hydrate

### Sulfate Type D and F

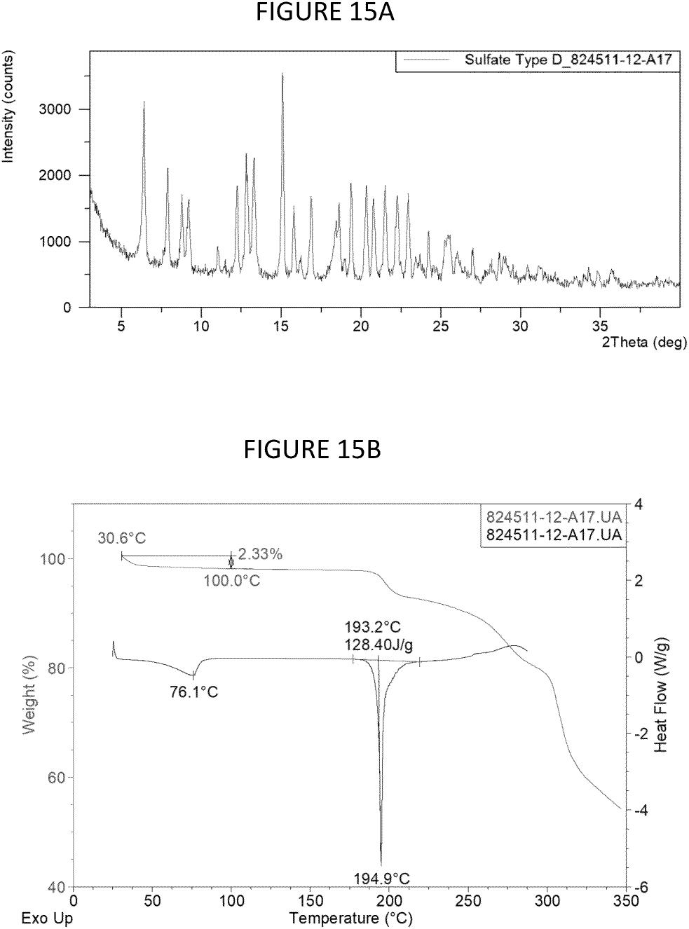

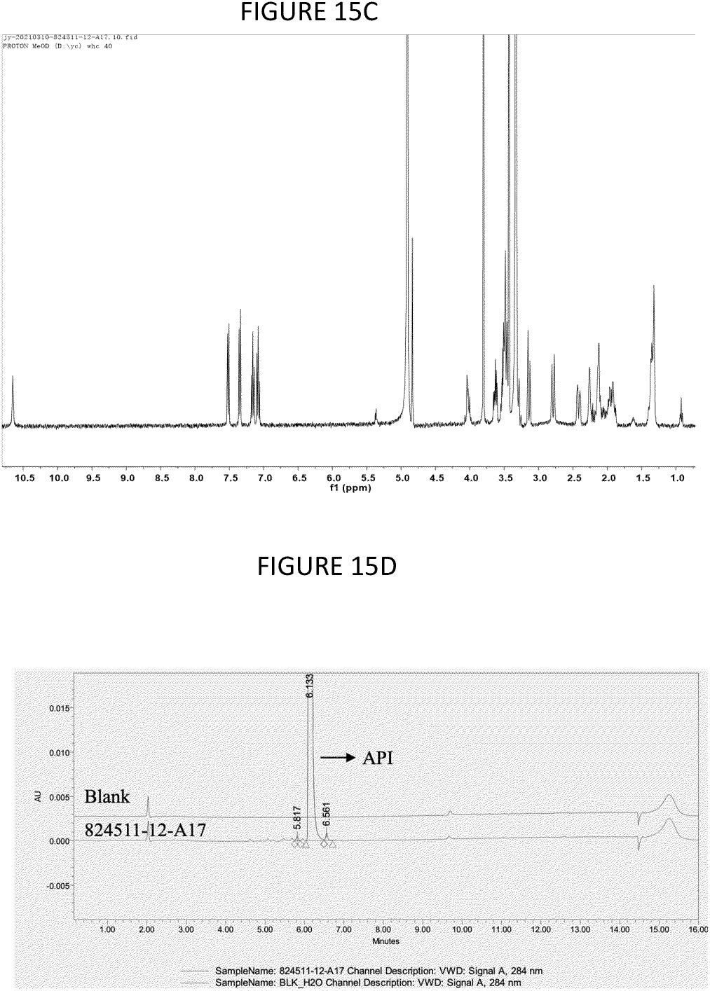

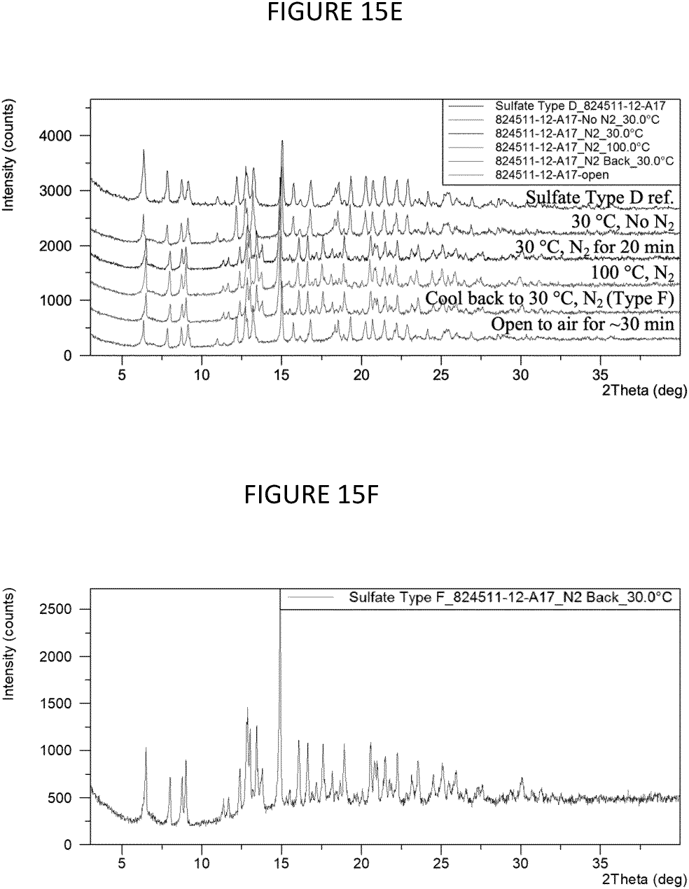

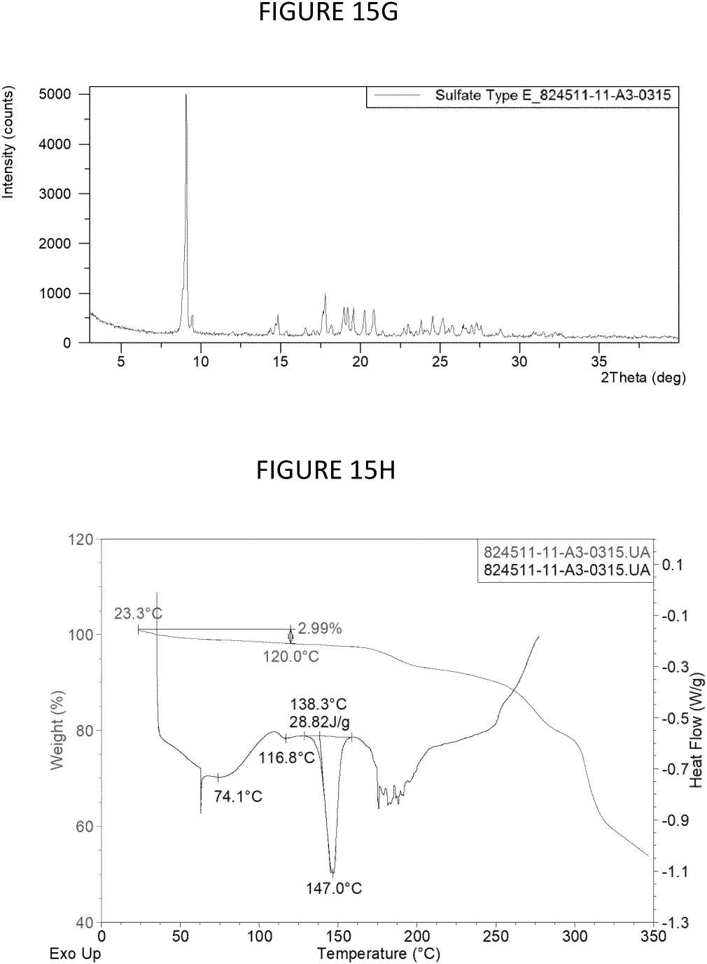

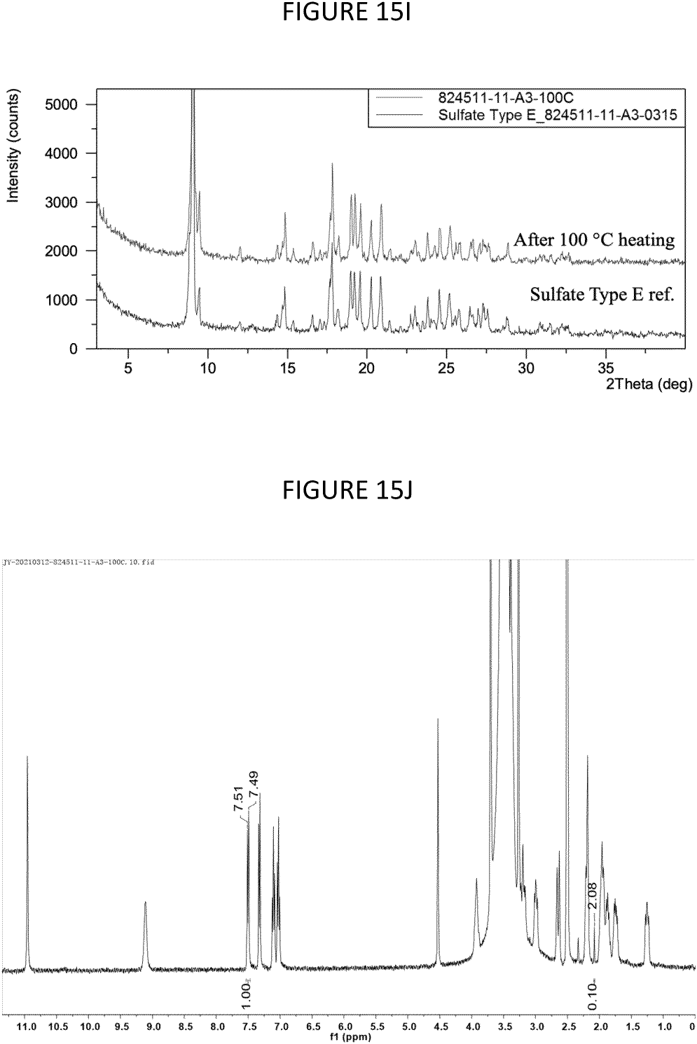

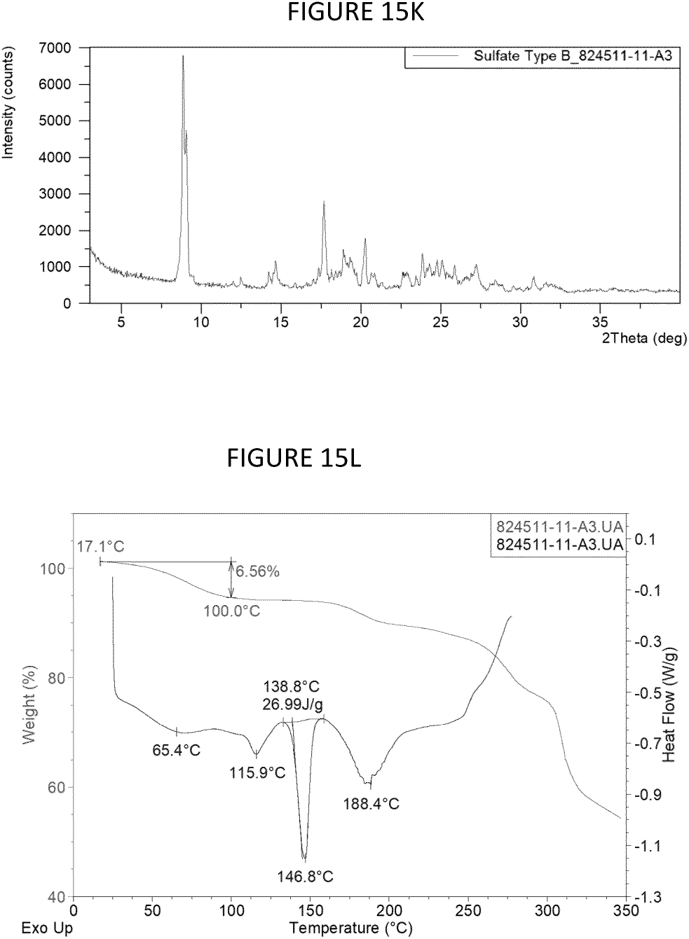

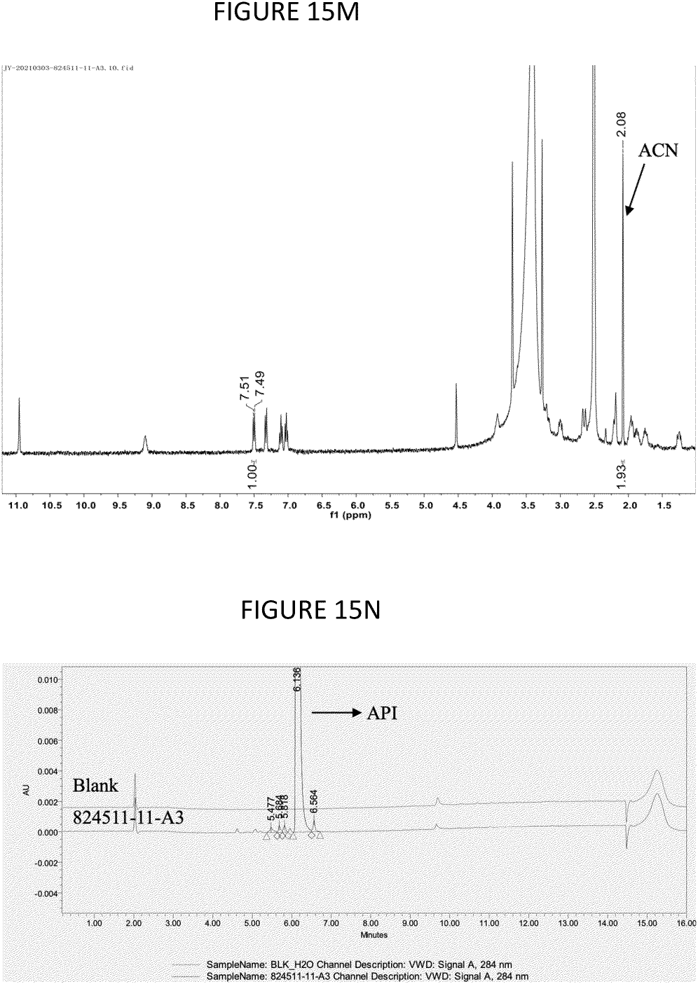

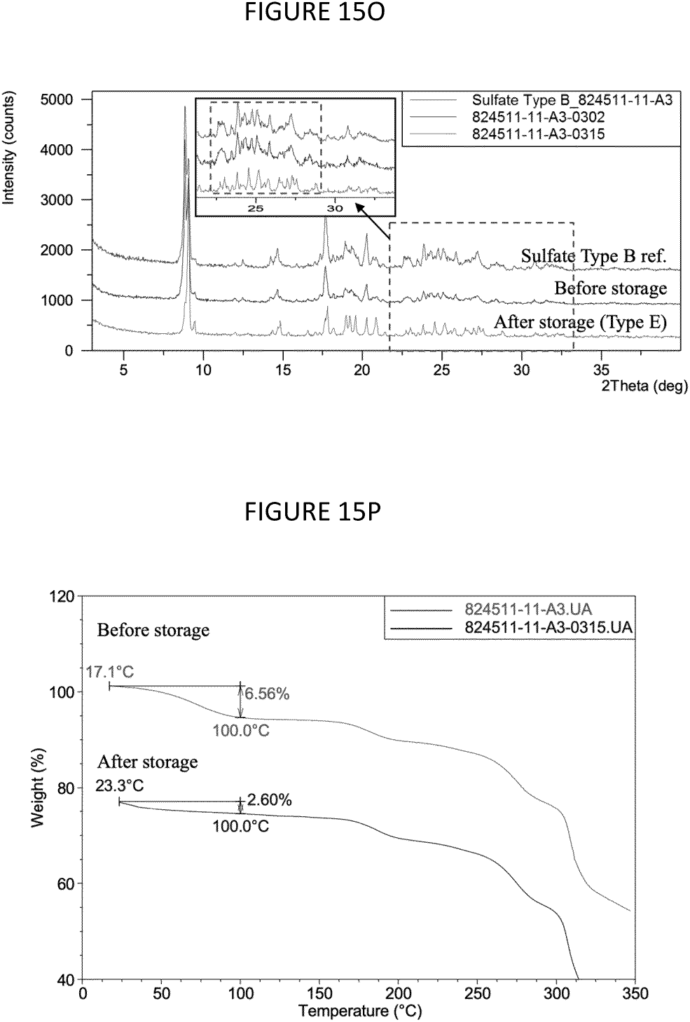

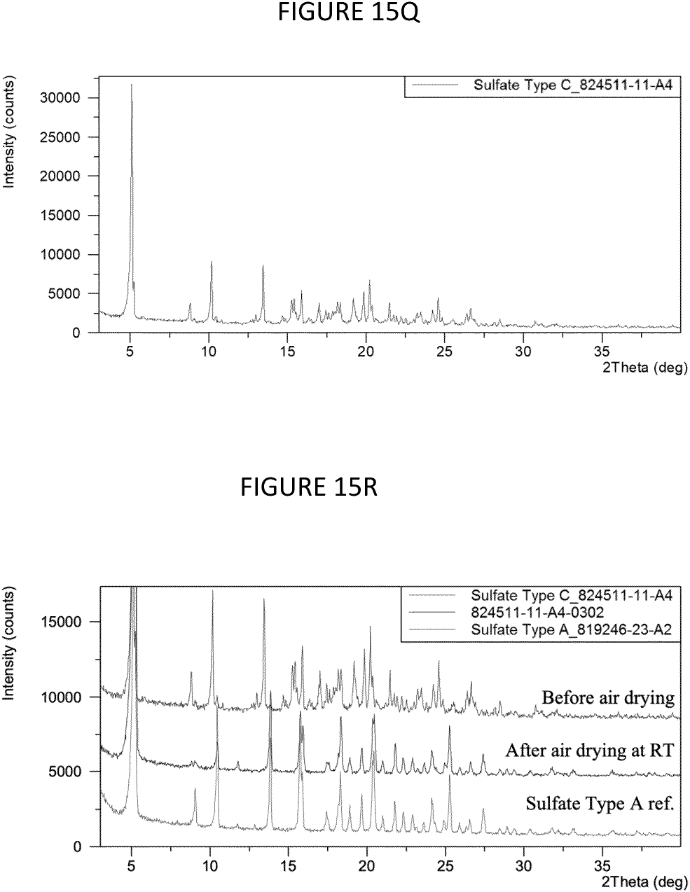

Sulfate Type D (824511-12-A17) was obtained by slurrying 20.3 mg sulfate Type A (824511-04-A) in THF/H₂O (981:19, v/v, aw≈0.2) at RT for about one week and isolating solids by centrifugation and air drying. The XRPD result is displayed in FIG. 15A and Table 17B. TGA/DSC results in FIG. 15B showed a weight loss of 2.3% up to 100.0° C. and two endothermic peaks at 76.1° C. and 194.9° C. (peak). Using MeOH-d4 as solvent, ¹ H NMR result in FIG. 15C showed that no peak of THF was observed. HPLC purity of the sample was determined as 99.79 area% (FIG. 15D and TABLE 17C). In FIG. 15E, XRPD result showed that after drying sulfate Type D by N₂ for 20 min at 30° C., a new form was observed and assigned as sulfate Type F (FIG. 15F and TABLE 17D). After heating to 100° C. and cooling back to 30° C. under N₂ protection, the sample remained sulfate Type F. After exposure to ambient condition for ~30 min, sulfate Type F converted back to sulfate Type D. Thus, sulfate Type D was speculated to be a hydrate (molar ratio of water to salt was 0.6, calculation based on TGA loss) and sulfate Type F was an anhydrate. Since sulfate Type F was not physically stable at ambient conditions, no more characterization was conducted.

TABLE 17D

XRPD peak list of sulfate Type D (824511-12-A17)

| Pos. [°2θ] | Height [cts] | FWHM Left [°2θ] | d-spacing [Å] | Rel. Int. [%] |
|---|---|---|---|---|
| 6.4028 | 900.11 | 0.1279 | 13.80 | 75.35 |
| 7.8820 | 574.06 | 0.1279 | 11.22 | 48.05 |
| 8.7830 | 429.72 | 0.1023 | 10.07 | 35.97 |
| 9.1967 | 404.66 | 0.1535 | 9.62 | 33.87 |
| 11.0244 | 150.87 | 0.1279 | 8.03 | 12.63 |
| 12.2344 | 516.31 | 0.1279 | 7.23 | 43.22 |
| 12.8206 | 655.09 | 0.1279 | 6.91 | 54.84 |
| 13.2880 | 679.03 | 0.1535 | 6.66 | 56.84 |
| 15.0875 | 1194.60 | 0.1279 | 5.87 | 100.00 |
| 15.7942 | 411.60 | 0.1279 | 5.61 | 34.46 |
| 16.2151 | 117.49 | 0.1279 | 5.47 | 9.84 |
| 16.8783 | 462.24 | 0.1279 | 5.25 | 38.69 |
| 18.4131 | 305.18 | 0.0768 | 4.82 | 25.55 |
| 18.6209 | 415.79 | 0.0768 | 4.77 | 34.81 |
| 18.9820 | 104.55 | 0.1023 | 4.68 | 8.75 |
| 19.3908 | 521.08 | 0.1535 | 4.58 | 43.62 |
| 20.3344 | 551.65 | 0.1279 | 4.37 | 46.18 |
| 20.7791 | 472.06 | 0.1791 | 4.27 | 39.52 |
| 21.5067 | 551.00 | 0.1535 | 4.13 | 46.12 |
| 22.2776 | 489.64 | 0.2047 | 3.99 | 40.99 |
| 22.9532 | 482.24 | 0.1791 | 3.87 | 40.37 |
| 23.4283 | 136.86 | 0.1279 | 3.80 | 11.46 |
| 23.5749 | 98.01 | 0.2047 | 3.77 | 8.20 |
| 24.2297 | 288.56 | 0.1023 | 3.67 | 24.15 |
| 25.2827 | 237.75 | 0.2558 | 3.52 | 19.90 |
| 25.5486 | 259.69 | 0.1023 | 3.49 | 21.74 |
| 26.0452 | 168.57 | 0.1791 | 3.42 | 14.11 |
| 26.9776 | 157.40 | 0.1535 | 3.31 | 13.18 |
| 28.1518 | 103.08 | 0.2558 | 3.17 | 8.63 |
| 28.6655 | 146.75 | 0.1279 | 3.11 | 12.28 |
| 28.9830 | 137.03 | 0.2047 | 3.08 | 11.47 |
| 29.4933 | 79.28 | 0.0768 | 3.03 | 6.64 |
| 30.4308 | 96.57 | 0.1279 | 2.94 | 8.08 |
| 31.1945 | 92.53 | 0.2558 | 2.87 | 7.75 |
| 32.0956 | 40.80 | 0.3070 | 2.79 | 3.42 |
| 33.4643 | 41.92 | 0.1535 | 2.68 | 3.51 |
| 34.2709 | 102.61 | 0.1023 | 2.62 | 8.59 |
| 34.8523 | 75.19 | 0.1535 | 2.57 | 6.29 |
| 35.7129 | 91.89 | 0.1791 | 2.51 | 7.69 |
| 38.5102 | 41.71 | 0.1535 | 2.34 | 3.49 |

TABLE 17C

HPLC results of sulfate Type D (824511-12-A17)

| # | RRT | Area (%) |
|---|---|---|
| 1 | 0.95 | 0.08 |
| 2 | 1.00 | 99.79 |
| 3 | 1.07 | 0.13 |

TABLE 17D

XRPD peak list of sulfate Type F (824511-12-A17_N₂ Back_30.0° C.)

| Pos. [°2θ] | Height [cts] | FWHM Left [°2θ] | d-spacing [Å] | Rel. Int. [%] |
|---|---|---|---|---|
| 6.4822 | 751.95 | 0.0669 | 13.64 | 31.48 |
| 8.0007 | 474.08 | 0.0669 | 11.05 | 19.85 |
| 8.7485 | 494.24 | 0.0502 | 10.11 | 20.69 |
| 9.0071 | 670.48 | 0.0669 | 9.82 | 28.07 |
| 11.3566 | 215.76 | 0.0669 | 7.79 | 9.03 |
| 11.6691 | 243.16 | 0.1004 | 7.58 | 10.18 |
| 12.3808 | 527.11 | 0.0836 | 7.15 | 22.07 |
| 12.8541 | 1210.42 | 0.1338 | 6.89 | 50.68 |
| 13.0166 | 949.07 | 0.0669 | 6.80 | 39.73 |
| 13.4278 | 993.93 | 0.0669 | 6.59 | 41.61 |
| 13.7788 | 532.21 | 0.0669 | 6.43 | 22.28 |
| 14.9011 | 2388.56 | 0.0836 | 5.95 | 100.00 |
| 15.5194 | 280.33 | 0.1171 | 5.71 | 11.74 |
| 16.0779 | 796.94 | 0.0836 | 5.51 | 33.36 |
| 16.6409 | 786.47 | 0.0669 | 5.33 | 32.93 |
| 16.9260 | 259.04 | 0.1004 | 5.24 | 10.84 |
| 17.1738 | 365.23 | 0.0836 | 5.16 | 15.29 |
| 17.5874 | 773.17 | 0.0669 | 5.04 | 32.37 |
| 18.1871 | 466.88 | 0.0669 | 4.88 | 19.55 |
| 18.4164 | 249.98 | 0.1004 | 4.82 | 10.47 |
| 18.6724 | 360.95 | 0.0836 | 4.75 | 15.11 |
| 18.9369 | 748.11 | 0.0836 | 4.69 | 31.32 |
| 19.6564 | 171.99 | 0.2007 | 4.52 | 7.20 |
| 20.0815 | 262.05 | 0.0836 | 4.42 | 10.97 |
| 20.5779 | 715.10 | 0.1004 | 4.32 | 29.94 |
| 20.8441 | 520.84 | 0.1004 | 4.26 | 21.81 |
| 20.9945 | 497.92 | 0.0669 | 4.23 | 20.85 |
| 21.4940 | 558.61 | 0.0836 | 4.13 | 23.39 |
| 21.7581 | 355.50 | 0.0669 | 4.08 | 14.88 |
| 22.2563 | 622.13 | 0.0836 | 3.99 | 26.05 |
| 22.8445 | 188.09 | 0.1338 | 3.89 | 7.87 |
| 23.1558 | 368.67 | 0.0669 | 3.84 | 15.43 |
| 23.5610 | 534.68 | 0.1004 | 3.78 | 22.38 |
| 24.5097 | 372.62 | 0.0669 | 3.63 | 15.60 |
| 25.0924 | 477.42 | 0.1171 | 3.55 | 19.99 |
| 25.4868 | 286.38 | 0.1338 | 3.49 | 11.99 |
| 25.9373 | 395.14 | 0.1004 | 3.44 | 16.54 |
| 26.5675 | 193.94 | 0.1338 | 3.36 | 8.12 |
| 27.3282 | 190.08 | 0.1338 | 3.26 | 7.96 |
| 27.5958 | 221.06 | 0.1004 | 3.23 | 9.25 |
| 28.8038 | 92.46 | 0.2007 | 3.10 | 3.87 |
| 29.4115 | 148.11 | 0.2676 | 3.04 | 6.20 |
| 30.0856 | 256.66 | 0.1004 | 2.97 | 10.75 |
| 30.6951 | 143.09 | 0.1004 | 2.91 | 5.99 |
| 31.2641 | 145.35 | 0.2007 | 2.86 | 6.09 |

### Sulfate Type E

Sulfate Type E (824511-11-A3-0315) was obtained by storing sulfate Type B (824511-11-A3) at RT in a sealed vial for ~12 days. The XRPD result was displayed in FIG. 15G and TABLE 17E. TGA/DSC curves in FIG. 15H showed a weight loss of 3.0% up to 120.0° C., three endothermic signals at 74.1° C., 116.8° C. and 147.0° C. (peak). XRPD overlay in FIG. 15l showed that after heating to 100° C. and cooling back to ambient conditions, no form change was observed. Using DMSO-d₆ as solvent, ¹H NMR result of sulfate Type E from heating experiment in FIG. 15J showed that the peak of ACN was observed. The molar ratio of ACN/API was 0.03:1 (theoretical weight=0.3 wt%). Combined with the results of heating experiment, sulfate Type E was speculated to be a hydrate or anhydrate. Since sulfate Type E was not obtained in the re-preparation trials, no more characterization data were collected.

TABLE 17E

XRPD peak list of sulfate Type E (824511-11-A3-0315)

| Pos. [°2θ] | Height [cts] | FWHM Left [°2θ] | d-spacing [Å] | Rel. Int. [%] |
|---|---|---|---|---|
| 9.0743 | 4585.43 | 0.1023 | 9.75 | 100.00 |
| 9.4495 | 369.06 | 0.1023 | 9.36 | 8.05 |
| 12.0063 | 66.31 | 0.1023 | 7.37 | 1.45 |
| 12.7318 | 30.52 | 0.3070 | 6.95 | 0.67 |
| 14.3589 | 147.88 | 0.0768 | 6.17 | 3.22 |
| 14.6986 | 235.98 | 0.0768 | 6.03 | 5.15 |
| 14.8248 | 373.28 | 0.0768 | 5.98 | 8.14 |
| 15.3581 | 94.89 | 0.1279 | 5.77 | 2.07 |
| 16.5642 | 154.49 | 0.0768 | 5.35 | 3.37 |
| 17.0472 | 110.64 | 0.1023 | 5.20 | 2.41 |
| 17.2988 | 90.47 | 0.0768 | 5.13 | 1.97 |
| 17.6353 | 444.91 | 0.0768 | 5.03 | 9.70 |
| 17.7951 | 804.53 | 0.1023 | 4.98 | 17.55 |
| 18.1725 | 209.89 | 0.1535 | 4.88 | 4.58 |
| 18.9649 | 578.74 | 0.0768 | 4.68 | 12.62 |
| 19.2087 | 569.58 | 0.1023 | 4.62 | 12.42 |
| 19.5550 | 549.90 | 0.1023 | 4.54 | 11.99 |
| 20.2549 | 512.95 | 0.1023 | 4.38 | 11.19 |
| 20.8487 | 526.68 | 0.1279 | 4.26 | 11.49 |
| 21.4058 | 104.21 | 0.1023 | 4.15 | 2.27 |
| 22.7450 | 155.18 | 0.1023 | 3.91 | 3.38 |
| 23.0061 | 241.46 | 0.1023 | 3.87 | 5.27 |
| 23.4958 | 108.80 | 0.0768 | 3.79 | 2.37 |
| 23.8093 | 337.21 | 0.1023 | 3.74 | 7.35 |
| 24.1491 | 105.64 | 0.1535 | 3.69 | 2.30 |
| 24.5379 | 412.82 | 0.1023 | 3.63 | 9.00 |
| 25.1671 | 371.58 | 0.1535 | 3.54 | 8.10 |
| 25.7731 | 224.25 | 0.1279 | 3.46 | 4.89 |
| 26.4613 | 251.68 | 0.1023 | 3.37 | 5.49 |
| 26.9900 | 232.45 | 0.1023 | 3.30 | 5.07 |
| 27.2910 | 289.04 | 0.1279 | 3.27 | 6.30 |
| 27.5766 | 209.20 | 0.1023 | 3.23 | 4.56 |
| 28.7954 | 136.40 | 0.1023 | 3.10 | 2.97 |
| 30.9018 | 86.57 | 0.2047 | 2.89 | 1.89 |
| 31.5125 | 95.59 | 0.1023 | 2.84 | 2.08 |
| 32.5473 | 63.89 | 0.2047 | 2.75 | 1.39 |

### Solvate

Sulfate Type B (824511-11-A3) was obtained by slurrying sulfate Type A (824511-04-A) in ACN with temperature cycling (suspend 20.3 mg sulfate in 0.5 mL ACN at RT and then transfer to temperature cycling (3 cycle): ramp to 50° C. at a rate of 1° C./min, isothermal for 120 min, cool to 5° C. at a rate of 0.1° C./min, isothermal for 120 min). Resulting solids were isolated by centrifugation and air drying. The XRPD result is displayed in FIG. 15K and TABLE 17F. TGA/DSC results in FIG. 15L showed a weight of 6.6% up to 100.0° C., four endothermic signals at 65.4° C., 115.9° C., 146.8° C. and 188.4° C. (peak). Using DMSO-d₆ as solvent, ¹H NMR result in FIG. 15M showed that the peak of ACN was observed. The molar ratio of ACN/API was 0.6:1 (theoretical weight=5.4 wt%, similar to weight loss in TGA before 100.0° C.). HPLC purity of the sample was determined as 99.64 area% (FIG. 15N and TABLE 17G). The molar ratio of acid/freebase was determined as 0.9:1 by HPLC/IC. XRPD overlay in FIG. 15O showed that after storage sulfate Type B at RT for ~12 days, form change to sulfate Type E was observed and weight loss before 100.0° C. decreased from 6.6% to 2.6% (FIG. 15P). Considering that sulfate Type B was only obtained in ACN conditions in the screening experiments, sulfate Type B was possibly an ACN solvate.

TABLE 17F

XRPD peak list of sulfate Type B (824511-11-A3)

| Pos. [°2θ] | Height [cts] | FWHM Left [°2θ] | d-spacing [Å] | Rel. Int. [%] |
|---|---|---|---|---|
| 8.8617 | 2443.03 | 0.0768 | 9.98 | 100.00 |
| 9.0536 | 1632.69 | 0.1279 | 9.77 | 66.83 |
| 11.9870 | 50.95 | 0.1535 | 7.38 | 2.09 |
| 12.4818 | 101.91 | 0.1023 | 7.09 | 4.17 |
| 14.2401 | 146.20 | 0.0768 | 6.22 | 5.98 |
| 14.6560 | 289.56 | 0.1023 | 6.04 | 11.85 |
| 15.8739 | 60.84 | 0.1023 | 5.58 | 2.49 |
| 17.0027 | 87.54 | 0.1023 | 5.21 | 3.58 |
| 17.3500 | 217.83 | 0.0768 | 5.11 | 8.92 |
| 17.6773 | 919.64 | 0.1535 | 5.02 | 37.64 |
| 18.1611 | 188.58 | 0.0768 | 4.88 | 7.72 |
| 18.4209 | 169.21 | 0.1023 | 4.82 | 6.93 |
| 18.9236 | 392.46 | 0.1023 | 4.69 | 16.06 |
| 19.3668 | 308.15 | 0.2047 | 4.58 | 12.61 |
| 20.2575 | 527.02 | 0.1279 | 4.38 | 21.57 |
| 20.6626 | 176.01 | 0.0768 | 4.30 | 7.20 |
| 21.2906 | 74.03 | 0.1535 | 4.17 | 3.03 |
| 22.6414 | 172.47 | 0.1023 | 3.93 | 7.06 |
| 22.8746 | 176.72 | 0.2047 | 3.89 | 7.23 |
| 23.4531 | 145.79 | 0.1023 | 3.79 | 5.97 |
| 23.8543 | 394.77 | 0.1279 | 3.73 | 16.16 |
| 24.2881 | 285.40 | 0.1279 | 3.66 | 11.68 |
| 24.7497 | 294.42 | 0.1279 | 3.60 | 12.05 |
| 25.0684 | 317.48 | 0.1279 | 3.55 | 13.00 |
| 25.8656 | 266.97 | 0.1023 | 3.44 | 10.93 |
| 26.6017 | 138.46 | 0.2558 | 3.35 | 5.67 |
| 27.2138 | 279.94 | 0.1535 | 3.28 | 11.46 |
| 28.1100 | 71.05 | 0.1535 | 3.17 | 2.91 |
| 28.7799 | 61.17 | 0.1535 | 3.10 | 2.50 |
| 29.5367 | 50.88 | 0.1535 | 3.02 | 2.08 |
| 30.8095 | 147.60 | 0.1535 | 2.90 | 6.04 |
| 31.5587 | 98.78 | 0.2047 | 2.84 | 4.04 |
| 31.8317 | 73.94 | 0.8187 | 2.81 | 3.03 |

TABLE 17G

HPLC results of sulfate Type B (824511-11-A3)

| # | RRT | Area (%) | # | RRT | Area (%) |
|---|---|---|---|---|---|
| 1 | 0.89 | 0.09 | 4 | 1.00 | 99.64 |
| 2 | 0.93 | 0.07 | 5 | 1.07 | 0.11 |
| 3 | 0.95 | 0.08 | -- | -- | -- |

### Metastable Form

Sulfate Type C (824511-11-A4) was obtained by slurrying sulfate Type A (824511-04-A) in 1,4-dioxane with temperature cycling (suspend 20.3 mg sulfate in 0.5 mL 1,4-dioxane at RT and then transfer to temperature cycling (3 cycle): ramp to 50° C. at a rate of 1° C./min, isothermal for 120 min, cool to 5° C. at a rate of 0.1° C./min, isothermal for 120 min). Resulting solids were isolated by centrifugation and air drying. The XRPD result is displayed in FIG. 15Q and TABLE 17H. The XRPD overlay in FIG. 15R showed that after air drying at RT for ~30 min, sulfate Type C converted to sulfate Type A, which indicated that sulfate Type C was a metastable form. Since sulfate Type C was not obtained in the re-preparation trials, no more characterization data were collected.

TABLE 17H

XRPD peak list of sulfate Type C (824511-11-A4)

| Pos. [°2θ] | Height [cts] | FWHM Left [°2θ] | d-spacing [Å] | Rel. Int. [%] |
|---|---|---|---|---|
| 5.0847 | 4310.60 | 0.0768 | 17.38 | 100.00 |
| 5.2481 | 607.60 | 0.0512 | 16.84 | 14.10 |
| 8.7925 | 346.08 | 0.0768 | 10.06 | 8.03 |
| 9.0655 | 44.07 | 0.1279 | 9.76 | 1.02 |
| 10.1545 | 1142.33 | 0.1023 | 8.71 | 26.50 |
| 10.4374 | 120.08 | 0.0768 | 8.48 | 2.79 |
| 12.9800 | 175.51 | 0.0768 | 6.82 | 4.07 |
| 13.4399 | 1138.95 | 0.1023 | 6.59 | 26.42 |
| 14.6743 | 146.18 | 0.0768 | 6.04 | 3.39 |
| 15.2524 | 445.52 | 0.0768 | 5.81 | 10.34 |
| 15.4119 | 494.37 | 0.0512 | 5.75 | 11.47 |
| 15.8787 | 651.18 | 0.1023 | 5.58 | 15.11 |
| 16.3314 | 109.59 | 0.1279 | 5.43 | 2.54 |
| 16.9984 | 370.20 | 0.1279 | 5.22 | 8.59 |
| 17.4383 | 261.06 | 0.0768 | 5.09 | 6.06 |
| 18.1718 | 427.90 | 0.0512 | 4.88 | 9.93 |
| 18.3467 | 428.27 | 0.0768 | 4.84 | 9.94 |
| 19.1841 | 501.00 | 0.1023 | 4.63 | 11.62 |
| 19.8325 | 634.99 | 0.1023 | 4.48 | 14.73 |
| 20.2154 | 805.12 | 0.0768 | 4.39 | 18.68 |
| 21.4685 | 436.15 | 0.0768 | 4.14 | 10.12 |
| 21.7230 | 189.88 | 0.1023 | 4.09 | 4.41 |
| 21.9248 | 166.23 | 0.0768 | 4.05 | 3.86 |
| 22.2266 | 151.16 | 0.1023 | 4.00 | 3.51 |
| 22.5221 | 133.11 | 0.0768 | 3.95 | 3.09 |
| 23.0063 | 111.16 | 0.0768 | 3.87 | 2.58 |
| 23.2244 | 229.81 | 0.0768 | 3.83 | 5.33 |
| 23.4632 | 252.26 | 0.1023 | 3.79 | 5.85 |
| 24.2313 | 269.67 | 0.1279 | 3.67 | 6.26 |
| 24.5769 | 515.12 | 0.1023 | 3.62 | 11.95 |
| 24.8191 | 146.82 | 0.1023 | 3.59 | 3.41 |
| 25.4924 | 98.35 | 0.2047 | 3.49 | 2.28 |
| 26.3865 | 243.12 | 0.1535 | 3.38 | 5.64 |
| 26.6446 | 345.13 | 0.0768 | 3.35 | 8.01 |
| 27.2963 | 52.03 | 0.1023 | 3.27 | 1.21 |
| 28.1370 | 67.33 | 0.1023 | 3.17 | 1.56 |
| 28.4909 | 143.13 | 0.1023 | 3.13 | 3.32 |
| 30.7511 | 98.61 | 0.0768 | 2.91 | 2.29 |
| 31.1559 | 52.65 | 0.1535 | 2.87 | 1.22 |
| 31.9786 | 39.43 | 0.5117 | 2.80 | 0.91 |

### Conclusion

A brief polymorph screening of 18-MC sulfate was performed and a total of six salt forms were obtained.

### Example 4 - Polymorph Screening of 18-MC Oxalate

18-MC oxalate Type A was first obtained as described in Example 1. Polymorph screening of 18-MC oxalate was performed to better understand polymorphism of the salt.

Oxalate material was prepared using 18-MC freebase and used as starting material for polymorph screening. X-ray powder diffraction (XRPD) result showed that the prepared oxalate displayed a different XRPD pattern from oxalate Type A, which was assigned as oxalate Type B, and further characterized by TGA, DSC and HPLCIC. Based on the limited weight loss in TGA and neat DSC before decomposition, both oxalate Type A and oxalate Type B were speculated as anhydrates.

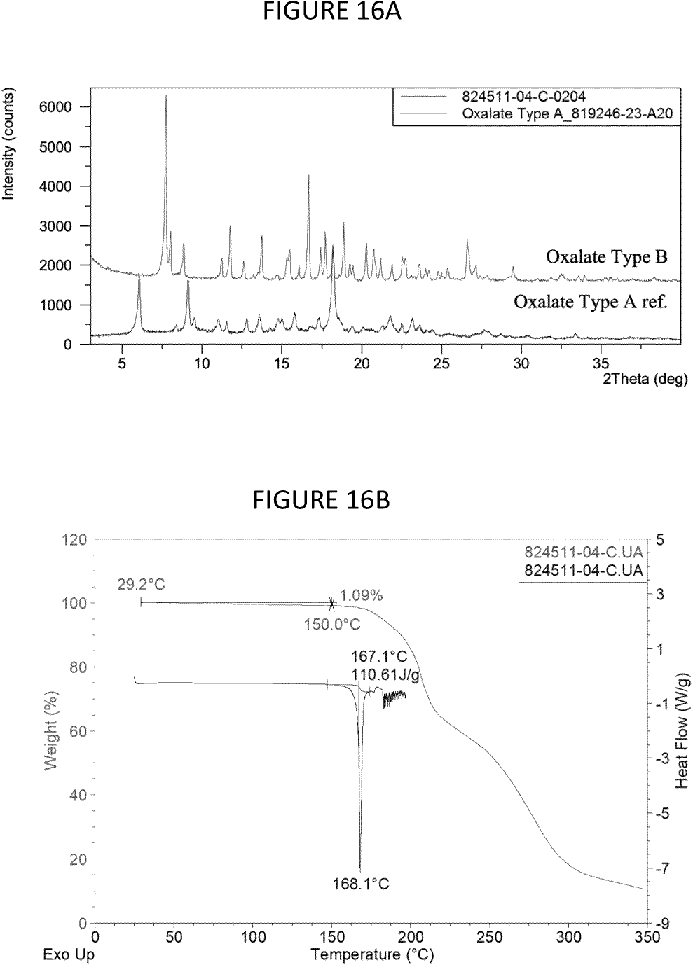

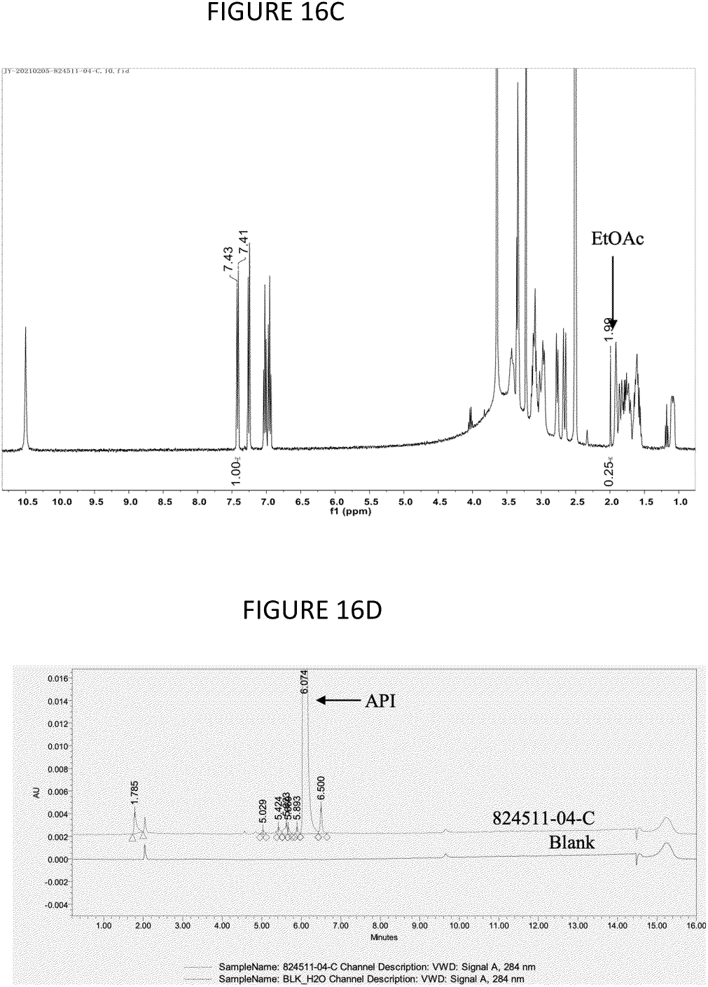

Using prepared oxalate Type B as the starting material, a total of 30 polymorph screening experiments were conducted with different crystallization methods including temperature cycling and slurry conversion at different temperatures. Solids from the screening experiments were isolated for XRPD testing. As the results showed, only oxalate Type B was obtained. Characterization results of oxalate Type A and B were summarized in TABLE 18 and XRPD patterns of the two forms were displayed in FIG. 16A.

To summarize, a brief polymorph screening experiments were performed and only oxalate Type B was observed.

TABLE 18

Characterization summary of oxalate salt forms

| Solid form (ID) | TGA loss (%) | Endother m (°C., peak) | Solvent residual (wt%) | Molar ratio^{#} (acid/FB) | HPLC purity (area%) | Speculated form |
|---|---|---|---|---|---|---|
| Oxalate Type A* (819246-23-A20) | 1.5 (up to 120° C.) | 170.6 | -- | 0.9 | 99.66 | Anhydrate |
| Oxalate Type B (824511-04-C) | 1.1 (up to 150° C.) | 168.1 | 1.5 (EtOAc) | 1.1 | 98.95 | Anhydrate |

FB: Freebase.
--: No data collected.
*: Also See Example 1.
^{#}: Determined by HPLC/IC.

### Preparation of Oxalate Starting Material

Preparation procedure of oxalate was as follows: weigh 2.0 g of freebase Type A (824509-03-A) and 0.7 g of oxalic acid dihydrate into 500 mL of EtOAc and transfer the suspension to slurry at RT for ~5 days. Resulting solids were isolated by vacuum filtration and vacuum drying at RT overnight. About 2.4 g of oxalate sample (824511-04-C) was obtained. XRPD comparison results in FIG. 16A showed that the obtained oxalate displayed a different pattern than oxalate Type A (819246-23-A20), and it was named as oxalate Type B, with XRPD peak list shown in TABLE 19A. TGA/DSC curves in FIG. 16B showed a weight loss of 1.1% up to 150.0° C. and one endothermic peak at 167.1° C. (onset). Proton nuclear magnetic resonance (¹H NMR) result in FIG. 16C showed that peak of EtOAc was observed. The molar ratio of EtOAc/API was 0.08:1 (theoretical weight=1.5 wt%). HPLC/IC results showed that the molar ratio of acid/freebase was determined as 1.08:1 and HPLC purity was 98.95 area% (FIG. 16D and TABLE 19B). Considering the limited weight loss and neat DSC curve before decomposition, oxalate Type B was speculated to be an anhydrate.

Approximate solubility of oxalate Type B (824511-04-C) was estimated in 10 solvents to guide the solvent selection in polymorph screening of oxalate, and the results were summarized in TABLE 19C.

TABLE 19A

XRPD peak list of oxalate Type B (824511-04-C)

| Pos. [°2θ] | Height [cts] | FWHM Left [°2θ] | d-spacing [Å] | Rel. Int. [%] |
|---|---|---|---|---|
| 7.7239 | 4334.43 | 0.0768 | 11.45 | 100.00 |
| 8.0135 | 1072.28 | 0.0768 | 11.03 | 24.74 |
| 8.8364 | 830.69 | 0.1023 | 10.01 | 19.16 |
| 11.2188 | 500.49 | 0.0768 | 7.89 | 11.55 |
| 11.7533 | 1278.57 | 0.1023 | 7.53 | 29.50 |
| 12.6140 | 427.26 | 0.1023 | 7.02 | 9.86 |
| 13.2194 | 114.28 | 0.0768 | 6.70 | 2.64 |
| 13.5007 | 149.12 | 0.1023 | 6.56 | 3.44 |
| 13.7314 | 1089.09 | 0.1023 | 6.45 | 25.13 |
| 14.7229 | 97.64 | 0.1791 | 6.02 | 2.25 |
| 15.2990 | 509.10 | 0.0768 | 5.79 | 11.75 |
| 15.4682 | 724.39 | 0.0768 | 5.73 | 16.71 |
| 16.0654 | 355.40 | 0.0768 | 5.52 | 8.20 |
| 16.6673 | 2545.47 | 0.1023 | 5.32 | 58.73 |
| 17.4245 | 766.29 | 0.1023 | 5.09 | 17.68 |
| 17.7222 | 1135.30 | 0.1023 | 5.00 | 26.19 |
| 18.1886 | 686.59 | 0.1023 | 4.88 | 15.84 |
| 18.8803 | 1445.67 | 0.1023 | 4.70 | 33.35 |
| 19.2535 | 397.25 | 0.0768 | 4.61 | 9.16 |
| 19.4491 | 341.44 | 0.0768 | 4.56 | 7.88 |
| 20.2915 | 864.04 | 0.1023 | 4.38 | 19.93 |
| 20.7591 | 752.54 | 0.1535 | 4.28 | 17.36 |
| 21.1907 | 535.52 | 0.1023 | 4.19 | 12.35 |
| 21.8972 | 388.31 | 0.0768 | 4.06 | 8.96 |
| 22.5655 | 530.31 | 0.0768 | 3.94 | 12.23 |
| 22.7316 | 530.03 | 0.1023 | 3.91 | 12.23 |
| 23.1168 | 94.32 | 0.2047 | 3.85 | 2.18 |
| 23.6083 | 390.08 | 0.1023 | 3.77 | 9.00 |
| 24.0026 | 316.43 | 0.1023 | 3.71 | 7.30 |
| 24.2042 | 239.03 | 0.0768 | 3.68 | 5.51 |
| 24.7960 | 211.35 | 0.1023 | 3.59 | 4.88 |
| 25.0017 | 176.50 | 0.0768 | 3.56 | 4.07 |
| 25.3834 | 295.12 | 0.0768 | 3.51 | 6.81 |
| 26.6208 | 1050.03 | 0.1279 | 3.35 | 24.23 |
| 27.1468 | 386.44 | 0.0768 | 3.28 | 8.92 |
| 27.4112 | 109.34 | 0.0768 | 3.25 | 2.52 |
| 27.8368 | 136.25 | 0.0768 | 3.21 | 3.14 |
| 29.4813 | 360.35 | 0.1023 | 3.03 | 8.31 |
| 31.0020 | 65.62 | 0.0768 | 2.88 | 1.51 |
| 31.8721 | 75.49 | 0.1023 | 2.81 | 1.74 |
| 32.5499 | 146.60 | 0.2558 | 2.75 | 3.38 |
| 33.5785 | 112.72 | 0.1791 | 2.67 | 2.60 |
| 33.9792 | 134.19 | 0.1023 | 2.64 | 3.10 |
| 35.2567 | 100.85 | 0.1279 | 2.55 | 2.33 |
| 35.5853 | 83.29 | 0.1023 | 2.52 | 1.92 |
| 38.3216 | 81.34 | 0.1535 | 2.35 | 1.88 |

TABLE 19B

HPLC results of oxalate Type B (824511-04-C)

| # | RRT | Area (%) | # | RRT | Area (%) |
|---|---|---|---|---|---|
| 1 | 0.29 | 0.32 | 5 | 0.93 | 0.06 |
| 2 | 0.83 | 0.05 | 6 | 0.97 | 0.08 |
| 3 | 0.89 | 0.10 | 7 | 1.00 | 98.95 |
| 4 | 0.93 | 0.16 | 8 | 1.07 | 0.28 |

TABLE 19C

Approximate solubility of oxalate Type B (824511-04-C) at RT

| | | | |
|---|---|---|---|
| Solvent | Solubility (mg/mL) | Solvent | Solubility (mg/mL) |
| MeOH | S>44.0 | THF | 6.3<S<19.0 |
| H₂O | S>38.0 | CHCl₃ | 2.3<S<7.7 |
| ACN | 7.7<S<23.0 | DCM | 2.1<S<7.0 |
| EtOH | 7.0<S<21.0 | n-Heptane | S<2.1 |
| Acetone | 7.0<S<21.0 | EtOAc | S<1.8 |

Procedure: weigh ~2 mg solids into each 3-mL glass vial, add in corresponding solvent stepwise and sonicate or oscillate to see if solids dissolved completely. Stop adding solvent till the solids dissolves or total volume reaches 1.0 mL. Calculate the approximate solubility based on solvent volume.

### Polymorph Screening

Using oxalate Type B (824511-04-C) as the starting material, a total of 30 experiments were conducted using different crystallization methods. XRPD results showed that oxalate Type B was the only form obtained from the screen. The results of polymorph screening are summarized in TABLE 20A, and only oxalate Type B was observed.

TABLE 20A

Summary of polymorph screening experiments of oxalate

| Method | No. of Experiment | Results |
|---|---|---|
| Temperature Cycling | 5 | Oxalate Type B |
| Slurry at RT | 20 | Oxalate Type B |
| Slurry at 50° C. | 5 | Oxalate Type B |
| Total | 30 | Oxalate Type B |

### Temperature Cycling (50° C. to 5° C.)

Temperature cycling experiments were conducted in 5 solvent systems. About 20 mg of starting material (824511-04-C) was suspended in an HPLC vial with 0.5 mL of corresponding solvents listed in TABLE 20B. Cycling procedure: ramped to 50° C. at a rate of 4.5° C./min, kept the temperature at 50° C. for 30 min; cooled down to 5° C. at a rate of 0.1° C./min and kept the temperature at 5° C. for 30 min. After three cycles, the obtained solids were isolated by centrifugation and air dried for XRPD analysis. Results were summarized in TABLE 20B, and only oxalate Type B was observed.

TABLE 20B

Summary of temperature cycling experiments of oxalate

| | | |
|---|---|---|
| Experiment ID | Solvent | Solid form |
| 824511-14-A1 | EtOH | Oxalate Type B |
| 824511-14-A2 | Acetone | Oxalate Type B |
| 824511-14-A3 | ACN | Oxalate Type B |
| 824511-14-A4 | 2-MeTHF | Oxalate Type B |
| 824511-14-A5 | CHCl₃ | Oxalate Type B |

### Slurry Conversion at RT

Slurry conversion experiments were conducted at RT in 20 different solvent systems. About 20 mg of starting material (824511-04-C) was suspended in an HPLC vial with 0.5 mL of corresponding solvents listed in TABLE 20C. If the solid dissolved then more solids were added until a suspension was obtained. The suspensions were stirred at RT using magnetic stirring with a speed of ~750 rpm. After about 5 days, the remaining solids were isolated by centrifugation and air dried for XRPD analysis. If a clear solution was obtained, the sample was cooled to 5° C. If it was still clear, the solution was cooled to -20° C. If there was still no precipitate, the solution was allowed to evaporate in an open vial at RT to obtain solids. Results are summarized in TABLE 20C, and only oxalate Type B was observed.

TABLE 20C

Summary of slurry conversion experiments at RT of oxalate

| | | |
|---|---|---|
| Experiment ID | Solvent, v:v | Solid form |
| 824511-15-A1 | EtOH | Oxalate Type B |
| 824511-15-A2 | IPA | Oxalate Type B |
| 824511-15-A3 | 2-BuOH | Oxalate Type B |
| 824511-15-A4 | MEK | Oxalate Type B |
| 824511-15-A5 | Acetone | Oxalate Type B |
| 824511-15-A6 | ACN | Oxalate Type B |
| 824511-15-A7 | MTBE | Oxalate Type B |
| 824511-15-A8 | DCM | Oxalate Type B |
| 824511-15-A9 | lPAc | Oxalate Type B |
| 824511-15-A10 | ACN/Toluene, 2:1 | Oxalate Type B |
| 824511-15-A11 | MlBK/n-heptane, 2:1 | Oxalate Type B |
| 824511-15-A12 | MeOH/EtOAc, 2:1 | Oxalate Type B |
| 824511-15-A13 | 1,4-Dioxane/CHCl₃, 1:4 | Oxalate Type B |
| 824511-15-A14 | EtOH/n-heptane, 1:4 | Oxalate Type B |
| 824511-15-A15 | ACN/Anisole, 1:4 | Oxalate Type B |
| 824511-15-A16 | THF | Oxalate Type B |
| 824511-15-A17 | THF/H₂O, 981:19 | Oxalate Type B |
| 824511-15-A18 | THF/H₂O, 957:43 | Oxalate Type B* |
| 824511-15-A19 | THF/H₂O, 924:76 | Oxalate Type B |
| 824511-15-A20 | THF/H₂O, 87:13 | Oxalate Type B** |

*: Clear solution was obtained at RT → transfer to slurry at 5° C.
**: Clear solution was obtained at RT → transfer to slurry at 5° C. → transfer to slurry at -20° C. → evaporation at RT.

### Slurry Conversion at 50° C.

Slurry conversion experiments were conducted at 50° C. in 5 solvent systems. About 20 mg of starting material (824511-04-C) was suspended in an HPLC glass vial with 0.5 mL of corresponding solvents listed in TABLE 20D. Samples were stirred at 50° C. for 5 days. If there was no solid after the slurry, the sample was cooled to 5° C. Resulting solids were isolated by centrifugation and air dried for XRPD analysis. Results are summarized in TABLE 20D, and only oxalate Type B was observed.

TABLE 20D

Summary of slurry conversion experiments at 50° C. of oxalate

| | | |
|---|---|---|
| Experiment ID | Solvent | Solid form |
| 824511-16-A1 | IPA | Oxalate Type B |
| 824511-16-A2 | Acetone | Oxalate Type B |
| 824511-16-A3 | Toluene | Oxalate Type B |
| 824511-16-A4 | EtOAc | Oxalate Type B |
| 824511-16-A5 | ACN | Oxalate Type B |

XPRD data for oxalate Type A is shown in TABLE 20E.

TABLE 20E

| Pos. [°2Th.] | Height [cts] | FWHM Left [°2Th.] | d-spacing [Å] | Rel. Int. [%] |
|---|---|---|---|---|
| 6.077817 | 1430.906000 | 0.100368 | 14.54211 | 61.97 |
| 8.377291 | 250.126300 | 0.066912 | 10.55492 | 10.83 |
| 9.134027 | 1363.833000 | 0.100368 | 9.68209 | 59.06 |
| 9.553869 | 388.156000 | 0.100368 | 9.25754 | 16.81 |
| 11.036050 | 407.482400 | 0.133824 | 8.01732 | 17.65 |
| 11.557700 | 329.334100 | 0.133824 | 7.65662 | 14.26 |
| 12.804420 | 433.000900 | 0.133824 | 6.91378 | 18.75 |
| 13.566870 | 520.062100 | 0.083640 | 6.52690 | 22.52 |
| 14.257180 | 205.103400 | 0.100368 | 6.21239 | 8.88 |
| 14.777540 | 435.414200 | 0.117096 | 5.99479 | 18.86 |
| 15.005280 | 407.003200 | 0.133824 | 5.90431 | 17.63 |
| 15.810090 | 562.427700 | 0.083640 | 5.60552 | 24.36 |
| 16.747330 | 241.901900 | 0.100368 | 5.29387 | 10.48 |
| 17.357210 | 452.678100 | 0.066912 | 5.10921 | 19.60 |
| 18.194850 | 2309.141000 | 0.150552 | 4.87584 | 100.00 |
| 19.427470 | 253.339600 | 0.167280 | 4.56917 | 10.97 |
| 20.093010 | 232.998000 | 0.100368 | 4.41931 | 10.09 |
| 21.301940 | 273.883900 | 0.167280 | 4.17116 | 11.86 |
| 21.787670 | 508.545900 | 0.083640 | 4.07925 | 22.02 |
| 22.489080 | 343.429600 | 0.066912 | 3.95359 | 14.87 |
| 23.202220 | 445.228100 | 0.133824 | 3.83366 | 19.28 |
| 23.631640 | 299.983900 | 0.066912 | 3.76496 | 12.99 |
| 24.435040 | 184.937300 | 0.167280 | 3.64296 | 8.01 |
| 25.444820 | 88.798480 | 0.401472 | 3.50063 | 3.85 |
| 26.446460 | 54.587300 | 0.234192 | 3.37028 | 2.36 |
| 27.686590 | 205.549900 | 0.133824 | 3.22208 | 8.90 |
| 28.706660 | 90.179000 | 0.267648 | 3.10986 | 3.91 |
| 29.283260 | 53.702470 | 0.200736 | 3.04993 | 2.33 |
| 30.403890 | 65.782810 | 0.267648 | 2.94002 | 2.85 |
| 31.792520 | 40.571410 | 0.401472 | 2.81470 | 1.76 |
| 33.393520 | 129.887600 | 0.150552 | 2.68333 | 5.62 |
| 35.220030 | 20.978200 | 0.535296 | 2.54824 | 0.91 |
| 36.602780 | 18.618700 | 0.401472 | 2.45509 | 0.81 |

### Conclusion

A brief polymorph screening of 18-MC oxalate was performed and one new form was obtained.

### Example 5 - Polymorph Screening of 18-MC Mesylate

18-MC mesylate Type A was first obtained as described in Example 1. Polymorph screening of 18-MC mesylate was performed to better understand polymorphism of the salt.

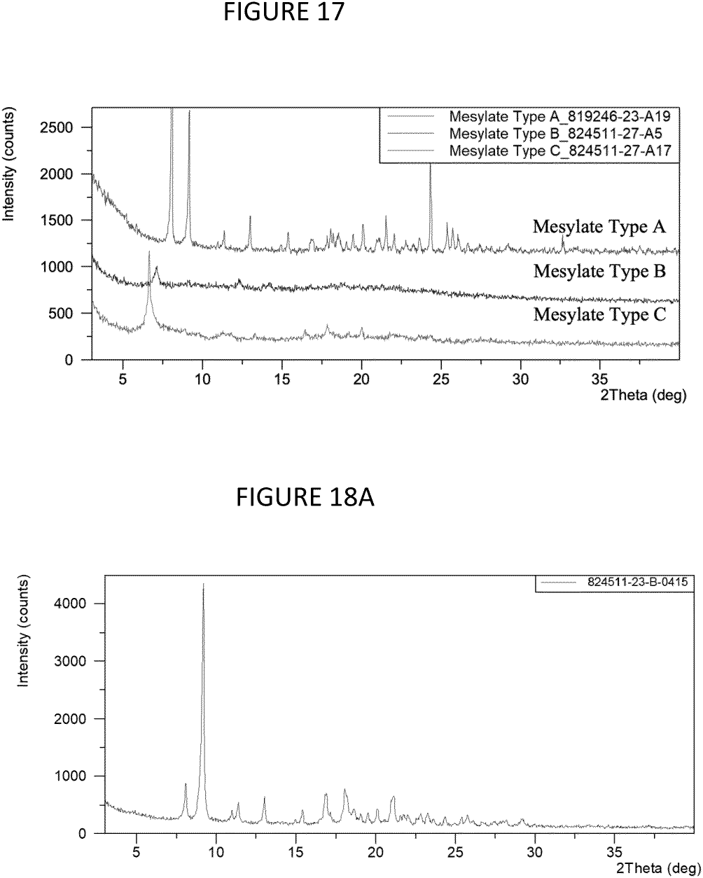

Mesylate material was first prepared using 18-MC freebase and used as starting material for polymorph screening. In the screening, different crystallization methods including temperature cycling and slurry conversion at different temperatures were used, and a total of 30 experiments were conducted. Solids from the screening were isolated for XRPD testing. As the results showed, two new forms, assigned as mesylate Types B and C, with weak crystallinity were observed. Attempts were made to reprepare the two new forms for further characterization. However, they were not found to be physically stable at ambient conditions and easily converted to gels/oils. Additional data were not collected. XRPD patterns of different forms are displayed in FIG. 17, and characterization results are summarized in TABLE 21.

To summarize, brief polymorph screening experiments were performed and a total of three forms of mesylate were discovered.

TABLE 21

Characterization summary of mesylate salt forms

| | | | | | | |
|---|---|---|---|---|---|---|
| Solid form (ID: 824511-) | TGA loss (%) | Endotherm (°C., peak) | Solvent residual (wt%)^{#} | Molar ratio^{#} (acid/FB) | HPLC purity (area%) | Speculated form |
| Mesylate Type A (23-B) | 1.7 up to 80° C. | 89.2, 171.5 | 0.5 (EtOAc) | 1.0 | 98.86 | Anhydrate |
| Mesylate Type B (32-A1) | -- | -- | -- | -- | -- | Metastable* |
| Mesylate Type C (32-A2) | -- | -- | -- | -- | -- | Metastable* |

FB: Freebase.
--: No data collected due to limited sample amount and failure of re-preparation trials.
^{#}: Calculation based on ¹ H NMR result.
*: The solid turn to gel-like after air drying at RT in a few minutes.

### Preparation of Mesylate Starting Material

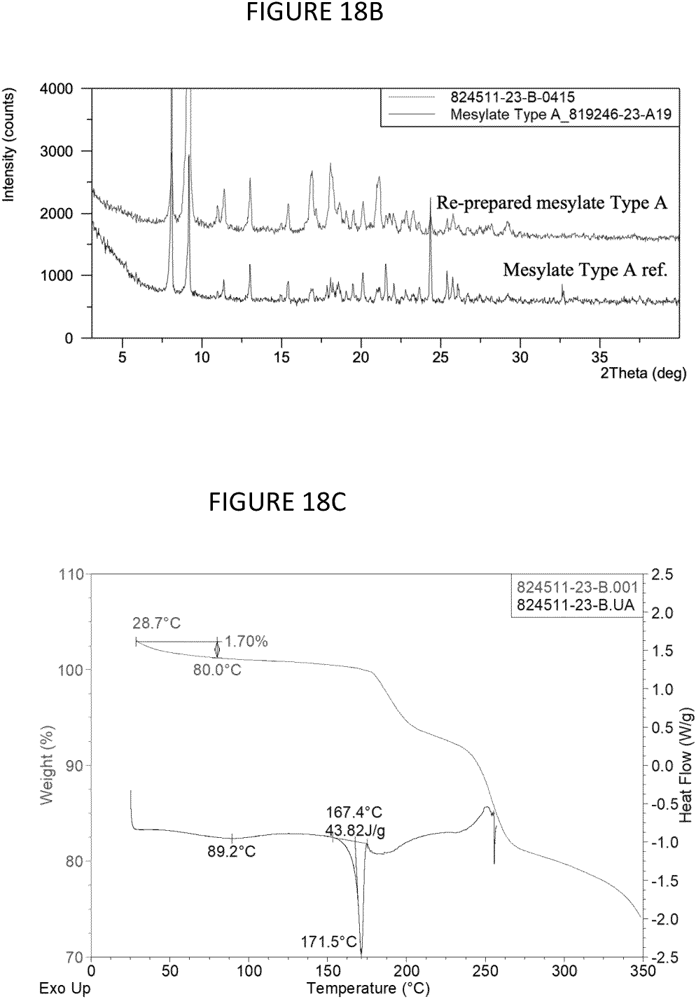

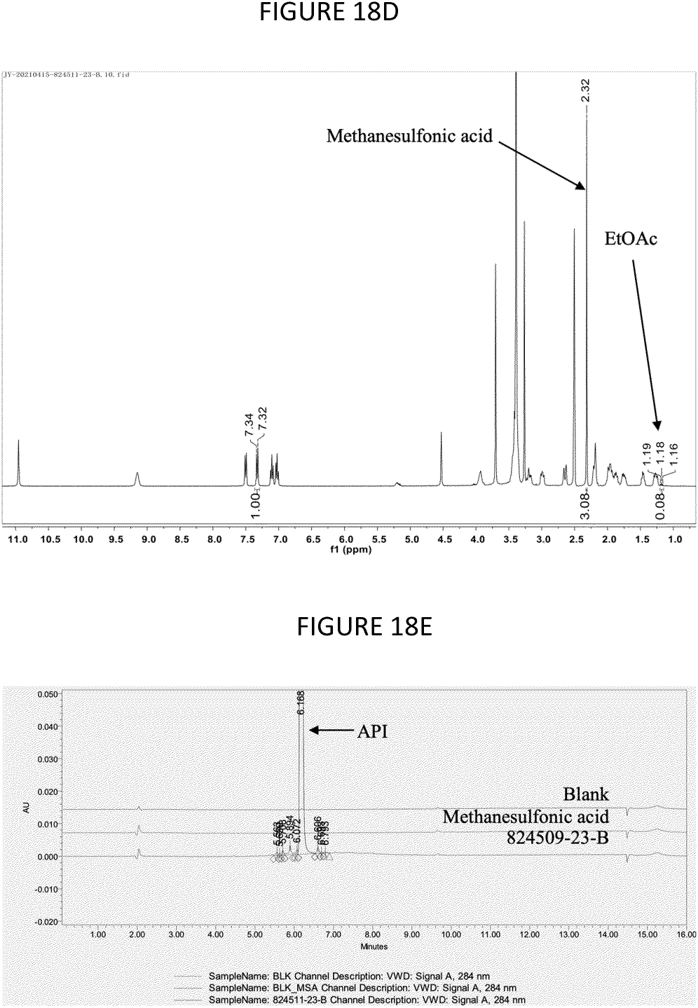

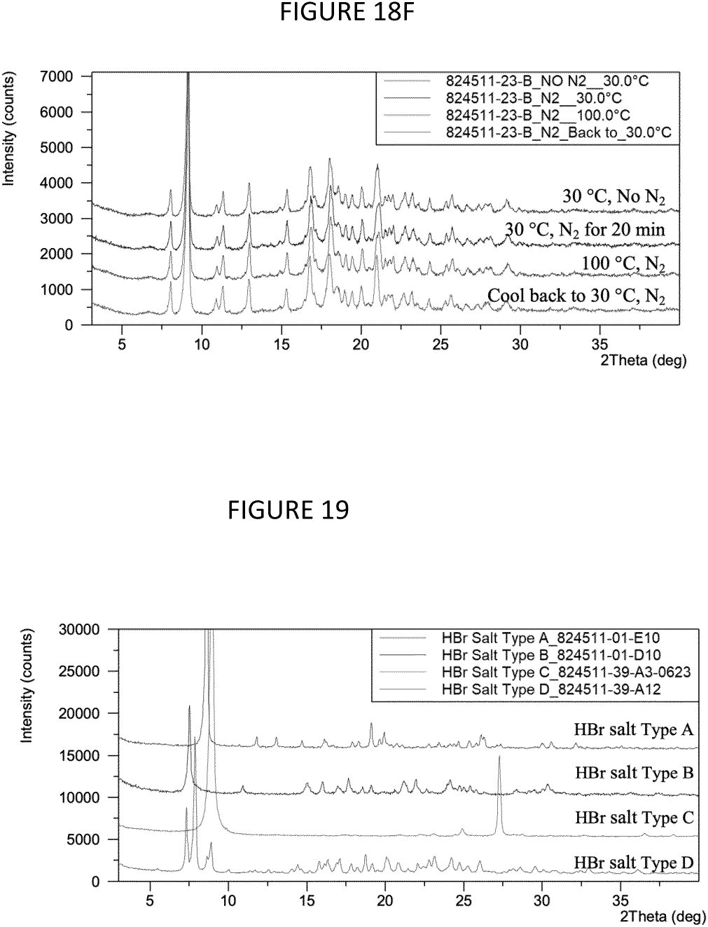

Mesylate Type A (824511-23-B) was prepared as follows: 1.0 g of freebase (824509-21-A) was weighed into a 20-mL glass vial. Approximately 5 mL of EtOAc was added to prepare a suspension. Methanesulfonic acid (264.6 mg) was mixed with 5 mL of EtOAc, and the acid solution was added to the freebase suspension dropwise while stirring with a magnetic stirrer. The sample became gel like, which was then stirred at RT for 3 days. The sample was then used for a temperature cycling experiment to improve crystallinity (50° C.~5° C., 2 cycles, one cycle: heat to 50° C. at 4.5° C./min, isothermal at 50° C. for 30 min; cool to 5° C. at 0.1° C./min, isothermal at 5° C. for 30 min). After XRPD confirmation on a slurry sample, the sample was centrifuged and vacuum dried at RT overnight. As a result, 1.11 g of mesylate Type A (824511-23-B) was obtained. The XRPD pattern of prepared mesylate Type A and reference is shown in FIG. 18A and FIG. 18B. XRPD peak list of (824511-23-B) is shown in TABLE 22A. Thermogravimetric analysis (TGA) and differential scanning calorimetry (DSC) curves in FIG. 18C showed a weight loss of 1.7% up to 80.0° C. and two endothermic signals at 89.2° C. and 171.5° C. (peak). Proton nuclear magnetic resonance (¹H NMR) result in FIG. 18D showed that the peak of methanesulfonic acid and EtOAc was observed. The molar ratio of methanesulfonic acid/API was 1:1, the molar ratio of EtOAc/API was 0.027:1(theoretical weight=0.5 wt%). HPLC purity was 98.86 area% (FIG. 18E and TABLE 22B).

VT-XRPD results in FIG. 18F showed that after N₂ drying mesylate Type A (824511-23-B) for 20 min at 30° C., no form change was observed. After heating mesylate Type A sample to 100° C. and cooling back to 30° C. under N₂ protection, no obvious form change was observed, indicating that mesylate Type A was an anhydrate.

The approximate solubility of mesylate Type A (824511-23-B) was estimated in 10 solvents to guide the solvent selection in polymorph screening of the mesylate, with results summarized in TABLE 22C.

TABLE 22A

XRPD peak list of mesylate Type A (824511-23-B)

| Pos. [°2θ] | Height [cts] | FWHM Left [°2θ] | d-spacing [Å] | Rel. Int. [%] |
|---|---|---|---|---|
| 8.0822 | 633.27 | 0.1023 | 10.94 | 15.88 |
| 9.1768 | 3987.36 | 0.1023 | 9.64 | 100.00 |
| 10.9766 | 194.61 | 0.1023 | 8.06 | 4.88 |
| 11.3843 | 343.91 | 0.1023 | 7.77 | 8.63 |
| 13.0182 | 448.24 | 0.1023 | 6.80 | 11.24 |
| 14.9677 | 63.45 | 0.1535 | 5.92 | 1.59 |
| 15.4138 | 241.00 | 0.1279 | 5.75 | 6.04 |
| 16.8862 | 525.00 | 0.2303 | 5.25 | 13.17 |
| 18.0647 | 606.04 | 0.1023 | 4.91 | 15.20 |
| 18.2310 | 449.85 | 0.1023 | 4.87 | 11.28 |
| 18.6339 | 268.18 | 0.1279 | 4.76 | 6.73 |
| 19.0577 | 190.74 | 0.1279 | 4.66 | 4.78 |
| 19.4994 | 187.88 | 0.1023 | 4.55 | 4.71 |
| 20.1093 | 273.82 | 0.1279 | 4.42 | 6.87 |
| 20.9768 | 428.96 | 0.1023 | 4.24 | 10.76 |
| 21.1254 | 501.60 | 0.1279 | 4.21 | 12.58 |
| 21.5600 | 161.23 | 0.1023 | 4.12 | 4.04 |
| 21.7595 | 171.84 | 0.1279 | 4.08 | 4.31 |
| 22.0380 | 157.86 | 0.1535 | 4.03 | 3.96 |
| 22.8149 | 198.52 | 0.1279 | 3.90 | 4.98 |
| 23.2673 | 206.20 | 0.1279 | 3.82 | 5.17 |
| 23.6240 | 102.54 | 0.1023 | 3.77 | 2.57 |
| 24.3530 | 148.92 | 0.1791 | 3.66 | 3.73 |
| 25.3825 | 115.00 | 0.1023 | 3.51 | 2.88 |
| 25.7648 | 183.22 | 0.1535 | 3.46 | 4.59 |
| 26.0968 | 74.23 | 0.1535 | 3.41 | 1.86 |
| 26.6726 | 62.62 | 0.1279 | 3.34 | 1.57 |
| 28.1868 | 100.52 | 0.1279 | 3.17 | 2.52 |
| 29.2014 | 118.99 | 0.2558 | 3.06 | 2.98 |

TABLE 22B

HPLC results of mesylate Type A (824511-23-B)

| # | RRT | Area (%) | # | RRT | Area (%) |
|---|---|---|---|---|---|
| 1 | 0.90 | 0.13 | 6 | 1.00 | 98.86 |
| 2 | 0.91 | 0.05 | 7 | 1.07 | 0.21 |
| 3 | 0.93 | 0.14 | 8 | 1.09 | 0.05 |
| 4 | 0.96 | 0.36 | 9 | 1.10 | 0.05 |
| 5 | 0.98 | 0.15 | -- | -- | -- |

TABLE 22C

Approximate solubility of mesylate Type A (824511-23-B) at RT

| | | | |
|---|---|---|---|
| Solvent | Solubility (mg/mL) | Solvent | Solubility (mg/mL) |
| CHCl₃ | S>42.0* | EtOH | 20.0<S<40.0 |
| H₂O | S>42.0* | Acetone | S<2.3 |
| MeOH | S>40.0 | EtOAc | S<2.2 |
| DCM | 23.0<S<46.0* | n-Heptane | S<2.0 |
| ACN | 21.1<S<42.0* | THF | S<2.0 |

Procedure: weigh ~2 mg solids into each 3-mL glass vial, add in corresponding solvent stepwise and sonicate or oscillate to see if solids dissolve completely. Stop adding solvent when the solids dissolves or total volume reaches 1.0 mL. Calculate the approximate solubility based on solvent volume.
*: most of the solids were dissolved with very limited solids on the wall of the vial (not dissolved when adding more solvent). The actual solubility might be different from the test value.

### Polymorph Screening

Using mesylate Type A (8245511-23-B) as the starting material, a total of 30 experiments of polymorph screening were conducted using different methods. Characterization results showed that a total of 3 forms (mesylate Types A, B, and C) were obtained, including one anhydrate (mesylate Type A) and two metastable forms (mesylate Types B and C). Since mesylate Types B and C were metastable forms and could not be re-prepared, additional data could not be collected. Results of the screening experiments are summarized in TABLE 23A. Characterization data of obtained forms was summarized in TABLE 21 and the XRPD overlays of these forms were displayed in FIG. 17.

TABLE 23A

Summary of polymorph screening experiments of mesylate

| Method | No. of Experiment | Results |
|---|---|---|
| Temperature cycling | 5 | Mesylate Type A, amorphous |
| Slurry at RT | 20 | Mesylate Type A/B/C, oil/gel, amorphous |
| Slurry at 50° C. | 5 | Mesylate Type A, oil/gel, amorphous |
| Total | 30 | Mesylate Type A/B/C, oil/gel, amorphous |

### Temperature Cycling (50° C. to 5° C.)

Temperature cycling experiments were conducted in 5 solvent systems. About 20 mg of starting material (824511-23-B) was suspended in an HPLC vial with 0.5 mL of corresponding solvents listed in TABLE 23B. Cycling procedure: ramped to 50° C. at a rate of 4.5° C./min, kept the temperature at 50° C. for 30 min; cooled down to 5° C. at a rate of 0.1 C/min and kept the temperature at 5° C. for 30 min. After three cycles, store the samples at 5° C. before isolation by centrifugation and air drying for XRPD analysis. Results are summarized in TABLE 23B. Results showed that mesylate Type A and amorphous were obtained.

TABLE 23B

Summary of temperature cycling experiments of mesylate

| | | |
|---|---|---|
| Exp. ID | Solvent | Results |
| 824511-26-A1 | EtOH | Amorphous* |
| 824511-26-A2 | lPAc | Mesylate Type A |
| 824511-26-A3 | ACN | Amorphous* |
| 824511-26-A4 | Acetone | Amorphous^{#} |
| 824511-26-A5 | THF | Mesylate Type A |

*: Clear solution after temperature cycle→slurry at -20° C.→evaporation at RT→vacuum dried.
^{#}: Solid was obtained after -20° C. stirring and it dissolved quickly during solid isolation for XRPD test at RT.

### Slurry Conversion at RT

Slurry conversion experiments were conducted at RT in 20 different solvent systems. About 20 mg of starting material (824511-23-B) was suspended in an HPLC vial with 0.5 mL of corresponding solvents listed in TABLE 23C. If the solid was dissolved then more solids were added until a suspension was obtained. The suspension was slurried at RT using magnetic stirring with the speed of ~750 rpm. After ~5 days, the remaining solids were isolated by centrifugation and air dried for XRPD analysis. If a clear solution were obtained, the sample was slurried at 5° C. If it remained clear, the sample was slurried at -20° C. If there was still no precipitate, the solution was evaporated in an open vial at RT. Results are summarized in TABLE 23C. Results showed that mesylate Type A, Type B, Type C, amorphous, gel and oil were obtained.

TABLE 23C

Summary of slurry conversion experiments at RT of mesylate

| | | |
|---|---|---|
| Experiment ID | Solvent, v:v | Results |
| 824511-27-A1 | EtOH | Amorphous* |
| 824511-27-A2 | IPA | Mesylate Type A |
| 824511-27-A3 | 2-BuOH | Mesylate Type A** |
| 824511-27-A4 | MEK | Mesylate Type A |
| 824511-27-A5 | Acetone | Mesylate Type B** |
| 824511-27-A6 | ACN | Amorphous* |
| 824511-27-A7 | MTBE | Mesylate Type A |
| 824511-27-A8 | DCM | Amorphous* |
| 824511-27-A9 | lPAc | Mesylate Type A |
| 824511-27-A10 | ACN/Toluene, 2:1 | Amorphous* |
| 824511-27-A11 | MlBK/n-heptane, 2:1 | Mesylate Type A |
| 824511-27-A12 | MeOH/EtOAc, 1:2 | Oil/gel* |
| 824511-27-A13 | 1,4-Dioxane/CHCl₃, 1:4 | Amorphous* |
| 824511-27-A14 | EtOH/n-heptane, 1:4 | Mesylate Type A |
| 824511-27-A15 | ACN/Anisole, 1:4 | Oil/gel* |
| 824511-27-A16 | THF | Mesylate Type A |
| 824511-27-A17 | THF/H₂O, 981:19 | Mesylate Type C** |
| 824511-27-A18 | THF/H₂O, 957:43 | Amorphous* |
| 824511-27-A19 | THF/H₂O, 924:76 | Amorphous* |
| 824511-27-A20 | THF/H₂O, 87:13 | Amorphous* |

*: Clear solution from slurry at RT→stir at 5° C.→stir at -20° C.→evaporation at RT→vacuum dry.
**: Clear solution from slurry at RT→ stir at 5° C.→stir at -20° C.

### Slurry Conversion at 50° C.

Slurry conversion experiments were conducted at 50° C. in 5 solvent systems. About 20 mg of starting material (824511-23-B) was suspended in an HPLC glass vial with 0.5 mL of corresponding solvents listed in TABLE 23D and stirred at 50° C. If there was no solid observed, the sample was slurried at 5° C. The resulting solids were isolated by centrifugation and air dried for XRPD analysis. Results are summarized in TABLE 23D. Results showed that mesylate Type A, amorphous, oil/gel were obtained.

TABLE 23D

Summary of slurry conversion experiments at 50° C. of mesylate

| | | |
|---|---|---|
| Experiment ID | Solvent, v:v | Results |
| 824511-28-A1 | CHCl₃/n-heptane, 1:1 | Oil/gel** |
| 824511-28-A2 | ACN | Amorphous* |
| 824511-28-A3 | IPA | Mesylate Type A^{#} |
| 824511-28-A4 | Toluene | Mesylate Type A |
| 824511-28-A5 | 2-MeTHF | Mesylate Type A |

*: Clear solution from 50° C. → slurry at 5° C. → slurry at -20° C. → evaporation at RT.
**: Turn to gel when slurry at 50° C. → transfer to temperature cycling.
^{#}: Solid was obtained after -20° C. stirring and it dissolved quickly during solid isolation for XRPD test at RT.

### Re-Preparation Trials of Mesylate Forms

The detailed procedures and the results of re-preparation trials of mesylate are summarized in TABLE 23E.

TABLE 23E

Summary of re-preparation trials of mesylate forms

| | | | |
|---|---|---|---|
| Target Form | Exp. ID (824511-) | Procedure | Results |
| Mesylate Type A | 44-A1 | Dissolve 26.5 mg methanesulfonic acid with 0.5 mL EtOAc, add the acid solution into the vial with 99.7 mg freebase Type A (824509-03-A) dissolved in 0.5 mL EtOAc dropwise, transfer it to slurry at RT for 2 days. | Mesylate Type A |
| Mesylate Type B | 32-A1 | Weigh ~50 mg of mesylate Type A (824511-23-B) in 0.5 mL of acetone and slurry at -20° C. for 5 days. | Mesylate Type B* |
| | 32-B1 | Dissolve ~8.3 mg methanesulfonic acid with 0.25 mL acetone, add the acid solution into the vial with ~30 mg freebase Type A (824509-24-A, refer to report CP827U04-01) dissolved in 0.25 mL acetone drop by drop, transfer it to slurry at -20° C., then clear solution was formed and transferred to evaporate at RT to obtain solids. | Gel |
| | 47-A1 | Mesylate Type A (824511-44-A1, 100-mg scale) was obtained by slurrying methanesulfonic acid and freebase (824509-03-A, refer to report CP827U04-01) in EtOAc (charge molar ratio 1:1). Weigh ~50 mg of the obtained mesylate Type A in 0.5 mL of acetone. Transfer the sample to slurry at -20° C. for 1 week. | Mesylate Type A |
| Mesylate Type C | 32-A2 | Weigh ~50 mg of mesylate Type A (824511-23-B) in 0.5 mL of THF:H₂O (981:19, v/v). Transfer the sample to slurry at -20° C. for 5 days. | Mesylate Type C* |
| | 32-B2 | Dissolve ~8.3 mg methanesulfonic acid with 0.25 mL THF:H₂O (v:v,981:19), add the acid solution into the vial with ~30 mg freebase Type A (824509-24-A) dissolved in 0.25 mL acetone dropwise quickly (completed in a few seconds), transfer it to slurry at 20° C. for 2 weeks. | Freebase Type A |
| | 47-A2 | Mesylate Type A (824511-44-A1, 100-mg scale) was obtained by slurrying methanesulfonic acid and freebase (824509-03-A) in EtOAc (charge molar ratio 1:1). Weigh ~50 mg of the obtained mesylate Type A in 0.5 mL of THF:H₂O (981:19, v/v). Transfer the sample to slurry at -20° C. for 1 week. | Mesylate Type A+C |

### Conclusion

A brief polymorph screening of 18-MC mesylate was performed and a total of three salt forms were obtained.

### Example 6 - Polymorph Screening of 18-MC HBr Salt

18-MC HBr salt Type A and Type B were obtained as described in Example 1. Polymorph screening of 18-MC HBr salt was performed to have better understand polymorphism of the salt.

The HBr salt was prepared using 18-MC freebase and used as the starting material for polymorph screening. In the screening, different crystallization methods including temperature cycling and slurry conversion at different temperatures were used, and a total of 30 experiments were conducted. Solids from screening were isolated for XRPD testing. New forms were further characterized by TGA, DSC, ¹H NMR and HPLC/IC. As the characterization results showed, two new forms (HBr salt Types C and D) were discovered. Identification results indicated that HBr salt Type A was an anhydrate, HBr salt Type B was a hydrate or solvate, HBr salt Type C was a hydrate or anhydrate and HBr salt Type D was a solvate. HBr salt Types B, C, and D could convert to HBr salt Type A after storage or heating experiments. Characterization results are summarized in TABLE 24 and XRPD patterns of different forms are displayed in FIG. 19.

To summarize, a brief polymorph screening experiments were performed and a total of four forms of HBr salt were discovered.

TABLE 24

Characterization summary of HBr salt forms

| Solid form (ID: 824511-) | TGA loss (%) | Endotherm (°C., peak) | Solvent residual (wt%)^ | Molar ratio^{&} (acid/FB) | HPLC purity (area%) | Speculated form |
|---|---|---|---|---|---|---|
| HBr salt Type A (01-E10) | 1.5 (up to 150° C.) | 208.5 | 0.6 (IPA) | 1.0 | 99.72 | Anhydrate |
| HBr salt Type B* (10-A1) | 14.2 (up to 150° C.) | 104.3, 140.3, 177.4^{#} | 10.8 (1,4-dioxane) | 0.9 | 99.27 | Solvate/ hydrate |
| HBr salt Type C (39-A3) | 2.0 (up to 100° C.) | 142.0, 208.0 | 1.12 (DCM) | 1.0 | 99.43 | Hydrate/ anhydrate |
| HBr salt Type D (39-A12) | 2.3 (up to 90° C.) | 70.1, 132.1, 200.8 | 7.58 (THF) | 1.1 | 99.48 | THF solvate |

FB: Freebase.
*: Partially converted to HBr salt Type A after storage at RT for ~20 days.
^{#}: Exothermic signal.
^{&}: Determined by HPLC/IC.
^: Determined by ¹H NMR.

### Preparation of HBr Salt Starting Material

Preparation procedure of HBr salt (824511-29-B) was as follows: 1.0 g of freebase (824509-24-A) was weighed into a 20-mL glass vial, and 7 mL of IPA were added to prepare a suspension. 554.8 mg of 40% HBr was added to 5 mL of IPA. The acid solution was added to the freebase suspension dropwise while stirring with a magnetic stirrer. The clear solution was slurried at RT for 3 days. The resulting sample was centrifuged and vacuum dried at RT overnight. About 1.08 g of HBr salt (824511-29-B) was obtained and it was consistent with HBr salt Type A reference (824511-01-E10), described in EXAMPLE 1. The XRPD result is shown in FIG. 20A, with an overly in FIG. 20B. TGA/DSC curves in FIG. 20C showed a weight loss of 3.9% up to 170.0° C. and one endothermic peak at 203.8° C. (peak). HPLC/IC results showed that the molar ratio of acid/freebase was determined as 1.0:1 and HPLC purity was 99.43 area% (FIG. 20D and TABLE 25A). Based on the VT-XRPD results in FIG. 20E, no form change of HBr salt Type A was observed when drying or heating the sample under N₂ to higher temperatures and cooling back to RT. Combining with neat DSC curve, HBr salt was speculated to be an anhydrate.

Approximate solubility of HBr salt Type A (824511-29-B) was estimated in 10 solvents to guide the solvent selection in polymorph screening of HBr salt, with data summarized in TABLE 25B.

TABLE 25A

HPLC results of HBr salt Type A (824511-29-B)

| # | RRT | Area (%) | # | RRT | Area (%) |
|---|---|---|---|---|---|
| 1 | 0.88 | 0.08 | 4 | 0.98 | 0.06 |
| 2 | 0.90 | 0.11 | 5 | 1.00 | 99.43 |
| 3 | 0.95 | 0.23 | 6 | 1.07 | 0.09 |

TABLE 25B

Approximate solubility of HBr salt Type A (824511-29-B) at RT

| | | | |
|---|---|---|---|
| Solvent | Solubility (mg/mL) | Solvent | Solubility (mg/mL) |
| MeOH | S>42.0 | EtOAc | S<2.1 |
| EtOH | 7.0<S<21.0 | CHCl₃ | S<2.1 * |
| Acetone | 2.2<S<7.3 | DCM | S<2.1 * |
| H₂O | S<2.1 | n-Heptane | S<2.0 |
| ACN | S<2.1 * | THF | S<2.0 |

*: limited solids observed after 1.0 mL solvent addition.
Procedure: weigh ~2 mg solids into each 3-mL glass vial, add in corresponding solvent stepwise and sonicate or oscillate to see if solids dissolved completely. Stop adding solvent till the solids dissolves or total volume reaches 1.0 mL. Calculate the approximate solubility based on solvent volume.

### Polymorph Screening

Using HBr salt Type A (824511-29-B) as the starting material, a total of 30 polymorph screening experiments were conducted via various crystallization methods. Results of the polymorph screening are summarized in TABLE 25C. The XRPD results showed that two new forms (HBr salt Types C and D) were obtained. Characterization data of the forms are summarized in TABLE 24 and the XRPD overlay of these forms are displayed in FIG. 19.

TABLE 25C

Summary of polymorph screening experiments of HBr salt

| Method | No. of Experiment | Results |
|---|---|---|
| Temperature cycling | 5 | HBr salt Type A |
| Slurry at RT | 13 | HBr salt Type A/C/D, freebase Type A+extra peak |
| Slurry at 50° C. | 12 | HBr salt Type A |
| Total | 30 | HBr salt Type A/C/D, freebase Type A+extra peak |

### HBr Salt Type A

HBr salt Type A was identified as an anhydrate. Characterization data on Type A reference was shown above.

### HBr Salt Type B

HBr salt Type B (824511-01-D10) was obtained as described in Example 1 by slurring freebase and HBr (charge molar ratio 1:1) in 1,4-dioxane at RT. Another batch of HBr salt Type B (824511-10-A1) was prepared using the same method for characterization, and the XRPD results are displayed in FIG. 20F and FIG. 20G. As TGA/DSC curves in FIG. 20H showed, a weight loss of 14.2% up to 150° C., two endothermic signals at 104.3° C., 140.3° C. (peak) and one exothermic signal at 177.4° C. (peak) were detected. ¹H NMR spectrum in FIGURE showed that peak of 1,4-dioxane was observed. The molar ratio of 1,4-dioxane/API was 0.6:1 (theoretical 10.8 wt%). HPLC/IC results showed that the molar ratio of acid/freebase was determined as 0.9:1 and HPLC purity was 99.27 area% (FIG. 20J and TABLE 25D). Considering that HBr salt Type B partially converted to HBr salt anhydrate Type A (peak marked) after RT storage for about 20 days (FIG. 20K), and amount of solvent detected in ¹H NMR was similar to TGA loss, HBr salt Type B was possibly a solvate or hydrate which could convert to HBr salt Type A after desolvation or dehydration during storage.

TABLE 25D

HPLC results of HBr salt Type B (824511-10-A1)

| # | RRT | Area (%) | # | RRT | Area (%) |
|---|---|---|---|---|---|
| 1 | 0.75 | 0.09 | 5 | 0.95 | 0.06 |
| 2 | 0.83 | 0.05 | 6 | 0.97 | 0.13 |
| 3 | 0.90 | 0.13 | 7 | 1.00 | 99.27 |
| 4 | 0.93 | 0.09 | 8 | 1.07 | 0.17 |

### HBr Salt Type C

HBr salt Type C (824511-39-A3) was obtained by slurrying 20.3 mg HBr salt Type A (824511-29-B) in 0.5 mL DCM at RT overnight. Since no solids precipitated, the clear solution was transferred to stir at 5° C. for about two weeks and then transferred to evaporation to dryness in desiccator at RT with silica gel. The XRPD result is displayed in FIG. 20L and TABLE 25E. As TGA/DSC curves in FIG. 20M showed, a weight loss of 2.0% up to 100° C. and two endothermic signals at 142.0° C. and 208.0° C. (peak) were observed. ¹H NMR spectrum in FIG. 20N showed that peak of DCM was observed. The molar ratio of DCM/API was 0.06:1 (theoretical weight=1.12 wt%). HPLC/IC results showed that the molar ratio of acid/freebase was determined as 1.0:1 and HPLC purity was 99.43 area% (FIG. 20O and TABLE 25F).

XRPD results in FIG. 20P showed that after storage and heating HBr salt Type C to 100° C., the peak of HBr salt Type A was observed, and after heating sample to 150° C., most diffraction peaks were consistent with HBr salt Type A. ¹H NMR result in FIG. 20Q showed that the peak of DCM was observed. The molar ratio of DCM/API was 0.005:1 (theoretical weight=0.09 wt%). Combined with the results of heating experiment and limited solvent amount in the sample, HBr salt Type C was speculated to be a hydrate (theoretical water content for a hemi-hydrate is 1.96%) or anhydrate.

TABLE 25E

XRPD peak list of HBr salt Type C (824511-39-A3)

| Pos. [°2θ] | Height [cts] | FWHM Left [°2θ] | d-spacing [Å] | Rel. Int. [%] |
|---|---|---|---|---|
| 8.9668 | 33315.25 | 0.1535 | 9.86 | 100.00 |
| 13.0371 | 30.93 | 0.6140 | 6.79 | 0.09 |
| 15.7616 | 13.47 | 0.8187 | 5.62 | 0.04 |
| 17.4479 | 81.74 | 0.3070 | 5.08 | 0.25 |
| 18.0992 | 64.07 | 0.2047 | 4.90 | 0.19 |
| 20.9546 | 98.65 | 0.3582 | 4.24 | 0.30 |
| 22.2069 | 169.35 | 0.1279 | 4.00 | 0.51 |
| 23.0999 | 231.83 | 0.2303 | 3.85 | 0.70 |
| 24.4367 | 148.71 | 0.1279 | 3.64 | 0.45 |
| 24.9087 | 678.69 | 0.1791 | 3.57 | 2.04 |
| 27.2755 | 7529.84 | 0.1791 | 3.27 | 22.60 |
| 28.1816 | 155.84 | 0.2558 | 3.17 | 0.47 |
| 28.7526 | 186.09 | 0.1535 | 3.10 | 0.56 |
| 30.5327 | 84.44 | 0.2047 | 2.93 | 0.25 |
| 32.6697 | 92.10 | 0.3070 | 2.74 | 0.28 |
| 33.8767 | 40.37 | 0.3070 | 2.65 | 0.12 |
| 36.5285 | 274.37 | 0.2303 | 2.46 | 0.82 |
| 37.5934 | 38.68 | 0.1535 | 2.39 | 0.12 |
| 38.3600 | 143.40 | 0.1791 | 2.35 | 0.43 |

TABLE 25F

HPLC results of HBr salt Type C (824511-39-A3)

| # | RRT | Area (%) | # | RRT | Area (%) |
|---|---|---|---|---|---|
| 1 | 0.91 | 0.08 | 4 | 0.98 | 0.14 |
| 2 | 0.92 | 0.05 | 5 | 1.00 | 99.43 |
| 3 | 0.96 | 0.17 | 6 | 1.07 | 0.14 |

### HBr Salt Type D

HBr salt Type D (824511-39-A12) was obtained by slurrying 20.0 mg HBr salt Type A (824511-29-B) in 0.5 mL THF:H₂O (924:76, v/v, aw≈0.6) at RT overnight. Since no solids precipitated, the clear solution was transferred to stir at 5° C. for about two weeks and then transferred to evaporation to dryness in desiccator at RT with silica gel. The XRPD result is displayed in FIG. 20R and TABLE 25G. As shown in FIG. 20S, TGA weight losses of 2.3% up to 90° C., 7.6% from 90° C. to 170° C. were observed, and three DSC endothermic signals at 70.1° C., 132.1° C. and 200.8° C. (peak) were observed. ¹H NMR spectrum in FIG. 20T showed that the peak of THF was observed. The molar ratio of THF/API was 0.51:1 (theoretical weight=7.58 wt%). HPLC/IC results showed that the molar ratio of acid/freebase was determined as 1.1:1 and HPLC purity was 99.48 area% (FIG. 20U and TABLE 25H).

XRPD results in FIG. 20V showed that after 100° C. heating of HBr salt Type D, the extra peak of HBr salt Type A was observed. After 150° C. heating, most diffraction peaks of the sample were consistent with HBr salt Type A. ¹H NMR result in FIG. 20W showed that no obvious peak of THF was observed. Combined with the results of heating experiment, HBr salt Type D was speculated as a THF solvate.

TABLE 25G

XRPD peak list of HBr salt Type D (824511-39-A12)

| Pos. [°2θ] | Height [cts] | FWHM Left [°2θ] | d-spacing [Å] | Rel. Int. [%] |
|---|---|---|---|---|
| 5.4959 | 225.70 | 0.1023 | 16.08 | 1.41 |
| 7.3265 | 7319.41 | 0.1023 | 12.07 | 45.82 |
| 7.8786 | 15974.78 | 0.1279 | 11.22 | 100.00 |
| 8.6494 | 1867.75 | 0.0768 | 10.22 | 11.69 |
| 8.8923 | 3484.11 | 0.1279 | 9.94 | 21.81 |
| 10.0085 | 349.58 | 0.1279 | 8.84 | 2.19 |
| 11.3843 | 195.47 | 0.1279 | 7.77 | 1.22 |
| 11.6963 | 320.41 | 0.1023 | 7.57 | 2.01 |
| 12.0863 | 164.99 | 0.1023 | 7.32 | 1.03 |
| 12.5554 | 354.61 | 0.1535 | 7.05 | 2.22 |
| 12.9318 | 153.66 | 0.1535 | 6.85 | 0.96 |
| 13.4213 | 133.88 | 0.1023 | 6.60 | 0.84 |
| 14.0485 | 448.50 | 0.1535 | 6.30 | 2.81 |
| 14.4185 | 982.26 | 0.1279 | 6.14 | 6.15 |
| 14.6467 | 463.08 | 0.1279 | 6.05 | 2.90 |
| 15.1962 | 235.00 | 0.1279 | 5.83 | 1.47 |
| 15.7961 | 1371.13 | 0.1279 | 5.61 | 8.58 |
| 16.1225 | 1086.79 | 0.0768 | 5.50 | 6.80 |
| 16.3253 | 1583.26 | 0.1023 | 5.43 | 9.91 |
| 16.9174 | 1217.03 | 0.1535 | 5.24 | 7.62 |
| 17.0997 | 1661.48 | 0.1279 | 5.19 | 10.40 |
| 17.8398 | 1073.96 | 0.1535 | 4.97 | 6.72 |
| 18.2168 | 671.03 | 0.1535 | 4.87 | 4.20 |
| 18.7397 | 2213.77 | 0.1535 | 4.74 | 13.86 |
| 19.1722 | 1141.76 | 0.1535 | 4.63 | 7.15 |
| 19.5205 | 355.86 | 0.0768 | 4.55 | 2.23 |
| 20.0863 | 1899.29 | 0.1535 | 4.42 | 11.89 |
| 20.2336 | 1411.93 | 0.1279 | 4.39 | 8.84 |
| 20.8353 | 1231.98 | 0.1791 | 4.26 | 7.71 |
| 21.4543 | 509.51 | 0.1279 | 4.14 | 3.19 |
| 22.0693 | 1140.52 | 0.1279 | 4.03 | 7.14 |
| 22.4194 | 793.14 | 0.1023 | 3.97 | 4.96 |
| 22.7933 | 1572.48 | 0.1279 | 3.90 | 9.84 |
| 23.1423 | 2002.63 | 0.1535 | 3.84 | 12.54 |
| 24.2230 | 1838.39 | 0.1791 | 3.67 | 11.51 |
| 24.7360 | 1257.95 | 0.1535 | 3.60 | 7.87 |
| 25.2704 | 819.85 | 0.2047 | 3.52 | 5.13 |
| 26.0362 | 1450.36 | 0.2047 | 3.42 | 9.08 |
| 26.9116 | 180.29 | 0.1535 | 3.31 | 1.13 |
| 27.9452 | 383.93 | 0.1535 | 3.19 | 2.40 |
| 28.1627 | 537.87 | 0.1279 | 3.17 | 3.37 |
| 28.5978 | 742.37 | 0.1535 | 3.12 | 4.65 |
| 29.5594 | 814.18 | 0.1791 | 3.02 | 5.10 |
| 30.0929 | 420.30 | 0.1535 | 2.97 | 2.63 |
| 30.8346 | 349.73 | 0.1535 | 2.90 | 2.19 |
| 31.4028 | 131.62 | 0.2047 | 2.85 | 0.82 |
| 32.1885 | 220.79 | 0.1023 | 2.78 | 1.38 |
| 32.4536 | 283.93 | 0.1279 | 2.76 | 1.78 |
| 32.9780 | 675.20 | 0.2047 | 2.72 | 4.23 |
| 34.2900 | 233.19 | 0.1023 | 2.62 | 1.46 |
| 35.2245 | 176.07 | 0.1535 | 2.55 | 1.10 |
| 36.1075 | 432.56 | 0.2047 | 2.49 | 2.71 |
| 37.1339 | 74.35 | 0.4093 | 2.42 | 0.47 |
| 38.5344 | 97.23 | 0.1535 | 2.34 | 0.61 |
| 38.9928 | 99.64 | 0.1791 | 2.31 | 0.62 |
| 39.5360 | 100.41 | 0.1535 | 2.28 | 0.63 |

TABLE 25H

HPLC results of HBr salt Type D (824511-39-A12)

| # | RRT | Area (%) | # | RRT | Area (%) |
|---|---|---|---|---|---|
| 1 | 0.90 | 0.12 | 4 | 1.00 | 99.48 |
| 2 | 0.95 | 0.18 | 5 | 1.07 | 0.14 |
| 3 | 0.98 | 0.08 | -- | -- | -- |

### Conclusion

A brief polymorph screening of 18-MC HBr salt was performed and a total of four salt forms were obtained.

### Example 7 - Polymorph Screening of 18-MC Tosylate

18-MC tosylate Type A and Type B were first obtained as described in Example 1. Polymorph screening of 18-MC tosylate was performed to better understand polymorphism of the salt.

Tosylate material was first prepared using 18-MC freebase and used as the starting material for the polymorph screening. In the screening, different crystallization methods including temperature cycling and slurry conversion at different temperatures were used, and a total of 30 experiments were conducted. Solids from screening were isolated for XRPD testing. New forms were further characterized by TGA, DSC, ¹H NMR and HPLC. As the characterization and identification results showed, seven new forms (tosylate Types C, D, E, F, G, H, and I) were discovered. Results indicated that tosylate Types A, B, and C were hydrates, Types D and I were anhydrates, Types E, F, and G were solvates and Type H was a metastable form. Characterization results are summarized in TABLE 26 and XRPD patterns of different forms are displayed in FIG. 21.

To summarize, a brief polymorph screen was performed and a total of nine forms of the tosylate salt were discovered.

TABLE 26

Form characterization summary of tosylate

| | | | | | | |
|---|---|---|---|---|---|---|
| Solid form (ID) | TGA loss (%) | Endotherm (°C., peak) | Solvent residual (wt%)^{#} | Molar ratio^{#} (acid/FB) | HPLC purity (area%) | Speculated form |
| Tosylate Type A (819246-23-A18) | 4.2 (120° C.) | 72.9, 114.1, 145.3° C. | 2.93 (EtOAc) | 0.9:1 | 99.56 | Hydrate |
| Tosylate Type B (819246-23-D18) | 5.8 (120° C.) | 93.9, 119.0, 183.5 | Not detected | 1.0:1 | 99.80 | Hydrate |
| Tosylate Type C (824511-23-A) | 4.6 (110° C.) | 93.8, 128.8 | 0.46 (THF) | 1.0:1 | 99.33 | Hydrate |
| Tosylate Type D (824511-23-A_N2_30.0° C. ) | -- | -- | -- | -- | -- | Anhydrate |
| Tosylate Type E (824528-05-A9) | 2.8% (up to 80° C.) 11.7% (80° C. to 130° C.) | 100.2, 105.4 | 11.84 (1,4-Dioxane) | 0.9:1 | 99.85 | 1,4-Dioxane solvate |
| Tosylate Type F (824528-06-B1) | 17.4% (up to 100° C.) | 74.1, 96.8, 122.5 | 8.22 (CHCl₃) | 1.0:1 | 99.52 | CHCl₃ solvate |
| Tosylate Type G (824528-06-A1) | 1.2% (up to 70° C.) 7.1% (70° C. to 120° C.) | 107.9 | 8.70 (Anisole) | 0.9:1 | 99.63 | Anisole solvate |
| Tosylate Type H (824528-05-A12) | -- | -- | -- | -- | -- | Metastable |
| Tosylate Type I (824528-09-A2_N2_100.0° C.) | -- | -- | -- | -- | -- | Anhydrate |

--: No data collected since form change after exposure to air.
^{#}: Determined based on ¹H NMR data.

### Preparation of Tosylate Starting Material

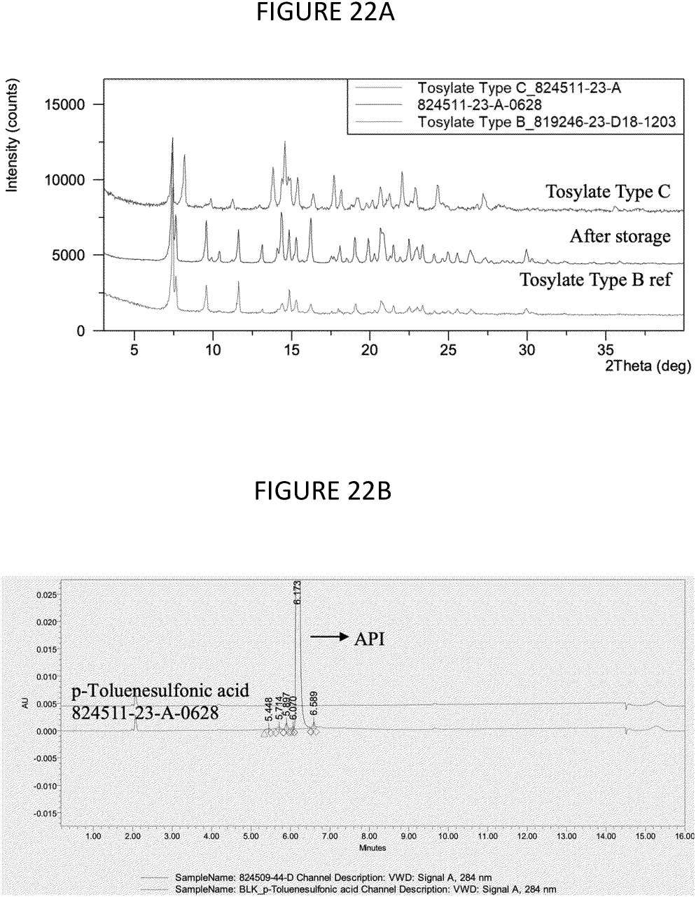

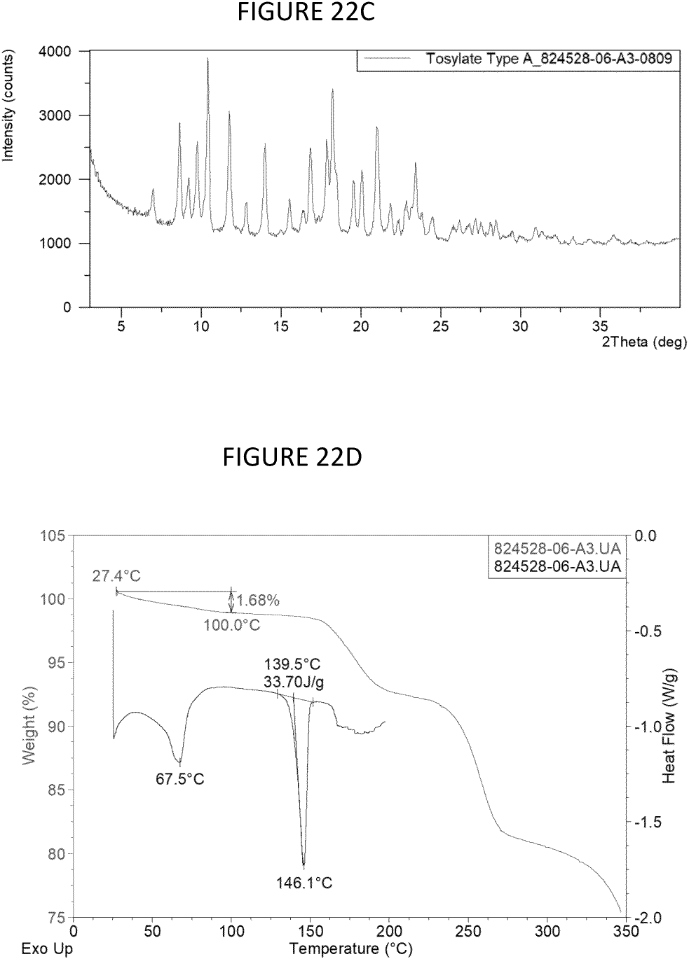

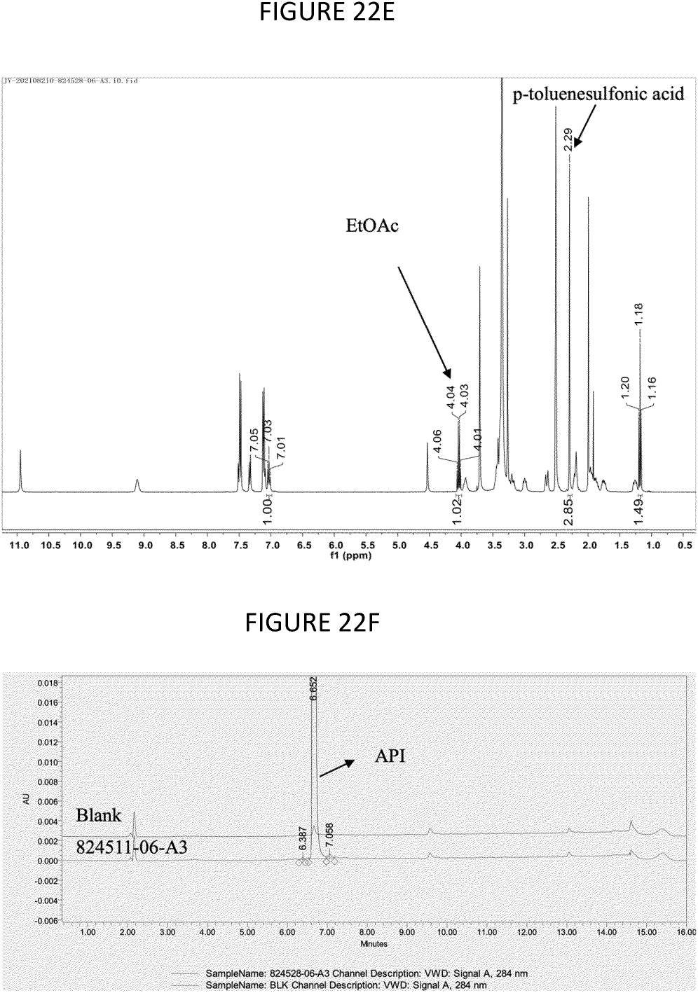

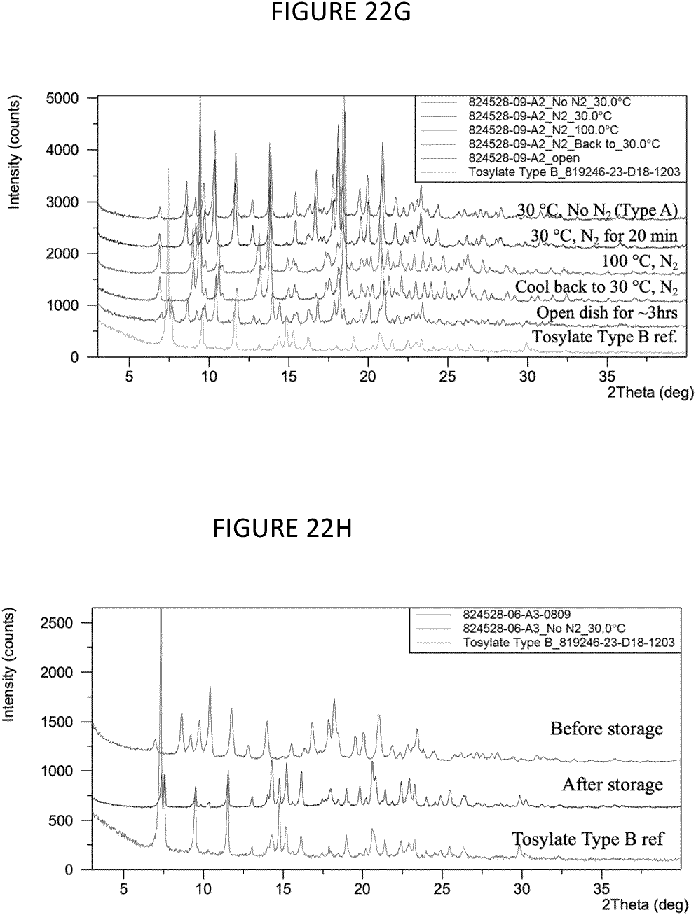

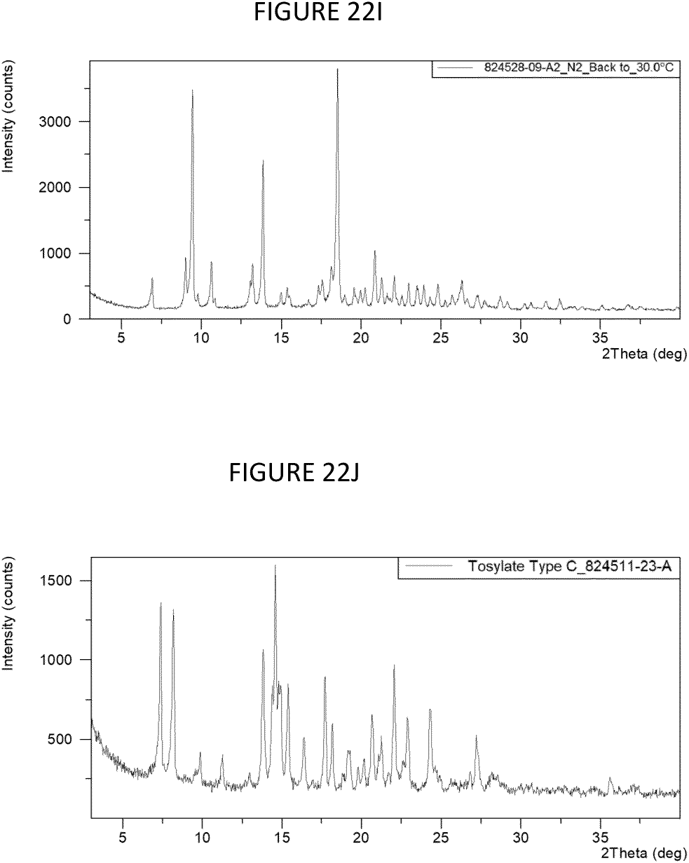

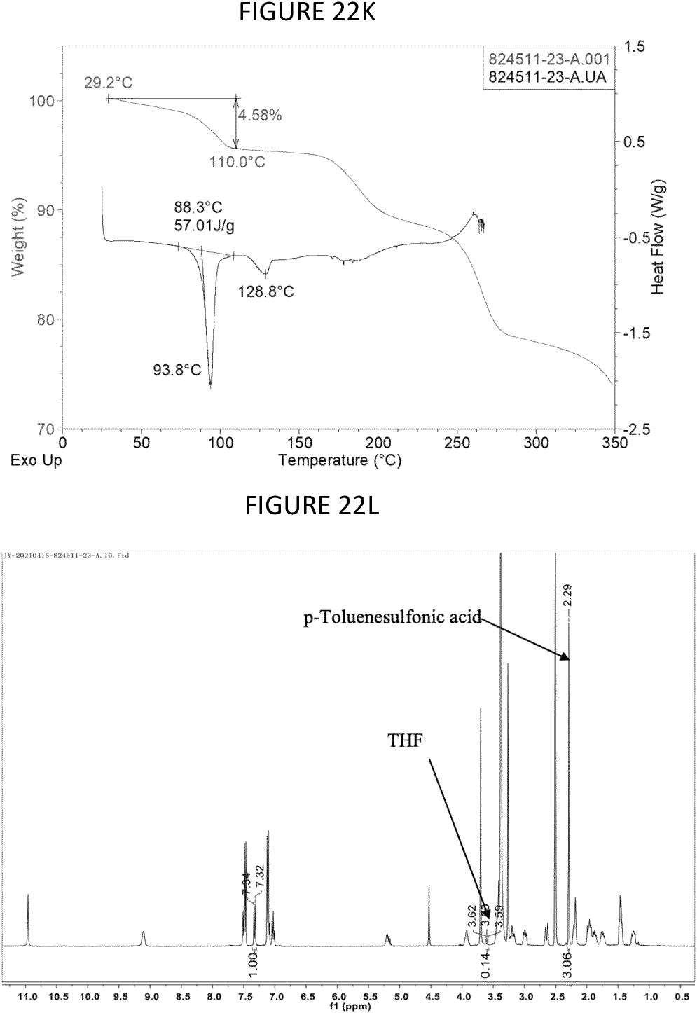

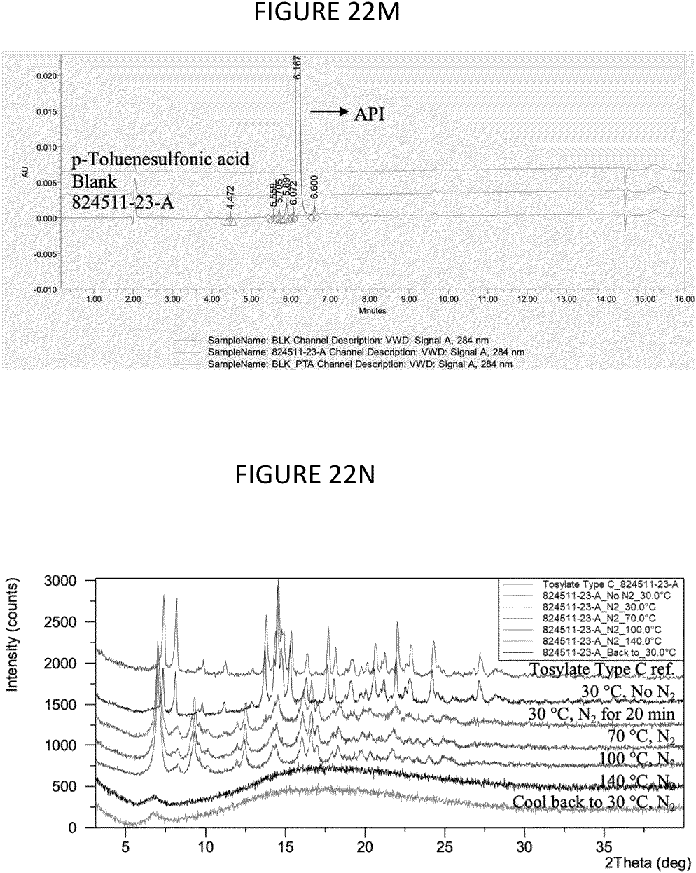

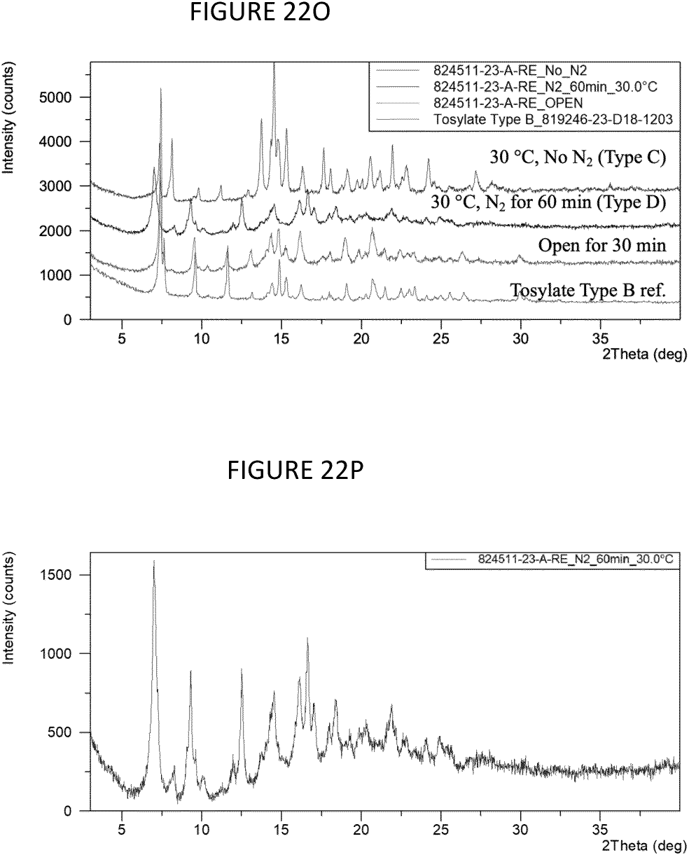

Preparation procedure of tosylate sample was as follows: 1.0 g of freebase (824509-21-A) was weighed into a 20-mL glass vial. 4 mL of THF was added to prepare a suspension. 527.5 mg of p-toluenesulfonic acid was dissolved in 4 mL of THF. The acid solution was added into the freebase suspension dropwise with stirring. The resulting clear solution was allowed to stir at 5° C. for 4 days. Solids were isolated from suspension by centrifugation and vacuum dried at RT overnight. As a result, about 1.23 g of tosylate (824511-23-A) was obtained and it showed different XRPD pattern from tosylate Type A or B, and was assigned as tosylate Type C. After storing tosylate Type C (824511-23-A) at RT for about 2.5 month, a form change to tosylate Type B (renamed as 824511-23-A-0628) was observed (FIG. 22A). The HPLC purity of tosylate Type B (824511-23-A-0628) was determined to be 99.43 area% (FIG. 22B and TABLE 27A) and it was used as starting material of polymorph screening experiments.

TABLE 27A

HPLC results of tosylate Type B (824511-23-A-0628)

| # | RRT | Area (%) | # | RRT | Area (%) |
|---|---|---|---|---|---|
| 1 | 0.88 | 0.05 | 4 | 0.98 | 0.06 |
| 2 | 0.93 | 0.11 | 5 | 1.00 | 99.43 |
| 3 | 0.96 | 0.18 | 6 | 1.07 | 0.18 |

Approximate solubility of tosylate Type B (824511-23-A-0628) was estimated in 40 solvents to guide the solvent selection in polymorph screening of tosylate, with data summarized in TABLE 27B.

TABLE 27B

Approximate solubility of tosylate Type B (824511-23-A-0628) at RT and 50° C.

| RT (mg/mL) | | | | 50° C. (mg/mL) | |
|---|---|---|---|---|---|
| Solvent | Solubility | Solvent (v:v) | Solubility | Solvent (v:v) | Solubility |
| MeOH | S>44.0 | ACN/toluene (4:1) | S>42.0 | n-Butanol | 22.0<S<44.0 |
| EtOH | S>40.0 | EtOH/n-hexane (3:1) | S>42.0 | IPA/EtOAc (1:4) | 21.0<S<42.0 |
| CHCl₃ | S>40.0 | THF/H₂O (87:13) | S>42.0 | ACN/EtOAc (1:2) | 19.0<S<38.0 |
| ACN | 22.0<S<44.0 | ACN/H₂O (1:3) | 20.0<S<40.0 | CHCl₃/n-hexane (1:1) | 7.3<S<22.0 |
| DCM | 7.3<S<22.0 | ACN/toluene (1:1) | 19.0<S<38.0 | Toluene/IPA (9:1) | 6.3<S<19.0 |
| Acetone | 6.7<S<20.0 | Acetone/n-heptane (9:1) | 6.3<S<19.0 | 2-MeTHF | 2.1<S<7.0^{&} |
| MEK | 6.7<S<20.0 | THF/H₂O (981:19) | 2.1<S<7.0 | MIBK | 2.0<S<6.7^{&} |
| THF | 6.7<S<20.0 | EtOH/n-hexane (1:2) | 2.1<S<7.0 | 1,4-Dioxane/n-heptane (9:1) | 2.2<S<7.3 |
| IPA | 6.3<S<19.0 | CHCl₃/toluene (1:1) | 1.9<S<6.3 | n-Heptane/EtOH (5:1) | 2.2<S<7.3 |
| EtOAc | S<2.1^{&} | MeOH/H₂O (1:3) | 1.9<S<6.3 | Anisole | 1.9<S<6.3 |
| 2-MeTHF | S<2.1 | Acetone/n-heptane (4:1) | S<2.0 | IPAc | S<2.2 |
| n-Heptane | S<2.0 | CHCl₃/toluene (1:4) | S<1.9 | MTBE | S<2.1 |
| H₂O | S<2.0 | -- | -- | n-Heptane | S<2.0 |
| -- | -- | -- | -- | Toluene | S<1.9 |
| -- | -- | -- | -- | 1,4-Dioxane/n-heptane (1:2) | S<1.9 |

^{&}: little solid in the vial. Procedure: weigh ~2 mg solids into each 3-mL glass vial, add in corresponding solvent stepwise and sonicate or oscillate to see if solids dissolved completely. Stop adding solvent till the solids dissolves or total volume reaches 1.0 mL. The above procedure was conducted at corresponding temperature. Calculate the approximate solubility based on solvent volume.

### Polymorph Screening

Using tosylate Type B (824511-23-A-0628) as the starting material, a total of 30 polymorph screening experiments were conducted via various crystallization methods. Results of polymorph screening are summarized in TABLE 27C. XRPD results showed that a total of nine forms (tosylate Types A to I) were obtained from the polymorph screening and characterization, including three hydrates (tosylate Types A, B, and C), two anhydrate (tosylate Types D and I), three solvates (tosylate Types E, F, and G) and one metastable form (tosylate Type H). Characterization data of obtained forms is summarized in TABLE 26 and the XRPD overlays of these forms are displayed in FIG. 21.

TABLE 27C

Summary of polymorph screening experiments of tosylate

| Method | No. of Experiment | Results |
|---|---|---|
| Temperature Cycling | 5 | Tosylate Type B |
| Slurry at RT | 20 | Tosylate Type B/C/B+F/B+C/B+C+extra peak/C+extra peak |
| Slurry at 50° C. | 5 | Tosylate Type B/E/F/G/H/A+H |
| Total | 30 | Tosylate Type A/B/C/D, amorphous, gel |

Note: Tosylate Type I was observed in form identification.

### Tosylate Type A/I

Tosylate Type A was first obtained in a previous experiment. For batch 824528-06-A3-0809, it was obtained by air drying of tosylate Type H (824528-06-A3, slurry 40.2 mg tosylate (824511-23-A-0628) in 0.5 mL IPA/EtOAc (1:4, v/v) at 50° C. for 3 days) at RT for ~3 hours. The XRPD result is displayed in FIG. 22C and TABLE 27D. TGA/DSC results in FIG. 22D showed a weight loss of 1.7% up to 100.0° C. and two endothermic peaks at 67.5° C. and 146.1° C. (peak). Using DMSO-d₆ as solvent, ¹H NMR results in FIG. 22E showed that the peak of p-toluenesulfonic acid and EtOAc were observed. The molar ratio of p-toluenesulfonic acid/API was 1:1, the molar ratio of EtOAc/API was 0.51:1 (theoretical weight=7.79 wt%, which was higher than TGA loss and speculated to be caused by inhomogeneity of the sample). HPLC purity of the sample was determined as 99.75 area% (FIG. 22F and TABLE 27E).

VT-XRPD results in FIG. 22G showed that after drying tosylate Type A (824528-06-A3) under N₂ for 30 min at 30° C., no form change was observed. After heating the sample to 100° C. under N₂ protection, a form change was observed, which was assigned as tosylate Type I (FIGURE and TABLE 27F). After cooling back to 30° C. under N₂ protection, no form change was observed for Type I. After open dish for ~3 hrs, tosylate Type I converted back to tosylate Type A with an extra peak of tosylate Type B. Thus, tosylate Type A was speculated as a hydrate and tosylate Type I was speculated as an anhydrate.

XRPD results in FIG. 22H showed that after storing tosylate Type A at RT for ~7 days, the peak of tosylate Type B was observed. After storage for 3 weeks, the tosylate Type A sample was totally converted to tosylate Type B, which indicated Type B was possibly a hydrate or anhydrate.

TABLE 27D

XRPD peak list of tosylate Type A (824528-06-A3)

| Pos. [°2θ] | Height [cts] | FWHM Left [°2θ] | d-spacing [Å] | Rel. Int. [%] |
|---|---|---|---|---|
| 6.9622 | 468.93 | 0.1279 | 12.70 | 17.93 |
| 8.6317 | 1581.15 | 0.1279 | 10.24 | 60.47 |
| 9.1979 | 753.24 | 0.1279 | 9.62 | 28.81 |
| 9.7358 | 1299.86 | 0.1535 | 9.08 | 49.71 |
| 10.4125 | 2614.89 | 0.1535 | 8.50 | 100.00 |
| 11.7576 | 1812.72 | 0.1791 | 7.53 | 69.32 |
| 12.7942 | 458.74 | 0.1279 | 6.92 | 17.54 |
| 13.9745 | 1373.53 | 0.1535 | 6.34 | 52.53 |
| 14.9772 | 66.42 | 0.1535 | 5.92 | 2.54 |
| 15.5244 | 552.21 | 0.1535 | 5.71 | 21.12 |
| 16.3842 | 385.20 | 0.2047 | 5.41 | 14.73 |
| 16.8287 | 1370.00 | 0.1791 | 5.27 | 52.39 |
| 17.8663 | 1466.05 | 0.1279 | 4.96 | 56.07 |
| 18.2132 | 2277.95 | 0.1279 | 4.87 | 87.11 |
| 18.4925 | 931.47 | 0.0768 | 4.80 | 35.62 |
| 19.5378 | 856.52 | 0.1791 | 4.54 | 32.76 |
| 20.0561 | 1027.28 | 0.1791 | 4.43 | 39.29 |
| 21.0144 | 1710.69 | 0.2047 | 4.23 | 65.42 |
| 21.8449 | 534.97 | 0.2303 | 4.07 | 20.46 |
| 22.3397 | 242.59 | 0.1535 | 3.98 | 9.28 |
| 22.8309 | 567.99 | 0.1023 | 3.90 | 21.72 |
| 23.4187 | 1185.47 | 0.1535 | 3.80 | 45.34 |
| 23.8035 | 408.26 | 0.1023 | 3.74 | 15.61 |
| 24.4660 | 313.92 | 0.2814 | 3.64 | 12.01 |
| 25.7923 | 191.83 | 0.2558 | 3.45 | 7.34 |
| 26.1632 | 306.22 | 0.1279 | 3.41 | 11.71 |
| 26.7918 | 266.62 | 0.1023 | 3.33 | 10.20 |
| 27.1629 | 343.00 | 0.1791 | 3.28 | 13.12 |
| 27.5145 | 288.26 | 0.1535 | 3.24 | 11.02 |
| 28.1122 | 290.73 | 0.1535 | 3.17 | 11.12 |
| 28.4622 | 305.94 | 0.1535 | 3.14 | 11.70 |
| 29.4615 | 135.24 | 0.1279 | 3.03 | 5.17 |
| 29.9497 | 88.57 | 0.1535 | 2.98 | 3.39 |
| 30.9402 | 222.69 | 0.2047 | 2.89 | 8.52 |
| 31.3403 | 170.16 | 0.2047 | 2.85 | 6.51 |
| 32.1670 | 117.06 | 0.2558 | 2.78 | 4.48 |
| 33.2789 | 106.34 | 0.1791 | 2.69 | 4.07 |
| 34.3218 | 72.89 | 0.2558 | 2.61 | 2.79 |
| 35.8382 | 138.59 | 0.1791 | 2.51 | 5.30 |
| 36.8770 | 56.38 | 0.1535 | 2.44 | 2.16 |
| 37.9470 | 36.56 | 0.3070 | 2.37 | 1.40 |

TABLE 27E

HPLC results of tosylate Type A (824528-06-A3)

| # | RRT | Area (%) |
|---|---|---|
| 1 | 0.96 | 0.06 |
| 2 | 1.00 | 99.75 |
| 3 | 1.06 | 0.19 |

TABLE 27F

XRPD peak list of tosylate Type I (824528-09-A2_N2_Back to_30° C.)

| Pos. [°2θ] | Height [cts] | FWHM Left [°2θ] | d-spacing [Å] | Rel. Int. [%] |
|---|---|---|---|---|
| 6.9108 | 469.69 | 0.0669 | 12.79 | 12.88 |
| 9.0215 | 765.20 | 0.0836 | 9.80 | 20.99 |
| 9.4463 | 3323.45 | 0.0836 | 9.36 | 91.17 |
| 9.7772 | 206.31 | 0.0669 | 9.05 | 5.66 |
| 10.6225 | 708.19 | 0.0836 | 8.33 | 19.43 |
| 10.8493 | 152.84 | 0.0669 | 8.15 | 4.19 |
| 13.0613 | 421.53 | 0.0669 | 6.78 | 11.56 |
| 13.2170 | 681.69 | 0.0836 | 6.70 | 18.70 |
| 13.8562 | 2254.71 | 0.1171 | 6.39 | 61.85 |
| 14.9977 | 248.57 | 0.1171 | 5.91 | 6.82 |
| 15.3695 | 305.76 | 0.1004 | 5.77 | 8.39 |
| 15.5526 | 162.72 | 0.0836 | 5.70 | 4.46 |
| 16.7185 | 147.84 | 0.0502 | 5.30 | 4.06 |
| 17.3501 | 344.18 | 0.0836 | 5.11 | 9.44 |
| 17.5733 | 426.65 | 0.1338 | 5.05 | 11.70 |
| 18.1424 | 610.37 | 0.1171 | 4.89 | 16.74 |
| 18.5308 | 3645.28 | 0.1171 | 4.79 | 100.00 |
| 18.9809 | 212.16 | 0.0836 | 4.68 | 5.82 |
| 19.5655 | 332.34 | 0.0669 | 4.54 | 9.12 |
| 19.9540 | 274.68 | 0.1004 | 4.45 | 7.54 |
| 20.2591 | 324.02 | 0.1004 | 4.38 | 8.89 |
| 20.8729 | 882.60 | 0.1171 | 4.26 | 24.21 |
| 21.2972 | 466.66 | 0.1338 | 4.17 | 12.80 |
| 21.6425 | 244.22 | 0.0669 | 4.11 | 6.70 |
| 22.0872 | 486.32 | 0.1004 | 4.02 | 13.34 |
| 22.5734 | 209.20 | 0.0836 | 3.94 | 5.74 |
| 22.9871 | 395.68 | 0.1004 | 3.87 | 10.85 |
| 23.5164 | 352.87 | 0.0836 | 3.78 | 9.68 |
| 23.9482 | 356.98 | 0.1004 | 3.72 | 9.79 |
| 24.3279 | 185.36 | 0.1004 | 3.66 | 5.08 |
| 24.8291 | 368.33 | 0.1171 | 3.59 | 10.10 |
| 25.3058 | 124.55 | 0.1004 | 3.52 | 3.42 |
| 25.7181 | 200.69 | 0.1004 | 3.46 | 5.51 |
| 26.3264 | 416.15 | 0.1004 | 3.39 | 11.42 |
| 26.6435 | 151.82 | 0.1338 | 3.35 | 4.16 |
| 27.2878 | 200.65 | 0.2007 | 3.27 | 5.50 |
| 27.7398 | 129.44 | 0.1673 | 3.22 | 3.55 |
| 28.7389 | 188.45 | 0.0669 | 3.11 | 5.17 |
| 29.1711 | 116.29 | 0.1506 | 3.06 | 3.19 |
| 30.2168 | 93.30 | 0.0836 | 2.96 | 2.56 |
| 30.6457 | 102.29 | 0.1338 | 2.92 | 2.81 |
| 31.5896 | 117.85 | 0.1673 | 2.83 | 3.23 |
| 32.4480 | 148.08 | 0.0836 | 2.76 | 4.06 |
| 33.3854 | 53.40 | 0.1004 | 2.68 | 1.46 |
| 33.8453 | 43.54 | 0.2007 | 2.65 | 1.19 |
| 35.1014 | 56.79 | 0.2007 | 2.56 | 1.56 |
| 36.7131 | 74.23 | 0.1673 | 2.45 | 2.04 |
| 37.4934 | 44.91 | 0.3011 | 2.40 | 1.23 |

### Tosylate Types B, C, and D

Tosylate Type C (824511-23-A) was obtained by slurrying 1.0 g freebase and 527.5 mg p-toluenesulfonic acid (charge molar ratio 1:1) in 8 mL THF at 5° C. for ~4 days. Resulting solids were isolated by centrifugation and vacuum drying at RT overnight. The XRPD result is displayed in FIG. 22J and TABLE 27G. TGA/DSC results in FIG. 22K showed a weight loss of 4.6% up to 110° C., two endothermic signals at 93.8° C. and 128.8° C. (peak). ¹H NMR result in FIG. 22L showed that the peak of p-toluenesulfonic acid and THF were observed. The molar ratio of p-toluenesulfonic acid/API was 1:1, the molar ratio of THF/API was 0.01:1 (theoretical weight=0.46 wt%). HPLC purity of the sample was determined as 99.33 area% (FIG. 22M and TABLE 27H).

VT-XRPD result in FIG. 22N showed that after N₂-drying for 30 min at 30° C., form change was observed for tosylate Type C. The new form was assigned as tosylate Type D (FIG. 22P and TABLE 271). After heating tosylate Type D to 100° C. under N₂ protection, no form change was observed. By heating the sample to 140° C. under N₂ protection, an amorphous pattern was observed. Considering limited solvent residual (much less than TGA loss), tosylate Type C was speculated to be a hydrate (theoretical water content for sesqui-hydrate is 4.76%) and tosylate Type D was an anhydrate.

XRPD result in FIG. 22O showed that after exposing tosylate Type D to ambient conditions for ~30 min, it converted to tosylate Type B. Thus, tosylate Type B was speculated as a hydrate.

TABLE 27G

XRPD peak list of tosylate Type C (824511-23-A)

| Pos. [°2θ] | Height [cts] | FWHM Left [°2θ] | d-spacing [Å] | Rel. Int. [%] |
|---|---|---|---|---|
| 7.3859 | 1079.28 | 0.1023 | 11.97 | 78.76 |
| 8.1737 | 1033.29 | 0.1023 | 10.82 | 75.40 |
| 9.8558 | 184.97 | 0.0768 | 8.97 | 13.50 |
| 11.2538 | 194.66 | 0.1535 | 7.86 | 14.21 |
| 12.9508 | 75.12 | 0.1535 | 6.84 | 5.48 |
| 13.8207 | 850.32 | 0.1279 | 6.41 | 62.05 |
| 14.3647 | 581.49 | 0.0768 | 6.17 | 42.43 |
| 14.5772 | 1370.36 | 0.1023 | 6.08 | 100.00 |
| 14.9343 | 607.44 | 0.0768 | 5.93 | 44.33 |
| 15.3821 | 648.13 | 0.1279 | 5.76 | 47.30 |
| 16.3938 | 307.68 | 0.1535 | 5.41 | 22.45 |
| 17.7059 | 695.75 | 0.1279 | 5.01 | 50.77 |
| 18.1559 | 391.97 | 0.1279 | 4.89 | 28.60 |
| 19.1587 | 235.95 | 0.1023 | 4.63 | 17.22 |
| 19.2676 | 231.61 | 0.1023 | 4.61 | 16.90 |
| 19.7819 | 136.11 | 0.1279 | 4.49 | 9.93 |
| 20.1463 | 185.68 | 0.1279 | 4.41 | 13.55 |
| 20.6660 | 459.89 | 0.1535 | 4.30 | 33.56 |
| 21.2491 | 329.72 | 0.1279 | 4.18 | 24.06 |
| 21.7257 | 93.21 | 0.1791 | 4.09 | 6.80 |
| 22.0494 | 740.50 | 0.1279 | 4.03 | 54.04 |
| 22.6024 | 167.75 | 0.1535 | 3.93 | 12.24 |
| 22.8891 | 452.88 | 0.1535 | 3.89 | 33.05 |
| 24.3077 | 503.75 | 0.1535 | 3.66 | 36.76 |
| 25.7249 | 41.22 | 0.3070 | 3.46 | 3.01 |
| 26.8248 | 120.69 | 0.1023 | 3.32 | 8.81 |
| 27.2130 | 308.53 | 0.1023 | 3.28 | 22.51 |
| 28.2757 | 86.19 | 0.6140 | 3.16 | 6.29 |
| 30.2890 | 13.32 | 0.6140 | 2.95 | 0.97 |
| 35.5856 | 99.61 | 0.1023 | 2.52 | 7.27 |
| 37.1139 | 43.19 | 0.5117 | 2.42 | 3.15 |

TABLE 27H

HPLC results of tosylate Type C (824511-23-A)

| # | RRT | Area (%) | # | RRT | Area (%) |
|---|---|---|---|---|---|
| 1 | 0.73 | 0.05 | 5 | 0.98 | 0.06 |
| 2 | 0.90 | 0.07 | 6 | 1.00 | 99.33 |
| 3 | 0.93 | 0.09 | 7 | 1.07 | 0.16 |
| 4 | 0.96 | 0.24 | -- | -- | -- |

TABLE 271

XRPD peak list of tosylate Type D (824511-23-A-RE_N2_60min_30.0° C.)

| Pos. [°2θ] | Height [cts] | FWHM Left [°2θ] | d-spacing [Å] | Rel. Int. [%] |
|---|---|---|---|---|
| 6.9971 | 1459.40 | 0.1004 | 12.63 | 100.00 |
| 8.2791 | 173.97 | 0.0669 | 10.68 | 11.92 |
| 9.3038 | 773.29 | 0.1004 | 9.51 | 52.99 |
| 9.5935 | 233.11 | 0.1004 | 9.22 | 15.97 |
| 10.0866 | 104.33 | 0.2007 | 8.77 | 7.15 |
| 11.9658 | 202.13 | 0.1338 | 7.40 | 13.85 |
| 12.5219 | 730.67 | 0.1004 | 7.07 | 50.07 |
| 13.7374 | 224.69 | 0.1673 | 6.45 | 15.40 |
| 14.5331 | 604.50 | 0.0836 | 6.10 | 41.42 |
| 16.1172 | 668.93 | 0.1004 | 5.50 | 45.84 |
| 16.6536 | 893.21 | 0.1506 | 5.32 | 61.20 |
| 17.0632 | 495.19 | 0.1673 | 5.20 | 33.93 |
| 17.9862 | 356.24 | 0.1338 | 4.93 | 24.41 |
| 18.4147 | 511.28 | 0.1673 | 4.82 | 35.03 |
| 19.2992 | 273.21 | 0.1673 | 4.60 | 18.72 |
| 19.9076 | 297.00 | 0.2007 | 4.46 | 20.35 |
| 20.3050 | 319.36 | 0.2007 | 4.37 | 21.88 |
| 21.9044 | 404.01 | 0.1171 | 4.06 | 27.68 |
| 22.7485 | 188.41 | 0.3346 | 3.91 | 12.91 |
| 24.0668 | 195.89 | 0.1673 | 3.70 | 13.42 |
| 24.9065 | 198.66 | 0.1673 | 3.58 | 13.61 |
| 25.5856 | 107.06 | 0.2676 | 3.48 | 7.34 |

Tosylate Type B XPRD data is shown in TABLE 27J.

TABLE 27J

| Pos. [°2Th.] | Height [cts] | FWHM Left [°2Th.] | d-spacing [Å] | Rel. Int. [%] |
|---|---|---|---|---|
| 7.427835 | 4483.403000 | 0.076752 | 11.90182 | 100.00 |
| 7.643535 | 1210.674000 | 0.076752 | 11.56643 | 27.00 |
| 9.583773 | 936.350600 | 0.102336 | 9.22872 | 20.88 |
| 10.411510 | 36.859850 | 0.204672 | 8.49680 | 0.82 |
| 11.627540 | 1124.000000 | 0.102336 | 7.61078 | 25.07 |
| 13.134850 | 154.837700 | 0.076752 | 6.74059 | 3.45 |
| 14.441570 | 313.115700 | 0.179088 | 6.13349 | 6.98 |
| 14.862090 | 868.059400 | 0.127920 | 5.96087 | 19.36 |
| 15.285470 | 495.064100 | 0.127920 | 5.79671 | 11.04 |
| 16.236920 | 347.993700 | 0.127920 | 5.45911 | 7.76 |
| 17.546190 | 73.476000 | 0.076752 | 5.05460 | 1.64 |
| 17.971910 | 146.026400 | 0.076752 | 4.93582 | 3.26 |
| 18.535320 | 47.922500 | 0.153504 | 4.78704 | 1.07 |
| 19.068530 | 365.595900 | 0.127920 | 4.65437 | 8.15 |
| 19.920000 | 60.421550 | 0.204672 | 4.45730 | 1.35 |
| 20.279040 | 138.386400 | 0.076752 | 4.37919 | 3.09 |
| 20.694820 | 482.273600 | 0.102336 | 4.29213 | 10.76 |
| 21.493670 | 314.622300 | 0.102336 | 4.13438 | 7.02 |
| 22.503910 | 275.740400 | 0.102336 | 3.95101 | 6.15 |
| 23.016230 | 257.202700 | 0.076752 | 3.86422 | 5.74 |
| 23.347380 | 318.374300 | 0.102336 | 3.81015 | 7.10 |
| 24.084190 | 116.820000 | 0.076752 | 3.69523 | 2.61 |
| 24.625780 | 78.845310 | 0.153504 | 3.61518 | 1.76 |
| 24.999660 | 129.500500 | 0.127920 | 3.56195 | 2.89 |
| 25.551130 | 190.643300 | 0.102336 | 3.48631 | 4.25 |
| 26.429750 | 185.538900 | 0.102336 | 3.37237 | 4.14 |
| 28.389800 | 25.934600 | 0.511680 | 3.14384 | 0.58 |
| 29.919260 | 224.944700 | 0.153504 | 2.98653 | 5.02 |
| 30.349960 | 76.220570 | 0.153504 | 2.94512 | 1.70 |
| 32.401000 | 52.747550 | 0.153504 | 2.76322 | 1.18 |

### Solvates

### Tosylate Type E

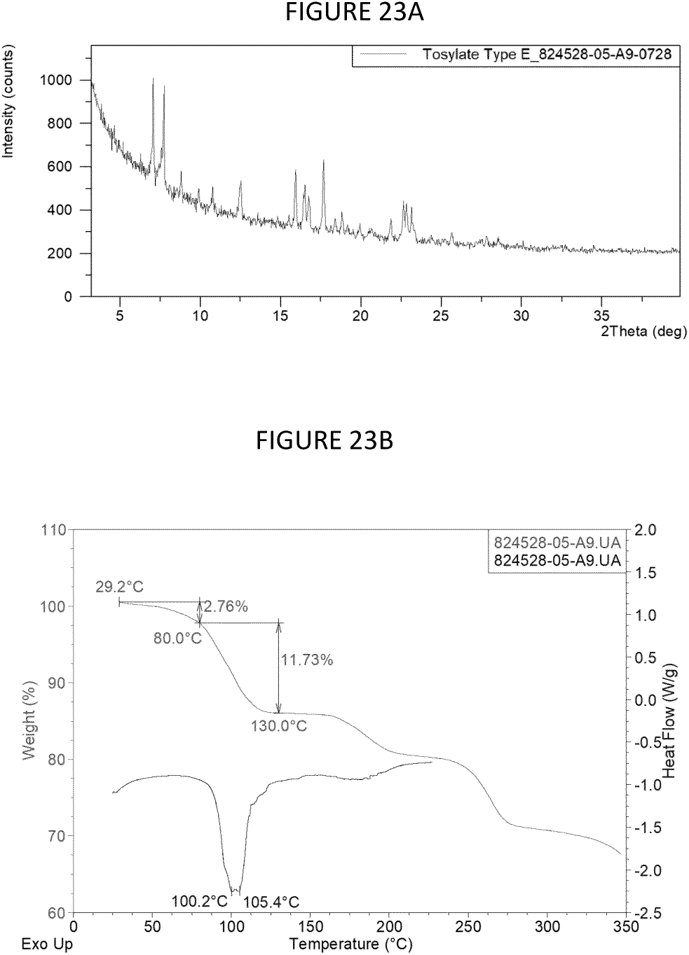

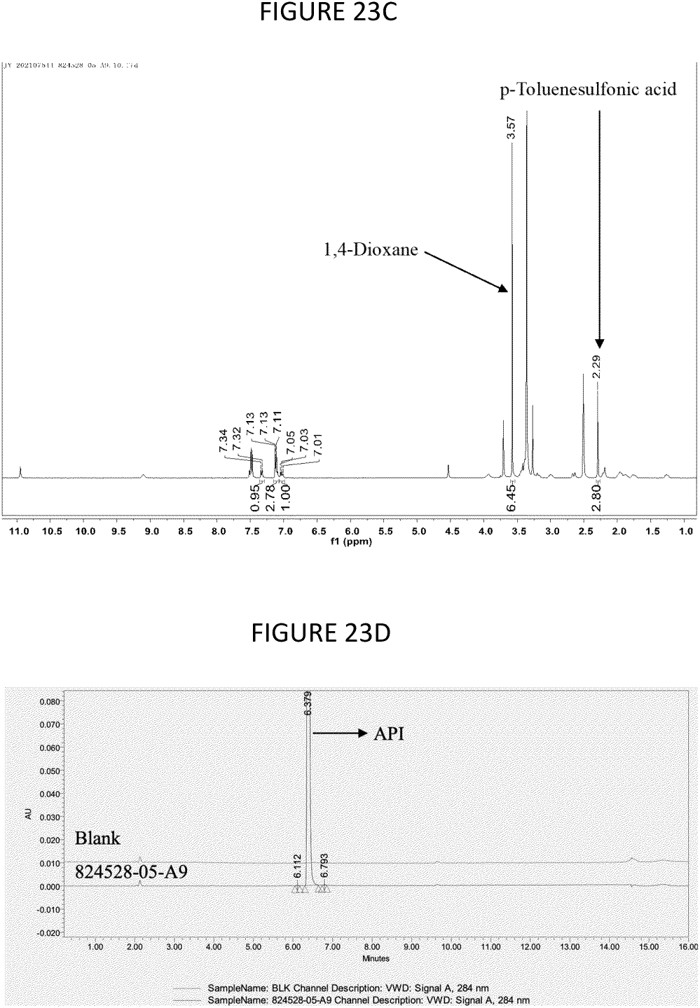

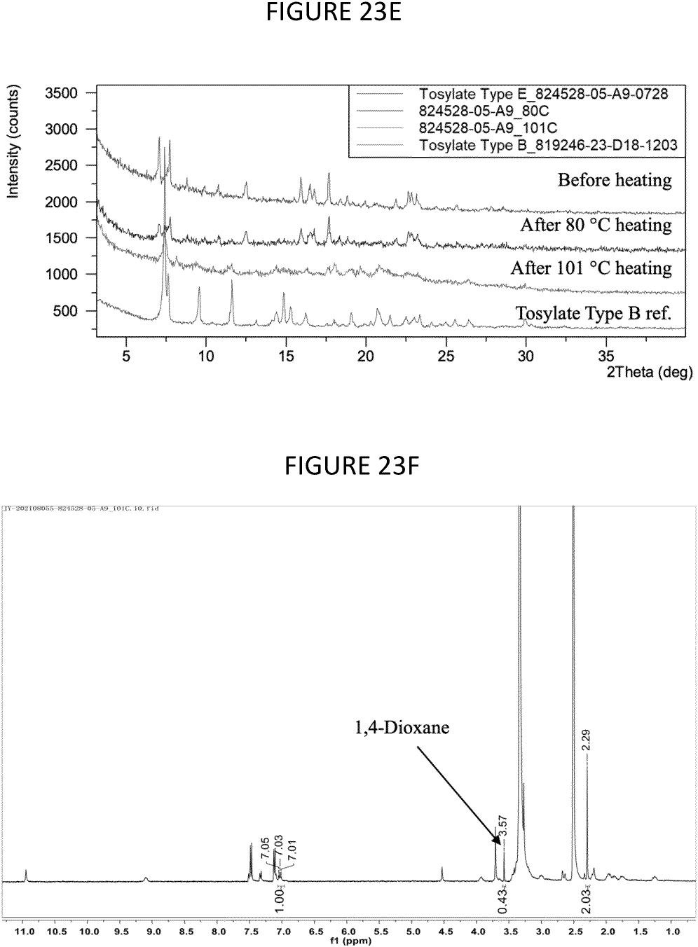

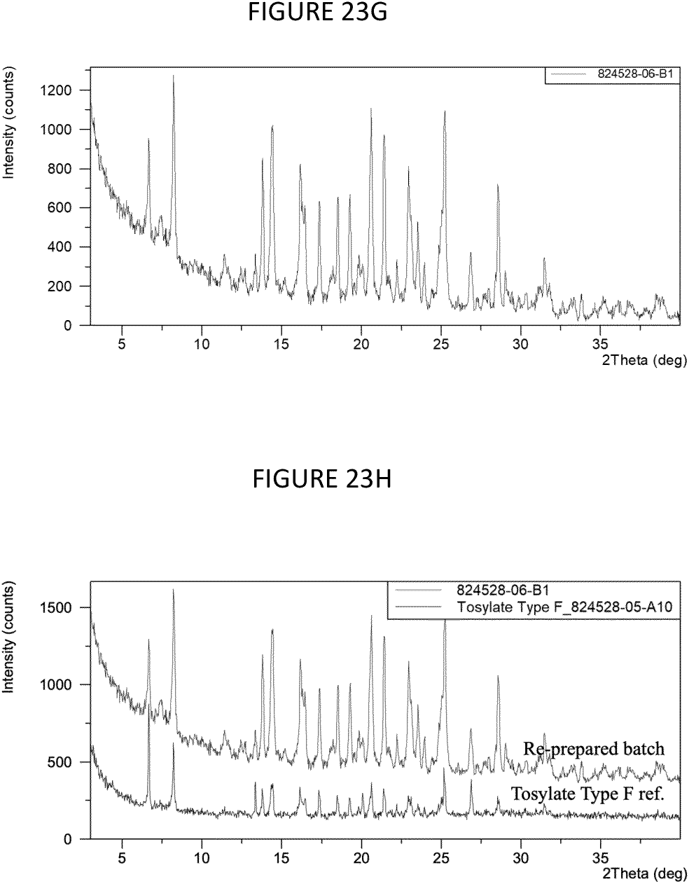

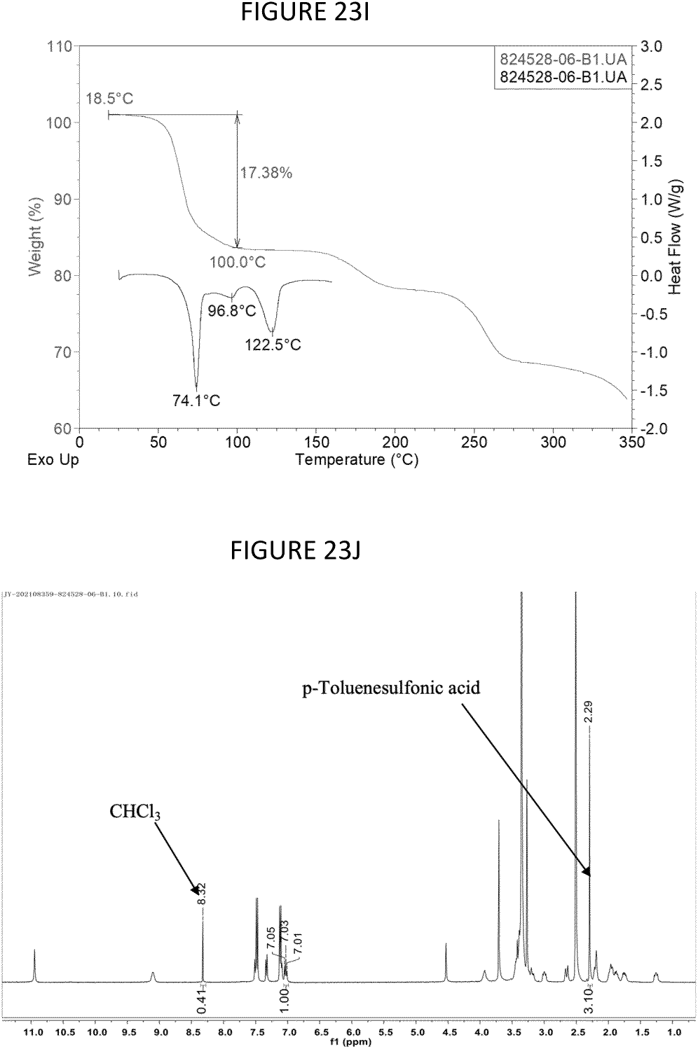

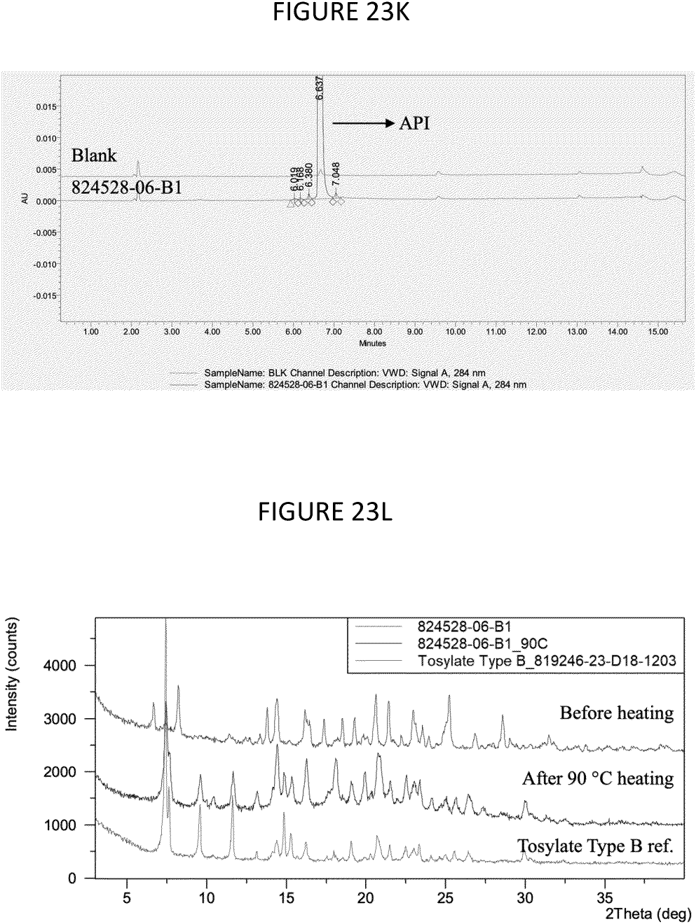

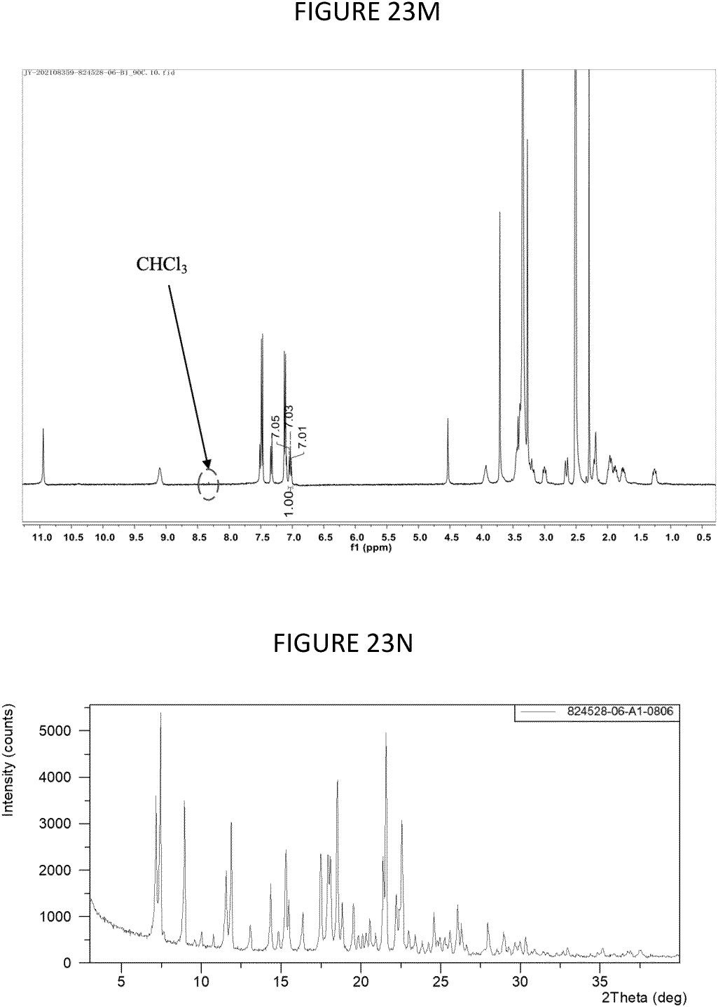

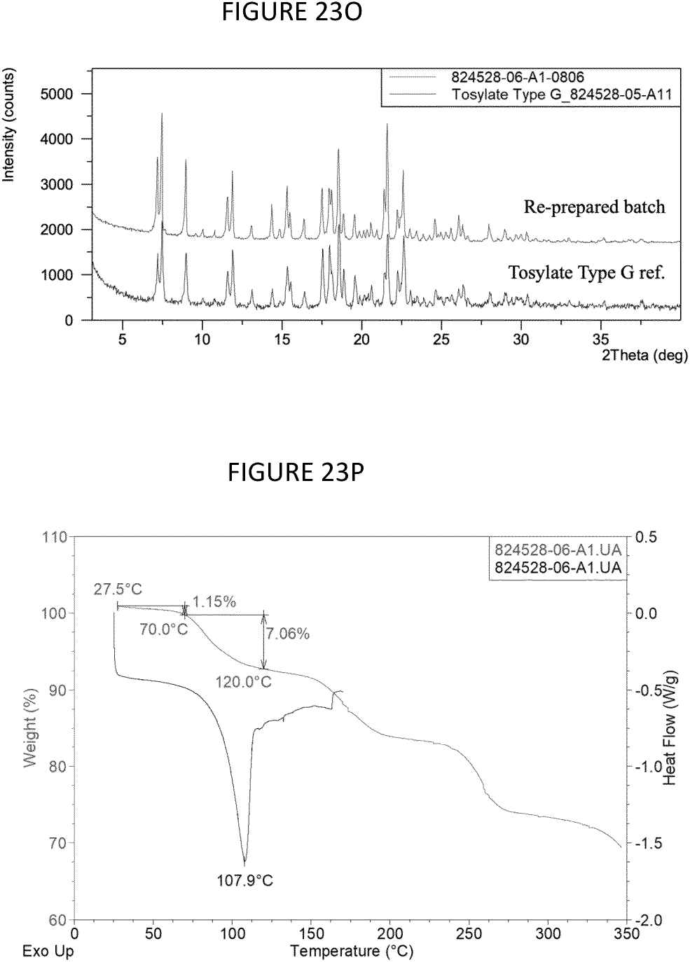

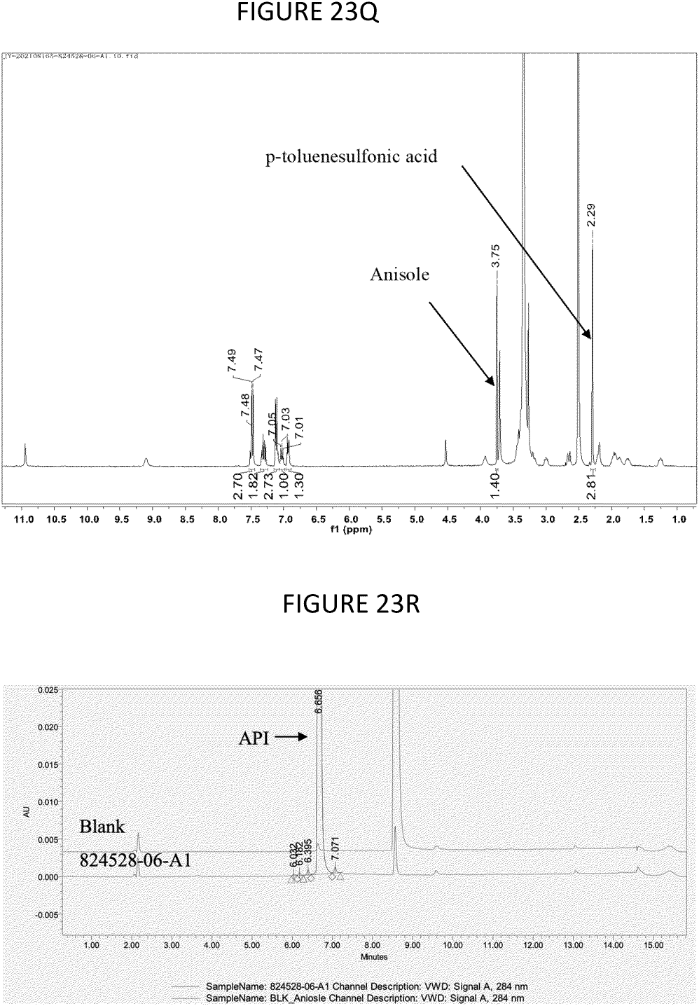

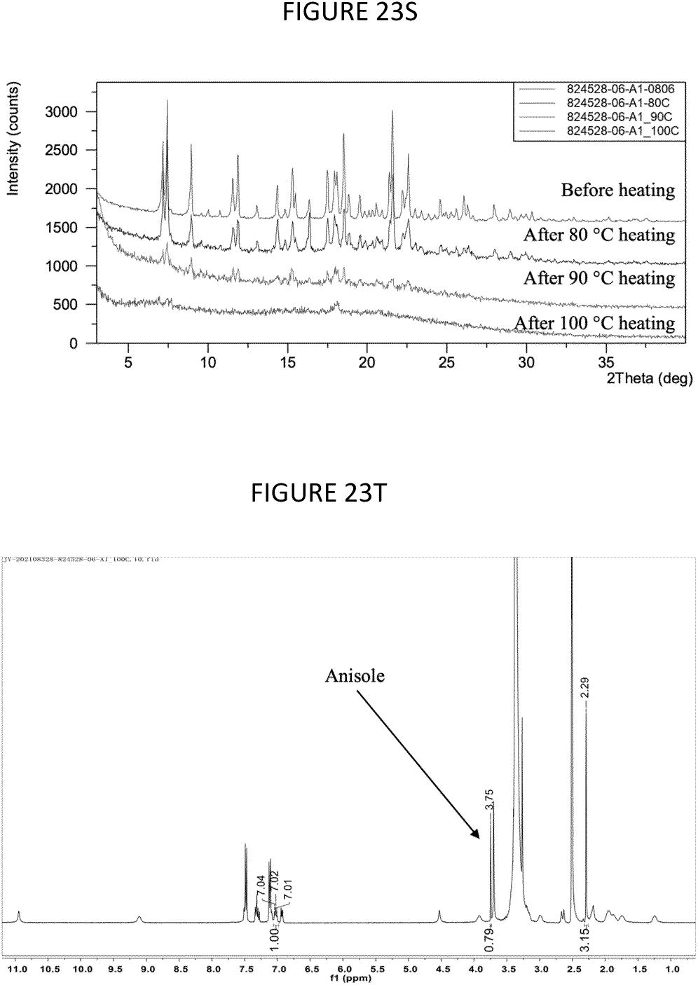

Tosylate Type E (824528-05-A9) was obtained by slurrying 19.9 mg tosylate Type B (824511-23-A-0618) in 0.5 mL 1,4-dioxane/n-heptane (9:1, v/v) at 50° C. for ~4 days. Resulting solids were isolated by centrifugation and air drying. The XRPD result is displayed in FIG. 23A and TABLE 28A. TGA/DSC results in FIG. 23B showed a weight loss of 2.8% up to 80° C., 11.7% from 80° C. up to 130° C., and two endothermic signals at 100.2° C. and 105.4° C. (peak). Using DMSO-d₆ as solvent, ¹H NMR result in FIG. 23C showed that the peaks of p-toluenesulfonic acid and 1,4-dioxane were observed. The molar ratio of p-toluenesulfonic acid/API was 0.9:1, the molar ratio of 1,4-dioxane/API was 0.8:1 (theoretical weight=11.84 wt%). HPLC purity of the sample was determined as 99.85 area% (FIG. 23D and TABLE 28B).

XRPD results in FIG. 23E showed that after heating tosylate Type E to 80° C., cooling back to RT under N2 (10° C./min heating and cooling rate) and re-exposing to ambient conditions for XRPD test, no form change was observed. After heating tosylate Type E to 101° C. and cooling back to RT, a sample with weak crystallinity and similar to tosylate Type B was obtained. ¹H NMR result in FIG. 23F showed that the amount of 1,4-dioxane decreased obviously (molar ratio of 1,4-Dioxane/API was 0.05:1, theoretical weight=0.88 wt%). Combined with the results of heating experiments, tosylate Type E was speculated as a 1,4-dioxane solvate.

TABLE 28A

XRPD peak list of tosylate Type E (824528-05-A9)

| Pos. [°2θ] | Height [cts] | FWHM Left [°2θ] | d-spacing [Å] | Rel. Int. [%] |
|---|---|---|---|---|
| 7.0641 | 565.35 | 0.0768 | 12.51 | 100.00 |
| 7.7278 | 508.69 | 0.0768 | 11.44 | 89.98 |
| 8.8122 | 139.67 | 0.0768 | 10.03 | 24.70 |
| 9.9012 | 73.42 | 0.0768 | 8.93 | 12.99 |
| 10.7435 | 110.24 | 0.2047 | 8.23 | 19.50 |
| 12.4957 | 188.07 | 0.1535 | 7.08 | 33.27 |
| 15.9333 | 335.37 | 0.1023 | 5.56 | 59.32 |
| 16.4934 | 235.77 | 0.1535 | 5.37 | 41.70 |
| 16.7668 | 171.43 | 0.1023 | 5.29 | 30.32 |
| 17.6748 | 424.04 | 0.1023 | 5.02 | 75.00 |
| 18.3896 | 74.28 | 0.1279 | 4.82 | 13.14 |
| 18.8260 | 121.32 | 0.1023 | 4.71 | 21.46 |
| 19.9372 | 68.37 | 0.0768 | 4.45 | 12.09 |
| 20.6139 | 31.21 | 0.2558 | 4.31 | 5.52 |
| 21.8675 | 114.68 | 0.1023 | 4.06 | 20.29 |
| 22.6526 | 224.06 | 0.1023 | 3.93 | 39.63 |
| 22.8435 | 197.49 | 0.1023 | 3.89 | 34.93 |
| 23.1606 | 183.82 | 0.0768 | 3.84 | 32.52 |
| 24.3574 | 24.50 | 0.3070 | 3.65 | 4.33 |
| 25.6476 | 66.87 | 0.1279 | 3.47 | 11.83 |
| 27.8366 | 52.00 | 0.1535 | 3.21 | 9.20 |
| 28.5704 | 45.97 | 0.1535 | 3.12 | 8.13 |

TABLE 28B

HPLC results of tosylate Type E (824528-05-A9)

| # | RRT | Area (%) |
|---|---|---|
| 1 | 0.96 | 0.07 |
| 2 | 1.00 | 99.85 |
| 3 | 1.06 | 0.09 |

### Tosylate Type F

Tosylate Type F (824528-06-B1) was prepared by slurrying 40.3 mg tosylate Type B (824511-23-A-0618) in 0.5 mL CHCl₃/n-hexane (1:1, v/v) at 50° C. for ~6 days. Resulting solids were isolated by centrifugation and air drying. The XRPD results are displayed in FIG. 23G, FIG. 23H and TABLE 28C. TGA/DSC results in FIG. 23I showed a weight loss of 17.4% up to 100° C., three endothermic signals at 74.1° C., 96.8° C. and 122.5° C. (peak). Using DMSO-d₆ as solvent, ¹H NMR result in FIG. 23J showed that the peak of p-toluenesulfonic acid and CHCl₃ were observed. The molar ratio of p-toluenesulfonic acid/API was 1:1, the molar ratio of CHCl₃/API was 0.41:1 (theoretical weight=8.22 wt%). HPLC purity of the sample was determined as 99.52 area% (FIG. 23K and TABLE 28C).

XRPD results in FIG. 23L showed that after heating tosylate Type F to 90° C., cooling back to RT under N2 (10° C./min heating and cooling rate) and re-exposing to ambient conditions for XRPD test, form change to tosylate Type B was observed. ¹H NMR result in FIG. 23M showed that no obvious peak of CHCl₃ was observed. Combined with the results of heating experiment, tosylate Type F was speculated as a CHCl₃ solvate.

TABLE 28C

XRPD peak list of tosylate Type F (824528-06-B1)

| Pos. [°2θ] | Height [cts] | FWHM Left [°2θ] | d-spacing [Å] | Rel. Int. [%] |
|---|---|---|---|---|
| 6.6797 | 458.34 | 0.1023 | 13.23 | 47.20 |
| 7.4136 | 150.42 | 0.1535 | 11.92 | 15.49 |
| 8.2308 | 898.66 | 0.1023 | 10.74 | 92.54 |
| 9.6387 | 31.67 | 0.6140 | 9.18 | 3.26 |
| 11.4165 | 143.84 | 0.1279 | 7.75 | 14.81 |
| 12.6894 | 76.42 | 0.1279 | 6.98 | 7.87 |
| 13.3588 | 172.53 | 0.1279 | 6.63 | 17.77 |
| 13.8073 | 649.99 | 0.1023 | 6.41 | 66.93 |
| 14.4115 | 822.86 | 0.2047 | 6.15 | 84.73 |
| 15.1895 | 69.10 | 0.1535 | 5.83 | 7.11 |
| 16.1769 | 659.25 | 0.1023 | 5.48 | 67.88 |
| 16.4597 | 447.23 | 0.1023 | 5.39 | 46.05 |
| 17.3697 | 487.09 | 0.1023 | 5.11 | 50.16 |
| 18.5280 | 516.74 | 0.1023 | 4.79 | 53.21 |
| 19.2847 | 530.21 | 0.1279 | 4.60 | 54.60 |
| 19.8413 | 207.75 | 0.1535 | 4.47 | 21.39 |
| 20.0932 | 173.16 | 0.0768 | 4.42 | 17.83 |
| 20.6185 | 944.94 | 0.2303 | 4.31 | 97.30 |
| 21.4296 | 857.17 | 0.1279 | 4.15 | 88.26 |
| 22.2331 | 190.58 | 0.1023 | 4.00 | 19.62 |
| 22.9657 | 690.38 | 0.1023 | 3.87 | 71.09 |
| 23.1489 | 407.48 | 0.0768 | 3.84 | 41.96 |
| 23.5529 | 413.45 | 0.1279 | 3.78 | 42.57 |
| 23.9520 | 204.54 | 0.1023 | 3.72 | 21.06 |
| 24.4316 | 83.59 | 0.1279 | 3.64 | 8.61 |
| 25.2207 | 971.15 | 0.1279 | 3.53 | 100.00 |
| 26.8584 | 283.10 | 0.2303 | 3.32 | 29.15 |
| 27.2544 | 54.00 | 0.1279 | 3.27 | 5.56 |
| 27.9846 | 104.07 | 0.1023 | 3.19 | 10.72 |
| 28.5855 | 617.07 | 0.1535 | 3.12 | 63.54 |
| 29.0418 | 199.04 | 0.1023 | 3.07 | 20.49 |
| 29.2200 | 77.73 | 0.2047 | 3.06 | 8.00 |
| 29.8838 | 63.61 | 0.1535 | 2.99 | 6.55 |
| 30.3219 | 91.55 | 0.1535 | 2.95 | 9.43 |
| 31.4812 | 280.84 | 0.1535 | 2.84 | 28.92 |
| 31.7976 | 133.24 | 0.1535 | 2.81 | 13.72 |
| 32.6368 | 54.10 | 0.1535 | 2.74 | 5.57 |
| 33.0314 | 35.84 | 0.4093 | 2.71 | 3.69 |
| 33.3112 | 70.23 | 0.1535 | 2.69 | 7.23 |
| 33.7994 | 84.00 | 0.1791 | 2.65 | 8.65 |
| 35.1997 | 78.82 | 0.3582 | 2.55 | 8.12 |
| 36.1332 | 65.82 | 0.2558 | 2.49 | 6.78 |
| 36.8559 | 61.68 | 0.4093 | 2.44 | 6.35 |
| 38.5022 | 103.89 | 0.1535 | 2.34 | 10.70 |

TABLE 28D

HPLC results of tosylate Type F (824528-06-B1)

| # | RRT | Area (%) | # | RRT | Area (%) |
|---|---|---|---|---|---|
| 1 | 0.91 | 0.06 | 4 | 1.00 | 99.52 |
| 2 | 0.93 | 0.06 | 5 | 1.06 | 0.21 |
| 3 | 0.96 | 0.16 | | -- | -- |

### Tosylate Type G

Tosylate Type G (824528-06-A1) was obtained by slurrying 50.2 mg tosylate Type B (824511-23-A-0618) in 0.5 mL anisole at 50° C. for ~3 days. Resulting solids were isolated by centrifugation and air drying. The XRPD results are displayed in FIG. 23N, FIG. 23O and TABLE 28E. TGA/DSC results in FIG. 23P showed a weight loss of 1.2% up to 70° C., 7.1% from 70° C. up to 120° C. and one endotherm ic signal at 107.9° C. (peak). Using DMSO-d₆ as solvent, ¹H NMR result in FIG. 23Q showed that the peak of p-toluenesulfonic acid and anisole were observed. The molar ratio of p-toluenesulfonic acid/API was 0.9:1, the molar ratio of anisole/API was 0.47:1 (theoretical weight=8.70 wt%). HPLC purity of the sample was determined as 99.63 area% (FIG. 23R and TABLE 28F).

XRPD results in FIG. 23S showed that after heating tosylate Type G to 80° C., cooling back to RT under N2 (10° C./min heating and cooling rate) and re-exposing to ambient conditions for XRPD test, no form change was observed. After heating tosylate Type G to 90° C. and 100° C., crystallinity of the sample decreased significantly (90° C.), and amorphous was obtained (100° C.). ¹H NMR result in FIG. 23T showed that along with the temperature increased, the solvent content decreased when the crystallinity of the sample decreased. The molar ratio of anisole/API was decreased to 0.26:1 (theoretical weight=5.10 wt%) after 100° C. heating. Tosylate Type G was speculated as an anisole solvate.

TABLE 28E

XRPD peak list of tosylate Type G (824528-06-A1)

| Pos. [°2θ] | Height [cts] | FWHM Left [°2θ] | d-spacing [Å] | Rel. Int. [%] |
|---|---|---|---|---|
| 7.1617 | 2966.34 | 0.0768 | 12.34 | 64.37 |
| 7.4344 | 4515.20 | 0.0768 | 11.89 | 97.98 |
| 8.9450 | 2878.33 | 0.0768 | 9.89 | 62.46 |
| 9.5862 | 94.99 | 0.0768 | 9.23 | 2.06 |
| 10.0090 | 243.30 | 0.0768 | 8.84 | 5.28 |
| 10.7520 | 259.97 | 0.0768 | 8.23 | 5.64 |
| 11.5547 | 1686.62 | 0.0768 | 7.66 | 36.60 |
| 11.8722 | 2699.54 | 0.1023 | 7.45 | 58.58 |
| 13.0707 | 524.24 | 0.1023 | 6.77 | 11.38 |
| 14.3453 | 1387.73 | 0.1023 | 6.17 | 30.11 |
| 14.8347 | 394.53 | 0.1023 | 5.97 | 8.56 |
| 15.2935 | 2059.90 | 0.1023 | 5.79 | 44.70 |
| 15.4851 | 1048.36 | 0.0768 | 5.72 | 22.75 |
| 16.3571 | 811.74 | 0.0768 | 5.42 | 17.62 |
| 17.4912 | 2040.28 | 0.1023 | 5.07 | 44.28 |
| 17.9519 | 2053.09 | 0.1023 | 4.94 | 44.55 |
| 18.0905 | 2011.75 | 0.0768 | 4.90 | 43.66 |
| 18.5279 | 3702.85 | 0.1023 | 4.79 | 80.35 |
| 18.8402 | 1079.29 | 0.1023 | 4.71 | 23.42 |
| 19.5303 | 1010.53 | 0.1023 | 4.55 | 21.93 |
| 19.8317 | 339.79 | 0.1023 | 4.48 | 7.37 |
| 20.1132 | 354.15 | 0.0768 | 4.41 | 7.69 |
| 20.3271 | 388.81 | 0.1023 | 4.37 | 8.44 |
| 20.5672 | 680.77 | 0.1023 | 4.32 | 14.77 |
| 20.9271 | 394.55 | 0.1023 | 4.25 | 8.56 |
| 21.4006 | 2016.44 | 0.0768 | 4.15 | 43.76 |
| 21.5807 | 4608.19 | 0.0768 | 4.12 | 100.00 |
| 22.2190 | 1245.70 | 0.0768 | 4.00 | 27.03 |
| 22.5815 | 2563.93 | 0.1023 | 3.94 | 55.64 |
| 23.0015 | 460.43 | 0.1023 | 3.87 | 9.99 |
| 23.4096 | 382.21 | 0.1023 | 3.80 | 8.29 |
| 23.8446 | 272.43 | 0.0768 | 3.73 | 5.91 |
| 24.2363 | 224.37 | 0.0768 | 3.67 | 4.87 |
| 24.5820 | 815.52 | 0.1023 | 3.62 | 17.70 |
| 24.9523 | 345.40 | 0.1023 | 3.57 | 7.50 |
| 25.2511 | 328.75 | 0.1023 | 3.53 | 7.13 |
| 25.5875 | 509.14 | 0.1279 | 3.48 | 11.05 |
| 26.0697 | 1017.71 | 0.1023 | 3.42 | 22.08 |
| 26.3149 | 643.33 | 0.0768 | 3.39 | 13.96 |
| 26.6345 | 191.44 | 0.1023 | 3.35 | 4.15 |
| 27.9679 | 611.25 | 0.1023 | 3.19 | 13.26 |
| 28.5305 | 110.32 | 0.1023 | 3.13 | 2.39 |
| 28.9774 | 414.78 | 0.1279 | 3.08 | 9.00 |
| 29.2895 | 158.43 | 0.1279 | 3.05 | 3.44 |
| 29.6658 | 277.83 | 0.1023 | 3.01 | 6.03 |
| 29.9745 | 275.59 | 0.1535 | 2.98 | 5.98 |
| 30.3368 | 375.93 | 0.1023 | 2.95 | 8.16 |
| 30.8934 | 138.58 | 0.0768 | 2.89 | 3.01 |
| 31.4315 | 68.40 | 0.1279 | 2.85 | 1.48 |
| 31.7050 | 47.14 | 0.1535 | 2.82 | 1.02 |
| 32.2702 | 44.59 | 0.1279 | 2.77 | 0.97 |
| 32.6728 | 88.64 | 0.1023 | 2.74 | 1.92 |
| 32.9611 | 166.09 | 0.1023 | 2.72 | 3.60 |
| 33.5415 | 54.15 | 0.0768 | 2.67 | 1.18 |
| 34.4121 | 33.71 | 0.1535 | 2.61 | 0.73 |
| 34.8429 | 83.15 | 0.1023 | 2.57 | 1.80 |
| 35.1522 | 175.17 | 0.1023 | 2.55 | 3.80 |
| 35.8658 | 57.11 | 0.2558 | 2.50 | 1.24 |
| 36.7202 | 121.16 | 0.0768 | 2.45 | 2.63 |
| 37.5161 | 135.41 | 0.2558 | 2.40 | 2.94 |
| 38.2025 | 26.13 | 0.1535 | 2.36 | 0.57 |

TABLE 28F

HPLC results of tosylate Type G (824528-06-A1)

| # | RRT | Area (%) | # | RRT | Area (%) |
|---|---|---|---|---|---|
| 1 | 0.91 | 0.05 | 4 | 1.00 | 99.63 |
| 2 | 0.93 | 0.07 | 5 | 1.06 | 0.14 |
| 3 | 0.96 | 0.12 | -- | -- | -- |

### Metastable Form

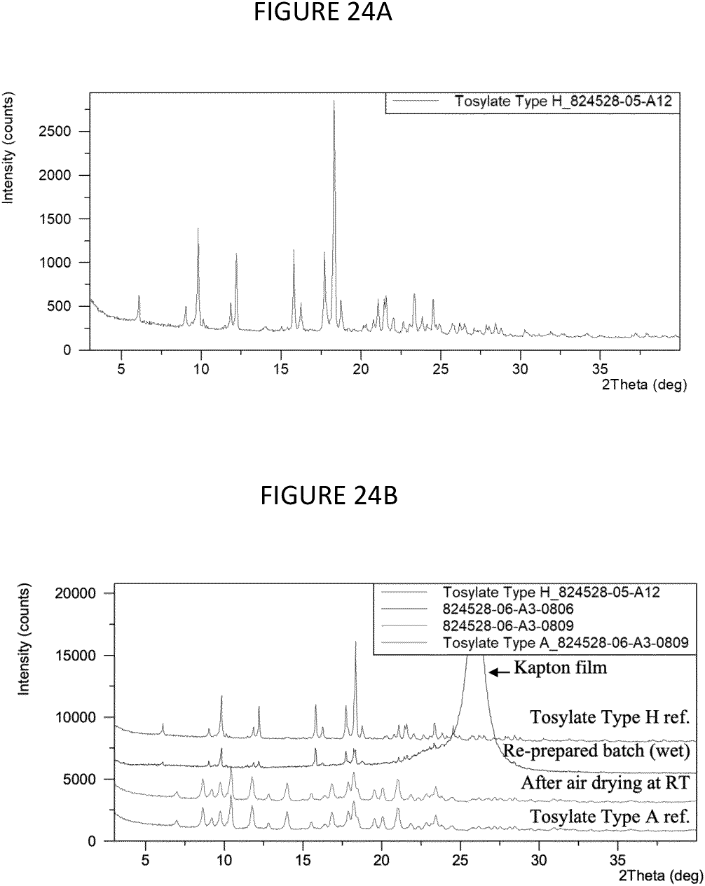

Tosylate Type H (824528-05-A12) was obtained by slurrying 23.5 mg tosylate Type B (824511-23-A-0618) in 0.5 mL IPA/EtOAc (1:4, v/v) at 50° C. for ~4 days. Resulting solids were isolated by centrifugation and air drying. The XRPD result is displayed in FIG. 24A and TABLE 29. The XRPD overlay in FIG. 24B showed that after air drying of tosylate Type H (824528-06-A3) for ~3 hours, a form change to tosylate Type A was observed, which indicated that tosylate Type H was a metastable form and no more characterization data were collected.

TABLE 29

XRPD peak list of tosylate Type H (824528-05-A12)

| Pos. [°2θ] | Height [cts] | FWHM Left [°2θ] | d-spacing [Å] | Rel. Int. [%] |
|---|---|---|---|---|
| 6.0878 | 879.91 | 0.0768 | 14.52 | 11.41 |
| 9.0212 | 668.03 | 0.0768 | 9.80 | 8.66 |
| 9.8009 | 3216.76 | 0.1023 | 9.02 | 41.72 |
| 10.1280 | 285.06 | 0.0768 | 8.73 | 3.70 |
| 11.8452 | 893.27 | 0.0768 | 7.47 | 11.58 |
| 12.1947 | 2445.31 | 0.0768 | 7.26 | 31.71 |
| 14.0186 | 130.51 | 0.2047 | 6.32 | 1.69 |
| 15.0239 | 147.68 | 0.1023 | 5.90 | 1.92 |
| 15.7958 | 2730.25 | 0.0768 | 5.61 | 35.41 |
| 16.2396 | 1008.67 | 0.0768 | 5.46 | 13.08 |
| 17.7214 | 2542.67 | 0.0768 | 5.01 | 32.98 |
| 18.3315 | 7710.80 | 0.1023 | 4.84 | 100.00 |
| 18.7501 | 1112.31 | 0.1023 | 4.73 | 14.43 |
| 19.5632 | 116.17 | 0.1535 | 4.54 | 1.51 |
| 20.2616 | 222.66 | 0.2558 | 4.38 | 2.89 |
| 20.7793 | 443.13 | 0.1023 | 4.27 | 5.75 |
| 21.0726 | 1125.15 | 0.0768 | 4.22 | 14.59 |
| 21.4460 | 1146.04 | 0.0512 | 4.14 | 14.86 |
| 21.5788 | 1265.35 | 0.0768 | 4.12 | 16.41 |
| 22.0255 | 512.11 | 0.1279 | 4.04 | 6.64 |
| 22.6581 | 399.11 | 0.0768 | 3.92 | 5.18 |
| 23.0672 | 303.63 | 0.1023 | 3.86 | 3.94 |
| 23.3476 | 1363.77 | 0.1023 | 3.81 | 17.69 |
| 23.8391 | 608.80 | 0.1023 | 3.73 | 7.90 |
| 24.1308 | 340.54 | 0.0768 | 3.69 | 4.42 |
| 24.5310 | 1019.36 | 0.1023 | 3.63 | 13.22 |
| 24.9259 | 340.72 | 0.1023 | 3.57 | 4.42 |
| 25.7325 | 383.81 | 0.1023 | 3.46 | 4.98 |
| 26.1917 | 330.23 | 0.1279 | 3.40 | 4.28 |
| 26.4916 | 394.34 | 0.1279 | 3.36 | 5.11 |
| 27.1038 | 191.12 | 0.1023 | 3.29 | 2.48 |
| 27.4419 | 178.55 | 0.1023 | 3.25 | 2.32 |
| 27.8712 | 286.86 | 0.0768 | 3.20 | 3.72 |
| 28.0370 | 264.57 | 0.1023 | 3.18 | 3.43 |
| 28.4280 | 412.42 | 0.1023 | 3.14 | 5.35 |
| 28.7776 | 262.06 | 0.1279 | 3.10 | 3.40 |
| 29.3651 | 60.49 | 0.1279 | 3.04 | 0.78 |
| 30.2812 | 214.03 | 0.0768 | 2.95 | 2.78 |
| 31.9033 | 185.01 | 0.0768 | 2.81 | 2.40 |
| 32.6291 | 82.21 | 0.3070 | 2.74 | 1.07 |
| 34.1711 | 118.79 | 0.1535 | 2.62 | 1.54 |
| 35.0207 | 48.02 | 0.3070 | 2.56 | 0.62 |
| 37.1878 | 152.26 | 0.1023 | 2.42 | 1.97 |
| 37.8954 | 147.70 | 0.1023 | 2.37 | 1.92 |
| 38.9781 | 43.11 | 0.3070 | 2.31 | 0.56 |

### Conclusion

A brief polymorph screening of 18-MC tosylate was performed and a total of nine salt forms were obtained.

### Example 8 - Polymorph Screening of 18-MC Besylate

18-MC besylate Type A and Type B were obtained as described in Example 1. Polymorph screening of 18-MC besylate was performed to better understand polymorphism of the salt.

The besylate material was first prepared using 18-MC freebase and then used as starting material for polymorph screening. In the screen, different crystallization methods, including temperature cycling and slurry conversion at different temperatures, were used, and a total of 30 experiments were conducted. Solids from screening were isolated for X-ray powder diffraction (XRPD) testing. From the results of characterization and form identification, a total of three besylate forms were obtained, including two anhydrates, besylate Types B and C, and one hydrate, besylate Type A. Characterization results are summarized in TABLE 30 and XRPD patterns of different forms are displayed in FIG. 25.

To summarize, a brief polymorph screen was performed and a total of three forms of besylate were discovered.

TABLE 30

Characterization summary of besylate forms

| | | | | | | |
|---|---|---|---|---|---|---|
| Solid form (ID) | TGA loss (%) | Endotherm (°C., peak) | Solvent residual (wt%)^{#} | Molar ratio^{#} (acid/FB) | HPLC purity (area%) | Speculated form |
| Besylate Type A (824511-35-A1) | 4.4% (up to 130° C.) | 117.4, 131.8 | Not detected | 1.0 | 99.71 | Hydrate |
| Besylate Type B (824511-44-C2) | 2.4% (up to 150° C.) | 181.1 | 2.5 (IPA) | 1.0 | 99.46 | Anhydrate |
| Besylate Type C (824529-04-A5_N2 Back_30° C.) | -- | -- | -- | -- | -- | Anhydrate |

FB: Freebase.
--: No data collected since besylate Type C converted to besylate Type A quickly after exposure to air.
^{#}: Calculation based on ¹H NMR result.

### Preparation of Besylate Starting Material

Preparation procedure of the besylate (824511-44-C2) was as follows: 1.0 g of freebase (824509-24-A) was weighed into a 20-mL glass vial along with 436.6 mg of benzenesulfonic acid. Then, 6 mL of IPA was added to the vial to produce a suspension which was slurried at RT for ~7 days. The resulting sample was centrifuged and vacuum dried at RT for 6 hours. As the results (FIGURE ) showed, about 1.15 g of besylate Type B (824511-44-C2) was obtained.

Approximate solubility of besylate Type B (824511-44-C2) was estimated in 38 solvents to guide the solvent selection in polymorph screening of besylate, with results shown in TABLE 31.

TABLE 31

Approximate solubility of besylate Type B (824511-44-C2) at RT and 50° C.

| RT (mg/mL) | | | | 50° C. (mg/mL) | |
|---|---|---|---|---|---|
| Solvent | Solubility | Solvent (v:v) | Solubility | Solvent (v:v) | Solubility |
| MeOH | S>44.0 | THF/H₂O (87:13, a_{W} 0.8) | S>42.0 | ACN/EtOAc (1:2) | 20.0<S<40.0 |
| DCM | S>40.0 | IPA/H₂O (847:153, a_{w} 0.8) | S>40.0 | IPA/EtOAc (1:2) | 7.0<S<21.0 |
| CHCl₃ | S>40.0 | THF/H₂O (981:19, a_{w} 0.2) | S>40.0 | IPAc/acetone (1:1) | 2.2<S<7.3 |
| ACN | S>40.0 | DCM/EtOAc (1:1) | 7.7<S<23.0 | n-Heptane /EtOH (1:1) | 2.2<S<7.3 |
| Acetone | 21.0<S<42.0 | EtOH/n-hexane (2:1) | 7.0<S<21.0 | 1,4-Dioxane/ n-heptane (9:1) | 2.1<S<7.0 |
| EtOH | 20.0<S<40.0 | ACN/toluene (1:4) | 6.3<S<19.0 | MTBE/CHCl₃ (1:1) | 2.0<S<6.7 |
| THF | 7.0<S<21.0 | MeOH/H₂O (2:1, a_{w} 0.6) | 2.0<S<6.7 | Anisole | 2.0<S<6.7 |
| MEK | 6.3<S<19.0 | 2-MeTHF/DCM (4:1) | 2.0<S<6.7 | Toluene/IPA (9:1) | 2.0<S<6.7 |
| IPA | 2.0<S<6.7 | IPA/H₂O (982:18, a_{w} 0.2) | 2.0<S<6.7 | CHCl₃/ n-hexane (1:1) | 1.9<S<6.3 |
| EtOAc | S<2.0 | CHCl₃/toluene (1:1) | 2.0<S<6.7 | MIBK | 1.9<S<6.3 |
| n-Heptane | S<2.0 | Acetone/ n-heptane (4:1) | 1.9<S<6.3 | n-Heptane | S<2.0 |
| H₂O | S<2.0 | ACN/H₂O (1:2, a_{w} 0.9) | S<1.9* | 2-MeTHF | S<1.9* |
| 2-MeTHF | S<1.9 | -- | -- | Toluene | S<1.9 |

*: Little solids in the vial.
Procedure: weigh ~2 mg solids into each 3-mL glass vial, add in corresponding solvent stepwise and sonicate or oscillate to see if solids dissolved completely. Stop adding solvent till the solids dissolves or total volume reaches 1.0 mL. Calculate the approximate solubility based on solvent volume.
Procedure: weigh ~2 mg solids into each 3-mL glass vial, add in corresponding solvent stepwise and sonicate or oscillate to see if solids dissolved completely. Stop adding solvent till the solids dissolves or total volume reaches 1.0 mL. Calculate the approximate solubility based on solvent volume.

### Polymorph Screening

Using besylate Type B (824511-44-C2) as the starting material, a total of 30 polymorph screening experiments were conducted via various crystallization methods. Results of polymorph screening is summarized in TABLE 32A. XRPD results showed that a total of 3 forms (besylate Types A, B, and C) were obtained from the screening and characterization studies, including two anhydrates (besylate Types B and C) and one hydrate (besylate Type A). Characterization data of obtained forms is summarized in TABLE 30 and the XRPD overlays of these forms are displayed in FIG. 25.

TABLE 32A

Summary of polymorph screening experiments of besylate

| Method | No. of Experiment | Results |
|---|---|---|
| Temperature cycling | 5 | Besylate Type A, Type B |
| Slurry at RT | 13 | Besylate Type A, Type B, Type A+B |
| Slurry at 50° C. | 12 | Besylate Type A, Type B, Type A+B+extra peak |
| Total | 30 | Besylate Type A, Type B, Type A+B, Type A+B+extra peak |

Note: Besylate Type C was discovered in form identification of besylate Type A.

### Besylate Type A and C

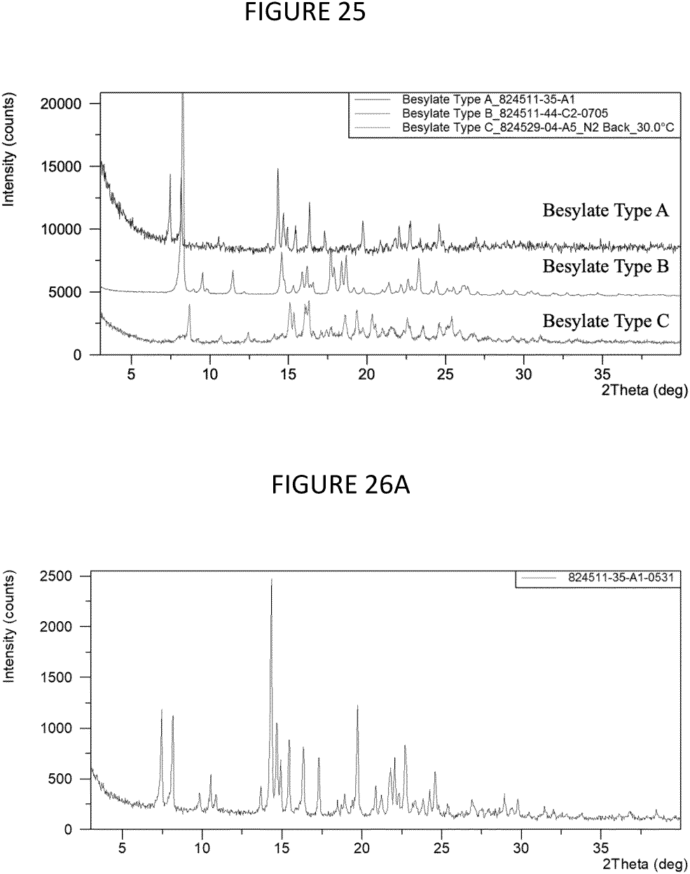

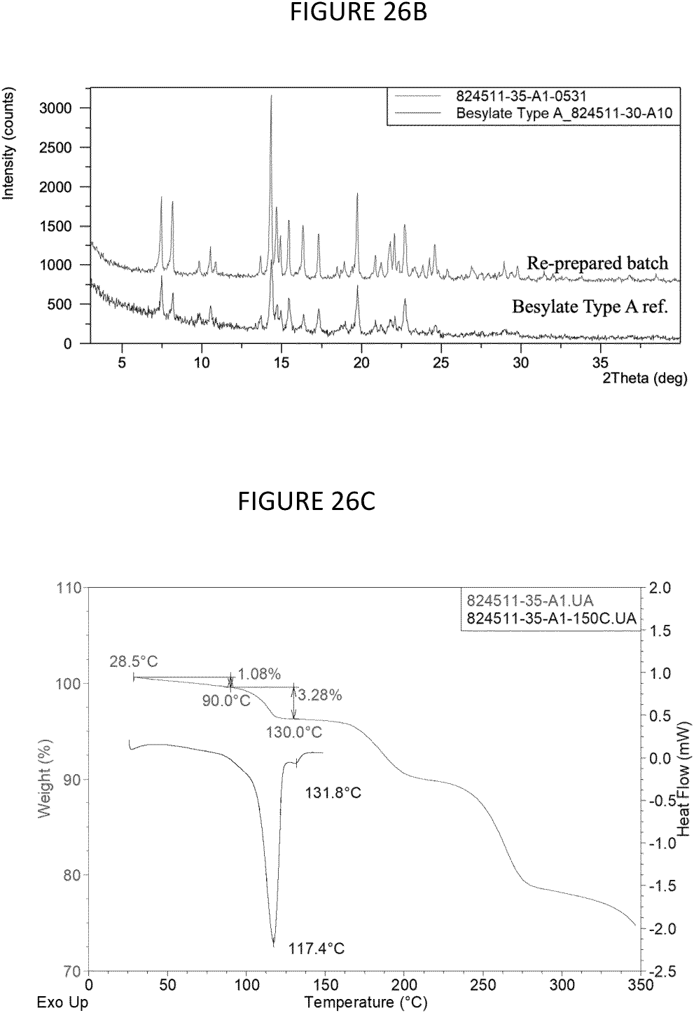

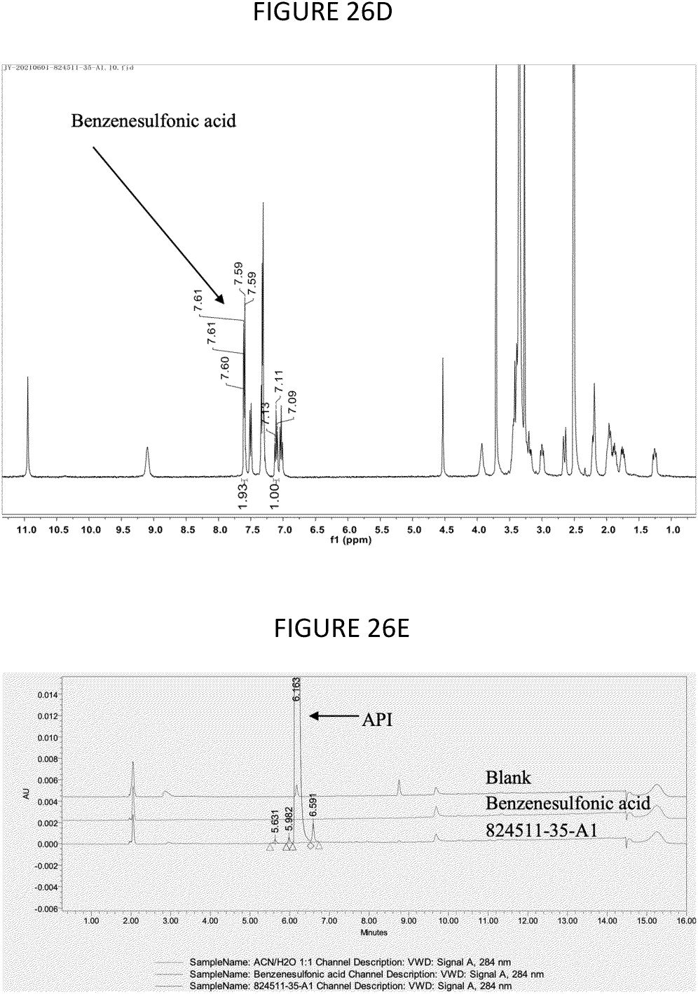

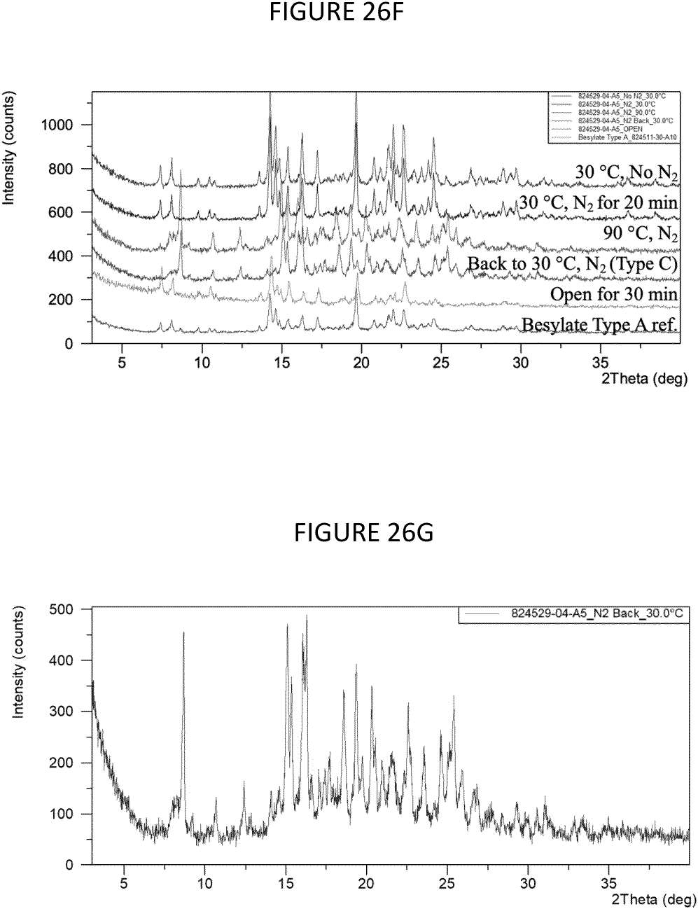

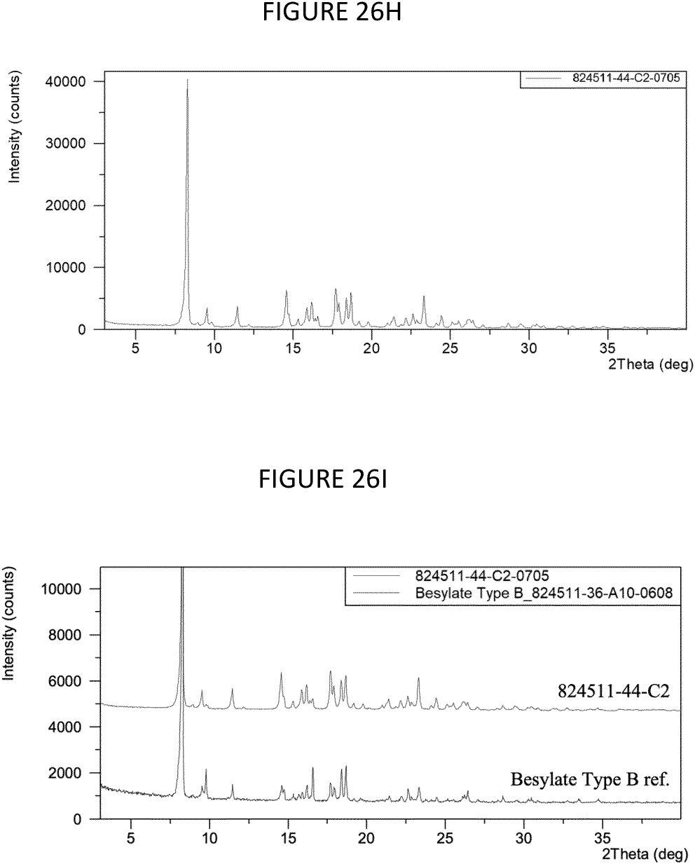

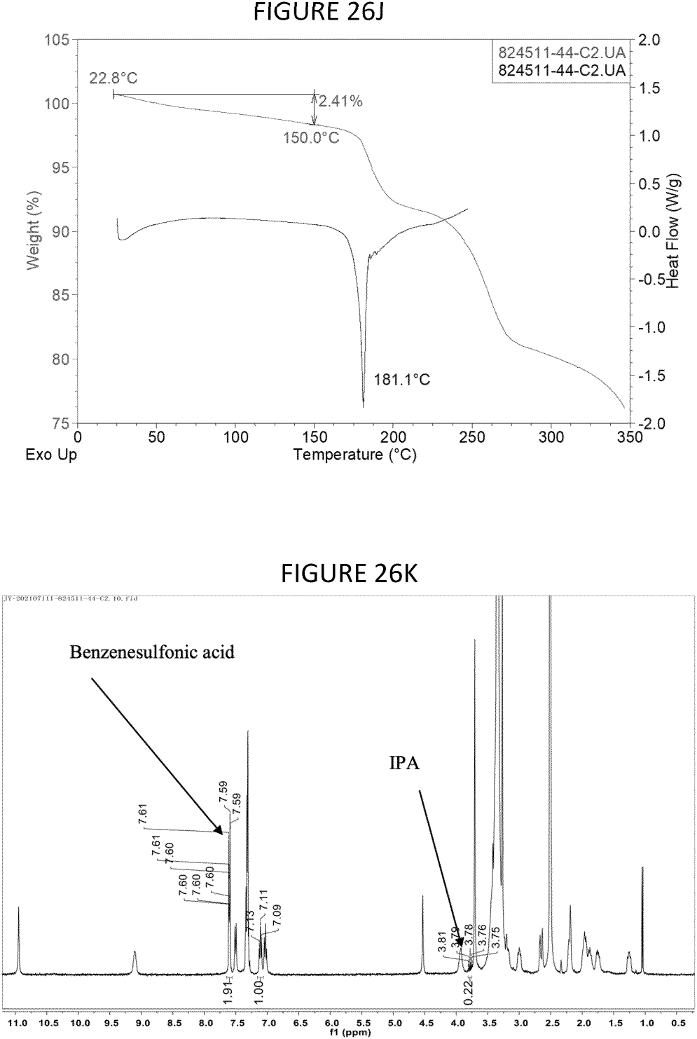

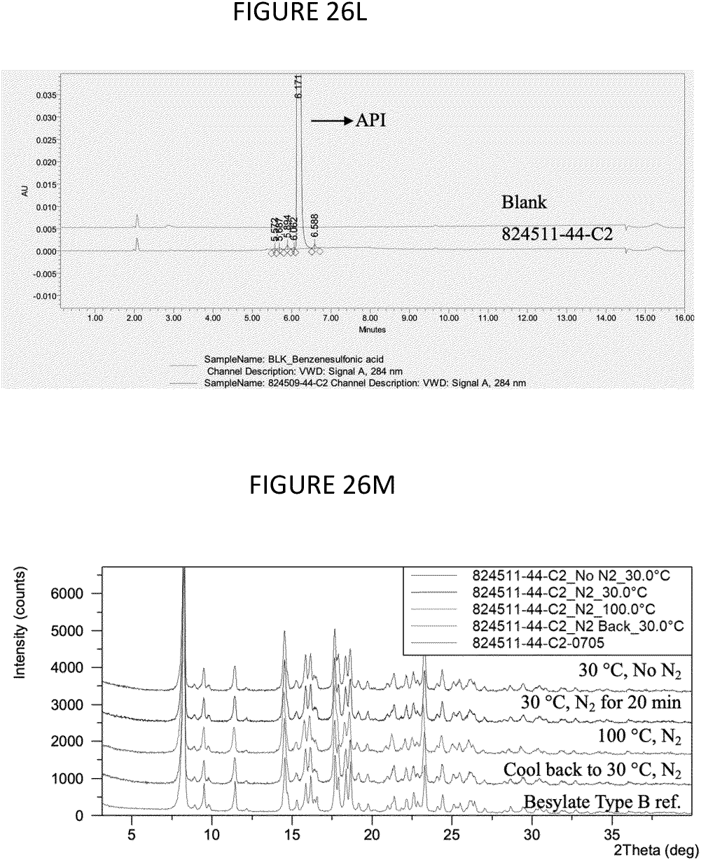

Besylate Type A (824511-35-A1) was obtained by slurrying 40.2 mg freebase and 17.4 mg benzenesulfonic acid (charge molar ratio of acid to freebase was 1:1) in 0.5 mL DCM/EtOAc (1:1, v/v) at RT for ~2 days. Resulting solids were isolated by centrifugation and vacuum drying at RT overnight. The XRPD results are displayed in FIG. 26A and FIG. 26B. Thermogravimetric analysis (TGA)/differential scanning calorimetry (DSC) curves in FIG. 26C showed a weight loss of 1.1% up to 90.0° C. and a weight loss of 3.3% from 90° C. to 130° C., and two endothermic DSC signals at 117.4° C. and 131.8° C. (peak). Using DMSO-d₆ as the solvent, proton nuclear magnetic resonance (¹H NMR) results in FIG. 26D showed that the peak of benzenesulfonic acid was observed. The molar ratio of acid/API was 1.0:1. No obvious solvent residual was detected. High performance liquid chromatography (HPLC) purity was 99.71 area% (FIG. 26E and TABLE 32B).

VT-XRPD test was performed using another batch of besylate Type A (824529-04-A5). As the results displayed in FIG. 26F, after heating besylate Type A to 100° C. and cooling back to 30° C. under N₂ protection, a new form was observed and assigned as besylate Type C (FIG. 26G and TABLE 32C). After exposure to ambient condition for ~30 min besylate Type C converted back to besylate Type A. Thus, besylate Type A was speculated as a hydrate (theoretical water content for monohydrate is 3.73%) and besylate Type C was speculated as an anhydrate.

TABLE 32B

HPLC results of besylate Type A (824511-35-A1)

| # | RRT | Area (%) |
|---|---|---|
| 1 | 0.91 | 0.05 |
| 2 | 0.97 | 0.05 |
| 3 | 1.00 | 99.71 |
| 4 | 1.07 | 0.18 |

TABLE 32C

XRPD peak list of besylate Type C (824529-04-A5_N2 Back_30.0° C.)

| Pos. [°2θ] | Height [cts] | FWHM Left [°2θ] | d-spacing [Å] | Rel. Int. [%] |
|---|---|---|---|---|
| 8.6804 | 396.21 | 0.0669 | 10.19 | 91.03 |
| 9.2192 | 33.41 | 0.1004 | 9.59 | 7.68 |
| 10.6935 | 76.15 | 0.0669 | 8.27 | 17.50 |
| 12.4194 | 105.07 | 0.0669 | 7.13 | 24.14 |
| 12.8308 | 35.15 | 0.1004 | 6.90 | 8.08 |
| 14.0886 | 78.16 | 0.1004 | 6.29 | 17.96 |
| 14.5891 | 88.02 | 0.1673 | 6.07 | 20.22 |
| 15.0973 | 411.44 | 0.1338 | 5.87 | 94.53 |
| 15.3499 | 305.57 | 0.0669 | 5.77 | 70.21 |
| 16.0594 | 354.30 | 0.0836 | 5.52 | 81.41 |
| 16.3001 | 435.23 | 0.0669 | 5.44 | 100.00 |
| 16.5731 | 104.61 | 0.1004 | 5.35 | 24.04 |
| 17.0829 | 102.57 | 0.1004 | 5.19 | 23.57 |
| 17.4106 | 127.76 | 0.1171 | 5.09 | 29.35 |
| 17.6999 | 145.72 | 0.1338 | 5.01 | 33.48 |
| 18.6018 | 273.17 | 0.1004 | 4.77 | 62.76 |
| 19.3536 | 326.01 | 0.1171 | 4.59 | 74.91 |
| 19.7368 | 154.63 | 0.1004 | 4.50 | 35.53 |
| 20.3333 | 279.02 | 0.1338 | 4.37 | 64.11 |
| 20.9852 | 141.19 | 0.1338 | 4.23 | 32.44 |
| 21.5465 | 148.13 | 0.2007 | 4.12 | 34.04 |
| 22.5610 | 246.35 | 0.1004 | 3.94 | 56.60 |
| 23.5628 | 162.05 | 0.1673 | 3.78 | 37.23 |
| 24.6117 | 182.21 | 0.2007 | 3.62 | 41.86 |
| 25.3971 | 265.99 | 0.1338 | 3.51 | 61.11 |
| 25.9192 | 130.32 | 0.1338 | 3.44 | 29.94 |
| 26.8482 | 93.21 | 0.1673 | 3.32 | 21.42 |
| 27.8124 | 42.58 | 0.2342 | 3.21 | 9.78 |
| 28.4145 | 40.45 | 0.2007 | 3.14 | 9.29 |
| 29.3021 | 69.25 | 0.1338 | 3.05 | 15.91 |
| 29.9029 | 31.61 | 0.2676 | 2.99 | 7.26 |
| 30.5608 | 45.44 | 0.1004 | 2.93 | 10.44 |
| 31.0514 | 61.01 | 0.1338 | 2.88 | 14.02 |
| 32.8352 | 24.02 | 0.2007 | 2.73 | 5.52 |
| 33.3407 | 25.63 | 0.3346 | 2.69 | 5.89 |
| 34.7376 | 12.24 | 0.2342 | 2.58 | 2.81 |

### Besylate Type B

Besylate Type B (824511-44-C2) was prepared by slurrying 1.0 g freebase and 436.6 mg benzenesulfonic acid (charge molar ratio of acid to freebase was 1:1) in 6.0 mL IPA at RT for ~7 days. Resulting solids were isolated by centrifugation and vacuum drying at RT for about 6 hrs. The XRPD result of besylate Type B (824511-44-C2) is shown in FIG. 26H, FIGURE and TABLE 32D. TGA/DSC curves in FIG. 26J showed a weight loss of 2.4% up to 150.0° C. and one endothermic peak at 181.1° C. (peak). ¹H NMR result in FIG. 26K showed that the peak of benzenesulfonic acid and IPA were observed. The molar ratio of benzenesulfonic acid/API was 1:1, the molar ratio of IPA/API was 0.22:1 (theoretical weight=2.50 wt%). HPLC purity was 99.73 area% (FIG. 26L and TABLE 32E).

VT-XRPD results in FIG. 26M showed that after drying besylate Type B (824511-44-C2) under N2 for 20 min at 30° C., or heating sample to 100° C. and cooling back to 30° C. under N₂ protection, no obvious form change was observed, indicating besylate Type B was an anhydrate.

TABLE 32D

XRPD peak list of besylate Type B (824511-44-C2)

| Pos. [°2θ] | Height [cts] | FWHM Left [°2θ] | d-spacing [Å] | Rel. Int. [%] |
|---|---|---|---|---|
| 8.2609 | 39438.94 | 0.1023 | 10.70 | 100.00 |
| 8.9280 | 549.21 | 0.1023 | 9.91 | 1.39 |
| 9.5109 | 2814.69 | 0.1023 | 9.30 | 7.14 |
| 9.7888 | 632.58 | 0.1535 | 9.04 | 1.60 |
| 11.4451 | 3240.85 | 0.1023 | 7.73 | 8.22 |
| 12.1723 | 308.75 | 0.0768 | 7.27 | 0.78 |
| 14.5598 | 5771.81 | 0.1023 | 6.08 | 14.63 |
| 14.7491 | 1877.15 | 0.0768 | 6.01 | 4.76 |
| 15.3084 | 1269.84 | 0.1023 | 5.79 | 3.22 |
| 15.8637 | 3165.65 | 0.1023 | 5.59 | 8.03 |
| 16.1762 | 4035.48 | 0.1023 | 5.48 | 10.23 |
| 16.3994 | 1368.20 | 0.0512 | 5.41 | 3.47 |
| 16.5565 | 1678.17 | 0.0768 | 5.35 | 4.26 |
| 17.7039 | 6236.18 | 0.1023 | 5.01 | 15.81 |
| 17.9049 | 3817.50 | 0.0768 | 4.95 | 9.68 |
| 18.3781 | 4667.88 | 0.1023 | 4.83 | 11.84 |
| 18.6653 | 5567.47 | 0.1023 | 4.75 | 14.12 |
| 19.1725 | 1001.70 | 0.1023 | 4.63 | 2.54 |
| 19.7624 | 900.40 | 0.1023 | 4.49 | 2.28 |
| 20.1567 | 207.40 | 0.1535 | 4.41 | 0.53 |
| 20.9871 | 731.99 | 0.1023 | 4.23 | 1.86 |
| 21.3935 | 1774.69 | 0.1023 | 4.15 | 4.50 |
| 21.8595 | 441.53 | 0.0768 | 4.07 | 1.12 |
| 22.1622 | 1450.81 | 0.1279 | 4.01 | 3.68 |
| 22.6129 | 2186.96 | 0.1279 | 3.93 | 5.55 |
| 22.8572 | 1192.75 | 0.0768 | 3.89 | 3.02 |
| 23.3040 | 5133.02 | 0.1279 | 3.82 | 13.02 |
| 24.0997 | 687.64 | 0.1023 | 3.69 | 1.74 |
| 24.4227 | 1919.39 | 0.1279 | 3.64 | 4.87 |
| 25.1206 | 913.09 | 0.1023 | 3.55 | 2.32 |
| 25.5163 | 1056.34 | 0.1023 | 3.49 | 2.68 |
| 26.0843 | 1309.38 | 0.0936 | 3.41 | 3.32 |
| 26.1963 | 1297.72 | 0.0768 | 3.40 | 3.29 |
| 26.4130 | 1248.83 | 0.1023 | 3.37 | 3.17 |
| 27.0476 | 475.36 | 0.1535 | 3.30 | 1.21 |
| 27.4971 | 62.76 | 0.1535 | 3.24 | 0.16 |
| 28.3069 | 264.38 | 0.1023 | 3.15 | 0.67 |
| 28.6580 | 747.74 | 0.1535 | 3.12 | 1.90 |
| 29.4349 | 656.11 | 0.1535 | 3.03 | 1.66 |
| 30.2564 | 402.86 | 0.1023 | 2.95 | 1.02 |
| 30.4695 | 618.37 | 0.1279 | 2.93 | 1.57 |
| 30.8863 | 351.75 | 0.1279 | 2.90 | 0.89 |
| 31.8539 | 327.85 | 0.1279 | 2.81 | 0.83 |
| 32.0545 | 285.83 | 0.0768 | 2.79 | 0.72 |
| 32.7410 | 362.66 | 0.1791 | 2.74 | 0.92 |
| 33.4593 | 169.90 | 0.1023 | 2.68 | 0.43 |
| 34.2293 | 214.85 | 0.2047 | 2.62 | 0.54 |
| 34.6983 | 387.99 | 0.0768 | 2.59 | 0.98 |
| 36.0609 | 264.30 | 0.0768 | 2.49 | 0.67 |
| 36.6796 | 124.71 | 0.1535 | 2.45 | 0.32 |
| 37.1312 | 193.63 | 0.1279 | 2.42 | 0.49 |
| 38.2610 | 175.10 | 0.0768 | 2.35 | 0.44 |
| 38.8348 | 122.32 | 0.1535 | 2.32 | 0.31 |
| 39.5360 | 70.81 | 0.1535 | 2.28 | 0.18 |

### Conclusion

A brief polymorph screen of 18-MC besylate was performed and a total of three polymorphs were obtained.

### Example 9

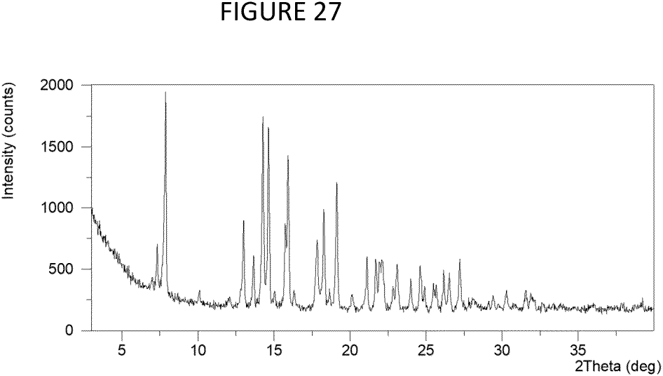

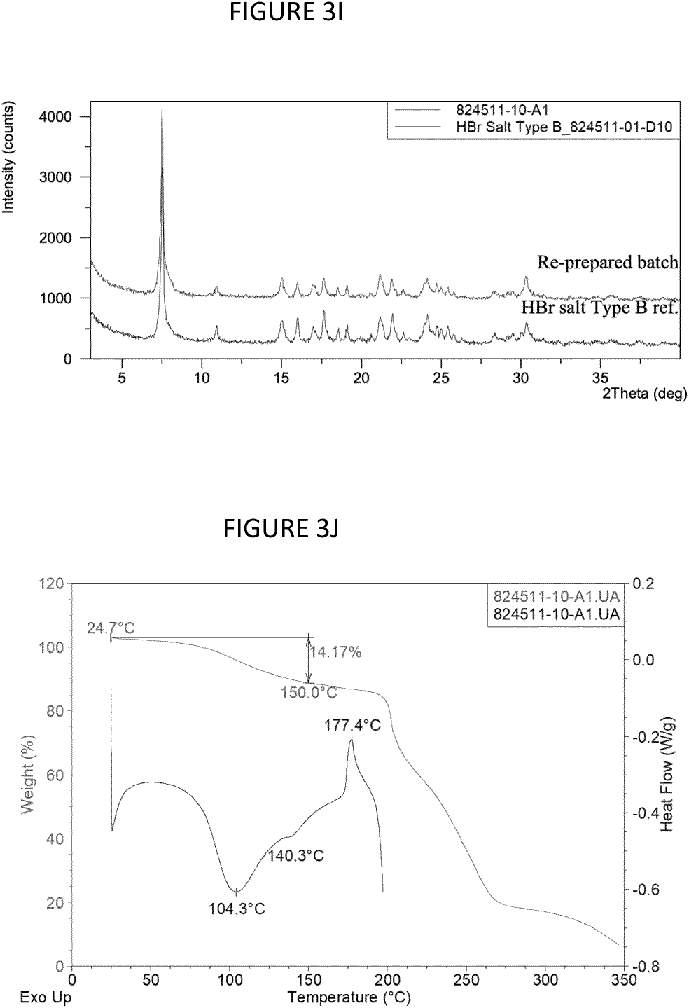

FIG. 27 shows XPRD data for maleate Type A. TABLE 33 shows XPRD peak data.

TABLE 33

| Pos. [°2Th.] | Height [cts] | FWHM Left [°2Th.] | d-spacing [Å] | Rel. Int. [%] |
|---|---|---|---|---|
| 7.327345 | 365.134000 | 0.102336 | 12.06482 | 22.50 |
| 7.876415 | 1623.091000 | 0.102336 | 11.22496 | 100.00 |
| 10.103920 | 95.921950 | 0.102336 | 8.75476 | 5.91 |
| 12.073950 | 56.701280 | 0.153504 | 7.33037 | 3.49 |
| 13.013730 | 695.625100 | 0.102336 | 6.80305 | 42.86 |
| 13.670060 | 411.313300 | 0.102336 | 6.47786 | 25.34 |
| 14.281320 | 1508.899000 | 0.102336 | 6.20195 | 92.96 |
| 14.659210 | 1438.587000 | 0.102336 | 6.04291 | 88.63 |
| 15.048350 | 128.700900 | 0.102336 | 5.88751 | 7.93 |
| 15.749820 | 668.126700 | 0.076752 | 5.62683 | 41.16 |
| 15.942700 | 1231.565000 | 0.076752 | 5.55919 | 75.88 |
| 16.341800 | 120.146300 | 0.127920 | 5.42431 | 7.40 |
| 17.851850 | 539.582900 | 0.127920 | 4.96874 | 33.24 |
| 18.292400 | 793.408400 | 0.102336 | 4.85006 | 48.88 |
| 18.677200 | 150.253900 | 0.076752 | 4.75099 | 9.26 |
| 19.139610 | 1015.371000 | 0.102336 | 4.63724 | 62.56 |
| 20.123950 | 102.424100 | 0.102336 | 4.41258 | 6.31 |
| 21.129300 | 420.379200 | 0.076752 | 4.20485 | 25.90 |
| 21.703030 | 398.425000 | 0.102336 | 4.09497 | 24.55 |
| 21.957830 | 369.128500 | 0.076752 | 4.04802 | 22.74 |
| 22.136070 | 383.434700 | 0.127920 | 4.01583 | 23.62 |
| 22.848160 | 165.009300 | 0.076752 | 3.89226 | 10.17 |
| 23.097360 | 338.352900 | 0.153504 | 3.85083 | 20.85 |
| 24.008200 | 233.990800 | 0.076752 | 3.70675 | 14.42 |
| 24.621070 | 345.082600 | 0.127920 | 3.61586 | 21.26 |
| 24.913620 | 171.992600 | 0.076752 | 3.57406 | 10.60 |
| 25.484310 | 184.601900 | 0.076752 | 3.49530 | 11.37 |
| 25.691250 | 182.730000 | 0.076752 | 3.46761 | 11.26 |
| 26.175050 | 276.701300 | 0.102336 | 3.40461 | 17.05 |
| 26.535790 | 285.330100 | 0.102336 | 3.35914 | 17.58 |
| 27.221300 | 383.892900 | 0.127920 | 3.27608 | 23.65 |
| 28.031390 | 53.299740 | 0.179088 | 3.18322 | 3.28 |
| 29.401780 | 101.635600 | 0.127920 | 3.03790 | 6.26 |
| 30.284150 | 134.196700 | 0.102336 | 2.95137 | 8.27 |
| 31.563120 | 145.138400 | 0.076752 | 2.83463 | 8.94 |
| 31.895260 | 124.251000 | 0.127920 | 2.80587 | 7.66 |
| 36.039850 | 34.649630 | 0.460512 | 2.49214 | 2.13 |
| 37.917300 | 36.694180 | 0.204672 | 2.37295 | 2.26 |
| 39.211670 | 52.464170 | 0.153504 | 2.29755 | 3.23 |

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

What is claimed is:

1. A composition comprising a salt of 18-MC, wherein the salt is chosen from gentisate, hydrobromide, besylate, napadisylate, hydrochloride, sulfate, oxalate, maleate, mesylate, and tosylate.

2. A composition comprising a polymorph of 18-MC, wherein the polymorph is chosen from HCI salt Type A, HCI salt Type B, HCI salt Type C, HCI salt Type D, HCI salt Type E, HCI salt Type F, HCI salt Type G, HCI salt Type H, HCI salt Type I, HCI salt Type J, HCI salt Type K, HCI salt Type L, HCI salt Type M, HCI salt Type N, HCI salt Type O, HCI salt Type P, HCI salt Type Q, HCI salt Type R, HCI salt Type S, HCI salt Type T, HCI salt Type U, HCI salt Type V, sulfate salt Type A, sulfate salt Type B, sulfate salt Type C, sulfate salt Type D, sulfate salt Type E, sulfate salt Type F, oxalate salt Type A, oxalate salt Type B, maleate salt Type A, mesylate salt Type A, mesylate salt Type B, mesylate salt Type C, HBr salt Type A, HBr salt Type B, HBr salt Type C, HBr salt Type D, tosylate salt Type A, tosylate salt Type B, tosylate salt Type C, tosylate salt Type D, tosylate salt Type E, tosylate salt Type F, tosylate salt Type G, tosylate salt Type H, tosylate salt Type I, besylate salt Type C, napadisylate salt Type A, napadisylate salt Type B, napadisylate salt Type C, napadisylate salt Type D, and gentisate salt Type A.

3. The composition of claim 2, wherein said polymorph is gentisate salt Type A and is characterized by an endothermic signal at 181.9° C.

4. The composition of claim 2, wherein said polymorph is HBr salt Type A and is characterized by an endothermic signal at 208.5° C.

5. The composition of claim 2, wherein said polymorph is HBr salt Type B and is characterized by endothermal signals at 104.3, 140.3, and 177.4° C.

6. The composition of claim 2, wherein said polymorph is besylate salt Type A and is characterized by endothermal signals at 117.4 and 131.8° C.

7. The composition of claim 2, wherein said polymorph is besylate salt Type B and is characterized by endothermal signals at 177.5 and 179.3° C.

8. The composition of claim 2, wherein said polymorph is napadisylate salt Type A and is characterized by endothermal signals at 96.6, 163.0, and 198.5° C.

9. The composition of claim 2, wherein said polymorph is napadisylate salt Type B and is characterized by endothermal signals at 81.7 and 206.0° C.

10. The composition of claim 2, wherein said polymorph is napadisylate salt Type C and is characterized by endothermal signals at 71.2, 117.0, and 191.0° C.

11. The composition of claim 2, wherein said polymorph is napadisylate salt Type D and is characterized by endothermal signals at 53.9, 89.0, and 178.3° C.

12. The composition of claim 2, wherein said polymorph is HCI salt Type A and is characterized by an endothermal signal at 211.6° C.

13. The composition of claim 2, wherein said polymorph is sulfate salt Type A and is characterized by endothermal signals at 150.4 and 185.5° C.

14. The composition of claim 2, wherein said polymorph is maleate salt Type A and is characterized by an endothermal signal at 180.4° C.

15. The composition of claim 2, wherein said polymorph is tosylate salt Type A and is characterized by endothermal signals at 72.9, 114.1, and 145.3° C.

16. The composition of claim 2, wherein said polymorph is tosylate salt Type B and is characterized by endothermal signals at 93.9, 119.0, and 183.5° C.

17. The composition of claim 2, wherein said polymorph is mesylate salt Type A and is characterized by endothermal signals at 76.0 and 161.1° C.

18. The composition of claim 2, wherein said polymorph is oxalate salt Type A and is characterized by an endothermal signal at 170.6° C.

19. The composition of claim 2, wherein said polymorph is oxalate salt Type B and is characterized by an endothermal signal at 167.1° C.

20. The composition of claim 2, wherein said polymorph is crystalline gentisate salt Type A and is characterized by an x-ray powder diffraction pattern having peaks expressed as 20 at about 10.3, about 11.1, about 16.3, about 20.6, about 21.0, and about 27.8.

21. The composition of claim 2, wherein said polymorph is crystalline HBr salt Type A and is characterized by an x-ray powder diffraction pattern having peaks expressed as 20 at about 8.6, about 13.1, about 19.1, about 19.9, about 26.1, and about 26.3.

22. The composition of claim 2, wherein said polymorph is crystalline HBr salt Type B and is characterized by an x-ray powder diffraction pattern having peaks expressed as 20 at about 7.5, about 15.0, about 21.2, about 21.9, about 24.1, and about 30.3.

23. The composition of claim 2, wherein said polymorph is crystalline besylate salt Type A and is characterized by an x-ray powder diffraction pattern having peaks expressed as 20 at about 7.4, about 8.1, about 14.3, about 14.7, about 19.7, and about 22.7.

24. The composition of claim 2, wherein said polymorph is crystalline besylate salt Type B and is characterized by an x-ray powder diffraction pattern having peaks expressed as 20 at about 8.3, about 9.8, about 16.6, about 17.7, about 18.4, and about 18.7.

25. The composition of claim 2, wherein said polymorph is crystalline napadisylate salt Type A and is characterized by an x-ray powder diffraction pattern having peaks expressed as 20 at about 7.6, about 8.1, about 12.2, about 12.7, about 14.6, and about 17.5.

26. The composition of claim 2, wherein said polymorph is crystalline napadisylate salt Type B and is characterized by an x-ray powder diffraction pattern having peaks expressed as 20 at about 8.2, about 10.6, about 17.8, about 19.3, about 20.0, and about 21.3.

27. The composition of claim 2, wherein said polymorph is crystalline napadisylate salt Type C and is characterized by an x-ray powder diffraction pattern having peaks expressed as 20 at about 7.3, about 9.6, about 15.2, about 18.4, about 19.1, and about 24.5.

28. The composition of claim 2, wherein said polymorph is crystalline napadisylate salt Type D and is characterized by an x-ray powder diffraction pattern having peaks expressed as 20 at about 7.3, about 7.5, about 15.0, about 17.8, about 18.0, and about 22.5.

29. The composition of claim 2, wherein said polymorph is crystalline HCI salt Type A and is characterized by an x-ray powder diffraction pattern having peaks expressed as 20 at about 8.8, about 11.0, about 13.4, about 16.2, about 16.5, and about 16.8.

30. The composition of claim 2, wherein said polymorph is crystalline sulfate salt Type A and is characterized by an x-ray powder diffraction pattern having peaks expressed as 20 at about 5.2, about 10.5, about 13.8, about 15.7, about 18.3, and about 20.4.

31. The composition of claim 2, wherein said polymorph is crystalline maleate salt Type A and is characterized by an x-ray powder diffraction pattern having peaks expressed as 20 at about 7.9, about 14.3, about 14.7, about 15.9, about 18.3, and about 19.1.

32. The composition of claim 2, wherein said polymorph is crystalline tosylate salt Type A and is characterized by an x-ray powder diffraction pattern having peaks expressed as 20 at about 8.6, about 10.4, about 11.8, about 17.9, about 18.2, and about 21.0.

33. The composition of claim 2, wherein said polymorph is crystalline tosylate salt Type B and is characterized by an x-ray powder diffraction pattern having peaks expressed as 20 at about 7.4, about 7.6, about 9.6, about 11.6, about 14.9, and about 15.3.

34. The composition of claim 2, wherein said polymorph is crystalline mesylate salt Type A and is characterized by an x-ray powder diffraction pattern having peaks expressed as 20 at about 8.1, about 9.2, about 13.0, about 16.9, about 18.2, and about 21.1.

35. The composition of claim 2, wherein said polymorph is crystalline oxalate salt Type A and is characterized by an x-ray powder diffraction pattern having peaks expressed as 20 at about 6.0, about 9.1, about 13.6, about 15.8, about 18.2, and about 21.8.

36. The composition of claim 2, wherein said polymorph is crystalline oxalate salt Type B and is characterized by an x-ray powder diffraction pattern having peaks expressed as 20 at about 7.7, about 11.8, about 13.7, about 16.7, about 17.7, and about 18.9.

37. The composition of claim 2, wherein said polymorph is a freebase and is characterized by an x-ray powder diffraction pattern having peaks expressed as 20 at about 11.0, about 11.7, about 14.0, about 15.5, about 18.3, and about 21.3.

38. The composition of claim 2, wherein said polymorph is crystalline.

39. The composition of claim 2, wherein said polymorph is amorphous.
